(19) United States
(12) Patent Application Publication
(10) Pub. No.: US 2025/0163130 A1
(43) Pub. Date: May 22, 2025

(54) LIGAND GATED ION CHANNELS AND METHODS OF USE

(71) Applicant: Trames Bio, Inc., Oakland, CA (US)

(72) Inventors: Anthony Lau, Redwood City, CA (US); Orion P. Keifer, Jr., South San Francisco, CA (US); Stefanie Makinson, Palo Alto, CA (US); Corey J. Cain, San Francisco, CA (US); Alexander Naka, Berkeley, CA (US)

(21) Appl. No.: 18/839,624

(22) PCT Filed: Feb. 21, 2023
(86) PCT No.: PCT/US2023/062943

§ 371 Date: Aug. 19, 2024

Related U.S. Application Data
(60) Provisional application No. 63/311,735, filed on Feb. 18, 2022.
(60) Provisional application No. 63/348,560, filed on Jun. 3, 2022.

(51) Int. Cl. C07K 14/705; A61K 38/00; A61K 48/00; A61P 25/00; C12N 15/86

(52) U.S. Cl. CPC C07K 14/70571; A61K 48/0058; A61P 25/00; C12N 15/86; A61K 38/00; C07K 2319/04; C07K 2319/09; C12N 2750/14143

(57) ABSTRACT

Compositions and methods are provided for modulating the activity of cells using engineered receptors, polynucleotide encoded engineered receptors, and gene therapy vectors comprising polynucleotides encoding engineered receptors. These compositions and methods find particular use in modulating the activity of neurons, for example in the treatment of disease or in the study of neuronal circuits.

### CROSS-REFERENCE TO RELATED APPLICATIONS

This application claims the benefit of U.S. Provisional Application No. 63/311,735, filed Feb. 18, 2022, and U.S. Provisional Application No. 63/348,560, filed Jun. 3, 2022, the contents of all of which are herein incorporated by reference in their entireties.

### REFERENCE TO AN ELECTRONIC SEQUENCE LISTING

The contents of the electronic sequence listing (SWCH_035_03WO_SeqList ST26.xml; Size: 183,826 bytes; and Date of Creation: Feb. 20, 2023) is herein incorporated by reference in its entirety.

### FIELD

This disclosure pertains to engineered receptors and the use of engineered receptors and small molecule ligands to modulate the activity of cells and treat diseases.

### BACKGROUND

Intractable neurological disease is often associated with aberrantly acting neurons. Attempts to develop therapies to treat these conditions have been hampered by a lack of tractable target proteins associated with the disease.

The most commonly used therapy for chronic pain is the application of opioid analgesics and nonsteroidal anti-inflammatory drugs, but these drugs can lead to addiction and may cause side effects, such as drug dependence, tolerance, respiratory depression, sedation, cognitive failure, hallucinations, and other systemic side effects. Despite the wide usage of pharmaceuticals, there is a strikingly low success rate for its effectiveness in pain relief. More invasive options for the treatment of pain include nerve blocks and electrical stimulation. The most invasive, and least preferred, method for managing pain is complete surgical removal of the nerve or section thereof that is causing the pain.

There are long-felt and unmet needs of safe, efficient and cost-effective treatment of neurological disorders. The disclosure provides a safe, efficient and cost-effective treatment of neurological disorders, including the management of pain.

### BRIEF DESCRIPTION OF THE DRAWINGS

The disclosure is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures:

FIG. 1 shows the heat map of the percent quench of YFP fluorescence of cells transfected with CODA71 or CODA1237 following stimulation by various doses of either acetylcholine or the indicated non-native ligand. Ligand doses are written across the top of each chart. Numbers in the boxes indicate the absolute amount of quench observed. Dark blue indicates strong quenching of YFP reporter. Light blue indicates moderate quenching. Orange suggest weak or minimal quenching. Negative values represent non-responders that have a negative quench due to a stimulation artifact.

FIG. 2 shows the heat map of the percent quench of YFP fluorescence of cells transfected with a CODA1237-based chimeric LGIC receptor comprising various amino acid mutations following stimulation by increasing doses of either acetylcholine or TC-5619. CODA75 is a non-responding chimera used as a negative control. Color coding and labels follow the same rule as in FIG. 1.

FIG. 3 shows the heat map of the percent quench of YFP fluorescence of cells transfected with CODA 64, CODA71, or a chimeric receptor comprising the ion pore domain of GlyRa3 (CODA1342, CODA1343, CODA1344, CODA1345) following stimulation by increasing doses of acetylcholine. CODA75 is a non-responding chimera used as a negative control. Color coding and labels follow the same rule as in FIG. 1.

FIG. 4A shows the percentage of α-bungarotoxin staining positive HEK293T cells transiently transfected with indicated receptor. FIG. 4B shows the mean fluorescent intensity (MFI) of α-bungarotoxin staining positive cells from FIG. 4A. FIG. 4C shows the percentage of α-bungarotoxin staining positive cells transiently transfected with indicated receptor in HEK293T cells transduced with Ric3, Nacho, and GCAMP6s. FIG. 4D shows the mean fluorescent intensity of α-bungarotoxin staining positive cells from FIG. 4C. Data is representative of n=2-6 replicate samples.

FIG. 5A shows the percentage of α-bungarotoxin staining positive HEK293T cells transiently transfected with indicated receptor. FIG. 5B shows the mean fluorescent intensity of α-bungarotoxin staining positive cells from FIG. 5A. Data is representative of n=2-6 replicate samples. FIG. 5C shows the dose response for TC-5619 and acetylcholine of CODA71 in HEK cells. FIG. 5D shows the dose response for TC-5619 and acetylcholine of CODA1055 in HEK cells. FIG. 5E shows the dose response for TC-5619 of CODA1316 in HEK cells. FIG. 5F shows the peak current of the indicated receptors in HEK cells when activated by TC-5619.

FIG. 6 shows sequence alignment of human α7-nAChR (SEQ ID NO:4), human GlyRα1 (SEQ ID NO:2), human GlyRα2 (SEQ ID NO:59), human GlyRα3 (isoform L, SEQ ID NO:61), human GABA-A ρ1 (SEQ ID NO:10), human GABA-A ρ2 (SEQ ID NO:12), and human GABA-A ρ3 (SEQ ID NO:14) protein sequences. β1-2 loop, Cys-loop, Pre-M1 linker and M2-M3 linker regions are labeled.

### SUMMARY

In one aspect, the disclosure provides engineered receptors, wherein the engineered receptor is a chimeric ligand gated ion channel (LGIC) receptor and comprises (a) a ligand binding domain derived from a first wild type Cys-loop LGIC receptor, and (b) an ion pore domain derived from a second wild type Cys-loop LGIC receptor. In some embodiments, the first wild type Cys-loop LGIC receptor comprises a nicotinic acetylcholine receptor family receptor. In some embodiments, the first wild type Cys-loop LGIC receptor is human α7 nicotinic acetylcholine receptor (α7-nAChR, SEQ ID NO:4). In some embodiments, the second wild type Cys-loop LGIC receptor is a chloride permeable Cys-loop ligand gated ion channel receptor. In some embodiments, the second wild type Cys-loop LGIC receptor is a glycine receptor or a GABA-A receptor. In some embodiments, the second wild type Cys-loop LGIC receptor comprises a Glycine receptor α1, a Glycine receptor α2, a Glycine receptor α3, a GABA-A receptor ρ1, a GABA-A receptor ρ2, or a GABA-A receptor ρ3. In some embodiments, the second wild type Cys-loop LGIC receptor is not a Glycine receptor α1. In some embodiments, part or all of Cys-loop domain of the ligand binding domain is derived from the second wild type Cys-loop LGIC receptor. In some embodiments, part or all of β1-2 loop domain of the ligand binding domain is derived from the second wild type Cys-loop LGIC receptor. In some embodiments, the engineered receptor comprises a pre-M1 linker derived from the first wild type Cys-loop LGIC receptor or the second wild type Cys-loop LGIC receptor. In some embodiments, the engineered receptor comprises a pre-M1 linker comprising a N-terminal segment derived from the first wild type Cys-loop LGIC receptor and a C-terminal segment derived from the second wild type Cys-loop LGIC receptor. In some embodiments, the pre-M1 linker comprises or consist of a sequence having at least 70% identity to any one of SEQ ID NOS: 72-91. In some embodiments, part or all of M2-M3 linker of the ion pore domain is derived from the first wild type Cys-loop LGIC receptor. In some embodiments, the M2-M3 linker of the ion pore domain comprises one or more mutations. In some embodiments, the M2-M3 linker of the ion pore domain comprises an amino acid sequence having at least 70% identity according to amino acids 283-295 of SEQ ID NO:4. In some embodiments, the engineered receptor forms homomeric ion channels when expressed on cell surface. In some embodiments, more than 50% of the engineered receptor expressed on cell surface form homomeric ion channels.

In some embodiments, the second wild type Cys-loop LGIC receptor is a human Glycine receptor α1 subunit (GlyRα1). In some embodiments, the ion pore domain comprises an amino acid sequence having at least 85% identity according to amino acids 248-457 of SEQ ID NO:2. In some embodiments, the Cys-loop domain of the ligand binding domain comprises an amino acid sequence having at least 80% identity according to amino acids 166-180 of SEQ ID NO:2. In some embodiments, the β1-2 loop domain of the ligand binding domain comprises an amino acid sequence having at least 80% identity according to amino acids 80-85 of SEQ ID NO:2. In some embodiments, the ion pore domain comprises no amino acid between the amino acid positions corresponding to K353 and E362 of SEQ ID NO:2. In some embodiments, the ion pore domain comprises an amino acid sequence having less than 50% sequence identity to SEQ ID NO: 96 between the amino acid positions corresponding to K353 and E362 of SEQ ID NO:2. In some embodiments, cell surface expression of such an engineered receptor is increased by at least 50% compared to a corresponding engineered receptor comprising an amino acid sequence according to SEQ ID NO: 96 between the amino acid positions corresponding to K353 and E362 of SEQ ID NO:2. In some embodiments, the ion pore domain comprises one or more mutations in a region corresponding to the nuclear localization signal (NLS)/ER retention signal (ERRS) sequence of the human GlyRα1. In some embodiments, the engineered receptor comprises an amino acid sequence having at least 90% identity to SEQ ID NO:16 or 33.

In some embodiments, the second wild type Cys-loop LGIC receptor is a human Glycine receptor α2 subunit (GlyRα2). In some embodiments, the ion pore domain comprises an amino acid sequence having at least 85% identity according to amino acids 254-452 of SEQ ID NO:59. In some embodiments, the Cys-loop domain of the ligand binding domain comprises an amino acid sequence having at least 80% identity according to amino acids 172-186 of SEQ ID NO:59. In some embodiments, the β1-2 loop domain of the ligand binding domain comprises an amino acid sequence having at least 80% identity according to amino acids 86-91 of SEQ ID NO:59. In some embodiments, the ion pore domain comprises one or more mutations in a region corresponding to the NLS/ERRS sequence of the human GlyRα2. In some embodiments, the engineered receptor comprises an amino acid sequence having at least 90% identity to SEQ ID NO:62 or 65.

In some embodiments, the second wild type Cys-loop LGIC receptor is the human Glycine receptor α3 subunit (GlyRα3). In some embodiments, the ion pore domain comprises an amino acid sequence having at least 85% identity according to amino acids 253-464 of SEQ ID NO:61 or amino acids 253-449 of SEQ ID NO: 69. In some embodiments, the Cys-loop domain of the ligand binding domain comprises an amino acid sequence having at least 80% identity according to amino acids 171-185 of SEQ ID NO:61 or 69. In some embodiments, the β1-2 loop domain of the ligand binding domain comprises an amino acid sequence having at least 80% identity according to amino acids 85-90 of SEQ ID NO:61 or 69. In some embodiments, the ion pore domain comprises no amino acid residue between the amino acid positions corresponding to K357 and D358 of SEQ ID NO:69. In some embodiments, the ion pore domain comprises an amino acid sequence having less than 50% sequence identity to SEQ ID NO: 95 between the amino acid positions corresponding to K357 and D358 of SEQ ID NO:69. In some embodiments, cell surface expression of such an engineered receptor is increased by at least 50% compared to a corresponding engineered receptor comprising an ion pore domain derived from the IPD of human GlyRα3 isoform L (SEQ ID NO: 61) comprising the 15 amino acid residues corresponding to amino acids 358-372 of SEQ ID NO:61. In some embodiments, the ion pore domain comprises one or more mutations in a region corresponding to the NLS/ERRS sequence of the human GlyRα3. In some embodiments, the engineered receptor comprises an amino acid sequence having at least 90% identity to any one of SEQ ID NO:63, 66, 70 and 71.

In some embodiments, the second wild type Cys-loop LGIC receptor is human GABA-A receptor ρ1 subunit (GABA-A ρ1). In some embodiments, the ion pore domain comprises an amino acid sequence having at least 85% identity according to amino acids 281-479 of SEQ ID NO:10. In some embodiments, the Cys-loop domain of the ligand binding domain comprises an amino acid sequence having at least 80% identity according to amino acids 198-212 of SEQ ID NO:10. In some embodiments, the β1-2 loop domain of the ligand binding domain comprises an amino acid sequence having at least 80% identity according to amino acids 112-117 of SEQ ID NO:10. In some embodiments, the engineered receptor comprises an amino acid sequence having at least 90% identity to SEQ ID NO:64 or 67.

In some embodiments, the pre-M1 linker of the ligand binding domain comprises one or more mutations. In some embodiments, the ligand binding domain is derived from human α7-nAChR, and the one or more mutations in the pre-M1 linker are at one or more positions corresponding to T225, M226, and/or T230 of human α7-nAChR. In some embodiments, the ligand binding domain is derived from human α7-nAChR, and the one or more mutations comprises a mutation corresponding to the T225I of human α7-nAChR. In some embodiments, the one or more mutations increases surface expression of the engineered receptor. In some embodiments, the surface expression is measured by α-bungarotoxin (a-BTX) assay.

In some embodiments, the ligand binding domain comprises a sequence having at least 85%, at least 90%, at least 95%, at least 96%, at least 97%, at least 98%, at least 99%, at least 99.5%, or 100%, identity to amino acids 23-220 of SEQ ID NO:4.

In some embodiments, the ligand binding domain comprises an amino acid substitution at one or more residues comprising W77, Y94, R101, W108, Y115, T128, N129, V130, L131, Q139, Y140, L141, Y151, S170, W171, S172, Y173, S188, Y190, Y210, C212, C213, E215, Y217, or any combination thereof, of human α7-nAChR. In some embodiments, the ligand binding domain comprises two amino acid substitutions at a pair of residues comprising R101 and L131, Y115 and Y210, or R101 and Y210, of human α7-nAChR. In some embodiments, the ligand binding domain comprises the amino acid substitutions corresponding to L131N, W77F, and S172D of SEQ ID NO:4. In some embodiments, the ligand binding domain comprises the amino acid substitutions corresponding to Q139W and S172D of SEQ ID NO:4. In some embodiments, the ligand binding domain comprises one or more amino acid substitutions listed in Table 12. In some embodiments, the ligand binding domain comprises the amino acid substitutions corresponding to R101W, Y115E, and Y210W of human α7-nAChR. In some embodiments, the engineered receptor comprises a sequence having at least 85%, at least 90%, at least 95%, at least 96%, at least 97%, at least 98%, or at least 99%, identity to SEQ ID NO:65 and comprises the amino acid substitutions corresponding to R101W, Y115E, Y210W, and T225I of SEQ ID NO:65.

In some embodiments, the potency of the engineered receptor to a native ligand of the first wild type Cys-loop LGIC receptor is lower than the potency of the first wild type Cys-loop LGIC receptor to the native ligand. In some embodiments, the potency of the engineered receptor to the native ligand is at least 2-fold lower than the potency of the first wild type Cys-loop LGIC receptor to the native ligand. In some embodiments, the potency of the engineered receptor to a non-native ligand is about the same as the potency of the first wild type Cys-loop LGIC receptor to the non-native ligand. In some embodiments, the potency of the engineered receptor to a non-native ligand is higher than the potency of the first wild type Cys-loop LGIC receptor to the non-native ligand. In some embodiments, the potency of the engineered receptor to the non-native ligand is at least 2-fold higher than the potency of the first wild type Cys-loop LGIC receptor to the non-native ligand. In some embodiments, determining the potency comprises determining the EC50. In some embodiments, the efficacy of the engineered receptor in the presence of a non-native ligand is higher than the efficacy of the first wild type Cys-loop LGIC receptor in presence of the non-native ligand. In some embodiments, the efficacy of the engineered receptor in the presence of a non-native ligand is at least 2-fold higher than the efficacy the first wild type Cys-loop LGIC receptor in presence of the non-native ligand. In some embodiments, determining the efficacy comprises determining the amount of current passed through the engineered receptor in vitro in the presence of the non-native ligand. In some embodiments, the non-native ligand is selected from the group consisting of AZD-0328, TC-6987, ABT-126, APN-1125, TC-5619, and Facinicline/RG3487. In some embodiments, the non-native ligand is selected from the group consisting of ABT-126, RG3487, and APN-1125. In some embodiments, the non-native ligand is TC-5619.

In one aspect, the disclosure provides polynucleotides comprising a nucleic acid encoding the engineered receptor of the disclosure. In some embodiments, the polynucleotide comprises a promoter operably linked to the nucleic acid encoding the engineered receptor. In some embodiments, the promoter is a regulatable promoter. In some embodiments, the regulatable promoter is active in an excitable cell. In some embodiments, the excitable cell is a neuron or a myocyte. In some embodiments, the excitable cell is a neuron.

In one aspect, the disclosure provides vectors comprising the polynucleotide of the disclosure. In some embodiments, the vector is a plasmid, or a viral vector. In some embodiments, the vector is a viral vector selected from the group consisting of an adenoviral vector, a retroviral vector, an adeno-associated viral (AAV) vector, and a herpes simplex-1 viral vector (HSV-1). In some embodiments, the viral vector is an AVV vector, and wherein the AAV vector is AAV5 or a variant thereof, AAV6 or a variant thereof or AAV9 or a variant thereof.

In one aspect, the disclosure provides compositions comprising the engineered receptor of the disclosure, the polynucleotide of the disclosure, or the vector of the disclosure.

In one aspect, the disclosure provides pharmaceutical compositions comprising the engineered receptor of the disclosure, the polynucleotide of the disclosure, or the vector of the disclosure, and a pharmaceutically acceptable carrier.

In one aspect, the disclosure provides methods of producing an engineered receptor in a neuron, comprising contacting the neuron with the polynucleotide of the disclosure, the vector of the disclosure, the composition of the disclosure, or the pharmaceutical composition of the disclosure. In some embodiments, the neuron is a neuron of the peripheral nervous system. In some embodiments, the neuron is a neuron of the central nervous system. In some embodiments, the neuron is a nociceptive neuron. In some embodiments, the neuron is a non-nociceptive neuron. In some embodiments, the neuron is a dorsal root ganglion (DRG) neuron, a trigeminal ganglion (TG) neuron, a motor neuron, an excitatory neuron, an inhibitory neuron, or a sensory neuron. In some embodiments, the neuron is an AS afferent fiber, a C fiber or an Aβ afferent fiber. In some embodiments, the neuron is Aβ afferent fiber. In some embodiments, Aβ afferent fiber is an injured Aβ afferent fiber. In some embodiments, Aβ afferent fiber is an uninjured Aβ afferent fiber. In some embodiments, the neuron expresses neurofilament 200 (NF200), piezo 2, and TLR-5. In some embodiments, the neuron does not express TrpV1, prostatic acid phosphatase, NaV1.1. In some embodiments, the contacting step is performed in vitro, ex vivo, or in vivo. In some embodiments, the contacting step is performed in vivo in a subject. In some embodiments, the contacting step comprises administering the polynucleotide, the vector, the composition, or the pharmaceutical composition to the subject. In some embodiments, the contacting step is performed in vitro or ex vivo. In some embodiments, the contacting step comprises lipofection, nanoparticle delivery, particle bombardment, electroporation, sonication, or microinjection. In some embodiments, the engineered receptor is capable of localizing to the cell surface of the neuron.

In one aspect, the disclosure provides methods of inhibiting the activity of a neuron, comprising (a) contacting the neuron with the engineered receptor of the disclosure, the polynucleotide of the disclosure, the vector of the disclosure, the composition of the disclosure, or the pharmaceutical composition of the disclosure, and (b) contacting the neuron with a non-native ligand of the engineered receptor. In some embodiments, neuron is a neuron of the peripheral nervous system. In some embodiments, the neuron is a neuron of the central nervous system. In some embodiments, the neuron is a nociceptive neuron. In some embodiments, the neuron is a non-nociceptive neuron. In some embodiments, the neuron is a dorsal root ganglion (DRG) neuron, a trigeminal ganglion (TG) neuron, a motor neuron, an excitatory neuron, an inhibitory neuron, or a sensory neuron. In some embodiments, the neuron is an Aδ afferent fiber, a C fiber or an Aβ afferent fiber. In some embodiments, the neuron is Aβ afferent fiber. In some embodiments, Aβ afferent fiber is an injured Aβ afferent fiber. In some embodiments, Aβ afferent fiber is an uninjured Aβ afferent fiber. In some embodiments, the neuron expresses neurofilament 200 (NF200), piezo 2, and TLR-5. In some embodiments, the neuron does not express TrpV1, prostatic acid phosphatase, NaV1.1. In some embodiments, the contacting step (a) is performed in vitro, ex vivo, or in vivo. In some embodiments, wherein the contacting step (b) is performed in vitro, ex vivo, or in vivo. In some embodiments, the contacting steps (a) and/or (b) are performed in vivo in a subject. In some embodiments, the contacting step (a) comprises administering the engineered receptor, the polynucleotide, the vector, or the pharmaceutical composition to the subject; and/or the contacting step (b) comprises administering the non-native ligand to the subject. In some embodiments, the contacting step (a) and/or (b) comprises lipofection, nanoparticle delivery, particle bombardment, electroporation, sonication, or microinjection. In some embodiments, the engineered receptor is capable of localizing to the cell surface of the neuron.

In one aspect, the disclosure provides methods of treating and/or delaying the onset of a neurological disorder in a subject, in need thereof, comprising: (a) administering to the subject, a therapeutically effective amount of the engineered receptor of the disclosure, the polynucleotide of the disclosure, the vector of the disclosure, the composition of the disclosure, or the pharmaceutical composition of the disclosure, and (b) administering to the subject a non-native ligand of the engineered receptor. In some embodiments, the subject is administered the non-native ligand after step (a). In some embodiments, the subject is administered the non-native ligand concurrently with step (a). In some embodiments, the neurological disorder is a seizure disorder, a movement disorder, an eating disorder, a spinal cord injury, neurogenic bladder, allodynia, a spasticity disorder, pruritus, Alzheimer's disease, Parkinson's disease, post-traumatic stress disorder (PTSD), gastroesophageal reflux disease (GERD), addiction, anxiety, depression, memory loss, dementia, sleep apnea, stroke, narcolepsy, urinary incontinence, essential tremor, trigeminal neuralgia, burning mouth syndrome, or atrial fibrillation. In some embodiments, the neurological disorder is allodynia. In some embodiments, the non-native ligand is selected from the group consisting of AZD-0328, ABT-126, TC6987, APN-1125, TC-5619, and Facinicline/RG3487. In some embodiments, the non-native ligand is administered orally, subcutaneously, topically, or intravenously. In some embodiments, the non-native ligand is administered orally. In some embodiments, the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna. In some embodiments, the engineered receptor, the polynucleotide, the vector, the composition, or the pharmaceutical composition is administered by transforaminal injection or intrathecally. In some embodiments, the subject suffers from trigeminal neuralgia, and wherein the engineered receptor, the polynucleotide, the vector, the composition, or the pharmaceutical composition is administered to the trigeminal ganglion (TG) of the subject. In some embodiments, the subject suffers from neuropathic pain, and wherein the engineered receptor, the polynucleotide, the vector, the composition, or the pharmaceutical composition is administered to the dorsal root ganglion (DRG) of the subject. In some embodiments, the subject is a human. In some embodiments, the therapeutically effectively amount diminishes the severity of a sign and/or or a symptom of the neurological disorder. In some embodiments, the therapeutically effectively amount delays the onset of a sign and/or or a symptom of the neurological disorder. In some embodiments, the therapeutically effectively amount eliminates a sign and/or or a symptom of the neurological disorder. In some embodiments, the sign of the neurological disorder is nerve damage, nerve atrophy, and/or seizure. In some embodiments, the nerve damage is peripheral nerve damage. In some embodiments, the symptom of the neurological disorder is pain.

In one aspect, the disclosure provides methods of treating and/or delaying the onset of pain in a subject in need thereof, comprising: (a) administering to the subject, a therapeutically effective amount of the engineered receptor of the disclosure, the polynucleotide of the disclosure, the vector of the disclosure, the composition of the disclosure, or the pharmaceutical composition of the disclosure, and (b) administering to the subject a non-native ligand of the engineered receptor. In some embodiments, the subject is administered the non-native ligand after step (a). In some embodiments, the subject is administered the non-native ligand concurrently with step (a). In some embodiments, the non-native ligand is selected from the group consisting of AZD-0328, ABT-126, TC6987, APN-1125, TC-5619, and Facinicline/RG3487. In some embodiments, the non-native ligand is administered orally, subcutaneously, topically, or intravenously. In some embodiments, the non-native ligand is administered orally. In some embodiments, the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna. In some embodiments, the engineered receptor, the polynucleotide, the vector, the composition, or the pharmaceutical composition is administered by transforaminal injection or intrathecally. In some embodiments, the subject suffers from trigeminal neuralgia, and wherein the engineered receptor, the polynucleotide, the vector, the composition, or the pharmaceutical composition is administered to the trigeminal ganglion (TG) of the subject. In some embodiments, the subject suffers from neuropathic pain, and wherein the engineered receptor, the polynucleotide, the vector, the composition, or the pharmaceutical composition is administered to the dorsal root ganglion (DRG) of the subject. In some embodiments, the subject is a human. In some embodiments, the pain is neuropathic pain. In some embodiments, the pain is associated with, caused by, or resulting from chemotherapy. In some embodiments, the pain is associated with, caused by, or resulting from trauma. In some embodiments, the subject suffers from allodynia. In some embodiments, the pain manifests after a medical procedure. In some embodiments, the pain is associated with, is caused by, or resulting from childbirth or Caesarean section. In some embodiments, the pain is associated with, is caused by, or resulting from migraine. In some embodiments, the therapeutically effectively amount diminishes pain in the subject transiently, diminishes pain in the subject permanently, prevents the onset of pain in the subject, and/or eliminates pain in the subject. In some embodiments, steps (a) and (b) are performed before the manifestation of pain in the subject.

### DETAILED DESCRIPTION

### Overview

Intractable neurological disease is often associated with aberrantly acting neurons. Attempts to develop therapies to treat these conditions have been hampered by a lack of tractable target proteins associated with the disease. For example, unrelieved chronic pain is a critical health problem in the US and worldwide. A report by the Institute of Medicine estimated that 116 million Americans suffer from pain that persists for weeks to years, with resulting annual costs exceeding $560 million. There are no adequate long-term therapies for chronic pain sufferers, leading to significant cost for both society and the individual. Pain often results in disability and, even when not disabling, it has a profound effect on the quality of life. Pain treatment frequently fails even when the circumstances of care delivery are optimal, such as attentive, well-trained physicians; ready access to opioids; use of adjuvant analgesics; availability of patient-controlled analgesia; and evidence-based use of procedures like nerve blocks and IT pumps.

The most commonly used therapy for chronic pain is the application of opioid analgesics and nonsteroidal anti-inflammatory drugs, but these drugs can lead to addiction and may cause side effects, such as drug dependence, tolerance, respiratory depression, sedation, cognitive failure, hallucinations, and other systemic side effects. Despite the wide usage of pharmaceuticals, there is a strikingly low success rate for its effectiveness in pain relief. A large randomized study with various medications found only one out of every two or three patients achieving at least 50% pain relief (Finnerup et al., 2005). A follow-up study using the most developed pharmacological treatments found the same results, indicating that there was no improvement in the efficacy of medications for pain (Finnerup et al., Pain, 150(3):573-81, 2010).

More invasive options for the treatment of pain include nerve blocks and electrical stimulation. A nerve block is a local anesthetic injection usually in the spinal cord to interrupt pain signals to the brain, the effect of which only lasts from weeks to months. Nerve blocks are not the recommended treatment option in most cases (Mailis and Taenzer, Pain Res Manag. 17(3):150-158, 2012). Electrical stimulation involves providing electric currents to block pain signals. Although the effect may last longer than a nerve block, complications arise with the electrical leads itself: dislocation, infection, breakage, or the battery dying. One review found that 40% of patients treated with electrical stimulation for neuropathy experienced one or more of these issues with the device (Wolter, 2014).

The most invasive, and least preferred, method for managing pain is complete surgical removal of the nerve or section thereof that is causing the pain. This option is only recommended when the patient has exhausted the former and other less invasive, treatments and found them ineffective. Radiofrequency nerve ablation uses heat to destroy problematic nerves and provides a longer pain relief than a nerve block. However, one study found no difference between the control and treatment groups in partial radiofrequency lesioning of the DRG for chronic lumbosacral radicular pain (Geurts et al., 2003). Other surgical methods for surgically removing the pain nerves suffer from similar shortcomings and have serious side effects long-term, including sensory or motor deficits, or cause pain elsewhere.

Compositions and methods are provided for modulating the activity of cells using engineered ligand gated ion channel (LGIC) receptors, polynucleotide encoded engineered LGIC receptors, and gene therapy vectors comprising polynucleotides encoding engineered LGIC receptors. These compositions and methods find particular use in modulating the activity of neurons, for example in the treatment of disease or in the study of neuronal circuits. In addition, reagents, devices and kits thereof that find use in practicing the subject methods are provided.

In particular, the present disclosure provides engineered receptors that bind to and signal in response to known drugs, ligands, and/or binding agents. In some embodiments, the engineered receptors described herein demonstrate increased affinity for an agonist or agonistic binding agent. In some embodiments, the engineered receptors described herein demonstrate an affinity for an antagonist or modulator binding agent and respond to the antagonist and/or modulator agents as if they were agonist agents. The present disclosure further provides for methods of treating neurological diseases in subjects in need thereof. The present disclosure increases the number of clinical indications that a known drug may be used for by utilizing engineered receptors that respond to a known drug in a manner that is distinct from the wild-type endogenous receptor.

Before the present methods and compositions are described, it is to be understood that this disclosure is not limited to particular method or composition described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

The publications discussed throughout the disclosure are provided solely for their disclosure prior to the filing date of the present application. Nothing in the disclosure is to be construed as an admission that the present disclosure is not entitled to antedate such publication by virtue of prior disclosure. Further, the dates of publication provided may be different from the actual publication dates which may need to be independently confirmed.

### Definitions

As used in this specification and the appended claims, the singular forms “a,” “an” and “the” include plural references unless the content clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

As used in this specification, the term “and/or” is used in this disclosure to either “and” or “or” unless indicated otherwise.

Throughout this specification, unless the context requires otherwise, the word “comprise”, or variations such as “comprises” or “comprising” refers to the inclusion of a stated element or integer or group of elements or integers but not the exclusion of any other element or integer or group of elements or integers. Further, the statement of numerical ranges throughout this specification specifically includes all integers and decimal points in between.

Throughout this specification, unless the context requires otherwise, the phrase “consisting essentially of” refers to a limitation of the scope of composition, method, or kit described to the specified materials or steps that do not materially affect the basic and novel characteristic(s) of the subject disclosure. For example, a ligand binding domain “consisting essentially of” a disclosed sequence has the amino acid sequence of the disclosed sequence plus or minus about 5 amino acid residues at the boundaries of the sequence, e.g. about 5 residues, 4 residues, 3 residues, 2 residues or about 1 residue less than the recited bounding amino acid residue, or about 1 residue, 2 residues, 3 residues, 4 residues, or 5 residues more than the recited bounding amino acid residue.

Throughout this specification, unless the context requires otherwise, the phrase “consisting of” refers to the exclusion from the composition, method, or kit of any element, step, or ingredient not specified in the claim. For example, a ligand binding domain “consisting of” a disclosed sequence consists only of the disclosed amino acid sequence.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used throughout the disclosure, the term “isolated” means material that is substantially or essentially free from components that normally accompany is as found in its native state. In some embodiments, the terms “obtained” or “derived” are used synonymously with isolated.

The terms “subject,” “individual,” and “patient” are used interchangeably to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant.

The term “sample” refers to a volume and/or mass of biological material that is subjected to analysis. In some embodiments, a sample comprises a tissue sample, cell sample, a fluid sample, and the like. In some embodiments, a sample is taken from or provided by a subject (e.g., a human subject). In some embodiments, a sample comprises a portion of tissue taken from any internal organ, a cancerous, pre-cancerous, or non-cancerous tumor, brain, skin, hair (including roots), eye, muscle, bone marrow, cartilage, white adipose tissue, and/or brown adipose tissue. In some embodiments, a fluid sample comprises buccal swabs, blood, cord blood, saliva, semen, urine, ascites fluid, pleural fluid, spinal fluid, pulmonary lavage, tears, sweat, and the like. Those of ordinary skill in the art will appreciate that, in some embodiments, a “sample” is a “primary sample” in that it is obtained directly from a source (e.g., a subject). In some embodiments, a “sample” is the result of processing of a primary sample, for example to remove certain potentially contaminating components, to isolate certain components, and/or to purify certain components of interest. In some embodiments, a sample is a cell or population of cells (e.g., a neuronal cell). A cell sample may be derived directly from a subject (e.g., a primary sample) or may be a cell line. Cell lines may include non-mammalian cells (e.g., insect cells, yeast cells, and/or bacterial cells) or mammalian cells (e.g., immortalized cell lines).

“Treating” or “treatment” as used throughout the disclosure refers to delivering a composition (e.g., an engineered receptor and/or a binding agent) to a subject and/or population of cells to affect a physiologic outcome. In particular embodiments, treatment results in an improvement (e.g., reduction, amelioration, or remediation) of one or more disease symptoms. The improvement may be an observable or measurable improvement, or may be an improvement in the general feeling of well-being of the subject. Treatment of a disease can refer to a reduction in the severity of disease symptoms. In some embodiments, treatment can refer to a reduction in the severity of disease symptoms to levels comparable to those prior to disease onset. In some embodiments, treatment may refer to a short-term (e.g., temporary or acute) and/or a long-term (e.g., sustained or chronic) reduction in disease symptoms. In some embodiments, treatment may refer to a remission of disease symptoms. In some embodiments, treatment may refer to the prophylactic treatment of a subject at risk of developing a particular disease in order to prevent disease development. Prevention of disease development can refer to complete prevention of the disease symptoms, a delay in disease onset, a lessening of the severity of the symptoms in a subsequently developed disease, or reducing the likelihood of disease development.

As used throughout the disclosure, “management” or “controlling” refers to the use of the compositions or methods contemplated in the disclosure, to improve the quality of life for an individual suffering from a particular disease. In certain embodiments, the compositions and methods of the disclosure provide analgesia to a subject suffering from pain.

A “therapeutically effective amount” is an amount of a composition required to achieve a desired therapeutic outcome. The therapeutically effective amount may vary according to factors such as, but not limited to, disease state and age, sex, and weight of the subject. Generally, a therapeutically effective amount is also one in which any toxic or detrimental effects of a composition are outweighed by the therapeutically beneficial effects. A “therapeutically effective amount” includes an amount of a composition that is effective to treat a subject.

An “increase” refers to an increase in a value (e.g., increased binding affinity, increased physiologic response, increased therapeutic effect, etc.) of at least 5% as compared to a reference or control level. For example, an increase may include a 5, 6, 7, 8, 9, 10, 15, 20, 25, 30, 35, 40, 45, 50, 60, 70, 80, 90, 100, 150, 200, 250, 500, 1000% or more increase. Increase also means an increase that is 1.1, 1.2, 1.5, 2, 3, 4, 5, 6, 7, 8, 9, 10, 15, 20, 30 or more times (e.g., 500, 1000 times) higher than a reference or control level.

A “decrease”, “reduce”, “diminish” or synonyms thereof refers to a decrease in a value (e.g., decreased binding affinity, decreased physiologic response, decreased therapeutic effect, decrease in pain in a subject etc.) of at least 5% as compared to a reference or control level. For example, a decrease may include a 5, 6, 7, 8, 9, 10, 15, 20, 25, 30, 35, 40, 45, 50, 60, 70, 80, 90, 100, 150, 200, 250, 500, 1000% or more decrease. Decrease also means a decrease that is 1.1, 1.2, 1.5, 2, 3, 4, 5, 6, 7, 8, 9, 10, 15, 20, 30 or more times (e.g., 500, 1000 times) lower than a reference or control level.

The terms “reference” or “control” level are used interchangeably throughout the disclosure and refer the value of a particular physiologic and/or therapeutic effect in a subject or sample that has not been treated with a composition of the disclosure, or a subject or sample that has been treated with a vehicle control. In some embodiments, a reference level refers to a value of a particular physiologic and/or therapeutic effect that is measure in a subject or sample prior to the administration of a composition of the disclosure (e.g., a baseline level).

As used throughout the disclosure, “ligand” refers to a molecule that binds to another, larger molecule. In some embodiments, the ligand binds to a receptor. In some embodiments, the binding of the ligand to the receptor alters the function of the receptor—to activate or repress its function. In some embodiments, the binding of the ligand to a receptor such a ligand gated ion channel (LGIC) leads to the opening or closing of the ion channel.

“Receptor-ligand binding” and “ligand binding” are used interchangeably throughout the disclosure and refer to the physical interaction between a receptor (e.g., a LGIC) and a ligand. The term “ligand” as used throughout the disclosure may refer to an endogenous or naturally occurring ligand. For example, in some embodiments, a ligand refers to a neurotransmitter (e.g., λ-aminobutyric acid (GABA), acetylcholine, serotonin, and others) and signaling intermediate (e.g., phosphatidylinositol 4,5-bisphosphate (PIP₂)), amino acids (e.g., glycine), or nucleotides (e.g., ATP). In some embodiments, a ligand may refer to a non-native, i.e. synthetic or non-naturally occurring, ligand (e.g., a binding agent). For example, in some embodiments, a ligand refers to a small molecule. Ligand binding can be measured by a variety of methods known in the art (e.g., detection of association with a radioactively labeled ligand).

“Binding affinity” generally refers to the strength of the sum total of noncovalent interactions between a single binding site of a receptor and a ligand. Unless indicated otherwise, as used throughout the disclosure, “binding affinity” refers to intrinsic binding affinity which reflects a 1:1 interaction between members of a binding pair (e.g., receptor and ligand). The affinity of a molecule X for its partner Y can generally be represented by the dissociation constant (K_{d}). Affinity can be measured by common methods known in the art, including those described in the disclosure.

The terms “specific binding affinity” or “specific binding” are used interchangeably throughout the specification and claims and refer to binding which occurs between a paired species of molecules, e.g., receptor and ligand. When the interaction of the two species produces a non-covalently bound complex, the binding which occurs is typically electrostatic, hydrogen-bonding, or the result of lipophilic interactions. In various embodiments, the specific binding between one or more species is direct. In one embodiment, the affinity of specific binding is about 2 times greater than background binding (non-specific binding), about 5 times greater than background binding, about 10 times greater than background binding, about 20 times greater than background binding, about 50 times greater than background binding, about 100 times greater than background binding, or about 1000 times greater than background binding or more.

“Signaling” refers to the generation of a biochemical or physiological response as a result of ligand binding to a receptor (e.g., as a result of a binding agent binding to an engineered receptor of the disclosure).

The term “wild type” or “native” is a term of the art understood by skilled persons and means the typical form of an organism, strain, gene, protein, or characteristic as it occurs in nature as distinguished from mutant or variant forms. For example, a wild type protein is the typical form of that protein as it occurs in nature.

The terms “non-native”, “variant”, and “mutant” are used interchangeably throughout the specification and the claims to refer to a mutant of a native, or wild type, composition, for example a variant polypeptide having less than 100% sequence identity with the native, or wild type, sequence.

Amino acid modifications may be amino acid substitutions, amino acid deletions and/or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g. charge, hydrophobicity and size). As used throughout the disclosure, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to praline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

The term “parental” or “starter” are used interchangeably throughout the specification and claims to refer to an initial composition, or protein that is mutated, modified, or derivatized, to create an engineered composition having novel properties. In some embodiments, the parental protein is a chimeric protein.

The term “engineered” is used throughout the specification and claims to refer to a non-naturally occurring composition, or protein having properties that are distinct from the parental composition, or protein from which it was derivatized.

In general, “sequence identity” or “sequence homology” refers to the nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and intervening integer values. Typically, the percent identities between a disclosed sequence and a claimed sequence are at least 80%, at least 85%, at least 90%, at least 95%, or at least 98%.

As used throughout the disclosure, “substantially identical” refers to having a sequence identity that is 85% or more, for example 90% or more, e.g. 95%, 96%, 97%, 98%, 99%, 99.5%, 99.9%, or 100%, wherein the activity of the composition is unaltered by the modifications in the sequence that result in the difference in sequence identity.

As used throughout the disclosure, the term “promoter” refers to one or more nucleic acid control sequences that direct transcription of an operably linked nucleic acid. Promoters may include nucleic acid sequences near the start site of transcription, such as a TATA element. Promoters may also include cis-acting polynucleotide sequences that can be bound by transcription factors. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used throughout the disclosure, the terms “virus vector,” “viral vector,” or “gene delivery vector” refer to a virus particle that functions as a nucleic acid delivery vehicle, and which comprises a nucleic acid (e.g., an AAV expression cassette) packaged within a virion. Exemplary virus vectors of the disclosure include adenovirus vectors, adeno-associated virus vectors (AAVs), lentivirus vectors, and retrovirus vectors.

As used throughout the disclosure, “neuronal activity”, “activity of a neuron”, “neuronal firing” and variations and synonyms thereof, refer to the electrical activity resulting from the stimulation or excitation of a neuron. In some embodiments, neuronal activity is measured using automated or manual patch clamp techniques. In some embodiments, determining the activity of a neuron comprises determining the excitatory postsynaptic potential (EPSP), inhibitory postsynaptic potential (IPSP), and/or action potential of the neuron. In some embodiments, the level of activity of a neuron depends on, or is affected by, the excitatory postsynaptic potential (EPSP), inhibitory postsynaptic potential (IPSP), and/or action potential.

As used throughout the disclosure, a “neurological disease” or “neurological disorder” refers to a disease or disorder of the nervous system. In some embodiments, the neurological disease is associated with, caused by, or results from structural, biochemical, and/or electrical abnormalities in the brain, spinal cord, a nerve, or any component of the nervous system.

As used throughout the disclosure, a “sign” of a disease refers to a physical or mental feature which is regarded as indicating a condition of disease. In some embodiments, a sign is an objective indication of the disease. In some embodiments, a sign is evaluated, examined, observed or measured objectively by a person other than the patient, such as a doctor.

As used throughout the disclosure, a “symptom” of a disease refers to a physical or mental feature which is regarded as indicating a condition of disease, particularly such a feature that is apparent to the patient. In some embodiments, the symptom is subjectively evaluated by the patient. For example, in some embodiments, the symptom is pain.

As used throughout the disclosure, “potency” refers to the amount of ligand required to produce a certain level of activity of a protein, such as a LGIC. In some embodiments, the activity of the protein, such as LGIC, refers to the opening or closing of the ion channel. In some embodiments, determining the potency comprises determining the half maximal effective concentration (EC50) of the protein, such as a LGIC, to a ligand under specific conditions. The EC50 refers to the concentration of the ligand which induces a response halfway between the baseline and maximum after a specific exposure time.

As used throughout the disclosure, “efficacy” refers to a measure of the activity of a protein, such as a LGIC, in the presence of a ligand. In some embodiments, the efficacy refers to the amount of current passed through the LGIC under specific conditions, such as in the presence of a specific concentration of the ligand. In some embodiments, determining the efficacy comprises determining the amount of current passed through the receptor, and/or the rheobase of the receptor.

As used throughout the disclosure, “responsiveness” refers to a measure of the overall function of a protein, such as a LGIC, in the presence of a ligand. Determining the responsiveness may include the determination and consideration of one or more factors, such as potency, efficacy, and the sub-cellular localization of the protein.

As used throughout the disclosure in relation to the position of an amino acid, the terms “corresponding to” or “correspond to” refer to an amino acid in a first polypeptide sequence that aligns with a given amino acid in a reference polypeptide sequence when the first polypeptide and reference polypeptide sequences are aligned. Alignment is performed by one of skill in the art using software designed for this purpose, for example, Clustal Omega version 1.2.4 with the default parameters for that version. As an example, the amino acid position D449 in SEQ ID NO: 69 corresponds to D464 of SEQ ID NO: 61.

As used throughout the disclosure, an “amino acid mutation” refers to any difference in an amino acid sequence relative to a corresponding parental sequence, e.g. an amino acid substitution, deletion, and/or insertion.

### Engineered Receptors

The present disclosure is directed to engineered receptors, engineered receptor mutants, and methods for their use. The term “receptor” as used herein refers to any protein that is situated on the surface of a cell and that can mediate signaling to and/or from the cell. The term “engineered receptor” is used herein to refer to a receptor that has been experimentally altered such that it is physically and/or functionally distinct from a corresponding parental receptor. In some embodiments, the parental receptor is a wild-type receptor. The term “wild-type receptor” is used herein to refer to a receptor having a polypeptide sequence that is identical to the polypeptide sequence of a protein found in nature. Wild-type receptors include receptors that naturally occur in humans as well as orthologs that naturally occur in other eukaryotes, e.g. protist, fungi, plants or animals, for example yeast, insects, nematodes, sponge, mammals, non-mammalian vertebrates. In some embodiments, the parental receptor is a non-native receptor; that is, it is a receptor that does not occur in nature, for example, a receptor that is engineered from a wild type receptor. For example, a parental receptor may be an engineered receptor comprising one or more subunits from one wild-type receptor with one or more subunits from a second wild-type receptor. The resulting proteins are therefore comprised of subunits from two or more wild-type receptors. Therefore, in some embodiments, the parental receptor is a chimeric receptor. Engineered receptors of the present disclosure include, for example, parental receptor mutants and switch receptors.

In some embodiments, an engineered receptor of the present disclosure comprises at least one amino acid mutation relative to the corresponding parental receptor, e.g. one or more mutations in one or more domains of a wild-type receptor. In some embodiments, the engineered receptor shares a sequence identity of about 99%, about 98%, about 95%, about 90%, about 85%, about 80%, about 70%, about 60%, about 50%, or less with the corresponding parental receptor, inclusive of all values and subranges that lie therebetween. In some embodiments, the engineered receptor has a sequence identity of 85% or more with the corresponding parental receptor, e.g. 90% or more or 95% or more, for example, about 96%, about 97%, about 98% or about 99% identity with the corresponding parental receptor, inclusive of all values and subranges that lie therebetween. In some embodiments, an engineered receptor (e.g., a parental receptor mutant) is generated by error prone PCR.

In some embodiments, the ligand binding domain (LBD) of the engineered receptor of the disclosure comprises at least one amino acid mutation relative to the corresponding ligand binding domain of the parental receptor, e.g. one or more mutations in the ligand binding domain of a wild-type receptor. In some embodiments, the ligand binding domain of the engineered receptor shares a sequence identity of about 99%, about 98%, about 95%, about 90%, about 85%, about 80%, about 70%, about 60%, about 50%, or less with the ligand binding domain of the corresponding parental receptor, inclusive of all values and subranges that lie therebetween. In some embodiments, the ligand binding domain of the engineered receptor has a sequence identity of 85% or more with the corresponding ligand binding domain of the parental receptor, e.g. 90% or more or 95% or more, for example, about 96%, about 97%, about 98% or about 99% identity with the corresponding ligand binding domain of the parental receptor, inclusive of all values and subranges that lie therebetween. In some embodiments, the ligand binding domain of the engineered receptor is generated by error prone PCR.

In some embodiments, the ion pore domain (IPD) of the engineered receptor of the disclosure comprises at least one amino acid mutation relative to the corresponding ion pore domain of the parental receptor, e.g. one or more mutations in the ion pore domain of a wild-type receptor. In some embodiments, the ion pore domain of the engineered receptor shares a sequence identity of about 99%, about 98%, about 95%, about 90%, about 85%, about 80%, about 70%, about 60%, about 50%, or less with the ion pore domain of the corresponding parental receptor, inclusive of all values and subranges that lie therebetween. In some embodiments, the ion pore domain of the engineered receptor has a sequence identity of 85% or more with the corresponding ion pore domain of the parental receptor, e.g. 90% or more or 95% or more, for example, about 96%, about 97%, about 98% or about 99% identity with the ion pore domain of the corresponding parental receptor, inclusive of all values and subranges that lie therebetween. In some embodiments, the ion pore domain of the engineered receptor is generated by error prone PCR.

In some embodiments, the amino acid mutation is a loss-of-function amino acid mutation relative to a corresponding parental receptor. “Loss-of-function” amino acid mutations refer to one or more mutations that reduce, substantially decrease, or abolish the function of the engineered receptor relative to the parental receptor, for example by reducing the binding of an endogenous ligand to an engineered receptor relative to the binding of endogenous ligand to the parental receptor, or by reducing the activity of signaling pathway(s) downstream of the engineered receptor that are typically activated in response to the binding of a binding agent to the corresponding parental receptor.

In some embodiments, the amino acid mutation is a gain-of-function amino acid mutation relative to a corresponding parental receptor. “Gain-of-function” amino acid mutations refer to one or more mutations that modify the function of the engineered receptor relative to the parental receptor, for example by altering or enhancing the affinity of an engineered receptor for a binding agent relative to the binding of endogenous ligand to the parental receptor, or by altering or enhancing the activity of the signaling pathways that are activated in response to the binding of a binding agent to an engineered receptor relative to the binding of the endogenous ligand to the corresponding parental receptor. In some embodiments, a gain-of-function mutation results in an increased affinity of the engineered receptor for a binding agent. In particular embodiments, a gain-of-function mutation results in an increased affinity of the engineered receptor for an agonist binding agent. In some embodiments, a gain-of-function mutation results in an antagonist binding agent acting as an agonist binding agent upon binding to the engineered receptor (e.g., results in the activation of agonist signaling pathways instead of antagonist signaling pathways). In some embodiments, a gain-of-function mutation results in a modulator binding agent acting as an agonist binding agent upon binding to the engineered receptor. In some embodiments, the subject engineered receptor of the present disclosure, or the ligand binding domain and/or the ion pore domain thereof, comprises one or more loss-of-function amino acid mutations and one or more gain-of-function amino acid mutations relative to a corresponding parental receptor.

In some embodiments, the loss of function mutation and the gain of function mutation are at the same residue, i.e. they are the same mutation. In other embodiments, the loss of function mutation and the gain of function mutation are mutations at different amino acid residues. In some embodiments, the subject engineered receptor (or the ligand binding domain and/or the ion pore domain thereof) comprising the loss of function mutation and/or gain of function mutation shares a sequence identity of about 99%, about 98%, about 95%, about 90%, about 85%, about 80%, about 70%, about 60%, about 50%, including all ranges and subranges therebetween, or less with the corresponding parental receptor, e.g. wild type receptor or non-native receptor (or the corresponding ligand binding domain and/or ion pore domain thereof). In some embodiments, the subject engineered receptor (or the ligand binding domain and/or the ion pore domain thereof) shares a sequence identity of 85% or more with the corresponding parental receptor (or the corresponding ligand binding domain and/or ion pore domain thereof), for example 85%, 90%, or 95% or more sequence identity, in some instances 96%, 97%, 98% or more sequence identity, e.g. 99% or 99.5% or more sequence identity, inclusive of all values and subranges that lie therebetween.

In some aspects, engineered receptors of the present disclosure include receptors produced by the combination of one or more amino acid sequences, e.g. subunits, from one wild-type receptor with one or more amino acid sequences, e.g. subunits, from a second wild-type receptor. In other words, the engineered receptor comprises amino acid sequences that are heterologous to one another, whereby “heterologous”, it is meant not occurring together in nature. Such receptors are referred to herein as “chimeric receptors”. In some embodiments, chimeric receptors serve as parental receptors from which an engineered receptor of the present disclosure is generated.

In some embodiments, a parental receptor mutant demonstrates increased affinity for an agonist binding agent. In some embodiments, a ligand or a binding agent that functions as an antagonist or modulator when binding to a wild type receptor functions as an agonist when binding to a parental receptor mutant.

In some embodiments, the engineered receptor is a “ligand-gated ion channel” or LGIC. An LGIC refers to a large group of transmembrane proteins that allow passage of ions upon activation by a specific ligand (e.g., chemical or binding agent). LGIC are composed of at least two domains: a ligand binding domain and a transmembrane ion pore domain. Ligand binding to an LGIC results in activation of the LGIC and opening of the ion pore. Ligand binding causes a drastic change in the permeability of the channel to a specific ion or ions; effectively no ions can pass through the channel when it is inactive or closed but up to 10⁷ ions/second can pass through upon ligand binding. In some embodiments, LGICs respond to extracellular ligands (e.g., neurotransmitters) and facilitate an influx of ions into the cytosol. In some embodiments, LGICs respond to intracellular ligands (e.g., nucleotides such at ATP and signaling intermediates such as PIP₂) and facilitate an efflux of ions from the cytosol into the extracellular environment. Importantly, activation of LGIC results in the transport of ions across the cellular membrane (e.g., Ca²⁺, Na⁺, K⁺, Cl⁻, etc.) and does not result in the transport of the ligand itself.

LGIC receptors are comprised of multiple subunits and can be either homomeric receptors or heteromeric receptors. A homomeric receptor is comprised of subunits that are all the same type. A heteromeric receptor is comprised of subunits wherein at least one subunit is different from at least one other subunit comprised within the receptor. For example, the glycine receptor is comprised of 5 subunits of which there are two types: α-subunits, of which there are four isoforms (α₁-α₄) and β-subunits, of which there is a single known isoform. An exemplary homomeric GlyR is a GlyR comprised of 5 α₁-GlyR subunits. Similarly, a homomeric GABA_{A} receptor may be comprised of β₃-GABA_{A} subunits, and an nAchR receptor may be comprised of α₇-nAchR subunits. An exemplary heteromeric GlyR may be comprised of one or more α-subunits and one or more of β-subunits (e.g., an α₁β-GlyR). Subunits of example LGIC receptors are shown in Table 1.

TABLE 1
LGIC Receptors and Subunits

| | | Subunit |
|---|---|---|
| Receptor | Subunits | Isoforms |
| GlyR | GLRA1 | |
| | GLRA2 | |
| | GLRB | |
| 5HT₃ | 5-HT3A | |
| | 5-HT3B | |
| | 5-HT3C | |
| | 5-HT3D | |
| | 5-HT3E | |
| nAChR | α | α1 |
| | | α2 |
| | | α3 |
| | | α4 |
| | | α5 |
| | | α6 |
| | | α7 |
| | | α8 |
| | | α9 |
| | | α10 |
| | β | β1 |
| | | β2 |
| | | β3 |
| | | β4 |
| | γ | γ |
| | δ | δ |
| | ε | ε |
| GABA_{A} | α | α1 |
| | | α2 |
| | | α3 |
| | | α4 |
| | | α5 |
| GABA_{A} | α | α6 |
| | β | β1 |
| | | β2 |
| | | β3 |
| | γ | γ1 |
| | | γ2 |
| | | γ3 |
| | δ | δ |
| | ε | ε |
| | π | π |
| | ρ | ρ1 |
| | | ρ2 |
| | | ρ3 |
| P2X | P2X1 | |
| | P2X2 | |
| | P2X3 | |
| | P2X4 | |
| | P2X5 | |
| | P2X6 | |
| | P2X7 | |
| KCNQ | α | Kᵥα1 |
| | | Kᵥα2 |
| | | Kᵥα3 |
| | | Kᵥα4 |
| | | Kᵥα5 |
| | | Kᵥα6 |
| | | Kᵥα7 |
| | | Kᵥα8 |
| | | Kᵥα9 |
| | | Kᵥα10 |
| | | Kᵥα11 |
| | | Kᵥα12 |
| | β | Kᵥβ1 |
| | | Kᵥβ2 |
| | | Kᵥβ3 |
| | | minK |
| | | MiRP1 |
| | | MiRP2 |
| | | MiRP3 |
| | | KCNE1-like |
| | | KCNIP1 |
| | | KCNIP2 |
| | | KCNIP3 |
| | | KCNIP4 |

Illustrative examples of families of LGICs suitable for use in particular embodiments include, but are not limited to Cys-loop receptors such as Glycine receptors (GlyR), serotonin receptors (e.g., 5-HT3 receptors), λ-Aminobutyric Acid A (GABA-A) receptors, and Nicotinic acetylcholine receptors (nAchR); as well as Acid-sensing (proton-gated) ion channels (ASICs), Epithelial sodium channels (ENaC), Ionotropic glutamate receptors, IP3 receptor, P2X receptors, the Ryanodine receptor, and Zinc activated channels (ZAC).

Specific non-limiting examples of LGICs that are suitable for use with the methods described herein include: HTR3A; HTR3B; HTR3C; HTR3D; HTR3E; ASIC1; ASIC2; ASIC3; SCNN1A; SCNN1B; SCNN1D; SCNN1G; GABRA1; GABRA2; GABRA3; GABRA4; GABRA5; GABRA6; GABRB1; GABRB2; GABRB3; GABRG1; GABRG2; GABRG3; GABRD; GABRE; GABRQ; GABRP; GABRR1; GABRR2; GABRR3; GLRA1; GLRA2; GLRA3; GLRA4; GLRB; GRIAl; GRIA2; GRIA3; GRIA4; GRID1; GRID2; GRIK1; GRIK2; GRIK3; GRIK4; GRIK5; GRIN1; GRIN2A; GRIN2B; GRIN2C; GRIN2D; GRIN3A; GRIN33B; ITPR1; ITPR2; ITPR3; CHRNA1; CHRNA2; CHRNA3; CHRNA4; CHRNA5; CHRNA6; CHRNA7; CHRNA9; CHRNA10; CHRNB1; CHRNB2; CHRNB3; CHRNB4; CHRNG; CHRND; CHRNE; P2RX1; P2RX2; P2RX3; P2RX4; P2RX5; P2RX6; P2RX7; RYR1; RYR2; RYR3; and ZACN.

TRPV1, TRPM8 and P2X₂ are members of large LGIC families that share structural features as well as gating principles. For example, TRPV4, similar to TRPV1, is also triggered by heat, but not by capsaicin; and P2X₃, is triggered by ATP, but desensitizes more rapidly than P2X₂. TRPV1, TRPM8 and P2X₂ are, therefore, non-limiting examples of LGIC suitable for use in particular embodiments.

In one embodiment, the engineered receptor is a TRPV1 or TRPM8 receptor or a mutein thereof. TRPV1 and TRPM8, are vanilloid and menthol receptors expressed by nociceptive neurons of the peripheral nervous system. Both channels are thought to function as non-selective, sodium- and calcium-permeable homotetramers. In addition, both channels and their principal agonists—capsaicin and cooling compounds, such as menthol, respectively—are virtually absent from the central nervous system. Capsaicin and some cooling compounds, including menthol and icilin, contain potential acceptor sites for photolabile blocking groups. Association of a photolabile blocking group with such an acceptor would result in a ligand-gated ion channel in which light acts as an indirect trigger by releasing the active ligand.

In one embodiment, the engineered receptor is a P2X₂ receptor or a mutein thereof. P2X₂ is an ATP-gated non-selective cation channel distinguished by its slow rate of desensitization. P2X₂ may be used as a selectively addressable source of depolarizing current and present a platform for the generation of engineered channel-ligand combinations that lack natural agonists altogether.

Non-limiting examples of sequences of wild-type LGIC receptor that find use in the generation of engineered receptors of the present disclosure include the following. In the sequences, the signal peptide is italicized, the ligand binding domain is bolded, and the ion pore domain is underlined:

In some embodiments, the wild-type LGIC receptor is a human alpha 1 glycine receptor (GlyRα1) (GenBank Accession No. NP_001139512.1, SEQ ID NO:2), encoded by the GLRA1 gene (GenBank Accession No. NM_001146040.1 (SEQ ID NO:1):

```
(SEQ ID NO: 2)
                10 20 30 40 50 60
MYSENTLRLY LWETIVEFSL AASKEAEAAR SAPKPMSPSD FLDKLMGRTS GYDARIRPNF
                70 80 90 100 110 120
KGPPVNVSCN IFINSFGSIA ETTMDYRVNI FLRQQWNDPR LAYNEYPDDS LDLDPSMLDS
              130 140 150 160 170 180
IWKPDLFFAN EKGAHFHEIT TDNKLLRISR NGNVLYSIRI TLTLACPMDL KNFPMDVQTC
              190 200 210 220 230 240
IMQLESFGYT MNDLIFEWQE QGAVQVADGL TLPQFILKEE KDLRYCTKHY NTGKFTCIEA
              250 260 270 280 290 300
RFHLERQMGY YLIQMYIPSL LIVILSWISF WINMDAAPAR VGLGITTVLT MTTQSSGSRA
              310 320 330 340 350 360
SLPKVSYVKA IDIWMAVCLL FVESALLEYA AVNEVSRQHK ELLRERRKRR HHKSPMLNLF
              370 380 390 400 410 420
QEDEAGEGRF NFSAYGMGPA CLQAKDGISV KGANNSNTTN PPPAPSKSPE EMRKLFIQRA
              430 440 450 457
KKIDKISRIG FPMAFLIENM FYWIIYKIVR REDVHNQ.
```

In some embodiments, the wild-type LGIC receptor is a human alpha 2 glycine receptor (GlyRα2) (GenBank Accession No. NP_001112357.1, SEQ ID NO: 59), encoded by the GLRA2 gene (GenBank Accession No. NM_001118885.1, SEQ ID NO: 58):

```
(SEQ ID NO: 59)
                  10 20 30 40 50 60
MNRQLVNILT ALFAFFLETN HERTAFCKDH DSRSGKQPSQ TLSPSDFLDK LMGRTSGYDA
                  70 80 90 100 110 120
RIRPNFKGPP VNVTCNIFIN SFGSVTETTM DYRVNIFLRQ QWNDSRLAYS EYPDDSLDLD
              130 140 150 160 170 180
PSMLDSIWKP DLFFANEKGA NFHDVTTDNK LLRISKNGKV LYSIRLTLTL SCPMDLKNEP
              190 200 210 220 230 240
ESFGYTMNDL IFEWLSDGPV QVAEGLTLPQ FILKEEKELG YCTKHYNTGK MDVQTCTMQL
              250 260 270 280 290 300
FTCIEVKFHL ERQMGYYLIQ MYIPSLLIVI LSWVSFWINM DAAPARVALG ITTVLTMTTQ
              310 320 330 340 350 360
SSGSRASLPK VSYVKAIDIW MAVCLLFVFA ALLEYAAVNE VSRQHKEFLR LRRRQKRQNK
              370 380 390 400 410 420
EEDVTRESRF NFSGYGMGHC LQVKDGTAVK ATPANPLPQP PKDGDAIKKK FVDRAKRIDT
              430 440 450 452
ISRAAFPLAF LIFNIFYWIT YKIIRHEDVH KK
```

In some embodiments, the wild-type LGIC receptor is a human alpha 3 glycine receptor (GlyRα3) isoform L (GenBank Accession No. NP_006520.2, SEQ ID NO: 61), encoded by the GLRA3 gene (GenBank Accession No. NM_006529.3, SEQ ID NO: 60):

```
(SEQ ID NO: 61)
                10 20 30 40 50 60
MAHVRHERTL VSGFYFWEAA LLLSLVATKE TDSARSRSAP MSPSDFLDKL MGRTSGYDAR
                  70 80 90 100 110 120
IRPNFKGPPV NVTCNIFINS FGSIAETTMD YRVNIFLRQK WNDPRLAYSE YPDDSLDLDP
              130 140 150 160 170 180
SMLDSIWKPD LFFANEKGAN FHEVTTDNKL LRIFKNGNVL YSIRLTLTLS CPMDLKNFPM
              190 200 210 220 230 240
DVQTCIMQLE SFGYTMNDLI FEWQDEAPVQ VAEGLTLPQF LLKEEKDLRY CTKHYNTGKF
              250 260 270 280 290 300
TCIEVRFHLE RQMGYYLIQM YIPSLLIVIL SWVSEWINMD AAPARVALGI TTVLTMTTQS
              310 320 330 340 350 360
SGSRASLPKV SYVKAIDIWM AVCLLFVESA LLEYAAVNEV SRQHKELLRF RRKRKNKTEA
              370 380 390 400 410 420
FALEKFYRES DMDDEVRESR FSFTAYGMGP CLQAKDGMTP KGPNHPVQVM PKSPDEMRKV
              430 440 450 460 464
FIDRAKKIDT ISRACFPLAF LIFNIFYWVI YKILRHEDIH QQQD
```

In some embodiments, the wild-type LGIC receptor is a human alpha 3 glycine receptor (GlyRα3) isoform K (GenBank Accession No. NP_001036008.1, SEQ ID NO: 69), encoded by the GLRA3 gene (GenBank Accession No. NM_001042543.3, SEQ ID NO: 68):

```
(SEQ ID NO: 69)
                10 20 30 40 50
MAHVRHERTL VSGFYFWEAA LLLSLVATKE TDSARSRSAP MSPSDFLDKL
                60 70 80 90 100
MGRTSGYDAR IRPNFKGPPV NVTCNIFINS FGSIAETTMD YRVNIFLRQK
              110 120 130 140 150
WNDPRLAYSE YPDDSLDLDP SMLDSIWKPD LFFANEKGAN FHEVTTDNKL
              160 170 180 190 200
LRIFKNGNVL YSIRLTLTLS CPMDLKNFPM DVQTCIMQLE SFGYTMNDLI
              210 220 230 240 250
FEWQDEAPVQ VAEGLTLPQF LLKEEKDLRY CTKHYNTGKF TCIEVRFHLE
              260 270 280 290 300
RQMGYYLIQM YIPSLLIVIL SWVSFWINMD AAPARVALGI TTVLTMTTQS
              310 320 330 340 350
SGSRASLPKV SYVKAIDIWM AVCLLEVESA LLEYAAVNEV SRQHKELLRE
              360 370 380 390 400
RRKRKNKDDE VRESRESFTA YGMGPCLQAK DGMTPKGPNH PVQVMPKSPD
              410 420 430 440
EMRKVFIDRA KKIDTISRAC FPLAFLIFNI FYWVIYKILR HEDIHQQQD
```

In some embodiments, the wild-type LGIC receptor is a human nicotinic cholinergic receptor alpha 7 subunit (α7-nAchR) (GenBank Accession No. NP_000737.1, SEQ ID NO:4), encoded by the CHRNA7 gene (GenBank Accession No. NM_000746.5 (SEQ ID NO:3):

```
(SEQ ID NO: 4)
                  10 20 30 40 50 60
MRCSPGGVWL ALAASLLHVS LQGEFQRKLY KELVKNYNPL ERPVANDSQP LTVYFSLSLL
                  70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRT LYYGLNLLIP
              250 260 270 280 290 300
CVLISALALL VELLPADSGE KISLGITVLL SLTVEMLLVA EIMPATSDSV PLIAQYFAST
              310 320 330 340 350 360
MIIVGLSVVV TVIVLQYHHH DPDGGKMPKW TRVILLNWCA WELRMKRPGE DKVRPACQHK
              370 380 390 400 410 420
QRRCSLASVE MSAVAPPPAS NGNLLYIGER GLDGVHCVPT PDSGVVCGRM ACSPTHDEHL
              430 440 450 460 470 480
LHGGQPPEGD PDLAKILEEV RYIANRERCQ DESEAVCSEW KFAACVVDRL CLMAFSVETI
              490 500 502
ICTIGILMSA PNFVEAVSKD FA.
```

In some embodiments, the wild-type LGIC receptor is a human 5-hydroxytryptamine receptor 3A (5HT3A, GenBank Accession No. NP_998786.2, SEQ ID NO:6), encoded by the HTR3A gene (GenBank Accession No. NM_213621.3, SEQ ID NO:5):

```
(SEQ ID NO: 6)
                  10 20 30 40 50 60
MLGKLAMLLW VQQALLALLL PTLLAQGEAR RSRNTTRPAL LRLSDYLLIN YRKGVRPVRD
                  70 80 90 100 110 120
WRKPTTVSID VIVYAILNVD EKNQVLTTYI WYRQYWTDEF LQWNPEDEDN ITKLSIPTDS
              130 140 150 160 170 180
IWVPDILINE FVDVGKSPNI PYVYIRHQGE VQNYKPLQVV TACSLDIYNF PFDVQNCSLT
              190 200 210 220 230 240
FTSWLHTIQD INISLWRLPE KVKSDRSVFM NQGEWELLGV LPYFREFSME SSNYYAEMKF
              250 260 270 280 290 300
YVVIRRRPLF YVVSLLLPSI FLMVMDIVGF YLPPNSGERV SFKITLLLGY SVELIIVSDT
              310 320 330 340 350 360
LPATAIGTPL IGKAPPGSRA QSGEKPAPSH LLHVSLASAL GCTGVYFVVC MALLVISLAE
              370 380 390 400 410 420
TIFIVRLVHK QDLQQPVPAW LRHLVLERIA WLLCLREQST SQRPPATSQA TKTDDCSAMG
              430 440 450 460 470 480
NHCSHMGGPQ DFEKSPRDRC SPPPPPREAS LAVCGLLQEL SSIRQFLEKR DEIREVARDW
              490 500 510 516
LRVGSVLDKL LFHIYLLAVL AYSITLVMLW SIWQYA.
```

In some embodiments, the wild-type LGIC receptor is a human 5-hydroxytryptamine receptor 3B (5HT3B GenBank Accession No. NP_006019.1, SEQ ID NO:57), encoded by the HTR3B gene (GenBank Accession No. NM_006028.4, SEQ ID NO:56):

```
(SEQ ID NO: 57)
                  10 20 30 40 50 60
MLSSVMAPLW ACILVAAGIL ATDTHHPQDS ALYHLSKQLL QKYHKEVRPV YNWTKATTVY
                  70 80 90 100 110 120
LDLFVHAILD VDAENQILKT SVWYQEVWND EFLSWNSSMF DEIREISLPL SAIWAPDIII
              130 140 150 160 170 180
NEFVDIERYP DLPYVYVNSS GTIENYKPIQ VVSACSLETY AFPFDVQNCS LTFKSILHTV
              190 200 210 220 230 240
EDVDLAFLRS PEDIQHDKKA FLNDSEWELL SVSSTYSILQ SSAGGFAQIQ FNVVMRRHPL
              250 260 270 280 290 300
VYVVSLLIPS IFLMLVDLGS FYLPPNCRAR IVEKTSVLVG YTVERVNMSN QVPRSVGSTP
              310 320 330 340 350 360
LIGHFFTICM AFLVLSLAKS IVLVKFLHDE QRGGQEQPFL CLRGDTDADR PRVEPRAQRA
              370 380 390 400 410 420
VVTESSLYGE HLAQPGTLKE VWSQLQSISN YLQTQDQTDQ QEAEWLVLLS REDRLLFQSY
              430 440 441
LEMLGIYTIT LCSLWALWGG V.
```

In some embodiments, the wild-type LGIC receptor is a human Gamma-aminobutyric acid receptor A (GABA-A), subunit beta-3 (GABA-A β3) (GenBank Accession No. NP_000805.1, SEQ ID NO:8), encoded by the GABRB3 gene (GenBank Accession No. NM_000814.5, SEQ ID NO:7):

```
(SEQ ID NO: 8)
                10 20 30 40 50 60
MWGLAGGRLF GIFSAPVLVA VVCCAQSVND PGNMSFVKET VDKLLKGYDI RLRPDFGGPP
                70 80 90 100 110 120
VCVGMNIDIA SIDMVSEVNM DYTLTMYFQQ YWRDKRLAYS GIPLNLTLDN RVADQLWVPD
              130 140 150 160 170 180
TYFLNDKKSF VHGVTVKNRM IRLHPDGTVL YGLRITTTAA CMMDLRRYPL DEQNCTLEIE
              190 200 210 220 230 240
SYGYTTDDIE FYWRGGDKAV TGVERIELPQ FSIVEHRLVS RNVVFATGAY PRLSLSFRLK
              250 260 270 280 290 300
RNIGYFILQT YMPSILITIL SWVSEWINYD ASAARVALGI TTVLTMTTIN THLRETLPKI
              310 320 330 340 350 360
PYVKAIDMYL MGCFVFVFLA LLEYAFVNYI FFGRGPQRQK KLAEKTAKAK NDRSKSESNR
              370 380 390 400 410 420
VDAHGNILLT SLEVHNEMNE VSGGIGDTRN SAISEDNSGI QYRKQSMPRE GHGRELGDRS
              430 440 450 460 470 473
LPHKKTHLRR RSSQLKIKIP DLTDVNAIDR WSRIVEPFTE SLENLVYWLY YVN.
```

In some embodiments, the wild-type LGIC receptor is a human GABA-A, subunit rho1 (ρ1) (GABA-A ρ1) (GenBank Accession No. NP_002033.2, SEQ ID NO:10), encoded by the GABRR1 gene (GenBank Accession No. NM_002042.4, SEQ ID NO:9):

```
(SEQ ID NO: 10)
                10 20 30 40 50 60
MLAVPNMRFG IFLLWWGWVL ATESRMHWPG REVHEMSKKG RPQRQRREVH EDAHKQVSPI
                70 80 90 100 110 120
LRRSPDITKS PLTKSEQLLR IDDHDFSMRP GFGGPAIPVG VDVQVESLDS ISEVDMDFTM
              130 140 150 160 170 180
TLYLRHYWKD ERLSFPSTNN LSMTFDGRLV KKIWVPDMFF VHSKRSFIHD TTTDNVMLRV
              190 200 210 220 230 240
QPDGKVLYSL RVTVTAMCNM DFSRFPLDTQ TCSLEIESYA YTEDDLMLYW KKGNDSLKTD
              250 260 270 280 290 300
ERISLSQFLI QEFHTTTKLA FYSSTGWYNR LYINFTLRRH IFFFLLQTYF PATLMVMLSW
              310 320 330 340 350 360
VSEWIDRRAV PARVPLGITT VLTMSTIITG VNASMPRVSY IKAVDIYLWV SFVEVELSVL
              370 380 390 400 410 420
EYAAVNYLTT VQERKEQKLR EKLPCTSGLP PPRTAMLDGN YSDGEVNDLD NYMPENGEKP
              430 440 450 460 470 479
DRMMVQLTLA SERSSPQRKS QRSSYVSMRI DTHAIDKYSR IIFPAAYILF NLIYWSIFS.
```

In some embodiments, the wild-type LGIC receptor is a human GABA-A, subunit rho2 (ρ2) (GABA-A ρ2) (GenBank Accession No. NP_002034.3, SEQ ID NO:12), encoded by the GABRR2 gene (GenBank Accession No. NM_002043.4, SEQ ID NO: 11):

```
(SEQ ID NO: 12)
                10 20 30 40 50 60
MPYFTRLILF LFCLMVLVES RKPKRKRWTG QVEMPKPSHL YKKNLDVTKI RKGKPQQLLR
                70 80 90 100 110 120
VDEHDFSMRP AFGGPAIPVG VDVQVESLDS ISEVDMDFTM TLYLRHYWKD ERLAFSSASN
              130 140 150 160 170 180
KSMTFDGRLV KKIWVPDVFF VHSKRSFTHD TTTDNIMLRV FPDGHVLYSM RITVTAMCNM
              190 200 210 220 230 240
DFSHFPLDSQ TCSLELESYA YTDEDLMLYW KNGDESLKTD EKISLSQFLI QKFHTTSRLA
              250 260 270 280 290 300
FYSSTGWYNR LYINFTLRRH IFFFLLQTYF PATLMVMLSW VSFWIDRRAV PARVSLGITT
              310 320 330 340 350 360
VLTMTTIITG VNASMPRVSY VKAVDIYLWV SFVEVELSVL EYAAVNYLTT VQERKERKLR
              370 380 390 400 410 420
EKFPCMCGML HSKTMMLDGS YSESEANSLA GYPRSHILTE EERQDKIVVH LGLSGEANAA
              430 440 450 460 465
RKKGLLKGQT GFRIFQNTHA IDKYSRLIFP ASYIFENLIY WSVES.
```

In some embodiments, the wild-type LGIC receptor is a human GABA-A, subunit rho3 (ρ3) (GABA-A ρ3) (GenBank Accession No. NP_001099050.1, SEQ ID NO: 14), encoded by the GABRR3 gene (GenBank Accession No. NM_001105580.2, SEQ ID NO:13):

```
(SEQ ID NO: 14)
                10 20 30 40 50 60
MVLAFQLVSF TYIWIILKPN VCAASNIKMT HQRCSSSMKQ TCKQETRMKK DDSTKARPQK
                70 80 90 100 110 120
YEQLLHIEDN DFAMRPGFGG SPVPVGIDVH VESIDSISET NMDFTMTFYL RHYWKDERLS
              130 140 150 160 170 180
FPSTANKSMT FDHRLTRKIW VPDIFFVHSK RSFIHDTTME NIMLRVHPDG NVLLSLRITV
              190 200 210 220 230 240
SAMCFMDFSR FPLDTQNCSL ELESYAYNED DLMLYWKHGN KSLNTEEHMS LSQFFIEDFS
              250 260 270 280 290 300
ASSGLAFYSS TGWYNRLFIN FVLRRHVFFF VLQTYFPAIL MVMLSWVSEW IDRRAVPARV
              310 320 330 340 350 360
SLGITTVLTM STIITAVSAS MPQVSYLKAV DVYLWVSSLF VELSVIEYAA VNYLTTVEER
              370 380 390 400 410 420
KQFKKTGKIS RMYNIDAVQA MAFDGCYHDS EIDMDQTSLS LNSEDEMRRK SICSPSTDSS
              430 440 450 460 467
RIKRRKSLGG HVGRIILENN HVIDTYSRIL FPIVYILENL FYWGVYV.
```

In some aspects, the subject engineered receptor is a chimeric receptor. In some embodiments, the chimeric receptor comprises a ligand binding domain sequence derived from at least a first LGIC and an ion pore conduction domain sequence, or more simply, “ion pore domain sequence” derived from at least a second LGIC. In some embodiments, the derived amino acid sequence is identical to the corresponding region of the original amino acid sequence. In some embodiments, the derived amino acid sequence may contain alterations in at least one amino acid position compared to the corresponding region of the original amino acid sequence. In some embodiments, an amino acid sequence derived from an original amino acid sequence differs by up to 1, 2, 3, 4, 5, 6, 7, 8, 9, or 10 amino acid residues from the corresponding region of the original amino acid sequence. In some embodiments, a derived amino acid sequence has at least 85%, at least 90%, at least 95%, at least 96%, at least 97%, at least 98%, at least 99%, or at least 99.5% (including all ranges and subranges therebetween) sequence identity to the corresponding region of the original amino acid sequence.

In some embodiments, the first and second LGIC are Cys-loop receptors. Ligand binding domain sequences and ion pore domain sequences of the Cys-loop receptors are well known in the art and can be readily identified from the literature by use of publicly available software, e.g. PubMed, Genbank, Uniprot, and the like. In some embodiments, the ligand binding domain of the chimeric receptor has at least 85%, at least 90%, at least 95%, at least 96%, at least 97%, at least 98%, at least 99%, at least 99.5%, or 100% sequence identity to the ligand binding domain of the first LGIC. In some embodiments, the ion pore domain of the chimeric receptor has at least 85%, at least 90%, at least 95%, at least 96%, at least 97%, at least 98%, at least 99%, at least 99.5%, or 100% sequence identity to the ion pore domain of the second LGIC. In the sequences described above, the ligand binding domain is bolded, and the ion pore domain is underlined.

In some embodiments, the ligand binding domain of the chimeric receptor is derived from the ligand binding domain sequence of a human glycine receptor. In some embodiments, the human glycine receptor is human GlyRα1 (SEQ ID NO:2). In some such embodiments, the ligand binding domain comprises an amino acid sequence having at least 85%, at least 90%, at least 95%, at least 96%, at least 97%, at least 98%, at least 99%, at least 99.5%, or 100% sequence identity to about amino acids 29-235 of GlyRα1, e.g. amino acids 29-235, amino acids 29-240, amino acids 29-246, amino acids 29-248, amino acids 29-250, or amino acids 29-252 of SEQ ID NO:2. In certain such embodiments, the ligand binding domain consists essentially of amino acids 29-235 of SEQ ID NO:2, consists essentially of amino acids 29-240 of SEQ ID NO:2, consists essentially of amino acids 29-246 of SEQ ID NO:2, consists essentially of amino acids 29-248 of SEQ ID NO:2, consists essentially of amino acids 29-250 of SEQ ID NO:2, consists essentially of amino acids 29-252 of SEQ ID NO:2. In some embodiments, the ion pore domain sequence is derived from a Cys-loop receptor other than the human GlyRα1.

In some embodiments, the ligand binding domain of the chimeric receptor comprises the ligand binding domain sequence of a human nicotinic cholinergic receptor. In some embodiments, the human nicotinic cholinergic receptor is human α7-nAChR. In some embodiments, the ligand binding domain comprises about amino acids 23-220 of human α7-nAChR (SEQ ID NO:4), e.g. amino acids 23-220, amino acids 23-221, amino acids 23-222, amino acids 23-223, amino acids 23-224, amino acids 23-225, amino acids 23-226, amino acids 23-227, amino acids 23-228, amino acids 23-229, amino acids 23-230, or amino acids 23-231 of SEQ ID NO:4. In some embodiments, the ligand binding domain consists essentially of amino acids 23-220, amino acids 23-221, amino acids 23-222, amino acids 23-223, amino acids 23-224, amino acids 23-225, amino acids 23-226, amino acids 23-227, amino acids 23-228, amino acids 23-229, amino acids 23-230, or amino acids 23-231 of SEQ ID NO:4. In some embodiments, the ion pore domain sequence is derived from a Cys-loop receptor other than the human α7-nAChR.

In some embodiments, the ligand binding domain of the chimeric receptor is derived from the ligand binding domain sequence of a human nicotinic cholinergic receptor. In some embodiments, the human nicotinic cholinergic receptor is human α7-nAChR. In some embodiments, the ligand binding domain comprises an amino acid sequence having at least 85%, at least 90%, at least 95%, at least 96%, at least 97%, at least 98%, at least 99%, at least 99.5%, or 100% sequence identity to about amino acids 23-220 of human α7-nAChR (SEQ ID NO:4), e.g. amino acids 23-220, amino acids 23-221, amino acids 23-222, amino acids 23-223, amino acids 23-224, amino acids 23-225, amino acids 23-226, amino acids 23-227, amino acids 23-228, amino acids 23-229, amino acids 23-230, or amino acids 23-231 of SEQ ID NO:4. In some embodiments, the ion pore domain sequence is derived from a Cys-loop receptor other than the human α7-nAChR.

In some embodiments, the ligand binding domain of the chimeric receptor is derived from the ligand binding domain sequence of a human serotonin receptor. In some embodiments, the human serotonin receptor is human 5HT3A or 5HT3B. In some such embodiments, the ligand binding domain comprises an amino acid sequence having at least 85%, at least 90%, at least 95%, at least 96%, at least 97%, at least 98%, at least 99%, at least 99.5%, or 100% sequence identity to about amino acids 23-247 of 5HT3A (SEQ ID NO:6), e.g. amino acids 23-240, amino acids 30-245, amino acids 23-247, amino acids 23-250, in some instances amino acids 30-255 of SEQ ID NO:6. In certain embodiments, the ligand binding domain consists essentially of amino acids 23-240 of SEQ ID NO:6, consists essentially of amino acids 23-245 of SEQ ID NO:6, consists essentially of amino acids 30-247 of SEQ ID NO:6, consists essentially of amino acids 23-250 of SEQ ID NO:6, consists essentially of amino acids 23-255 of SEQ ID NO:6. In some such embodiments, the ligand binding domain comprises about amino acids 21-239 of 5HT3B (SEQ ID NO:57), e.g. amino acids 21-232, amino acids 21-235, amino acids 21-240, amino acids 21-245, in some instances amino acids 21-247 of SEQ ID NO:57. In certain embodiments, the ligand binding domain consists essentially of amino acids 21-239 of SEQ ID NO:57, consists essentially of amino acids 21-232 of SEQ ID NO:57, consists essentially of amino acids 21-235 of SEQ ID NO:57, consists essentially of amino acids 21-240 of SEQ ID NO:57, consists essentially of amino acids 21-245 of SEQ ID NO:57. In some embodiments, the ion pore domain sequence is derived from a Cys-loop receptor other than the human 5-hydroxytryptamine receptor 3.

In some embodiments, the ligand binding domain of the chimeric receptor is derived from the ligand binding domain sequence of a human GABA receptor. In some embodiments, the human GABA receptor is human GABA-A β3. In some such embodiments, the ligand binding domain comprises an amino acid sequence having at least 85%, at least 90%, at least 95%, at least 96%, at least 97%, at least 98%, at least 99%, at least 99.5%, or 100% sequence identity to about amino acids 26-245 of GABA-A 33 (SEQ ID NO:8), e.g. amino acids 26-240, amino acids 26-245, amino acids 26-248, amino acids 26-250, in some instances amino acids 26-255 of SEQ ID NO:8. In certain such embodiments, the ligand binding domain consists essentially of amino acids 26-240 of SEQ ID NO:8, consists essentially of amino acids 26-245 of SEQ ID NO:8, consists essentially of amino acids 26-248 of SEQ ID NO:8, consists essentially of amino acids 26-250 of SEQ ID NO:8, or consists essentially of amino acids 26-255 of SEQ ID NO:8. In some embodiments, the ion pore domain sequence is derived from a Cys-loop receptor other than the human GABA-A receptor.

In some embodiments, the ion pore domain to which the ligand binding domain is fused conducts anions, e.g. it comprises an ion pore domain sequence of a human glycine receptor or a human serotonin receptor. In other embodiments, the ion conduction pore domain to which the ligand binding domain is fused conducts cations, e.g. it comprises an ion pore domain sequence of a human acetylcholine receptor or a human gamma-aminobutyric acid receptor A.

In some embodiments, the ion pore domain of the engineered receptor is derived from the ion pore domain sequence of a human glycine receptor. In some embodiments, the human glycine receptor is human GlyRα1. In some embodiments, the ion pore domain comprises an amino acid sequence having at least 85%, at least 90%, at least 95%, at least 96%, at least 97%, at least 98%, at least 99%, at least 99.5%, or 100% sequence identity to about amino acids 245-457 of GlyRα1 (SEQ ID NO:2), e.g. amino acids 240-457, amino acids 245-457, amino acids 248-457, amino acids 249-457, amino acids 250-457, amino acids 255-457, or amino acids 260-457 of SEQ ID NO:2. In some embodiments, the ion pore domain consists essentially of amino acids 245-457 of SEQ ID NO:2, consists essentially of amino acids 248-457 of SEQ ID NO:2, consists essentially of amino acids 249-457 of SEQ ID NO:2, or consists essentially of amino acids 250-457 of SEQ ID NO:2.

In some embodiments, the ion pore domain of the chimeric receptor comprises the ion pore domain sequence of human GlyRα2 (SEQ ID NO: 59). In some embodiments, the ion pore domain of the chimeric receptor comprises, consists essentially of, or consists of an amino acid sequence derived from the ion pore domain sequence of human GlyRα2 (SEQ ID NO: 59). In some embodiments, the ion pore domain of the chimeric receptor comprises, consists essentially of, or consists of an amino acid sequence having at least 85%, at least 90%, at least 95%, at least 96%, at least 97%, at least 98%, at least 99%, or at least 99.5% sequence identity to the ion pore domain sequence of human GlyRα2 (SEQ ID NO: 59). In some embodiments, the ion pore domain of the chimeric receptor comprises, consists essentially of, or consists of an amino acid sequence identical to the ion pore domain sequence of human GlyRα2 (SEQ ID NO: 59). In some embodiments, the ion pore domain sequence of human GlyRα2 comprises, consists essentially of, or consists of amino acids 254-452 of SEQ ID NO: 59. In some embodiments, the ion pore domain sequence of human GlyRα2 comprises, consists essentially of, or consists of amino acids 254-452 of SEQ ID NO: 59. In some embodiments, the ion pore domain sequence of human GlyRα2 comprises, consists essentially of, or consists of amino acids 258-452 of SEQ ID NO: 59. In some embodiments, the ion pore domain sequence of human GlyRα2 comprises, consists essentially of, or consists of amino acids 260-452 of SEQ ID NO: 59.

In some embodiments, the ion pore domain of the chimeric receptor comprises the ion pore domain sequence of human GlyRα3 isoform L (SEQ ID NO: 61). In some embodiments, the ion pore domain of the chimeric receptor comprises, consists essentially of, or consists of an amino acid sequence derived from the ion pore domain sequence of human GlyRα3 isoform L (SEQ ID NO: 61). In some embodiments, the ion pore domain of the chimeric receptor comprises, consists essentially of, or consists of an amino acid sequence having at least 85%, at least 90%, at least 95%, at least 96%, at least 97%, at least 98%, at least 99%, or at least 99.5% sequence identity to the ion pore domain sequence of human GlyRα3 isoform L (SEQ ID NO: 61). In some embodiments, the ion pore domain of the chimeric receptor comprises, consists essentially of, or consists of an amino acid sequence identical to the ion pore domain sequence of human GlyRα3 isoform L (SEQ ID NO: 61). In some embodiments, the ion pore domain sequence of human GlyRα3 isoform L comprises, consists essentially of, or consists of amino acids 253-464 of SEQ ID NO: 61. In some embodiments, the ion pore domain sequence of human GlyRα3 isoform L comprises, consists essentially of, or consists of amino acids 257-464 of SEQ ID NO: 61. In some embodiments, the ion pore domain sequence of human GlyRα3 isoform L comprises, consists essentially of, or consists of amino acids 259-464 of SEQ ID NO: 61.

In some embodiments, the ion pore domain of the chimeric receptor comprises the ion pore domain sequence of human GlyRα3 isoform K (SEQ ID NO: 69). In some embodiments, the ion pore domain of the chimeric receptor comprises, consists essentially of, or consists of an amino acid sequence derived from the ion pore domain sequence of human GlyRα3 isoform K (SEQ ID NO: 69). In some embodiments, the ion pore domain of the chimeric receptor comprises, consists essentially of, or consists of an amino acid sequence having at least 85%, at least 90%, at least 95%, at least 96%, at least 97%, at least 98%, at least 99%, or at least 99.5% sequence identity to the ion pore domain sequence of human GlyRα3 isoform K (SEQ ID NO: 69). In some embodiments, the ion pore domain of the chimeric receptor comprises, consists essentially of, or consists of an amino acid sequence identical to the ion pore domain sequence of human GlyRα3 isoform K (SEQ ID NO: 69). In some embodiments, the ion pore domain sequence of human GlyRα3 isoform K comprises, consists essentially of, or consists of amino acids 253-449 of SEQ ID NO: 69. In some embodiments, the ion pore domain sequence of human GlyRα3 isoform K comprises, consists essentially of, or consists of amino acids 257-449 of SEQ ID NO: 69. In some embodiments, the ion pore domain sequence of human GlyRα3 isoform K comprises, consists essentially of, or consists of amino acids 259-449 of SEQ ID NO: 69.

In some embodiments, the ion pore domain is derived from the ion pore domain sequence of a human nicotinic cholinergic receptor. In some embodiments, the human nicotinic cholinergic receptor is human α7-nAChR. In some embodiments, the ion pore domain comprises an amino acid sequence having at least 85%, at least 90%, at least 95%, at least 96%, at least 97%, at least 98%, at least 99%, at least 99.5%, or 100% sequence identity to about amino acids 230-502 of α7-nAChR (SEQ ID NO:4), e.g. amino acids 227-502, amino acids 230-502, amino acids 231-502, amino acids 232-502, or amino acids 235-502. In certain such embodiments, the ion pore domain consists essentially of amino acids 227-502 of SEQ ID NO:4, consists essentially of amino acids 230-502 of SEQ ID NO:4, consists essentially of amino acids 231-502 of SEQ ID NO:4, consists essentially of amino acids 232-502 of SEQ ID NO:4, or consists essentially of amino acids 235-502 of SEQ ID NO:4.

In some embodiments, the ion pore domain is derived from the ion pore domain sequence of a human serotonin receptor. In some embodiments, the human serotonin receptor is human 5HT3A or 5HT3B. In some embodiments, the ion pore domain comprises an amino acid sequence having at least 85%, at least 90%, at least 95%, at least 96%, at least 97%, at least 98%, at least 99%, at least 99.5%, or 100% sequence identity to about amino acids 248-516 of 5HT3A (SEQ ID NO:6), e.g. amino acids 240-516, amino acids 245-516, amino acids 248-516, amino acids 250-516, or amino acids 255-516 of SEQ ID NO:6. In certain such embodiments, the ion pore domain consists essentially of amino acids 240-516 of SEQ ID NO:6, consists essentially of amino acids 245-516 of SEQ ID NO:6, consists essentially of amino acids 248-516 of SEQ ID NO:6, consists essentially of amino acids 250-516 of SEQ ID NO:6, or consists essentially of amino acids 253-516. In some such embodiments, the ion pore domain comprises an amino acid sequence having at least 85%, at least 90%, at least 95%, at least 96%, at least 97%, at least 98%, at least 99%, at least 99.5%, or 100% sequence identity to about amino acids 240-441 of 5HT3B (SEQ ID NO:57), e.g. amino acids 230-441, amino acids 235-441, amino acids 240-441, amino acids 245-441, or amino acids 250-441 of SEQ ID NO:57. In certain such embodiments, the ion pore domain consists essentially of amino acids 230-441 of SEQ ID NO:57, consists essentially of amino acids 235-441 of SEQ ID NO:57, consists essentially of amino acids 240-441 of SEQ ID NO:57, consists essentially of amino acids 245-441 of SEQ ID NO:57, or consists essentially of amino acids 250-441.

In some embodiments, the ion pore domain is derived from the ion pore domain sequence of a human GABA receptor. In some embodiments, the human GABA receptor is human GABA-A β3. In some embodiments, the ion pore domain comprises an amino acid sequence having at least 85%, at least 90%, at least 95%, at least 96%, at least 97%, at least 98%, at least 99%, at least 99.5%, or 100% sequence identity to about amino acids 246-473 of GABA-A 33 (SEQ ID NO:8), e.g. amino acids 240-473, amino acids 245-473, amino acids 247-473, amino acids 250-473, or amino acids 253-473 of SEQ ID NO:8. In certain such embodiments, the ion pore domain consists essentially of amino acids 240-473 of SEQ ID NO:8, amino acids 245-473 of SEQ ID NO:8, amino acids 247-473 of SEQ ID NO:8, amino acids 250-473 of SEQ ID NO:8, or amino acids 253-473 of SEQ ID NO:8.

In some embodiments, the ion pore domain of the chimeric receptor comprises the ion pore domain sequence of human GABA-A ρ1 (GABRR1, SEQ ID NO: 10). In some embodiments, the ion pore domain of the chimeric receptor comprises, consists essentially of, or consists of an amino acid sequence derived from the ion pore domain sequence of human GABA-A ρ1 (SEQ ID NO: 10). In some embodiments, the ion pore domain of the chimeric receptor comprises, consists essentially of, or consists of an amino acid sequence having at least 85%, at least 90%, at least 95%, at least 96%, at least 97%, at least 98%, at least 99%, or at least 99.5% sequence identity to the ion pore domain sequence of human GABA-A ρ1 (SEQ ID NO: 10). In some embodiments, the ion pore domain of the chimeric receptor comprises, consists essentially of, or consists of an amino acid sequence identical to the ion pore domain sequence of human GABA-A ρ1 (SEQ ID NO: 10). In some embodiments, the ion pore domain sequence of human GABA-A ρ1 comprises, consists essentially of, or consists of amino acids 284-479 of SEQ ID NO: 10. In some embodiments, the ion pore domain sequence of human GABA-A ρ1 comprises, consists essentially of, or consists of amino acids 288-479 of SEQ ID NO: 10. In some embodiments, the ion pore domain sequence of human GABA-A ρ1 comprises, consists essentially of, or consists of amino acids 290-479 of SEQ ID NO: 10.

In some embodiments, the ion pore domain of the chimeric receptor comprises the ion pore domain sequence of human GABA-A ρ2 (GABRR2, SEQ ID NO: 12). In some embodiments, the ion pore domain of the chimeric receptor comprises, consists essentially of, or consists of an amino acid sequence derived from the ion pore domain sequence of human GABA-A ρ2 (SEQ ID NO: 12). In some embodiments, the ion pore domain of the chimeric receptor comprises, consists essentially of, or consists of an amino acid sequence having at least 85%, at least 90%, at least 95%, at least 96%, at least 97%, at least 98%, at least 99%, or at least 99.5% sequence identity to the ion pore domain sequence of human GABA-A ρ2 (SEQ ID NO: 12). In some embodiments, the ion pore domain of the chimeric receptor comprises, consists essentially of, or consists of an amino acid sequence identical to the ion pore domain sequence of human GABA-A ρ2 (SEQ ID NO: 12). In some embodiments, the ion pore domain sequence of human GABA-A ρ2 comprises, consists essentially of, or consists of amino acids 265-466 of SEQ ID NO: 12. In some embodiments, the ion pore domain sequence of human GABA-A ρ2 comprises, consists essentially of, or consists of amino acids 269-466 of SEQ ID NO: 12. In some embodiments, the ion pore domain sequence of human GABA-A ρ2 comprises, consists essentially of, or consists of amino acids 271-466 of SEQ ID NO: 12.

In some embodiments, the ion pore domain of the chimeric receptor comprises the ion pore domain sequence of human GABA-A ρ3 (GABRR3, SEQ ID NO: 14). In some embodiments, the ion pore domain of the chimeric receptor comprises, consists essentially of, or consists of an amino acid sequence derived from the ion pore domain sequence of human GABA-A ρ3 (SEQ ID NO: 14). In some embodiments, the ion pore domain of the chimeric receptor comprises, consists essentially of, or consists of an amino acid sequence having at least 85%, at least 90%, at least 95%, at least 96%, at least 97%, at least 98%, at least 99%, or at least 99.5% sequence identity to the ion pore domain sequence of human GABA-A ρ3 (SEQ ID NO: 14). In some embodiments, the ion pore domain of the chimeric receptor comprises, consists essentially of, or consists of an amino acid sequence identical to the ion pore domain sequence of human GABA-A ρ3 (SEQ ID NO: 14). In some embodiments, the ion pore domain sequence of human GABA-A ρ3 comprises, consists essentially of, or consists of amino acids 271-468 of SEQ ID NO: 14. In some embodiments, the ion pore domain sequence of human GABA-A ρ3 comprises, consists essentially of, or consists of amino acids 275-468 of SEQ ID NO: 14. In some embodiments, the ion pore domain sequence of human GABA-A ρ3 comprises, consists essentially of, or consists of amino acids 277-467 of SEQ ID NO: 14.

The terms “pre-M1 linker”, “pre-M1 linker” and “pre-M1 linker region”, refer to the sequence within a LGIC receptor that is flanked at its amino (N) terminus by the C-terminal end of 010 region of the receptor and at its carboxy (C) terminus by the N-terminal end of transmembrane region 1 (M1) of the receptor. The pre-M1 linker of a LGIC may be readily determined from the art and/or by using any publicly available protein analysis tool, e.g. Expasy, uniProt, etc. In some embodiments, the pre-M1 linker of the LGIC receptor connects the ligand binding domain and the ion pore domain. In some embodiments, the pre-M1 linker of the human α7-nAchR corresponds to amino acids 224-233 of SEQ ID NO:4. In some embodiments, the pre-M1 linker of the human GlyR receptor corresponds to amino acids 242-251 of SEQ ID NO:2, 248-257 of SEQ ID NO:59, or amino acids 247-256 of SEQ ID NO:61 or 69. In some embodiments, the human GlyR receptor is GlyRα1, GlyRα2 or GlyRα3. In some embodiments, the the pre-M1 linker of the GABA-A receptors corresponds to amino acids 275-284 of SEQ ID NO:10. In some embodiments, the GABA-A receptor is GABA-A receptor ρ1, GABA-A receptor ρ2, or GABA-A receptor ρ3.

In some embodiments, the pre-M1 linker of the chimeric LGIC receptor comprises or consists of the pre-M1 linker sequence of one of the wildtype LGIC receptors from which the ligand binding domain or the ion pore domain of the chimeric LGIC receptor is derived. In some embodiments, the chimeric LGIC receptor comprises a pre-M1 linker sequence that is different from both of the pre-M1 linker sequences of the wildtype LGIC receptors from which the ligand binding domain and the ion pore domain of the chimeric LGIC receptor is derived. In some embodiments, the pre-M1 linker of the chimeric LGIC receptor comprises or consists of a pre-M1 linker that is a chimeric sequence based on those of the wildtype LGIC receptors.

In some embodiments, the chimeric LGIC receptor comprises a ligand binding domain sequence derived from a first LGIC and an ion pore domain sequence derived from a second LGIC, and the pre-M1 linker of the chimeric LGIC receptor comprises or consists of an N-term part derived from the corresponding region of the first LGIC and a C-term part derived from the corresponding region of the second LGIC.

In some embodiments, the chimeric LGIC receptor comprises a ligand binding domain sequence derived from α7-nAchR and an ion pore domain sequence derived from a GlyRα family receptor (e.g., GlyRα1, GlyRα2 or GlyRα3), and the pre-M1 linker of the chimeric LGIC receptor comprises or consists of any one of the sequences in Table 10 below. In some embodiments, the pre-M1 linker comprises or consists of a sequence having at least 70%, at least 80%, or at least 90% identity to any one of the sequences in Table 10 below.

TABLE 10
List of Pre-M1 Linker

| Name | Sequence | SEQ ID NO: |
|---|---|---|
| α7-nAchR Pre-M1 linker | VTMRRRTLYY | 72 |
| GlyRα1, α2 and α3 Pre-M1 | FHLERQMGYY | 73 |
| linker | | |
| Pre-M1 linker #1 | VTMRRRTGYY | 74 |
| Pre-M1 linker #2 | VTMRRRMGYY | 75 |
| Pre-M1 linker #3 | VTMRRQMGYY | 76 |
| Pre-M1 linker #4 | VTMERQMGYY | 77 |
| Pre-M1 linker #5 | VTLERQMGYY | 78 |
| Pre-M1 linker #6 | VHLERQMGYY | 79 |

In some embodiments, the chimeric LGIC receptor comprises a ligand binding domain sequence derived from α7-nAchR and an ion pore domain sequence derived from a GABA-A family receptor selected from GABA-A ρ1 receptor, GABA-A ρ2 receptor and GABA-A ρ3 receptor, and the pre-M1 linker of the chimeric LGIC receptor comprises or consists of any one of the sequences in Table 11 below. In some embodiments, the pre-M1 linker comprises or consists of a sequence having at least 70%, at least 80%, or at least 90% identity to any one of the sequences in Table 11 below.

TABLE 11
List of Pre-M1 Linker

| Name | Sequence | SEQ ID NO: |
|---|---|---|
| α7-nAchR Pre-M1 linker | VTMRRRTLYY | 72 |
| GABA-A ρ1, ρ2 Pre-M1 linker | FTLRRHIFFF | 80 |
| GABA-A ρ3 pre-M1 linker | FVLRRHVFFF | 81 |
| Pre-M1 linker #7 | VTMRRRTLYF | 82 |
| Pre-M1 linker #8 | VTMRRRTLFF | 83 |
| Pre-M1 linker #9 | VTMRRRTFFF | 84 |
| Pre-M1 linker #10 | VTMRRRIFFF | 85 |
| Pre-M1 linker #11 | VTMRRHIFFF | 86 |
| Pre-M1 linker #12 | VTLRRHIFFF | 87 |
| Pre-M1 linker #13 | VTMRRRVFFF | 88 |
| Pre-M1 linker #14 | VTMRRHVFFF | 89 |
| Pre-M1 linker #15 | VTLRRHVFFF | 90 |
| Pre-M1 linker #16 | VVLRRHVFFF | 91 |

In some embodiments, the ion pore domain of the subject chimeric ligand-gated ion channel comprises an M2-M3 linker domain that is heterologous to the M2-M3 linker domain of the ion pore domain. By an “M2-M3 linker domain”, or “M2-M3 linker”, it is meant the sequence within an ion pore domain of a LGIC that is flanked at its amino (N) terminus by the C-terminal end of transmembrane domain 2 (M2) of the receptor and at its carboxy (C) terminus by the N-terminal end of transmembrane domain 3 (M3) of the receptor. The M2-M3 linker of a LGIC may be readily determined from the art and/or by using any publicly available protein analysis tool, e.g. Expasy, uniProt, etc. In some embodiments, when the ion pore domain of a chimeric receptor comprises a heterologous M2-M3 linker, the M2-M3 linker is derived from the same receptor as the ligand binding domain of the chimeric receptor. For example, when the subject ligand-gated ion channel comprises a ligand binding domain from an AChR and an ion pore domain from a GlyR, its ion pore domain sequence may comprise a M2-M3 linker sequence derived from the AChR. In some embodiments, the ion pore domain is derived from GlyRα1 and the M2-M3 linker is derived from α7-nAChR. In some embodiments, the M2-M3 linker sequence that is removed from the ion pore domain corresponds to about amino acids 302-313 of GlyRα1 (SEQ ID NO:2). In some embodiments, the ion pore domain is derived from GlyRα2 and the M2-M3 linker is derived from α7-nAChR. In some embodiments, the M2-M3 linker sequence that is removed from the ion pore domain corresponds to about amino acids 308-319 of GlyRα2 (SEQ ID NO:59). In some embodiments, the ion pore domain is derived from GlyRα3 and the M2-M3 linker is derived from α7-nAChR. In some embodiments, the M2-M3 linker sequence that is removed from the ion pore domain corresponds to about amino acids 307-318 of GlyRα3 (SEQ ID NO:61 or 69). In some embodiments, the ion pore domain is derived from GABA-A ρ1, and the M2-M3 linker is derived from α7-nAChR. In some embodiments, the M2-M3 linker sequence that is removed from the ion pore domain corresponds to about amino acids 335-346 of GABA-A ρ1 (SEQ ID NO:10). In some embodiments, the ion pore domain is derived from GABA-A ρ2, and the M2-M3 linker is derived from α7-nAChR. In some embodiments, the M2-M3 linker sequence that is removed from the ion pore domain corresponds to about amino acids 315-326 of GABA-A ρ2 (SEQ ID NO:12). In some embodiments, the ion pore domain is derived from GABA-A ρ3, and the M2-M3 linker is derived from α7-nAChR. In some embodiments, the M2-M3 linker sequence that is removed from the ion pore domain corresponds to about amino acids 321-332 of GABA-A ρ3 (SEQ ID NO:14). In some embodiments, the M2-M3 linker that is inserted is derived from about amino acids 283-295 of α7-nAChR (SEQ ID NO:4), e.g. amino acids 290-295, 283-290, 283-295, 287-292, etc. or a sequence at least 50%, at least 55%, at least 60%, at least 65%, at least 70%, at least 75%, at least 80%, at least 85%, at least 90%, at least 95%, or 100% identical to amino acids 283-295 of α7-nAChR, including all ranges and subranges therebetween. In some embodiments, the M2-M3 linker that is inserted is 1, 2, 3, 4, 5, 6, 7, 8, 9, 10, 11, 12, 13, 14, 15, 16, 17, 18, 19, or 20 amino acids in length. In some embodiments, the length of the M2-M3 linker that is inserted is from 9 to 16 amino acids, from 10 to 15 amino acids, from 11 to 14 amino acids, or from 12 to 13 amino acids, including all ranges and subranges therebetween.

In some embodiments, the ligand binding domain of the subject chimeric ligand-gated ion channel comprises a Cys-loop domain sequence that is heterologous to the Cys-loop sequence of the ligand binding domain. By a “Cys-loop domain sequence”, or “Cys-loop sequence”, it is meant the domain within a ligand binding domain of a Cys-loop LGIC that forms a loop structure flanked by a cysteine at the N-terminus and the C-terminus. Without wishing to be bound by theory, it is believed that upon binding of the ligand to the ligand binding domain, the Cys-loop structurally moves to be in close proximity to the M2-M3 loop, this movement mediating the biophysical translation of ligand binding in the extracellular domain to signal transduction in the ion pore domain (as reviewed in Miller and Smart, Trends in Pharmacological Sci 2009:31(4)). The substitution of an endogenous Cys-loop sequence with a heterologous Cys-loop sequence may increase the conductivity of the LGIC by 1.5-fold or more, e.g. at least 2-fold, 3-fold or 4-fold, in some instances at least 5-fold or 6-fold, and at certain doses, at least 7-fold, 8-fold, 9-fold or 10-fold. The Cys-loop domain of a Cys-loop receptor may be readily determined from the art and/or by using any publicly available protein analysis tool, e.g. Expasy, uniProt, etc. Typically, when the ligand binding domain of a chimeric receptor comprises a heterologous Cys-loop sequence, the Cys-loop sequence is derived from the same receptor as the ion pore domain of the chimeric receptor. For example, when the subject chimeric ligand-gated ion channel comprises a ligand binding domain from an AChR and an ion pore domain from a GlyR, the subject ligand-gated ion channel may comprise ligand binding domain sequence from an AChR except for the sequence of the Cys-loop domain, which is instead derived from a GlyR. In some embodiments, the ligand binding domain is derived from α7-nAChR and the Cys-loop sequence is from GlyRα1, GlyRα2 or GlyRα3. In some such embodiments, the Cys-loop sequence that is removed from the ligand binding domain corresponds to about amino acids 150-164 of α7-nAChR (SEQ ID NO:4), e.g. amino acids 150-157 of α7-nAChR. In some embodiments, the Cys loop sequence that is inserted is derived from about amino acids 166-180 of GlyRα1 (SEQ ID NO:2), e.g. amino acids 166-172 of GlyRα1, or a sequence at least 80%, at least 85%, at least 90%, or at least 95% identical to amino acids 166-180 of GlyRα1. In some embodiments, the Cys loop sequence that is inserted is derived from about amino acids 172-186 of GlyRα2 (SEQ ID NO:59), e.g. amino acids 172-178 of GlyRα2, or a sequence at least 80%, at least 85%, at least 90%, or at least 95% identical to amino acids 172-186 of GlyRα2. In some embodiments, the Cys loop sequence that is inserted is derived from about amino acids 171-185 of GlyRα3 (SEQ ID NO:61 or 69), e.g. amino acids 171-177 of GlyRα3, or a sequence at least 80%, at least 85%, at least 90%, or at least 95% identical to amino acids 171-185 of GlyRα3. In some embodiments, the Cys loop sequence that is inserted is derived from about amino acids 198-212 of GABA-A ρ1 (SEQ ID NO:10), e.g. amino acids 198-204 of GABA-A ρ 1, or a sequence at least 80%, at least 85%, at least 90%, or at least 95% identical to amino acids 198-212 of GABA-A ρ1. In some embodiments, the Cys loop sequence that is inserted is derived from about amino acids 178-192 of GABA-A ρ2 (SEQ ID NO:12), e.g. amino acids 178-184 of GABA-A ρ2, or a sequence at least 80%, at least 85%, at least 90%, or at least 95% identical to amino acids 178-192 of GABA-A ρ2. In some embodiments, the Cys loop sequence that is inserted is derived from about amino acids 184-198 of GABA-A ρ3 (SEQ ID NO:14), e.g. amino acids 184-190 of GABA-A ρ3, or a sequence at least 80%, at least 85%, at least 90%, or at least 95% identical to amino acids 184-198 of GABA-A ρ3.

In some embodiments, the ligand binding domain of the subject chimeric ligand-gated ion channel comprises a β1-2 loop domain sequence that is heterologous to the β1-2 loop domain sequence of the ligand binding domain. By a “β1-2 loop domain sequence”, or “β1-2 loop, or β1-β2 loop”, it is meant the domain within a ligand binding domain of a Cys-loop LGIC that is flanked at its N-terminus by the C-terminus of the β1 sheet and, at its C-terminus, by the N-terminus of the β2 sheet. Without wishing to be bound by theory, it is believed that the β1-2 loop helps to mediate biophysical translation of ligand binding in the extracellular domain to the ion pore domain and subsequent signal transduction (i.e. chloride influx in case of GlyR). It is believed that upon binding of ligand, the β1-2 loop, together with the Cys-loop, come in close proximity to the M2-M3 loop to mediate the biophysical translation of ligand binding in the extracellular domain to signal transduction in the ion pore domain where the M2-M3 loop resides (as reviewed in Miller and Smart, supra). The substitution of an endogenous β1-2 loop sequence with a heterologous β1-2 loop sequence may increase the conductivity of the LGIC by 1.5-fold or more, e.g. at least 2-fold, 3-fold or 4-fold, in some instances at least 5-fold or 6-fold, and at certain doses, at least 7-fold, 8-fold, 9-fold or 10-fold. The β1-2 loop of a Cys-loop receptor may be readily determined from the art and/or by using any publicly available protein analysis tool, e.g. Expasy, uniProt, etc. Typically, when the ligand binding domain of a chimeric receptor comprises a heterologous β1-2 loop sequence, the ββ1-2 loop sequence is derived from the same receptor as the ion pore domain of the chimeric receptor. For example, when the subject chimeric ligand-gated ion channel comprises a ligand binding domain derived from an AChR and an ion pore domain derived from a GlyR, the sequence of the ββ1-2 loop domain of the ligand binding domain may be derived from the GlyR. In some embodiments, the ligand binding domain is derived from α7-nAChR. In some embodiments, the β1-2 loop sequence that is removed from the ligand binding domain correspond to about amino acids 67-70 of α7-nAChR (SEQ ID NO:4), e.g. amino acids 67-70, 66-71 or 64-72 of α7-nAChR. In some embodiments, the β1-2 loop sequence that is removed from the ligand binding domain correspond to about amino acids 66-71 of α7-nAChR (SEQ ID NO:4), In some embodiments, the ion pore domain is derived from GlyRα1 and the β1-2 loop that is inserted corresponds to about amino acids 80-85 of GlyRα1 (SEQ ID NO:2) with at most 3, at most 2, at most 1, or no amino acid mutations. In some embodiments, the ion pore domain is derived from GlyRα2 and the β1-2 loop that is inserted corresponds to about amino acids 86-91 of GlyRα2 (SEQ ID NO:59) with at most 3, at most 2, at most 1, or no amino acid mutations. In some embodiments, the ion pore domain is derived from GlyRα3 and the β1-2 loop that is inserted corresponds to about amino acids 85-90 of GlyRα3 (SEQ ID NO:61 or 69) with at most 3, at most 2, at most 1, or no amino acid mutations. In some embodiments, the ion pore domain is derived from GABA-A ρ1 and the β1-2 loop that is inserted corresponds to about amino acids 112-117 of GABA-A ρ1 (SEQ ID NO:10) with at most 3, at most 2, at most 1, or no amino acid mutations. In some embodiments, the ion pore domain is derived from GABA-A ρ2 and the β1-2 loop that is inserted corresponds to about amino acids 92-97 of GABA-A ρ2 (SEQ ID NO:12) with at most 3, at most 2, at most 1, or no amino acid mutations. In some embodiments, the ion pore domain is derived from GABA-A ρ3 and the the β1-2 loop that is inserted corresponds to about amino acids 98-103 of GABA-A ρ3 (SEQ ID NO:14) with at most 3, at most 2, at most 1, or no amino acid mutations.

Non-limiting examples of sequences of chimeric LGIC receptors of the present disclosure include the sequences disclosed herein as SEQ ID NO:15-SEQ ID NO:52. In some embodiments, the chimeric LGIC receptor or the polynucleotide that encodes it has a sequence identity of 85% or more to a sequence provided in SEQ ID NO:15-SEQ ID NO:52 herein, e.g. a sequence identity of 90% or more, 93% or more, or 95% or more, i.e. about 96%, about 97%, about 98%, about 99% or about 100% to a sequence provided in SEQ ID NO:15-SEQ ID NO:52. In the sequences, the signal peptide is italicized, the ligand binding domain is bolded, and the ion pore domain is underlined.

In some embodiments, the chimeric LGIC receptor is a CHRNA7/GLRA1 chimera (R229 junction), comprising the human α7-nAChR signal peptide (italics) and ligand binding domain (bold), fused to the human GlyRα1 ion pore domain (underlined):

```
  (SEQ ID NO: 16, encoded by SEQ ID NO: 15)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SFWINMDAAP ARVGLGITTV LTMTTQSSGS RASLPKVSYV KAIDIWMAVC
              310 320 330 340 350 360
LLEVESALLE YAAVNEVSRQ HKELLRERRK RRHHKSPMLN LFQEDEAGEG RENESAYGMG
              370 380 390 400 410 420
PACLQAKDGI SVKGANNSNT INPPPAPSKS PEEMRKLFIQ RAKKIDKISR IGFPMAFLIF
              430 439
NMFYWIIYKI VRREDVHNQ.
```

In some embodiments, the chimeric LGIC receptor is a CHRNA7/GLRA1 (R228 junction) chimera, comprising the human α7-nAChR signal peptide (italics) and ligand binding domain (bold), fused to the human GlyRα1 ion pore domain (underlined):

```
  (SEQ ID NO: 17)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRQM GYYLIQMYIP
              250 260 270 280 290 300
SLLIVILSWI SEWINMDAAP ARVGLGITTV LTMTTQSSGS RASLPKVSYV KAIDIWMAVC
              310 320 330 340 350 360
LLEVESALLE YAAVNEVSRQ HKELLRERRK RRHHKSPMLN LFQEDEAGEG RENESAYGMG
              370 380 390 400 410 420
PACLQAKDGI SVKGANNSNT TNPPPAPSKS PEEMRKLFIQ RAKKIDKISR IGFPMAFLIE
              430 439
NMFYWIIYKI VRREDVHNQ.
```

In some embodiments, the chimeric LGIC receptor is a CHRNA7/GLRA1 (V224 junction) chimera, comprising the human α7-nAChR signal peptide (italics) and ligand binding domain (bold), fused to the human GlyRα1 ion pore domain (underlined):

```
  (SEQ ID NO: 18)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVHLERQM GYYLIQMYIP
              250 260 270 280 290 300
SLLIVILSWI SEWINMDAAP ARVGLGITTV LTMTTQSSGS RASLPKVSYV KAIDIWMAVC
              310 320 330 340 350 360
LLFVFSALLE YAAVNEVSRQ HKELLRERRK RRHHKSPMLN LFQEDEAGEG RFNFSAYGMG
              370 380 390 400 410 420
PACLQAKDGI SVKGANNSNT TNPPPAPSKS PEEMRKLFIQ RAKKIDKISR IGFPMAFLIE
              430 439
NMFYWIIYKI VRREDVHNQ.
```

In some embodiments, the chimeric LGIC receptor is a CHRNA7/GLRA1 (Y233 junction) chimera, comprising the human α7-nAChR signal peptide (italics) and ligand binding domain (bold), fused to the human GlyRα1 ion pore domain (underlined):

```
(SEQ ID NO: 19)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRT LYYLIQMYIP
              250 260 270 280 290 300
SLLIVILSWI SFWINMDAAP ARVGLGITTV LTMTTQSSGS RASLPKVSYV KAIDIWMAVC
              310 320 330 340 350 360
LLEVESALLE YAAVNEVSRQ HKELLRERRK RRHHKSPMLN LFQEDEAGEG RENESAYGMG
              370 380 390 400 410 420
PACLQAKDGI SVKGANNSNT TNPPPAPSKS PEEMRKLFIQ RAKKIDKISR IGFPMAFLIE
              430 439
NMFYWIIYKI VRREDVHNQ.
```

In some embodiments, the chimeric LGIC receptor is a CHRNA7/GLRA1 chimera (R229 junction), comprising the human α7-nAChR signal peptide (italics) and ligand binding domain (bold), fused to the human GlyRα1 ion pore domain (underlined) comprising an α7-nAChR M2-M3 linker (lowercase):

```
(a)
  (SEQ ID NO: 21, encoded by SEQ ID NO: 20)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SEWINMDAAP ARVGLGITTV LTMTTQSSGS eimpatsdsv SYVKAIDIWM
              310 320 330 340 350 360
AVCLLEVESA LLEYAAVNEV SRQHKELLRF RRKRRHHKSP MLNLFQEDEA GEGRENESAY
              370 380 390 400 410 420
GMGPACLQAK DGISVKGANN SNTTNPPPAP SKSPEEMRKL FIQRAKKIDK ISRIGEPMAF
              430 440 442
LIFNMFYWII YKIVRREDVH NQ;
(b)
  (SEQ ID NO: 23, encoded by SEQ ID NO: 22)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SFWINMDAAP ARVGLGITTV LTMTTQSSGS eimpatsdsv pliaqAIDIW
              310 320 330 340 350 360
MAVCLLEVES ALLEYAAVNE VSRQHKELLR FRRKRRHHKS PMLNLFQEDE AGEGRENESA
              370 380 390 400 410 420
YGMGPACLQA KDGISVKGAN NSNTTNPPPA PSKSPEEMRK LFIQRAKKID KISRIGEPMA
              430 440 443
FLIFNMFYWI IYKIVRREDV HNQ;
(c)
(SEQ ID NO: 25, encoded by SEQ ID NO: 24)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SEWINMDAAP ARVGLGITTV LTMTTQSSGS RASLPKVsds vplIDIWMAV
              310 320 330 340 350 360
CLLFVFSALL EYAAVNEVSR QHKELLRERR KRRHHKSPML NLFQEDEAGE GRENESAYGM
              370 380 390 400 410 420
GPACLQAKDG ISVKGANNSN TTNPPPAPSK SPEEMRKLFI QRAKKIDKIS RIGFPMAFLI
              430 440
FNMFYWIIYK IVRREDVHNQ;
(d)
  (SEQ ID NO: 27, encoded by SEQ ID NO: 26)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMERQM GYYLIQMYIP
              250 260 270 280 290 300
SLLIVILSWI SEWINMDAAP ARVGLGITTV LTMTTQSSGS eimpatsdsv pliaqAIDIW
              310 320 330 340 350 360
MAVCLLEVES ALLEYAAVNE VSRQHKELLR FRRKRRHHKS PMLNLFQEDE AGEGRENESA
              370 380 390 400 410 420
YGMGPACLQA KDGISVKGAN NSNTTNPPPA PSKSPEEMRK LFIQRAKKID KISRIGFPMA
              430 440 443
FLIENMFYWI IYKIVRREDV HNQ;
(e)
  (SEQ ID NO:29, encoded by SEQ ID NO: 28)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRT GYYLIQMYIP
              250 260 270 280 290 300
SLLIVILSWI SFWINMDAAP ARVGLGITTV LTMTTQSSGS eimpatsdsv pliaqAIDIW
              310 320 330 340 350 360
MAVCLLEVES ALLEYAAVNE VSRQHKELLR FRRKRRHHKS PMLNLFQEDE AGEGRENESA
              370 380 390 400 410 420
YGMGPACLQA KDGISVKGAN NSNTTNPPPA PSKSPEEMRK LFIQRAKKID KISRIGFPMA
              430 440 443
FLIFNMFYWI IYKIVRREDV HNQ;
or
(f)
  (SEQ ID NO: 31, encoded by SEQ ID NO: 30)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRT LYYLIQMYIP
              250 260 270 280 290 300
SLLIVILSWI SFWINMDAAP ARVGLGITTV LTMTTQSSGS eimpatsdsv pliaqAIDIW
              310 320 330 340 350 360
MAVCLLEVES ALLEYAAVNE VSRQHKELLR FRRKRRHHKS PMLNLFQEDE AGEGRENESA
              370 380 390 400 410 420
YGMGPACLQA KDGISVKGAN NSNTINPPPA PSKSPEEMRK LFIQRAKKID KISRIGEPMA
              430 440 443
FLIFNMFYWI IYKIVRREDV HNQ.
```

In some embodiments, the chimeric LGIC receptor is a CHRNA7/GLRA1 chimera comprising the human α7-nAChR signal peptide (italics) and ligand binding domain (bold) comprising an GlyRα1 Cys-loop sequence (lowercase); fused to the human GlyRα1 ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:33:

```
  (SEQ ID NO: 33, encoded by SEQ ID NO: 32)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknfpmd vqtcKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SFWINMDAAP ARVGLGITTV LTMTTQSSGS RASLPKVSYV KAIDIWMAVC
              310 320 330 340 350 360
LLEVESALLE YAAVNEVSRQ HKELLRERRK RRHHKSPMLN LFQEDEAGEG RENESAYGMG
              370 380 390 400 410 420
PACLQAKDGI SVKGANNSNT TNPPPAPSKS PEEMRKLFIQ RAKKIDKISR IGFPMAFLIF
              430 439
NMFYWIIYKI VRREDVHNQ.
(a)
  (SEQ ID NO: 35, encoded by SEQ ID NO: 34)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SFWINMDAAP ARVGLGITTV LTMTTQSSGS RASLPKVSYV KAIDIWMAVC
              310 320 330 340 350 360
LLEVESALLE YAAVNEVSRQ HKELLRERRK RRHHKSPMLN LFQEDEAGEG RENESAYGMG
              370 380 390 400 410 420
PACLQAKDGI SVKGANNSNT TNPPPAPSKS PEEMRKLFIQ RAKKIDKISR IGFPMAFLIF
              430 439
NMFYWIIYKI VRREDVHNQ.
```

In some embodiments, the chimeric LGIC receptor is a CHRNA7/GLRA1 chimera comprising the human α7-nAChR signal peptide (italics) and ligand binding domain (bold) comprising an GlyRα1 β1-2 loop sequence (lowercase); fused to the human GlyRα1 ion pore domain (underlined):

```
(a)
  (SEQ ID NO: 37, encoded by SEQ ID NO: 36)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDettm VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SFWINMDAAP ARVGLGITTV LTMTTQSSGS RASLPKVSYV KAIDIWMAVC
              310 320 330 340 350 360
LLEVESALLE YAAVNEVSRQ HKELLRERRK RRHHKSPMLN LFQEDEAGEG RFNFSAYGMG
              370 380 390 400 410 420
PACLQAKDGI SVKGANNSNT TNPPPAPSKS PEEMRKLFIQ RAKKIDKISR IGFPMAFLIF
              430 439
NMFYWIIYKI VRREDVHNQ.
```

In some embodiments, the chimeric LGIC receptor is a CHRNA7/GLRA1 chimera comprising the human α7-nAChR signal peptide (italics) and ligand binding domain (bold) comprising an GlyRα1 β1-2 loop sequence (lowercase) and Cys-loop sequence (lowercase); fused to the human GlyRα1 ion pore domain (underlined):

```
(a)
  (SEQ ID NO: 39, encoded by SEQ ID NO: 38)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDiaettm dLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknfpmd vqtcKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SFWINMDAAP ARVGLGITTV LTMTTQSSGS RASLPKVSYV KAIDIWMAVC
              310 320 330 340 350 360
LLEVESALLE YAAVNEVSRQ HKELLRERRK RRHHKSPMLN LFQEDEAGEG RENESAYGMG
              370 380 390 400 410 420
PACLQAKDGI SVKGANNSNT TNPPPAPSKS PEEMRKLFIQ RAKKIDKISR IGFPMAFLIF
              430 439
NMFYWIIYKI VRREDVHNQ.
(b)
  (SEQ ID NO: 41, encoded by SEQ ID NO: 40)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDettm VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknfpmd vqtcKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SEWINMDAAP ARVGLGITTV LTMTTQSSGS RASLPKVSYV KAIDIWMAVC
              310 320 330 340 350 360
LLEVESALLE YAAVNEVSRQ HKELLRERRK RRHHKSPMLN LFQEDEAGEG RENESAYGMG
              370 380 390 400 410 420
PACLQAKDGI SVKGANNSNT TNPPPAPSKS PEEMRKLFIQ RAKKIDKISR IGFPMAFLIF
              430 439
NMFYWIIYKI VRREDVHNQ.
(c)
  (SEQ ID NO: 43, encoded by SEQ ID NO: 42)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDettm VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknfpmd vqtcKLKFGS WSYGGWSLDL
              190 200 210 220 230 240
QMQEADISGY IPNGEWDLVG IPGKRSERFY ECCKEPYPDV TFTVTMERQM GYYLIQMYIP
              250 260 270 280 290 300
SLLIVILSWI SEWINMDAAP ARVGLGITTV LTMTTQSSGS RASLPKVSYV KAIDIWMAVC
              310 320 330 340 350 360
LLEVESALLE YAAVNEVSRQ HKELLRERRK RRHHKSPMLN LFQEDEAGEG RENESAYGMG
              370 380 390 400 410 420
PACLQAKDGI SVKGANNSNT TNPPPAPSKS PEEMRKLFIQ RAKKIDKISR IGFPMAFLIF
              430 439
NMFYWIIYKI VRREDVHNQ.
(d)
  (SEQ ID NO: 45, encoded by SEQ ID NO: 44)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDiaettm dLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknfpmd vqtcKLKFGS WSYGGWSLDL
              190 200 210 220 230 240
QMQEADISGY IPNGEWDLVG IPGKRSERFY ECCKEPYPDV TFTVTMERQM GYYLIQMYIP
              250 260 270 280 290 300
SLLIVILSWI SEWINMDAAP ARVGLGITTV LTMTTQSSGS RASLPKVSYV KAIDIWMAVC
              310 320 330 340 350 360
LLFVFSALLE YAAVNEVSRQ HKELLRERRK RRHHKSPMLN LFQEDEAGEG RENESAYGMG
              370 380 390 400 410 420
PACLQAKDGI SVKGANNSNT TNPPPAPSKS PEEMRKLFIQ RAKKIDKISR IGFPMAFLIE
              430 439
NMFYWIIYKI VRREDVHNQ.
```

In some embodiments, the chimeric LGIC receptor is a CHRNA7/GLRA1 chimera comprising the human α7-nAChR signal peptide (italics) and ligand binding domain (bold) comprising an GlyRα1 β1-2 loop sequence (lowercase); fused to the human GlyRα1 ion pore domain (underlined) comprising human α7-nAChR M2-M3 linker (lowercase):

```
(a)
  (SEQ ID NO:47, encoded by SEQ ID NO: 46)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDettm VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SFWINMDAAP ARVGLGITTV LTMTTQSSGS eimpatsdsv pliaqAIDIW
              310 320 330 340 350 360
MAVCLLEVES ALLEYAAVNE VSRQHKELLR FRRKRRHHKS PMLNLFQEDE AGEGRENESA
              370 380 390 400 410 420
YGMGPACLQA KDGISVKGAN NSNTTNPPPA PSKSPEEMRK LFIQRAKKID KISRIGEPMA
              430 440 443
FLIENMEYWI IYKIVRREDV HNQ.
```

In some embodiments, the chimeric LGIC receptor is a CHRNA7/GLRA1 chimera comprising the human α7-nAChR signal peptide (italics) and ligand binding domain (bold) comprising a GlyRα1 Cys-loop sequence (lowercase); fused to the human GlyRα1 ion pore domain (underlined) comprising a human α7-nAChR M2-M3 linker (lowercase):

```
(a)
(SEQ ID NO: 49, encoded by SEQ ID NO: 48)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknfpmd vqtcKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWI SFWINMDAAP ARVGLGITTV LTMTTQSSGS eimpatsdsv pliaqAIDIW
              310 320 330 340 350 360
MAVCLLEVES ALLEYAAVNE VSRQHKELLR FRRKRRHHKS PMLNLFQEDE AGEGRENESA
              370 380 390 400 410 420
YGMGPACLQA KDGISVKGAN NSNTTNPPPA PSKSPEEMRK LFIQRAKKID KISRIGFPMA
              430 440 443
FLIENMFYWI IYKIVRREDV HNQ.
```

In some embodiments, the chimeric LGIC receptor is a HTR3A/GLRA1 chimera (R241 junction), comprising the human 5HT3A serotonin receptor signal peptide (italics) and ligand binding domain (bold), fused to the human GlyRα1 ion pore domain (underlined):

```
(a)
(SEQ ID NO: 50)
                10 20 30 40 50 60
MLLWVQQALL ALLLPTLLAQ GEARRSRNTT RPALLRLSDY LLTNYRKGVR PVRDWRKPTT
                70 80 90 100 110 120
VSIDVIVYAI LNVDEKNQVL TTYIWYRQYW TDEFLQWNPE DFDNITKLSI PTDSIWVPDI
              130 140 150 160 170 180
LINEFVDVGK SPNIPYVYIR HQGEVQNYKP LQVVTACSLD IYNFPFDVQN CSLTFTSWLH
              190 200 210 220 230 240
TIQDINISLW RLPEKVKSDR SVEMNQGEWE LLGVLPYFRE FSMESSNYYA EMKFYVVIRR
              250 260 270 280 290 300
RMGYYLIQMY IPSLLIVILS WISEWINMDA APARVGLGIT TVLTMTTQSS GSRASLPKVS
              310 320 330 340 350 360
YVKAIDIWMA VCLLEVESAL LEYAAVNEVS RQHKELLRER RKRRHHKSPM LNLFQEDEAG
              370 380 390 400 410 420
EGRENFSAYG MGPACLQAKD GISVKGANNS NTTNPPPAPS KSPEEMRKLF IQRAKKIDKI
              430 440 450 451
SRIGFPMAFL IFNMFYWIIY KIVRREDVHN Q.
```

In some embodiments, the chimeric LGIC receptor is a HTR3A/GLRA1 chimera (V236 junction) comprising the human 5HT3A serotonin receptor signal peptide (italics) and ligand binding domain (bold), fused to the human GlyRα1 ion pore domain (underlined):

```
(a)
(SEQ ID NO: 51)
                10 20 30 40 50 60
MLLWVQQALL ALLLPTLLAQ GEARRSRNTT RPALLRLSDY LLTNYRKGVR PVRDWRKPTT
                70 80 90 100 110 120
VSIDVIVYAI LNVDEKNQVL TTYIWYRQYW TDEFLQWNPE DFDNITKLSI PTDSIWVPDI
              130 140 150 160 170 180
LINEFVDVGK SPNIPYVYIR HQGEVQNYKP LQVVTACSLD IYNFPFDVQN CSLTFTSWLH
              190 200 210 220 230 240
TIQDINISLW RLPEKVKSDR SVEMNQGEWE LLGVLPYFRE FSMESSNYYA EMKFYVHLER
              250 260 270 280 290 300
QMGYYLIQMY IPSLLIVILS WISEWINMDA APARVGLGIT TVLTMTTQSS GSRASLPKVS
              310 320 330 340 350 360
YVKAIDIWMA VCLLEVESAL LEYAAVNEVS RQHKELLRER RKRRHHKSPM LNLFQEDEAG
              370 380 390 400 410 420
EGRENFSAYG MGPACLQAKD GISVKGANNS NTTNPPPAPS KSPEEMRKLF IQRAKKIDKI
              430 440 450 451
SRIGFPMAFL IFNMFYWIIY KIVRREDVHN Q.
```

In some embodiments, the chimeric LGIC receptor is a GABRB3/GLRA1 chimera (Y245 junction), comprising the human GABA-A β3 signal peptide (italics) and ligand binding domain (bold), fused to the human GlyRα1 ion pore domain (underlined):

```
(a)
(SEQ ID NO: 52)
                10 20 30 40 50 60
MWGLAGGRLF GIFSAPVLVA VVCCAQSVND PGNMSFVKET VDKLLKGYDI RLRPDFGGPP
                70 80 90 100 110 120
VCVGMNIDIA SIDMVSEVNM DYTLTMYFQQ YWRDKRLAYS GIPLNLTLDN RVADQLWVPD
              130 140 150 160 170 180
TYFLNDKKSF VHGVTVKNRM IRLHPDGTVL YGLRITTTAA CMMDLRRYPL DEQNCTLEIE
              190 200 210 220 230 240
SYGYTTDDIE FYWRGGDKAV TGVERIELPQ FSIVEHRLVS RNVVFATGAY PRLSLSFRLK
              250 260 270 280 290 300
RNIGYMGYYL IQMYIPSLLI VILSWISFWI NMDAAPARVG LGITTVLTMT TOSSGSRASL
              310 320 330 340 350 360
PKVSYVKAID IWMAVCLLFV FSALLEYAAV NEVSRQHKEL LRFRRKRRHH KSPMLNLFQE
              370 380 390 400 410 420
DEAGEGRENF SAYGMGPACL QAKDGISVKG ANNSNTINPP PAPSKSPEEM RKLFIQRAKK
              430 440 450 455
IDKISRIGFP MAFLIENMEY WIIYKIVRRE DVHNQ.
```

In some embodiments, the chimeric LGIC receptor is a CHRNA7/GLRA2 (R229 junction) chimera, comprising the human α7-nAChR signal peptide (italics) and ligand binding domain (bold), fused to the human GlyRα2 ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:62:

```
(a)
(SEQ ID NO: 62)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWV SFWINMDAAP ARVALGITTV LTMTTQSSGS RASLPKVSYV KAIDIWMAVC
              310 320 330 340 350 360
LLFVFAALLE YAAVNFVSRQ HKEFLRLRRR QKRQNKEEDV TRESRENESG YGMGHCLQVK
              370 380 390 400 410 420
DGTAVKATPA NPLPQPPKDG DAIKKKFVDR AKRIDTISRA AFPLAFLIEN IFYWITYKII
                428
RHEDVHKK
```

In some embodiments, the chimeric LGIC receptor is a CHRNA7/GLRA3 (R229 junction) chimera, comprising the human α7-nAChR signal peptide (italics) and ligand binding domain (bold), fused to the human GlyRα3 isoform L ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:63:

```
(a)
(SEQ ID NO: 63)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWV SFWINMDAAP ARVALGITTV LTMTTQSSGS RASLPKVSYV KAIDIWMAVC
              310 320 330 340 350 360
LLEVESALLE YAAVNEVSRQ HKELLRERRK RKNKTEAFAL EKFYRESDMD DEVRESRESE
              370 380 390 400 410 420
TAYGMGPCLQ AKDGMTPKGP NHPVQVMPKS PDEMRKVFID RAKKIDTISR ACEPLAFLIF
              430 440 441
NIFYWVIYKI LRHEDIHQQQ D.
```

In some embodiments, the chimeric LGIC receptor is a CHRNA7/GLRA3 (R229 junction) chimera, comprising the human α7-nAChR signal peptide (italics) and ligand binding domain (bold), fused to the human GlyRα3 isoform K ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:70:

```
(a)
  (SEQ ID NO: 70)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWV SEWINMDAAP ARVALGITTV LTMTTQSSGS RASLPKVSYV KAIDIWMAVC
              310 320 330 340 350 360
LLEVESALLE YAAVNEVSRQ HKELLRERRK RKNKDDEVRE SRESFTAYGM GPCLQAKDGM
              370 380 390 400 410 420
TPKGPNHPVQ VMPKSPDEMR KVFIDRAKKI DTISRACEPL AFLIFNIFYW VIYKILRHED
            426
IHQQQD.
```

In some embodiments, the chimeric LGIC receptor is a CHRNA7/GABRR1 (R229 junction) chimera, comprising the human α7-nAChR signal peptide (italics) and ligand binding domain (bold), fused to the human GABA-A ρ1 ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:64:

```
(a)
(SEQ ID NO: 64)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSC YIDVRWFPFD VQHCKLKFGS WSYGGWSLDL
              190 200 210 220 230 240
QMQEADISGY IPNGEWDLVG IPGKRSERFY ECCKEPYPDV TFTVTMRRRI FFFLLQTYFP
              250 260 270 280 290 300
ATLMVMLSWV SFWIDRRAVP ARVPLGITTV LTMSTIITGV NASMPRVSYI KAVDIYLWVS
              310 320 330 340 350 360
FVEVELSVLE YAAVNYLTTV QERKEQKLRE KLPCTSGLPP PRTAMLDGNY SDGEVNDLDN
              370 380 390 400 410 420
YMPENGEKPD RMMVQLTLAS ERSSPQRKSQ RSSYVSMRID THAIDKYSRI IFPAAYILEN
                428
LIYWSIFS.
```

In some embodiments, the chimeric LGIC receptor is a CHRNA7/GLRA2 (R229 junction) chimera, comprising the human α7-nAChR signal peptide (italics) and ligand binding domain (bold) comprising a GlyRα2 Cys-loop sequence (lowercase), fused to the human GlyRα2 ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:65:

```
(a)
(SEQ ID NO: 65)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknfpmd vqtcKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWV SFWINMDAAP ARVALGITTV LTMTTQSSGS RASLPKVSYV KAIDIWMAVC
              310 320 330 340 350 360
LLEVFAALLE YAAVNEVSRQ HKEFLRLRRR QKRQNKEEDV TRESRENESG YGMGHCLQVK
              370 380 390 400 410 420
DGTAVKATPA NPLPQPPKDG DAIKKKFVDR AKRIDTISRA AFPLAFLIEN IFYWITYKII
                428
RHEDVHKK.
```

In some embodiments, the chimeric LGIC receptor is a CHRNA7/GLRA3 (R229 junction) chimera, comprising the human α7-nAChR signal peptide (italics) and ligand binding domain (bold) comprising a GlyRα3 Cys-loop sequence (lowercase), fused to the human GlyRα3 isoform L ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:66:

```
(a)
  (SEQ ID NO: 66)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknfpmd vqtcKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWV SFWINMDAAP ARVALGITTV LTMTTQSSGS RASLPKVSYV KAIDIWMAVC
              310 320 330 340 350 360
LLEVESALLE YAAVNEVSRQ HKELLRERRK RKNKTEAFAL EKFYRESDMD DEVRESRESE
              370 380 390 400 410 420
TAYGMGPCLQ AKDGMTPKGP NHPVQVMPKS PDEMRKVFID RAKKIDTISR ACFPLAFLIF
              430 440 441
NIFYWVIYKI LRHEDIHQQQ D.
```

In some embodiments, the chimeric LGIC receptor is a CHRNA7/GLRA3 (R229 junction) chimera, comprising the human α7-nAChR signal peptide (italics) and ligand binding domain (bold) comprising a GlyRα3 Cys-loop sequence (lowercase), fused to the human GlyRα3 isoform K ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:71:

```
(a)
(SEQ ID NO: 71)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc pmdlknfpmd vqtcKLKFGS WSYGGWSLDL
              190 200 210 220 230 240
QMQEADISGY IPNGEWDLVG IPGKRSERFY ECCKEPYPDV TFTVTMRRRM GYYLIQMYIP
              250 260 270 280 290 300
SLLIVILSWV SEWINMDAAP ARVALGITTV LTMTTQSSGS RASLPKVSYV KAIDIWMAVC
              310 320 330 340 350 360
LLEVESALLE YAAVNEVSRQ HKELLRERRK RKNKDDEVRE SRESFTAYGM GPCLQAKDGM
              370 380 390 400 410 420
TPKGPNHPVQ VMPKSPDEMR KVEIDRAKKI DTISRACEPL AFLIFNIFYW VIYKILRHED
            426
IHQQQD.
```

In some embodiments, the chimeric LGIC receptor is a CHRNA7/GABRR1 (R229 junction) chimera, comprising the human α7-nAChR signal peptide (italics) and ligand binding domain (bold) comprising a GABA-A ρ1 Cys-loop sequence (lowercase), fused to the human GABA-A ρ1 ion pore domain (underlined). In some embodiments, the chimeric LGIC receptor comprises an amino acid sequence having a sequence identity of 80% or more, 85% or more, 90% or more, 95% or more, 97% or more, 98% or more, 99% or more, or 100%, to SEQ ID NO:67:

```
(a)
(SEQ ID NO: 67)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
              130 140 150 160 170 180
RFDATFHTNV LVNSSGHCQY LPPGIFKSSc nmdfsrfpld tqtcKLKFGS WSYGGWSLDL
              190 200 210 220 230 240
QMQEADISGY IPNGEWDLVG IPGKRSERFY ECCKEPYPDV TFTVTMRRRI FFFLLQTYFP
              250 260 270 280 290 300
ATLMVMLSWV SEWIDRRAVP ARVPLGITTV LTMSTIITGV NASMPRVSYI KAVDIYLWVS
              310 320 330 340 350 360
FVFVELSVLE YAAVNYLTTV QERKEQKLRE KLPCTSGLPP PRTAMLDGNY SDGEVNDLDN
              370 380 390 400 410 420
YMPENGEKPD RMMVQLTLAS ERSSPQRKSQ RSSYVSMRID THAIDKYSRI IFPAAYILEN
                428
LIYWSIFS.
```

### Cell Surface Expression

In some embodiments, the engineered receptor comprises one or more amino acid mutations as compared to the corresponding region of the wildtype receptor, which alter the expression level of the engineered receptor on the cell surface. In some embodiments, the one or more amino acid mutations increase the expression level of the engineered receptor on the cell surface as compared to the corresponding engineered receptor without such one or more mutations. In some embodiments, the one or more amino acid mutations that alter the surface expression of the engineered receptor do not negatively affect the potency, efficacy, and/or responsiveness of the engineered receptor.

In some embodiments, the one or more amino acid mutations are at the pre-M1 linker of the engineered receptor. In some embodiment, the one or more amino acid mutations are 1, 2, 3, 4, 5, or more than 5, amino acid mutations. In some embodiments, the one or more amino acid mutations are 1 amino acid mutation.

In some embodiments, the pre-M1 linker of the engineered receptor comprises amino acid mutation(s) at one or more positions comprising those corresponding to T225, M226, and/or T230 of human α7-nAChR (SEQ ID NO:4). In some embodiments, the pre-M1 linker of the engineered receptor comprises amino acid mutation(s) corresponding to one or more of T225I, M226I, and/or T230P of human α7-nAChR (SEQ ID NO:4).

In some embodiments, the ligand binding domain of the engineered receptor is derived from human α7-nAChR (SEQ ID NO:4) and comprises a mutation at amino acid position corresponding to T225 of SEQ ID NO:4. In some embodiments, the ligand binding domain of the engineered receptor comprises a mutation at the position corresponding to T225 of human α7-nAChR (SEQ ID NO:4), wherein the ligand binding domain of the engineered receptor has at least 60%, at least 65%, at least 70%, at least 75%, at least 80%, at least 85%, at least 90%, at least 95%, at least 96%, at least 97%, at least 98%, at least 99%, at least 99.5%, or 100% (including all ranges and subranges therebetween) sequence identity to the ligand binding domain of human α7-nAChR (SEQ ID NO:4). In some embodiments, the mutation corresponds to T225A, T225C, T225D, T225E, T225F, T225G, T225H, T225I, T225K, T225L, T225M, T225N, T225P, T225Q, T225R, T225S, T225V, T225W, or T225Y, of SEQ ID NO:4.

In some embodiments, the ligand binding domain of the engineered receptor is derived from human α7-nAChR (SEQ ID NO:4) and comprises a mutation at amino acid position corresponding to M226 of SEQ ID NO:4. In some embodiments, the ligand binding domain of the engineered receptor comprises a mutation at the position corresponding to M226 of human α7-nAChR (SEQ ID NO:4), wherein the ligand binding domain of the engineered receptor has at least 60%, at least 65%, at least 70%, at least 75%, at least 80%, at least 85%, at least 90%, at least 95%, at least 96%, at least 97%, at least 98%, at least 99%, at least 99.5%, or 100% (including all ranges and subranges therebetween) sequence identity to the ligand binding domain of human α7-nAChR (SEQ ID NO:4). In some embodiments, the mutation corresponds to M226A, M226C, M226D, M226E, M226F, M226G, M226H, M226I, M226K, M226L, M226N, M226P, M226Q, M226R, M226S, M226T, M226V, M226W, or M226Y, of SEQ ID NO:4.

In some embodiments, the ligand binding domain of the engineered receptor is derived from human α7-nAChR (SEQ ID NO:4) and comprises a mutation at amino acid position corresponding to T230 of SEQ ID NO:4. In some embodiments, the ligand binding domain of the engineered receptor comprises a mutation at the position corresponding to T230 of human α7-nAChR (SEQ ID NO:4), wherein the ligand binding domain of the engineered receptor has at least 60%, at least 65%, at least 70%, at least 75%, at least 80%, at least 85%, at least 90%, at least 95%, at least 96%, at least 97%, at least 98%, at least 99%, at least 99.5%, or 100% (including all ranges and subranges therebetween) sequence identity to the ligand binding domain of human α7-nAChR (SEQ ID NO:4). In some embodiments, the mutation corresponds to T230A, T230C, T230D, T230E, T230F, T230G, T230H, T230I, T230K, T230L, T230M, T230N, T230P, T230Q, T230R, T230S, T230V, T230W, or T230Y, of SEQ ID NO:4.

In some embodiments, the ligand binding domain of the engineered receptor is derived from human α7-nAChR (SEQ ID NO:4) and comprises a mutation corresponding to T225I of SEQ ID NO:4. In some embodiments, the ligand binding domain of the engineered receptor comprises a mutation corresponding to the T225I mutation of human α7-nAChR (SEQ ID NO:4), wherein the ligand binding domain of the engineered receptor has at least 60%, at least 65%, at least 70%, at least 75%, at least 80%, at least 85%, at least 90%, at least 95%, at least 96%, at least 97%, at least 98%, at least 99%, at least 99.5%, or 100% (including all ranges and subranges therebetween) sequence identity to the ligand binding domain of human α7-nAChR (SEQ ID NO:4).

In some embodiments, the ligand binding domain of the engineered receptor is derived from human α7-nAChR (SEQ ID NO:4) and comprises a mutation corresponding to M226I of SEQ ID NO:4. In some embodiments, the ligand binding domain of the engineered receptor comprises a mutation corresponding to the M226I mutation of human α7-nAChR (SEQ ID NO:4), wherein the ligand binding domain of the engineered receptor has at least 60%, at least 65%, at least 70%, at least 75%, at least 80%, at least 85%, at least 90%, at least 95%, at least 96%, at least 97%, at least 98%, at least 99%, at least 99.5%, or 100% (including all ranges and subranges therebetween) sequence identity to the ligand binding domain of human α7-nAChR (SEQ ID NO:4).

In some embodiments, the ligand binding domain of the engineered receptor is derived from human α7-nAChR (SEQ ID NO:4) and comprises a mutation corresponding to T230P of SEQ ID NO:4. In some embodiments, the ligand binding domain of the engineered receptor comprises a mutation corresponding to the T230P mutation of human α7-nAChR (SEQ ID NO:4), wherein the ligand binding domain of the engineered receptor has at least 60%, at least 65%, at least 70%, at least 75%, at least 80%, at least 85%, at least 90%, at least 95%, at least 96%, at least 97%, at least 98%, at least 99%, at least 99.5%, or 100% (including all ranges and subranges therebetween) sequence identity to the ligand binding domain of human α7-nAChR (SEQ ID NO:4).

In some embodiments, the cell surface expression of the engineered receptor comprising the one or more mutations is increased by 10% or higher, 20% or higher, 30% or higher, 40% or higher, 50% or higher, 60% or higher, 70% or higher, 80% or higher, 90% or higher or 100% or higher, including all ranges and subranges therebetween, as compared to the cell surface expression of the corresponding engineered receptor without such one or more mutations. In some embodiments, the cell surface expression of the engineered receptor comprising the one or more mutations is increased by at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, as compared to the cell surface expression of the corresponding engineered receptor without such one or more mutations.

In some embodiments, the ion pore domain of the engineered receptor is derived from human GlyRα2 (SEQ ID NO: 59), human GlyRα3 (SEQ ID NO: 61 or 69), human GABA-A ρ1 (SEQ ID NO:10), human GABA-A ρ2 (SEQ ID NO:12), or human GABA-A ρ3 (SEQ ID NO: 14). In some embodiments, such an engineered receptor has a higher surface expression compared to the corresponding engineered receptor having an ion pore domain derived from human GlyRα1 (SEQ ID NO: 2). In some embodiments, the ion pore domain of such an engineered receptor is derived from human GlyRα2 (SEQ ID NO: 59). In some embodiments, the ion pore domain of such an engineered receptor is derived from human GlyRα3 (SEQ ID NO: 61 or 69). In some embodiments, the ion pore domain of such an engineered receptor is derived from human GABA-A ρ1 (SEQ ID NO:10).

In some embodiments, cell surface expression of the engineered receptor can be measured using α-bungarotoxin (a-BTX) binding assay. For example, relative cell surface expression can be evaluated according to the fluorescence intensity of α-bungarotoxin staining positive cells using fluorescently labelled α-bungarotoxin.

Without being bound by any particular theory, the increase of cell surface expression of the engineered receptor may be attributed to one or more of the following factors:
(a) increased trafficking of the engineered receptor to the cell surface;
(b) decreased trafficking of the engineered receptor to a non-cell surface destination;
(c) longer retention of the engineered receptor on the cell surface;
(d) decreased internalization of the engineered receptor from the cell surface;
(e) reduced degradation of the engineered receptor; and/or
(f) increase of total expression of the engineered receptor.
   In some embodiments, the increase of cell surface expression of the engineered receptor lowers the stress of endoplasmic reticulum (ER). In some embodiments, the increase of cell surface expression of the engineered receptor lowers the stress of cellular degradation machinery. In some embodiments, the increase of cell surface expression of the engineered receptor lowers the stress of cellular degradation machinery. In some embodiments, the increase of cell surface expression of the engineered receptor increases the viability (e.g., long term viability) of the cells expressing the engineered receptor. In some embodiments, the increase of cell surface expression of the engineered receptor increases the sensitivity of the cells expressing the engineered receptor to one or more ligand (e.g., non-native ligand). In some embodiments, more than 10%, more than 20%, more than 30%, more than 40%, more than 50%, more than 60%, more than 70%, more than 80%, more than 90% or 100% (including all ranges and subranges therebetween) of total engineered receptors in the cells are expressed on the cell surface.

In some embodiments, the engineered receptor forms homomeric receptor on the cell surface. In some embodiments, the engineered receptor forms pentameric channel that contains homomeric subunits. In some embodiments, the engineered receptor that forms homomeric channel comprises an ion pore domain derived from the ion pore domain of human GlyRα1. In some embodiments, the engineered receptor that forms homomeric channel comprises an ion pore domain derived from the ion pore domain of human GlyRα2. In some embodiments, the engineered receptor that forms homomeric channel comprises an ion pore domain derived from the ion pore domain of human GlyRα3. In some embodiments, the engineered receptor that forms homomeric channel comprises an ion pore domain derived from the ion pore domain of human GABA-A ρ1. In some embodiments, the engineered receptor that forms homomeric channel comprises an ion pore domain derived from the ion pore domain of human GABA-A ρ2. In some embodiments, the engineered receptor that forms homomeric channel comprises an ion pore domain derived from the ion pore domain of human GABA-A ρ3. In some embodiments, more than 10%, more than 20%, more than 30%, more than 40%, more than 50%, more than 60%, more than 70%, more than 80%, more than 90% or 100% (including all ranges and subranges therebetween) of the engineered receptor expressed on cell surface form homomeric ion channels.

### Mutations of the Engineered Receptor

In some embodiments, the engineered receptor comprises an ion pore domain derived from the IPD of human GlyRα1 (SEQ ID NO:2), and wherein the eight amino acids corresponding to amino acids 354-361 of SEQ ID NO:2 (“SPMILNLFQ”, SEQ ID NO: 96) are removed from the ion pore domain. In some embodiments, the engineered receptor excluding these eight amino acids displays higher cell surface expression, enhanced exportation to cell surface, slower internalization from cell surface and/or slower degradation compared to a corresponding engineered receptor containing these eight amino acids. In some embodiments, the engineered receptor excluding these eight amino acids displays higher potency, higher efficacy and/or high responsiveness compared to a corresponding engineered receptor containing these eight amino acids. In some embodiments, the engineered receptor comprises an ion pore domain derived from the IPD of human GlyRα1, wherein the ion pore domain comprises no amino acid between the amino acid positions corresponding to K353 and E362 of SEQ ID NO:2. In some embodiments, the engineered receptor comprises an ion pore domain derived from the IPD of human GlyRα1, wherein the ion pore domain comprise at most 1, at most 2, at most 3, at most 4, at most 5, at most 6, or at most 7, amino acids between the amino acid positions corresponding to K353 and E362 of SEQ ID NO: 2. In some embodiments, the engineered receptor comprises an ion pore domain derived from the IPD of human GlyRα1, wherein the ion pore domain comprises an amino acid sequence having less than 10%, less, than 20%, less than 30%, less than 40%, less than 50%, less than 60%, less than 70%, less than 80%, or less than 90% (including all ranges and subranges therebetween) sequence identity to SEQ ID NO: 96 between the amino acid positions corresponding to K353 and E362 of SEQ ID NO: 2. In some embodiments, such an engineered receptor comprises a ligand binding domain derived from human α7-nAChR, wherein the Cys-loop domain is optionally replaced by a sequence derived from the corresponding Cys-loop domain of GlyRα1. In some embodiments, such an engineered receptor displays higher cell surface expression compared to a corresponding engineered receptor comprising an ion pore domain derived from the IPD of human GlyRα1 (SEQ ID NO: 2) and contains these eight amino acids. In some embodiments, cell surface expression of such an engineered receptor is increased by at least 10%, at least 20%, at least 30%, at least 40%, at least 50%, at least 60%, at least 70%, at least 80%, at least 90%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, compared to a corresponding engineered receptor comprising an ion pore domain derived from the IPD of human GlyRα1 (SEQ ID NO: 2) and contains these eight amino acids. In some embodiments, cell surface expression of such an engineered receptor is increased by at least 50% compared to a corresponding engineered receptor comprising an ion pore domain derived from the IPD of human GlyRα1 (SEQ ID NO: 2) and contains these eight amino acids.

In some embodiments, the engineered receptor comprises an ion pore domain derived from the IPD of human GlyRα3 isoform K (SEQ ID NO: 69). In some embodiments, the engineered receptor comprises an ion pore domain derived from the IPD of human GlyRα3 isoform L (SEQ ID NO:61). The difference between these two GlyRα3 isoforms is that human GlyRα3 isoform L (SEQ ID NO:61) comprises extra 15 amino acid residues corresponding to amino acids 358-372 of SEQ ID NO:61 (“TEAFALEKFYRFSDM”, SEQ ID NO: 95) in the ion pore domain compared with human GlyRα3 isoform K (SEQ ID NO:69). In some embodiments, the engineered receptor comprising an IPD derived from the IPD of GlyRα3 isoform K (i.e., excluding this 15 amino acids segment) displays higher cell surface expression, enhanced exportation to cell surface, slower internalization from cell surface and/or slower degradation compared to a corresponding engineered receptor comprising an IPD derived from the IPD of GlyRα3 isoform L (i.e., comprising this 15 amino acids segment). In some embodiments, the engineered receptor excluding this 15 amino acids segment displays higher potency, higher efficacy and/or high responsiveness compared to a corresponding engineered receptor comprising this 15 amino acids segment.

In some embodiments, the engineered receptor comprises an ion pore domain derived from the IPD of human GlyRα3 isoform K (SEQ ID NO: 69), wherein the ion pore domain comprise no amino acid residue between the amino acid positions corresponding to K357 and D358 of SEQ ID NO:69. In some embodiments, the engineered receptor comprises an ion pore domain derived from the IPD of human GlyRα3 isoform K (SEQ ID NO: 69), wherein the ion pore domain comprise at most 1, at most 2, at most 3, at most 4, at most 5, at most 6, at most 7, at most 8, at most 9, at most 10, at most 11, at most 12, at most 13, or at most 14 amino acids between the amino acid positions corresponding to K357 and D358 of SEQ ID NO: 69. In some embodiments, the engineered receptor comprises an ion pore domain derived from the IPD of human GlyRα3 isoform K (SEQ ID NO: 69), wherein the ion pore domain comprises an amino acid sequence having less than 10%, less, than 20%, less than 30%, less than 40%, less than 50%, less than 60%, less than 70%, less than 80%, or less than 90% (including all ranges and subranges therebetween) sequence identity to SEQ ID NO: 95 between the amino acid positions corresponding to K357 and D358 of SEQ ID NO: 69. In some embodiments, such an engineered receptor comprises a ligand binding domain derived from human α7-nAChR, wherein the Cys-loop domain is optionally replaced by a sequence derived from the corresponding Cys-loop domain of GlyRα3. In some embodiments, such an engineered receptor displays higher cell surface expression compared to a corresponding engineered receptor comprising an ion pore domain derived from the IPD of human GlyRα3 isoform L (SEQ ID NO: 61). In some embodiments, cell surface expression of such an engineered receptor is increased by at least 10%, at least 20%, at least 30%, at least 40%, at least 50%, at least 60%, at least 70%, at least 80%, at least 90%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, compared to a corresponding engineered receptor comprising an ion pore domain derived from the IPD of human GlyRα3 isoform L (SEQ ID NO: 61). In some embodiments, cell surface expression of such an engineered receptor is increased by at least 50% compared to a corresponding engineered receptor comprising an ion pore domain derived from the IPD of human GlyRα3 isoform L (SEQ ID NO: 61) comprising the 15 amino acid residues corresponding to amino acids 358-372 of SEQ ID NO:61.

In some embodiments, the engineered receptor comprises an ion pore domain derived from the IPD of human GlyRα1 (SEQ ID NO:2), and wherein the nuclear localization signal (NLS)/ER retention signal (ERRS) of the ion pore domain comprises one or more mutations. In some embodiments, the NLS/ERRS sequence comprises, consists of, or is within the 6 amino acids corresponding to amino acids 346-351 of SEQ ID NO:2 (“RRKRRH”, SEQ ID NO: 97). In some embodiments, the mutations comprise 1, 2, 3, 4, 5, or more than 5 non-lysine, non-arginine substitutions. In some embodiments, the mutations comprise deletion of 1, 2, 3, 4, 5, or more than 5 amino acids within the NLS/ERRS sequence. In some embodiments, the NLS/ERRS sequence of the engineered receptor corresponding to amino acids 346-351 of SEQ ID NO:2 is replaced by the amino acid sequence of SEQ ID NO: 98 (“RRRQKR”), or an amino acid sequence having at most 1 or at most 2 mutations according to SEQ ID NO: 98. In some embodiments, the NLS/ERRS sequence of the engineered receptor corresponding to amino acids 346-350 of SEQ ID NO:2 (“RRKRR”) is replaced by the first five amino acids of SEQ ID NO: 98 (“RRRQK”). In some embodiments, the engineered receptor having the one or more mutations in the NLS/ERRS sequence displays higher cell surface expression, enhanced exportation to cell surface, slower internalization from cell surface and/or slower degradation compared to a corresponding engineered receptor containing the intactNLS/ERRS sequence. In some embodiments, the engineered receptor having the one or more mutations in the NLS/ERRS sequence displays higher potency, higher efficacy and/or high responsiveness compared to a corresponding engineered receptor containing the intact NLS/ERRS sequence.

In some embodiments, the engineered receptor comprises an ion pore domain derived from the IPD of human GlyRα2 (SEQ ID NO:59), and wherein the nuclear localization signal (NLS)/ER retention signal (ERRS) of the ion pore domain comprises one or more mutations. In some embodiments, the NLS/ERRS sequence comprises, consists of, or is within the 6 amino acids corresponding to amino acids 352-357 of SEQ ID NO:59 (“RRRQKR”, SEQ ID NO: 98). In some embodiments, the mutations comprise 1, 2, 3, 4, 5, or more than 5 non-lysine, non-arginine substitutions. In some embodiments, the mutations comprise deletion of 1, 2, 3, 4, 5, or more than 5 amino acids within the NLS/ERRS sequence. In some embodiments, the engineered receptor having the one or more mutations in the NLS/ERRS sequence displays higher cell surface expression, enhanced exportation to cell surface, slower internalization from cell surface and/or slower degradation compared to a corresponding engineered receptor containing the intact NLS/ERRS sequence. In some embodiments, the engineered receptor having the one or more mutations in the NLS/ERRS sequence displays higher potency, higher efficacy and/or high responsiveness compared to a corresponding engineered receptor containing the intact NLS/ERRS sequence.

In some embodiments, the engineered receptor comprises an ion pore domain derived from the IPD of human GlyRα3 (SEQ ID NO:61 or 69), and wherein the nuclear localization signal (NLS)/ER retention signal (ERRS) of the ion pore domain comprises one or more mutations. In some embodiments, the NLS/ERRS sequence comprises, consists of, or is within the 6 amino acids corresponding to amino acids 351-356 of SEQ ID NO:61 or 69 (“RRKRKN”, SEQ ID NO: 99). In some embodiments, the mutations comprise 1, 2, 3, 4, 5, or more than 5 non-lysine, non-arginine substitutions. In some embodiments, the mutations comprise deletion of 1, 2, 3, 4, 5, or more than 5 amino acids within the NLS/ERRS sequence. In some embodiments, the NLS/ERRS sequence of the engineered receptor corresponding to amino acids 351-356 of SEQ ID NO:61 or 69 is replaced by the amino acid sequence of SEQ ID NO: 98 (“RRRQKR”), or an amino acid sequence having at most 1 or at most 2 mutations according to SEQ ID NO: 98. In some embodiments, the NLS/ERRS sequence of the engineered receptor corresponding to amino acids 351-355 of SEQ ID NO:61 or 69 (“RRKRK”) is replaced by the first five amino acids of SEQ ID NO: 98 (“RRRQK”). In some embodiments, the engineered receptor having the one or more mutations in the NLS/ERRS sequence displays higher cell surface expression, enhanced exportation to cell surface, slower internalization from cell surface and/or slower degradation compared to a corresponding engineered receptor containing the intact NLS/ERRS sequence. In some embodiments, the engineered receptor having the one or more mutations in the NLS/ERRS sequence displays higher potency, higher efficacy and/or high responsiveness compared to a corresponding engineered receptor containing the intact NLS/ERRS sequence.

### Amino Acid Mutations

As discussed above, in some aspects, the subject engineered receptor comprises at least one amino acid mutation that alters the potency of a ligand on the engineered receptor relative to its potency on the unmutated parental receptor. Put another way, the one or more amino acid mutations, e.g. a loss-of-function mutations or a gain-of-function mutations, shift the responsiveness of the engineered receptor to the ligand relative to the responsiveness of the unmutated parental receptor. In some such embodiments the one or more mutations is in the ligand binding domain of the engineered receptor. In some embodiments, as when the ligand binding domain of the engineered receptor is a Cys-loop receptor protein, the one or more amino acid mutations is a substitution at a residue corresponding to a residue of α7-nAChR (SEQ ID NO: 4) selected from the group consisting of W77, Y94, R101, W108, Y115, T128, N129, V130, L131, Q139, L141, Y151, S170, W171, S172, S188, Y190, Y210, C212, C213 and Y217. In some embodiments, one residue is substituted. In some embodiments, 2, 3, 4, or 5 or more residues are substituted, e.g. 6, 7, 8, 9 or 10 residues are substituted. In certain embodiments, the residue corresponds to a residue of α7-nAChR (SEQ ID NO: 4) that is selected from the group consisting of W77, R101, Y115, N129, L131, S170, S172, and S188. In certain embodiments, the one or more substitutions is within an α7-nAChR sequence.

In some embodiments, the one or more substitutions decreases, e.g. 2-fold or more, 3-fold or more, 4-fold or more. 5-fold or more, 10-fold or more, 20-fold or more, 30-fold or more, 50-fold or more, or 100-fold, the responsiveness of an engineered receptor to acetylcholine and a non-native ligand. In certain embodiments, the one or more substitutions is a substitution corresponding to R101I, R101S, R101D, Y115L, Y115M, Y115D, Y115T, T128M, T128R, T128I, N129I, N129V, N129P, N129W, N129T, N129D, N129E, L131E, L131P, L131T, L131D, L131S, L141S, L141R, W171F, W171H, S172F, S172Y, S172R, S172D, C212A, C212L, or C213P of α7-nAChR. In other instances, the one or more substitutions decreases the potency of acetylcholine on the engineered receptor selectively. In other words, the one or more substitutions decreases the responsiveness of the engineered receptor to acetylcholine while essentially maintaining responsiveness to non-native ligand or otherwise decreasing the responsiveness of the engineered receptor to acetylcholine 2-fold or more, e.g. 3-fold, 4-fold, 5-fold or more, in some instances 10-fold, 20-fold, 50-fold, or 100-fold or more, than it decreases the responsiveness of the engineered receptor to non-native ligand. Exemplary substitutions, namely, a substitution corresponding to L131E, L131S, L131T, L131D, or S172D of α7-nAChR. In yet other embodiments, the one or more substitutions decreases the potency of a non-native ligand on the engineered receptor selectively. In other words, the one or more substitutions decreases the responsiveness of the engineered receptor to non-native ligand while essentially maintaining responsiveness to acetylcholine or otherwise decreasing the responsiveness of the engineered receptor to non-native ligand 2-fold or more, e.g. 3-fold, 5-fold or more, in some instances 10-fold, 20-fold or 50-fold or more, than it decreases the responsiveness of the engineered receptor to acetylcholine. Exemplary substitutions include a substitution corresponding to W77M, Y115W, S172T, or S172C of α7-nAChR. In certain embodiments, the one or more substitutions is within an α7-nAChR sequence. In certain embodiments, the non-native ligand is selected from AZD-0328, TC6987, ABT-126 and Facinicline/RG3487.

In other embodiments, the one or more substitutions increases, e.g. 2-fold or more, 3-fold or more, 4-fold or more. 5-fold or more, 10-fold or more, 20-fold or more, 30-fold or more, 50-fold or more, or 100-fold, the responsiveness of the engineered receptor to acetylcholine and/or non-native ligand. Exemplary substitutions include a substitution corresponding to L131N, L141W, S170G, S170A, S170L, S170I, S170V, S170P, S170F, S170M, S170T, S170C, S172T, S172C, S188I, S188V, S188F, S188M, S188Q, S188T, S188P or S188W. In some instance, the one or more substitutions increases potency of both acetylcholine and non-native ligand, e.g. substitutions corresponding to L131N, S170G, S170A, S170L, S170I, S170V, S170P, S170F, S170M, S170T, S170C, S172T, S188I, S188V, S188F, S188M, S188Q and S188T of α7-nAChR. In other instances, the one or more substitutions increases the potency of acetylcholine on the engineered receptor selectively. In other words, the one or more substitutions increases the responsiveness of the engineered receptor to acetylcholine 2-fold or more, e.g. 3-fold, 4-fold, or 5-fold or more, in some instances 10-fold, 20-fold, 50-fold, or 100-fold, than it increases the responsiveness of the engineered receptor to non-native ligand, e.g. substitutions corresponding to L141W, S172T, S172C, S188P or S188W, of α7-nAChR. In certain embodiments, the one or more substitutions is within an α7-nAChR sequence. In certain embodiments, the non-native ligand is selected from AZD-0328, TC6987, ABT-126 and Facinicline/RG3487. In yet other instances, the one or more substitutions increases the potency of the non-native ligand on the engineered receptor selectively. In other words, the one or more substitutions increases the responsiveness of the engineered receptor to non-native ligand 2-fold or more, e.g. 3-fold, 5-fold or more, in some instances 10-fold, 20-fold or 50-fold or more, than it increases the responsiveness of the engineered receptor to acetylcholine.

In some embodiments, the amino acid residue that is mutated in the subject engineered receptor is not an amino acid corresponding to R27, E41, Q79, Q139, L141, G175, Y210, P216, Y217, or D219 of wild type α7 nAChR (SEQ ID NO:4). In some embodiments, the amino acid residue that is mutated in the subject engineered receptor is an amino acid corresponding to R27, E41, Q79, Q139, L141, G175, Y210, P216, Y217, or D219 of wild type α7 nAChR (SEQ ID NO:4). In some embodiments, the substitution is not a substitution corresponding to W77F, W77Y, W77M, Q79A, Q79Q, Q79S, Q79G, Y115F, L131A, L131G, L131M, L131N, L131Q, L131V, L131F, Q139G, Q139L, G175K, G175A, G175F, G175H, G175M, G175R, G175S, G175V, Y210F, P216I, Y217F, or D219A in wild type α7 nAChR. In some embodiments, the substitution is a substitution corresponding to W77F, W77Y, W77M, Q79A, Q79Q, Q79S, Q79G, Y115F, L131A, L131G, L131M, L131N, L131Q, L131V, L131F, Q139G, Q139L, G175K, G175A, G175F, G175H, G175M, G175R, G175S, G175V, Y210F, P216I, Y217F, or D219A in wild type α7 nAChR. In some embodiments, when such a substitution exists within the engineered receptor, it exists in combination with one or more of the amino acid mutations described herein.

For example, it has been discovered that residues Y94, Y115, Y151, and Y190 of α7-nAChR (SEQ ID NO:4) mediate binding of the native ligand acetylcholine. Mutations at these residues will reduce binding of acetylcholine and hence are loss of function mutations. In contrast, residues W77, Y115, N129, V130, L131, Q139, L141, S170, Y210, C212, C213 and Y217 of the α7-nAChR mediate the binding of non-native ligand AZD0328 to this receptor, and mutation of these residues may increase the affinity of AZD0328 and/or other ligands for this receptor and hence be gain-of-function mutations. In some embodiments, the subject engineered receptor comprises a mutation in one or more amino acid residues of the ligand binding domain region of α7-nAChR (SEQ ID NO:4) or the ligand binding domain of a chimeric receptor that comprises the ligand binding domain region of α7-nAChR, wherein the one or more amino acid residues is selected from the group consisting of W77, Y94, Y115, N129, V130, L131, Q139, L141, Y151, S170, Y190, Y210, C212, C213 and Y217. In certain embodiments, the mutation in the one or more amino acid residues of the ligand binding domain region of α7-nAChR (SEQ ID NO.4) or the ligand binding domain of a chimeric receptor that comprises the ligand binding domain region of α7-nAChR is a substitution at one or more amino acid residues selected from the group consisting of W77, Y94, Y115, N129, V130, L131, Q139, L141, Y151, S170, Y190, Y210, C212, C213 and Y217.

As another example, it has been discovered that residues Y115, L131, L141, S170, W171, S172, C212, and Y217 of α7-nAChR (SEQ ID NO.4) mediate binding of acetylcholine and/or nicotine, and mutations at one or more of these residues will reduce binding of acetylcholine and/or nicotine. R101, Y115, L131, L141, W171, S172, S188, Y210, and Y217 of α7-nAChR mediate binding of the non-native ligand ABT126, and mutation of one or more of these residues is expected to increase the affinity of ABT126 and/or other ligands for α7-nAChR. R101, Y115, T128, N129, L131, L141, W171, S172, Y210, C212, C213 and Y217 of α7-nAChR mediate binding of the non-native ligand TC6987, and mutation of one or more of these residues is expected to increase the affinity of TC6987 and/or other ligands for α7-nAChR. R101, N120, L131, L141, S170, W171, S172, Y210, and Y217 of α7-nAChR mediate binding of the non-native ligand Facinicline/RG3487, and mutation of one or more of these residues is expected to increase the affinity of Facinicline/RG3487 and/or other ligands for α7-nAChR. In some embodiments, the subject engineered receptor comprises a mutation in one or more amino acid residues of the ligand binding domain region of α7-nAChR or the ligand binding domain of a chimeric receptor that comprises the ligand binding domain region of α7-nAChR, where the one or more amino acid residues is selected from the group consisting of R101, Y115, T128, N120, N129, L131, L141, S170, W171, S172, S188, Y210, C212, C213 and Y217. In some embodiments, the one or more amino acid residues alters the binding of acetylcholine and/or nicotine to α7-nAChR, wherein the amino acid is selected from the group consisting of Y115, L131, L141, S170, W171, S172, C212 and Y217 of α7-nAChR. In certain such embodiments, the amino acid is selected from C212 and S170. In some embodiments, the mutation in the one or more amino acid residues alters the binding of ABT126 to α7-nAChR, wherein one or more amino acid residues is selected from the group consisting of R101, Y115, L131, L141, W171, S172, S188, Y210, and Y217 of α7-nAChR. In certain such embodiments, the amino acid is selected from R101, S188, and Y210. In some embodiments, the mutation in the one or more amino acid residues alters the binding of TC6987 to α7-nAChR, wherein one or more amino acid residues is selected from the group consisting of R101, Y115, T128, N129, L131, L141, W171, S172, Y210, C212, C213 and Y217 of α7-nAChR. In certain such embodiments, the amino acid is selected from R101, T128, N129, Y210 and C213. In some embodiments, the mutation in the one or more amino acid residues alters the binding of Facinicline/RG3487 to α7-nAChR, wherein one or more amino acid residues is selected from the group consisting R101, N120, L131, L141, 5170, W171, 5172, Y210, and Y217 of α7-nAChR. In certain such embodiments, the amino acid is selected from Y210, R101, and N129.

As another example, it has been discovered that residues W85, R87, Y136, Y138, G146, N147, Y148, K149, S177, S178, L179, Y228, and Y229 of 5HT3 (SEQ ID NO:6) mediate binding of serotonin, and mutations at one or more of these residues will reduce binding of serotonin to 5HT3. D64, I66, W85, R87, Y89, N123, G146, Y148, T176, S177, S178, W190, R191, F221, E224, Y228, Y229 and E231 of 5HT3 mediate binding of the non-native ligand Cilansetron, and mutation of one or more of these residues is expected to increase the affinity of Cilansetron and/or other ligands for 5HT3. In some embodiments, the subject engineered receptor comprises a mutation in one or more amino acid residues of the ligand binding domain region 5HT3A or the ligand binding domain of a chimeric receptor that comprises the ligand binding domain region of 5HT3, where the one or more amino acid residues is selected from the group consisting of D64, I66, W85, R87, Y89, N123, Y136, Y138, G146, N147, Y148, K149, T176, S177, S178, L179, W190, R191, F221, E224, Y228, Y229, and E231. In some embodiments, the mutation in the one or more amino acid residues alters the binding of serotonin to 5HT3, wherein the amino acid is selected from the group consisting of W85, R87, Y136, Y138, G146, N147, Y148, K149, S177, S178, L179, Y228, and Y229 of 5HT3A. In certain such embodiments, the amino acid is selected from Y136, Y138, N147, K149, and L179. In some embodiments, the mutation in the one or more amino acid residues alters the binding of Cilansetron to 5HT3 wherein one or more amino acid residues is selected from the group consisting of D64, I66, W85, R87, Y89, N123, G146, Y148, T176, S177, S178, W190, R191, F221, E224, Y228, Y229 and E231 of 5HT3A. In certain such embodiments, the amino acid is selected from D64, 166, Y89, N123, T176, W190, R191, F221, E224, and E231.

In some embodiment, the one or more mutations that affects the ability of a ligand to modulate the activity of the LGIC is located in the ion pore domain of the LGIC. For example, residue T279 of the serotonin receptor 5HT3A mediates the way in which the ligand modulates the activity of the channel, such that mutation of this residue to, e.g. serine (T279S), converts the effect from being antagonistic (i.e., reducing the activity of the LGIC) to agonistic (i.e. promoting the activity of the channel). In some embodiments, the subject ligand gated ion channel comprises a mutation in one or more amino acid residues of the ion pore domain of the human 5HT3A (SEQ ID NO:6) or the ion pore domain of a chimeric LGIC receptor that comprises the ion pore domain of 5HT3A, where the substitution is in an amino acid corresponding to 279 of SEQ ID NO:6. In certain embodiments, the substitution is a T279S substitution relative to SEQ ID NO:6.

The disclosure provides engineered receptors having two or more mutations, such as amino acid substitutions, as compared to the parental receptor. In some embodiments, the parental receptor is a chimeric receptor. In some embodiments, the parental receptor comprises an amino acid sequence of SEQ ID NO: 33. In some embodiments, the engineered receptors comprise two amino acid substitutions as compared to the parental receptor comprising an amino acid sequence of SEQ ID NO: 33.

In some embodiments, the two amino acid substitutions are at a pair of amino acid residues selected from the group consisting of L131 and S172, Y115 and S170, and Y115 and L131. In some embodiments, the ligand binding domain comprises two amino acid substitutions at a pair of amino acids residues selected from the group consisting of L131 and S172, Y115 and S170, and Y115 and L131. In some embodiments, the ligand binding domain comprises a pair of amino acid substitutions selected from the group consisting of L131S and S172D, L131T and S172D, L131D and S172D, Y115D and S170T, Y115D and L131Q, and Y115D and L131E. In some embodiments, the ligand binding domain comprises an amino acid substitution of L131E.

The disclosure provides engineered receptors, wherein the engineered receptor is a chimeric ligand gated ion channel (LGIC) receptor and comprises: (a) a ligand binding domain derived from the human α7 nicotinic acetylcholine receptor (α7-nAChR) and comprising a Cys-loop domain from the human Glycine receptor α1 subunit; and (b) an ion pore domain derived from the human Glycine receptor α1 subunit, wherein the ligand binding domain comprises: one or more amino acid substitutions at amino acids residues selected from the group consisting of Y140, R101, L131, Y115, and Y210, wherein the amino acid residues correspond to the amino acid residues of α7-nAChR. In some embodiments, the engineered receptor comprises an amino acid sequence of SEQ ID NO: 33, wherein the amino acid sequence further comprises the one or more amino acid substitutions at amino acids residues selected from the group consisting of Y140, R101, L131, Y115, and Y210, wherein the amino acid residues correspond to the amino acid residues of α7-nAChR.

In some embodiments, the ligand binding domain comprises an amino acid substitution at the amino acid residue Y140. In some embodiments, the amino acid substitution is Yl40I, or Yl40C.

In some embodiments, the ligand binding domain comprises an amino acid substitution of R101W and/or Y210V. In some embodiments, the ligand binding domain comprises two or more amino acid substitutions at amino acid residues selected from the group consisting of R101, L131, Y115, Y210, and Y140. In some embodiments, the ligand binding domain comprises two amino acid substitutions at amino acid residues selected from the group consisting of R101, L131, Y115, Y210, and Y140. In some embodiments, the ligand binding domain comprises two amino acid substitutions at a pair of amino acid residues selected from the group consisting of: R101 and L131, Y115 and Y210, R101 and Y210. In some embodiments, the ligand binding domain comprises a pair of amino acid substitutions selected from the group consisting of R101F and L131G, R101F and L131D, Y115E and Y210W, R101W and Y210V, R101F and Y210V, R101F and Y210F, R101M and L131A, and R101M and L131F. In some embodiments, the ligand binding domain comprises three amino acid substitutions at the amino acid residues R101, Y115, and Y210. In some embodiments, the ligand binding domain comprises amino acid substitutions R101W, Y115E, and Y210W, or the amino acid substitutions R101F, Y115E, and Y210W.

In some embodiments, the potency of the engineered receptor to acetylcholine is lower than the potency of the human α7 nicotinic acetylcholine receptor (α7-nAChR) to acetylcholine. In some embodiments, the potency of the engineered receptor to acetylcholine is at least about 1.5-fold (for example, about 2-fold lower, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, about 60-fold, about 70-fold, about 80-fold, about 90-fold, or about 100-fold, including all subranges and values that lie therebetween) lower than the potency of the human α7 nicotinic acetylcholine receptor (α7-nAChR) to acetylcholine.

In some embodiments, the potency of the engineered receptor to a non-native ligand is about the same as the potency of the human α7 nicotinic acetylcholine receptor (α7-nAChR) to the non-native ligand. In some embodiments, the potency of the engineered receptor to a non-native ligand is higher than the potency of the human α7 nicotinic acetylcholine receptor (α7-nAChR) to the non-native ligand. In some embodiments, the potency of the engineered receptor to the non-native ligand is at least about 1.5-fold (for example, about 2-fold lower, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, about 60-fold, about 70-fold, about 80-fold, about 90-fold, or about 100-fold, including all subranges and values that lie therebetween) higher than the potency of the human α7 nicotinic acetylcholine receptor (α7-nAChR) to the non-native ligand. In some embodiments, determining the potency comprises determining the EC50.

In some embodiments, the efficacy of the engineered receptor in the presence of a non-native ligand is higher than the efficacy the human α7 nicotinic acetylcholine receptor (α7-nAChR) in presence of the non-native ligand. In some embodiments, the efficacy of the engineered receptor in the presence of a non-native ligand is at least about 1.5-fold (for example, about 2-fold lower, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, about 60-fold, about 70-fold, about 80-fold, about 90-fold, or about 100-fold, including all subranges and values that lie therebetween) higher than the efficacy the human α7 nicotinic acetylcholine receptor (α7-nAChR) in presence of the non-native ligand. In some embodiments, determining the efficacy comprises determining the amount of current passed through the engineered receptor in vitro in the presence of the non-native ligand.

In some aspects, the subject ligand-gated ion channel comprises one or more non-desensitizing mutations. When used in the context of a ligand-gated ion channel, “desensitization” refers to the progressive reduction in ionic flux in the prolonged presence of agonist. This results in a progressive loss of responsiveness of the neuron to the ligand. By a non-desensitizing mutation, it is meant an amino acid mutation that prevents the LGIC from becoming desensitized to ligand, thereby preventing the neuron from becoming less responsive or nonresponsive to ligand. Non-desensitizing mutations can be readily identified by introducing the LGIC carrying the mutation into a neuron and analyzing the current flux over time during prolonged exposure to ligand. If the LGIC does not comprise a non-desensitizing mutation, the current will restore from peak to steady state during prolonged exposure, whereas if the LGIC comprises a non-desensitizing mutation, the current will remain at peak flux for the duration of exposure to ligand. Exemplary amino acid mutations that result in desensitization include a V322L mutation in the human GlyRα1 (V294L post-processing of the pro-protein to remove the signal peptide) and a L321V mutation in human GABA-A receptor GABRB3 (L296V post-processing of the pro-protein to remove the signal peptide). In some embodiments, the desensitizing mutation is the replacement of amino acid residues at or near the C-terminus of the LGIC with a desensitizing sequence, for example, a sequence having 90% identity or more to IDRLSRIAFPLLFGIFNLVYWATYLNREPQL (SEQ ID NO:53) derived from the C terminus of the protein encoded by GABARI, e.g. the replacement of residues 455-479 in GABRR1 with IDRLSRIAFPLLFGIFNLVYWATYLNREPQL (SEQ ID NO:53). LGIC desensitization, methods for measuring desensitization of LGICs, and mutations that are non-desensitizing are well known in the art; see, e.g. Gielen et al. Nat Commun 2015 Apr. 20, 6:6829, and Keramidas et al. Cell Mol Life Sci. 2013 April; 70(7):1241-53, the full disclosures of which are incorporated herein by reference.

In some aspects, the subject ligand-gated ion channel comprises one or more conversion mutations. By a conversion mutation, it is meant a mutation that changes the permeability of the ion pore domain of the LGIC such that it becomes permissive to the conductance of a non-native ion, i.e. an ion that does not naturally allow to pass through. In some cases, the mutation converts the permeability from cation to anion, for example the replacement of amino acid residues 260-281 in human α7-nAChR (CHRNA7) (EKISLGITVLLSLTVFMLLVAE, SEQ ID NO:54) or the corresponding amino acids in another cation-permeable LGIC with the peptide sequence PAKIGLGITVLLSLTTFMSGVAN (SEQ ID NO:55). In some cases, the mutation converts the permeability from anion to cation, for example, the substitution of amino acid residue 279 of GLRA1 or the corresponding amino acid in another anion-permeable LGIC to glutamic acid (E), (which, as an A293E substitution in GLRA1 converts the LGIC from being anion-permissive to calcium-permissive), or the deletion of amino acid residue 278 of GLRA1 or the corresponding amino acid in another anion-permeable LGIC, the substitution of amino acid residue 279 of GLRA1 or the corresponding amino acid in another anion-permeable LGIC to glutamic acid (E), and the substitution of amino acid residue 293 of GLRA1 or the corresponding amino acid in another anion-permeable LGIC to valine (V) (which, as a P278A, A279E, T293V in GLRA1 converts the LGIC from being anion-permissive to cation-permissive).

Additional engineered receptors beyond those described herein can be readily identified by in vitro screening and validation methods. In some embodiments, a library of parental receptor mutants is generated from a limited number of parental receptors. The parental receptors can be mutated using methods known in the art, including error prone PCR. In some embodiments, the library of parental receptor mutants is then transfected into yeast or mammalian cells and screened in high throughput to identify functional receptors (e.g., to identify parental receptor mutants that are capable of signaling in response to a binding agent or ligand). In some embodiments, the functional parental receptor mutants identified in this primary screen is then expressed in mammalian cells and screened for responsiveness to binding agents or ligands, e.g. by the plate reader and/or electrophysiology assays described herein. The parental receptor mutants that demonstrate either increased binding affinity for agonist binding agents, or that enable the use of antagonist or modulator binding agents as agonists in the secondary screen can then be selected and carried though further in vitro and/or in vivo validation and characterization assays. Such screening assays are known in the art, for example Armbruster, B. N. et al. (2007) PNAS, 104, 5163-5168; Nichols, C. D. and Roth, B. L. (2009) Front. Mol. Neurosci. 2, 16; Dong, S. et al. (2010) Nat. Protoc. 5, 561-573; Alexander, G. M. et al. (2009) Neuron 63, 27-39; Guettier, J. M. et al. (2009) PNAS 106, 19197-19202; Ellefson J. W. et al. (2014) Nat Biotechnol. 32(1):97-101; Maranhao AC and Ellington AD. (2017) ACS Synth Biol. 20; 6(1):108-119; Talwar S et al. (2013) PLoS One; 8(3):e58479; Gilbert D. F. et al. (2009) Front Mol Neurosci. 30; 2:17; Lynagh and Lynch, (2010), Biol Chem. 14:285(20), 14890-14897; Islam R. et al (2016) ACS Chern Neurosci. 21; 7(12):1647-1657; and Myers et al. (2008) Neuron. 8:58(3): 362-373.

### Exemplary LGIC Receptors

Exemplary LGIC receptors of the disclosure are provided in Table 12 below. Chimeric LGIC receptors are described by their ligand binding domain (LBD) and ion pore domain (IPD) separated by colon mark. “Gly-cys” indicates that the Cys-loop of the ligand binding domain is replaced with the corresponding Cys-loop of the Glycine receptor from which the ion pore domain is derived. Some of the LGIC receptors comprises one or more amino acid mutations as indicated in the table.

For example, CODA1298 in Table 12 comprises the ligand binding domain derived from human α7-nAChR receptor and the ion pore domain derived from GlyRα2, wherein the Cys-loop of the ligand binding domain is replaced by the Cys-loop of GlyRα2, and wherein the receptor comprises a T225I mutation at the amino acid position corresponding to T225 of human α7-nAChR receptor (SEQ ID NO: 4).

TABLE 12
Exemplary LGIC Receptors

| | | Mutation | Mutation | Mutation | Amino Acid |
|---|---|---|---|---|---|
| NAME | Description | #1 | #2 | #3 | Sequence |
| CODA64 | α7-nAChR LBD: | | | | SEQ ID NO: 16 |
| | GlyRα1 IPD | | | | |
| CODA71 | α7-nAChR(Gly-cys) | | | | SEQ ID NO: 33 |
| | LBD: GlyRα1 IPD | | | | |
| CODA75 | Non-expressing | | | | SEQ ID NO: 29 |
| | α7-nAChR LBD: | | | | |
| | GlyRα1 IPD | | | | |
| CODA102 | α7-nAChR | | | | SEQ ID NO: 4 |
| CODA409 | α7-nAChR(Gly-cys) | Y210W | | | SEQ ID NO: 33 |
| | LBD: GlyRα1 IPD | | | | comprising Y210W |
| | | | | | mutation |
| CODA829 | α7-nAChR(Gly-cys) | T225I | | | SEQ ID NO: 33 |
| | LBD: GlyRα1 IPD | | | | comprising T225I |
| | | | | | mutation |
| CODA1055 | α7-nAChR(Gly-cys) | R101W | Y115E | Y210W | SEQ ID NO: 33 |
| | LBD: GlyRα1 IPD | | | | comprising R101W, |
| | | | | | Y115E, Y210W |
| | | | | | mutations |
| CODA1237 | α7-nAChR(Gly-cys) | | | | SEQ ID NO: 65 |
| | LBD:GlyRα2 IPD | | | | |
| CODA1238 | α7-nAChR LBD: | | | | SEQ ID NO: 62 |
| | GlyRα2 IPD | | | | |
| CODA1239 | α7-nAChR LBD: | | | | SEQ ID NO: 64 |
| | GABArhol IPD | | | | |
| CODA1298 | α7-nAChR(Gly-cys) | T225I | | | SEQ ID NO: 65 |
| | LBD: GlyRα2 IPD | | | | comprising T225I |
| | | | | | mutation |
| CODA1297 | α7-nAChR(Gly-cys) | Y210W | | | SEQ ID NO: 65 |
| | LBD: GlyRα2 IPD | | | | comprising Y210W |
| | | | | | mutation |
| CODA1299 | α7-nAChR(Gly-cys) | Y210V | R101W | | SEQ ID NO: 65 |
| | LBD:GlyRα2 IPD | | | | comprising Y210V, |
| | | | | | R101W mutations |
| CODA1300 | α7-nAChR(Gly-cys) | L131F | R101M | | SEQ ID NO: 65 |
| | LBD: GlyRα2 IPD | | | | comprising L131F, |
| | | | | | R101M mutations |
| CODA1301 | α7-nAChR(Gly-cys) | Y210W | R101W | Y115E | SEQ ID NO: 65 |
| | LBD: GlyRα2 IPD | | | | comprising Y210W, |
| | | | | | R101W, Y115E |
| | | | | | mutations |
| CODA1302 | α7-nAChR(Gly-cys) | Y210W | R101F | Y115E | SEQ ID NO: 65 |
| | LBD: GlyRα2 IPD | | | | comprising Y210W, |
| | | | | | R101F, Y115E |
| | | | | | mutations |
| CODA1303 | α7-nAChR(Gly-cys) | L131N | W77F | S172D | SEQ ID NO: 65 |
| | LBD: GlyRα2 IPD | | | | comprising L131N, |
| | | | | | W77F, S172D |
| | | | | | mutations |
| CODA1304 | α7-nAChR(Gly-cys) | Y210W | S172D | | SEQ ID NO: 65 |
| | LBD: GlyRα2 IPD | | | | comprising Y210W, |
| | | | | | S172D mutations |
| CODA1305 | α7-nAChR(Gly-cys) | L131G | R101F | | SEQ ID NO: 65 |
| | LBD: GlyRα2 IPD | | | | comprising L131G, |
| | | | | | R101F mutations |
| CODA1306 | α7-nAChR(Gly-cys) | Y115E | Y210W | | SEQ ID NO: 65 |
| | LBD: GlyRα2 IPD | | | | comprising Y115E, |
| | | | | | Y210W mutations |
| CODA1307 | α7-nAChR(Gly-cys) | R101F | Y210V | | SEQ ID NO: 65 |
| | LBD: GlyRα2 IPD | | | | comprising R101F, |
| | | | | | Y210V mutations |
| CODA1308 | α7-nAChR(Gly-cys) | R101F | Y210F | | SEQ ID NO: 65 |
| | LBD: GlyRα2 IPD | | | | comprising R101F, |
| | | | | | Y210F mutations |
| CODA1309 | α7-nAChR(Gly-cys) | R101F | W77F | L131D | SEQ ID NO: 65 |
| | LBD: GlyRα2 IPD | | | | comprising R101F, |
| | | | | | W77F, L131D |
| | | | | | mutations |
| CODA1310 | α7-nAChR(Gly-cys) | Q139E | S172D | | SEQ ID NO: 65 |
| | LBD: GlyRα2 IPD | | | | comprising Q139E, |
| | | | | | S172D mutations |
| CODA1311 | α7-nAChR(Gly-cys) | Q139W | S172D | | SEQ ID NO: 65 |
| | LBD: GlyRα2 IPD | | | | comprising Q139W, |
| | | | | | S172D mutations |
| CODA1316 | α7-nAChR(Gly-cys) | T225I | R101W | Y115E, | SEQ ID NO: 65 |
| | LBD: GlyRα2 IPD | | | Y210W | comprising R101W, |
| | | | | | Y115E, Y210W, |
| | | | | | T225I mutations |
| CODA1342 | α7-nAChR(Gly-cys) | | | | SEQ ID NO: 66 |
| | LBD: GlyRα3 isoform | | | | |
| | L IPD | | | | |
| CODA1343 | α7-nAChR(Gly-cys) | | | | SEQ ID NO: 71 |
| | LBD: GlyRα3 isoform | | | | |
| | K IPD | | | | |
| CODA1344 | α7-nAChR LBD: GlyRα3 | | | | SEQ ID NO: 63 |
| | isoform L IPD | | | | |
| CODA1345 | α7-nAChR LBD: GlyRα3 | | | | SEQ ID NO: 70 |
| | isoform K IPD | | | | |

### Binding Agents

The terms “binding agent” or “agent” are used interchangeably herein and refer to exogenous drugs or compounds with a known mechanism of action on a mammalian cell (e.g., are known to act as an agonist, antagonist, or modulator of a receptor). Binding agents can include proteins, lipids, nucleic acids, and/or small molecules. In some embodiments, binding agents include drugs or compounds that have been approved by the US Food and Drug Administration (FDA) for clinical use in the treatment of a particular disease (e.g., a neurological disease). In some embodiments, binding agents include drugs or compounds that have not been approved by the FDA for clinical use, but have been tested in one or more clinical trials, are currently being tested in one or more clinical trials, and/or are anticipated to be tested in one or more clinical trials. In some embodiments, binding agents include drugs or compounds that have not been approved by the FDA for clinical use, but are routinely used in laboratory research. In some embodiments, the binding agent is an analog of one of the aforementioned agents. In particular embodiments, a binding agent is selected from any one of the agents in Tables 2-9. In some embodiments, the binding agent is selected from the group consisting of AZD0328, ABT-126, AQW-051, Cannabidiol, Cilansetron, PH-399733, FACINICLINE/RG3487/MEM-3454, TC-6987, APN-1125, and TC-5619/AT-101. In some embodiments, the binding agent is selected from the group consisting of ABT-126, AZD-0328, APN-1125, RG3487, TC-6987, and TC-5619.

In particular embodiments, the binding agent is an analog of Cilansetron, e.g. as described by one of the compound formulas 2-7 below in either its R or S enantiomer:

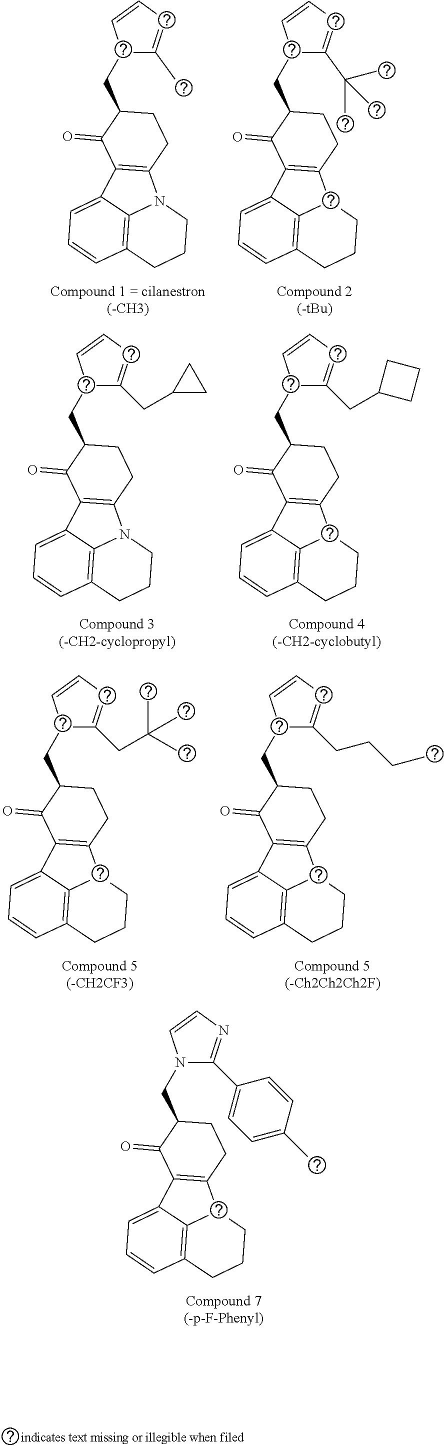

In some embodiments, the binding agent acts as an agonist. The term “agonist” as used herein refers to a ligand or binding agent that induces a signaling response. In some embodiments, the binding agent acts as an antagonist. The term antagonist is used herein to refer to an agent that inhibits a signaling response.

In some embodiments, the binding agent is an anxiolytic, anticonvulsant, antidepressant, antipsychotic, antiemetic, nootropic, antibiotic, antifungal, antiviral, or an antiparasitic.

TABLE 2
Binding agents for Glycine Receptor (GlyR)

| Agonists | | Modulators/Binders | Antagonists | |
|---|---|---|---|---|
| Bilobalide | L-Serine | Gavestinel | 468816 | Lindane |
| Cannabidiol | MDL-27531 | Halothane | ACEA-2085 | MDL-100748 |
| D-Alanine | Methoxyflurane | | Bicuculline | MDL-102288 |
| D-Serine | Milacemide | | Brucine | MDL-105519 |
| Desflurane | Moxidectin | | Caffeine | PD-165650 |
| Doramectin | NRX-1050 | | Gavestinel | Picrotoxin |
| Emamectin | NRX-1060 | | Ginkgo biloba | Strychnine |
| Eprinomectin | P-9939 | | GV-196771 | Thiocolchicoside |
| Ethanol | Quisqualamine | | GW 468816 | Tutin |
| Glycine | Rapastinel | | HMR-2371 | UK-315716 |
| Hypotaurine | S-18841 | | L-695902 | ZD-9379 |
| Isoflurane | Sarcosine | | L-701324 | |
| Ivermectin | Sevoflurane | | | |
| L-Alanine | Taurine | | | |
| L-Proline | β-Alanine | | | |

TABLE 3
Binding Agents for λ-Aminobutyric Acid A Receptor (GABA-A)

| Agonists | Modulators/Binders | | | Antagonists |
|---|---|---|---|---|
| 3-acyl-4- | (−)Epigallo- | Etifoxine | Pentobarbital | (±)-cis-(3- |
| quinolone | catechin-3- | | | Aminocyclopentyl) |
| | Gallate | | | butylphosphinic acid |
| Acamprosate | 10-Methoxy- | Etizolam | Petrichloral | (S)-(4-Aminocyclopent-1- |
| | yangonin | | | enyl)butylphosphinic acid |
| Alfadolone | 11-Hydroxy- | Etomidate | PF-4480682 | Amoxapine |
| | yangonin | | | |
| Bamaluzole | 11-Methoxy-12- | Evt-201 | Phenazepam | Bicuculline |
| | Hydroxy- | | | |
| | dehydrokavain | | | |
| Basmisanil | 11-Methoxy- | Fasiplon | Phenobarbital | CGP-36742 (3- |
| | yangonin | | | aminopropyl-n-butyl- |
| | | | | phosphinic acid) |
| Bretazenil | 123i-Iomazenil | Fg-8205 | Pinazepam | Flumazenil |
| CACA | 2-Oxoquazepam | Fletazepam | Pipequaline | Gabazine |
| CAMP | 3-Hydroxy- | Flubromazepam | Pivoxazepam | Ginkgo biloba |
| | phenazepam | | | |
| CP-409092 | 5-Hydroxykavain | Flubromazolam | Potassium | Lindane |
| | | | Bromide | |
| Doramectin | 5,6-Dehydro- | Fludiazepam | Prazepam | Methohexital |
| | methysticin | | | |
| Emamectin | 5,6-Dihydro- | Flumazenil | Premazepam | Picrotoxin |
| | yangonin | | | |
| Eprinomectin | 5,6,7,8- | Flunitrazepam | Primidone | SKF-97541 (3- |
| | Tetrahydro- | | | Aminopropyl |
| | yangonin | | | (methyl)phosphinic acid) |
| Eszopiclone | 7,8- | Flurazepam | Proflazepam | TPMPA |
| | Dihydrokavain | | | |
| Ethanol | 7,8-Dihydro- | Flutazolam | Propanidid | ZAPA ((Z)-3- |
| | methysticin | | | [(Aminoiminomethyl)thio]prop- |
| | | | | 2-enoic acid) |
| Etomidate | 7,8-Dihydro- | Flutemazepam | Propofol | |
| | yangonin | | | |
| Flunitrazepam | Abecarnil | Flutoprazepam | PWZ-007A | |
| GABA | Adinazolam | Fosazepam | PWZ-009A1 | |
| Gabamide | Allobarbital | Fospropofol | Pwz-029 | |
| GABOB | Allo- | Ganaxolone | Pyrazolam | |
| | pregnanolone | | | |
| Gaboxadol | Alphaxolone | Gbld-345 | PZ-II-028 | |
| Gamma | Alphenal | Gedocarnil | PZ-II-029 | |
| Hydroxybutyric | | | | |
| Acid | | | | |
| Glutethimide | Alpidem | Gidazepam | Qh-Ii-66 | |
| Ibotenic acid | Alprazolam | Girisopam | Quazepam | |
| Imidazenil | Amentoflavone | Glutethimide | Quinidine | |
| | | | Barbiturate | |
| Isoflurane | Amobarbital | Gyki-52466 | Reclazepam | |
| Isoguvacine | Apigenin | Gyki-52895 | Remimazolam | |
| Isonipecotic | Aprobarbital | Halazepam | Rilmazafone | |
| acid | | | | |
| Ivermectin | Arfendazam | Haloxazolam | Ripazepam | |
| F-830982 | Avizafone | Heptabarbital | Ro15-4513 | |
| Meprobamate | AZD7325 | Hexobarbital | Ro48-6791 | |
| Methoxyflurane | Baicalein | Iclazepam | Ro48-8684 | |
| MK- | Baicalin | Imidazenil | Ro4938581 | |
| 0777/L83098 | | | | |
| Methyprylon | Barbital | Indiplon | Rwj-51204 | |
| Moxidectin | Barbituric Acid | Irazepine | Saripidem | |
| | Derivative | | | |
| Muscimol | Bentazepam | Kavain | Sarmazenil | |
| N4- | Brallobarbital | Kenazepine | Sb-205,384 | |
| Chloroacetyl- | | | | |
| cytosine | | | | |
| arabinoside | | | | |
| Pagoclone | Bretazenil | Ketazolam | Scutellarein | |
| Phenibut | Bromazepam | L-655708 | Secobarbital | |
| Picamilon | Brotizolam | L-838,417 | Sevoflurane | |
| Piperazine | Butalbital | Lanthanum | Sh-053-R-Ch3- | |
| | | | 2′F | |
| Piperidine-4- | Butethal | LAU 156 | Skull- | |
| sulfonic acid | | | capflavone II | |
| Progabide | Butobarbital | LAU 157 | Sl-651,498 | |
| QH-ii-066 | Camazepam | LAU 159 | Sodium Amytal | |
| Quisqualamine | Carburazepam | LAU 161 | Sodium | |
| | | | Pentothal | |
| Sevoflurane | Carisoprodol | LAU 162 | Stiripentol | |
| SL 75102 | CGS 20625 | LAU 163 | Sulazepam | |
| SL-651,498 | CGS 20625 | LAU 176 | Sulfonmethane | |
| Thiamylal | CGS 8216 | LAU 177 | Suproclone | |
| Thiomuscimol | CGS 9895 | LAU 206 | Suriclone | |
| Tolgabide | CGS 9896 | Lofendazam | Sx-3228 | |
| Topiramate | Chloral Hydrate | Lopirazepam | Talampanel | |
| Zolpidem | Chloralose | Loprazolam | Talbutal | |
| α5IA | Chlordiazepoxide | Lorazepam | Taniplon | |
| | Chlormezanone | Lorbamate | Temazepam | |
| | Chloroform | Loreclezole | Tetrazepam | |
| | Ciclotizolam | Lorediplon | Tetronal | |
| | Cinazepam | Lormetazepam | Thdoc | |
| | Cinolazepam | Meclonazepam | Theanine | |
| | Cl-218,872 | Medazepam | Thiamylal | |
| | Clazolam | Menitrazepam | Thieno- | |
| | | | diazepine | |
| | Climazolam | Mephobarbital | Thiopental | |
| | Clobazam | Meprobamate | Tofisopam | |
| | Clomethiazole | Metaclazepam | Tolufazepam | |
| | Clonazepam | Methaqualone | Tp-003 | |
| | Clonazolam | Metharbital | Tp-13 | |
| | Clorazepate | Methohexital | Tpa-023 | |
| | Clotiazepam | Methyl- | Triazolam | |
| | | phenobarbital | | |
| | Cloxazolam | Methyprylon | Triflubazam | |
| | Cp-1414s | Methysticin | Triflunor- | |
| | | | dazepam | |
| | CTP-354 | Metizolam | Trional | |
| | Cyclobarbital | Mexazolam | Tuclazepam | |
| | Cyprazepam | Midazolam | Uldazepam | |
| | Delorazepam | Motrazepam | Valerenic Acid | |
| | Demoxepam | N-Desalkyl- | Valeric Acid | |
| | | flurazepam | | |
| | Deschloro- | Necopidem | Wogonin | |
| | etizolam | | | |
| | Desmethoxy- | Nerisopam | XHe-II-006 | |
| | yangonin | | | |
| | Desmethyl- | Niacin | XHe-II-019 | |
| | flunitrazepam | | | |
| | Diazepam | Niacinamide | XHe-II-087c | |
| | Diclazepam | Nifoxipam | XHe-II-094 | |
| | Diethyl Ether | Nimetazepam | XHe-II-098b | |
| | Dihydro- | Nitrazepam | XHe-II-17 | |
| | ergotoxine | | | |
| | Dihydroquinidine | Nitrazepate | XHe-III-006c | |
| | Barbiturate | | | |
| | Diproqualone | Nitrazolam | XHe-III-063 | |
| | Divaplon | Nordazepam | XHe-III-24 | |
| | Doxefazepam | Nortetrazepam | XHe/ON-I | |
| | Elb-139 | Ns-2664 | Y-23684 | |
| | Elfazepam | Ns-2710 | Yangonin | |
| | Estazolam | Ocinaplon | Zaleplon | |
| | Eszopiclone | Oroxylin A | Zapizolam | |
| | Etaqualone | Oxazepam | Zinc | |
| | Etazepine | Oxazolam | Zk-93423 | |
| | Etazolate | Pagoclone | Zolazepam | |
| | Ethyl | Panadiplon | Zolpidem | |
| | Carfluzepate | | | |
| | Ethyl Dirazepate | Pazinaclone | Zomebazam | |
| | Ethyl Loflazepate | PB-XHe | Zopiclone | |

TABLE 4
Binding Agents for 5-Hydroxytryptamine Receptor (5-HT3)

| Agonists | Modulators/Binders | Antagonists | |
|---|---|---|---|
| 2-methyl-5-HT | 5-chloroindole | 3-Tropanyl indole-3- | Mianserin |
| | | carboxylate | |
| Alpha-Methyltryptamine | Trimipramine | Adr-851 | Mirisetron Maleate |
| Bufotenin | | Adr-882 | Mirtazapine |
| Chlorophenyl-biguanide | | Alosetron | Ml-1035 |
| Cisapride | | Amoxapine | Mm-218 |
| DDP733 | | Anpirtoline | N-3256 |
| Ethanol | | Aripiprazole | Netupitant |
| Ethanol | | AS-8112 | Olanzapine |
| Ibogaine | | Azasetron | Ondansetron |
| Metoclopramide | | Batanopride | Org-4419 |
| Phenylbiguanide | | Bimu-1 | Org-4419-2 |
| Quipazine | | Chloroprocaine | Palonosetron |
| RS-56812 | | Cilansetron | Pancopride |
| SR-57227 | | Clozapine | Procaine |
| Tapentadol | | Cp-81386 | Quetiapine |
| Varenicline | | Cp-93318 | R-093777 |
| YM-31636 | | Cr-3124 | R-Zacopride |
| | | DA-9701 | Ramosetron |
| | | Daizac | Renzapride |
| | | Dat-582 | Rg-12915 |
| | | Dau-6285 | Ricasetron |
| | | Ddp-225 | Rocuronium |
| | | Dolasetron | Rs-16566 |
| | | E-3620 | Rs-33800 |
| | | Fabesetron | Rs-56532 |
| | | Facinicline/RG3487 | Rs-56812 |
| | | Hydrochloride | |
| | | Galdansetron | S-21007 |
| | | Gastroprokinetics | Sc-50410 |
| | | Gk-128 | Sc-52150 |
| | | Gr-65630 | Sc-52246 |
| | | Granisetron | Sc-52491 |
| | | Gyki-46903 | Sc-54750 |
| | | Itasetron | Sdz-Icm-567 |
| | | Kb-6806 | Sep-226332 |
| | | Kf-18259 | Srss-021 |
| | | Kf-20170 | Tedatioxetine |
| | | Kga-0941 | Thujone |
| | | L-683877 | Topiramate |
| | | Lamotrigine | Tropisetron |
| | | Lerisetron | Tubocurarine |
| | | Lintopride | Tzb-30878 |
| | | Litoxetine | Va-21B7 |
| | | Loxapine | Vi-0134 |
| | | Lurosetron | Vortioxetine |
| | | Fy-278584 | Vortioxetine |
| | | | Hydrobromide |
| | | M1, the major active | Way-100289 |
| | | metabolite of mosapride | |
| | | Mci-225 | Ym-114 |
| | | Mci-225 | Ym-26103-2 |
| | | Memantine | Ym-26308-2 |
| | | Menthol | Zacopride |
| | | Methadone | Zatosetron |
| | | Metoclopramide | Ziprasidone |

TABLE 5
Binding Agents for Nicotinic Acetylcholine Receptor (nAchR)

| Agonists | Modulators/Binders | Antagonists |
|---|---|---|
| (+)-N-(1-azabicyclo[2.2.2]oct-3- | A-867744 | (−)-7-methyl-2-exo-[3′-(6- |
| yl)benzo[b]furan-2-carboxamide | | [18F]fluoropyridin-2-yl)-5′- |
| | | pyridinyl]-7-azabicyclo[2.2.1]heptane |
| 3-Bromocytisine | AQW-051 | 2-fluoro-3-(4-nitro- |
| | | phenyl)deschloroepibatidine |
| A-366,833 | AVL-3288 | Acv-1 |
| A-582941 | Desformylflustrabromine | ACVx |
| A-82695 | Ethanol | Amobarbital |
| A-84,543 | Galantamine | Anandamide |
| ABT-089 | Ivermectin | Anq-9040 |
| ABT-126 | Nefiracetam | Aprobarbital |
| Abt-202 | NS-1738 | ATG-001 |
| ABT-418 | NS-9283 | ATG003 |
| Abt-418 | PNU-120,596 | Atracurium besylate |
| Abt-560 | Tetraethylammonium | Barbital |
| ABT-894 | | Barbituric acid derivative |
| Acetylcholine | | Biperiden |
| Altinicline | | Bupropion |
| Altinicline | | Butabarbital |
| Aminoethoxypyridine | | Butalbital |
| Anabasine | | Butethal |
| AR-R17779 | | Chloroprocaine |
| Asm-024 | | Cisatracurium Besilate |
| AZD-0328 | | Cisatracurium besylate |
| Bupropion Hydrochloride | | Coclaurine |
| Carbachol | | Dehydronorketamine |
| Choline | | Deuterated Bupropion |
| Cm-2433 | | Dextromethorphan |
| CP-601927 | | Doxacurium chloride |
| CP-601932 | | Ethanol |
| Cp-810123 | | Gallamine Triethiodide |
| Cytisine | | Heptabarbital |
| DBO-83 | | Hexobarbital |
| Decamethonium | | Hydroxybupropion |
| Dianicline | | Hydroxynorketamine |
| Dianicline | | Inaperisone |
| Encenicline | | Iptakalim |
| Epibatidine | | Isoflurane |
| EVP-6124 | | Ketamine |
| Evp-6124 | | Kynurenic acid |
| Galantamine | | Levomethadyl Acetate |
| GTS-21 | | Lobeline |
| Gts-21 | | Mecamylamine |
| ICH-3 | | Mecamylamine |
| Ispronicline | | Memantine |
| Levamisole | | Methadone |
| Lobeline | | Metharbital |
| Lobeline Sulphate | | Methyllycaconitine |
| Mem-3454 | | Methylphenobarbital |
| MEM-63908 | | Metocurine |
| Mem-63908 | | Metocurine Iodide |
| N-(3-pyridinyl)-bridged bicyclic | | Mivacurium |
| diamine | | |
| Nicotine | | Neramexane |
| PH-399733 | | Nic-002 |
| Ph-399733 | | Nitrous oxide |
| PHA-543,613 | | Norketamine |
| Pha-543613 | | Org-9991 |
| PHA-709829 | | Pancuronium |
| PNU-282,987 | | Pentobarbital |
| Pnu-282987 | | Pentolinium |
| Pozanicline | | Phenobarbital |
| Pozanicline | | Pipecuronium |
| Rivanicline | | Pipecuronium |
| Rivanicline | | PNU-120,596 |
| Rjr-1401 | | Primidone |
| Sar-130479 | | Procaine |
| Sazetidine A | | Quinolizidine (−)-1-epi-207I |
| Sib-1663 | | RJR-2531 |
| Sib-1765f | | Rocuronium |
| Sib-3182 | | RPI-78M |
| Sofinicline | | Secobarbital |
| SSR-180,711 | | Talbutal |
| Succinylcholine | | Thiopental |
| Suvn-F90101 | | Trimethaphan |
| Suvn-F91201 | | Tubocurarine |
| Tacrine-huperzine A | | U-2902 |
| TC-1698 | | Vecuronium |
| TC-1827 | | α-Bungarotoxin |
| Tc-2216 | | α-Conotoxin |
| Tc-2403 | | |
| Tc-2429 | | |
| Tc-2559 | | |
| Tc-2696 | | |
| TC-5619 | | |
| Tebanicline | | |
| Tropisetron | | |
| Ub-165 | | |
| Varenicline | | |
| WAY-317,538 | | |
| APN-1125 | | |

TABLE 6
Binding Agents for ATP-Gated P2X Receptor Cation Channel (P2X)

| Agonists | Modulators/Binders | Antagonists | |
|---|---|---|---|
| ATP | Ivermectin | 4-benzoyl-1-substituted- | MK-801 |
| | | piperazin-2-ones | |
| BzATP | | 5-methyl-6,7-dihydro- | MRS2159 |
| | | 5Hcyclopentapyrazine | |
| α,β-meATP | | 5-oxo-3- | NF023 |
| | | pyrrolidinecarboxamides | |
| | | 5,6,7,8-tetrahydropyrido[4,3,d]- | NF279 |
| | | pyrimidines | |
| | | A-317491 | NF449 |
| | | Amitriptyline | Nortriptyline |
| | | Azaindole-3-carboxamides | Oxoisoquinoline carboxamides |
| | | AZD-9056 | Paroxetine |
| | | Benzamide 6 | Piperazines |
| | | Benzofuro-1,4-diazepin-2-ones | Polycyclic guanines |
| | | Benzoimidazoles | PPADS |
| | | Biaryl benzamides | PPNDS |
| | | Carboxamides | Pyrazolo-[1,5,a]-pyridine |
| | | | carboxamides |
| | | CE-224535 | Pyridazinones |
| | | Desipramine | Pyridoxal-5-phosphate |
| | | Diaminopyridines | Pyrrolinones |
| | | Doxepin | Pyrrolo-[2,3,b]-pyridine |
| | | | carboxamides |
| | | Eslicarbazepine acetate | Pyrrolopyrimidin-7-ones |
| | | EVT-401 | Quinoline carboxamides |
| | | Fluoxetine | RO-3 |
| | | Hyaluronic acid derivatives | RO-4 |
| | | Imipramine | RO-51 |
| | | Indole carboxamides | Spinorphins |
| | | Indole-3-carboxamides | Suramin |
| | | Ip5I | Tetrahydro-2H-1,2-thiazine |
| | | | 1,1-dioxides |
| | | Isoquinoline carboxamides | TNP-ATP |
| | | Isothiazolidine 1,1-dioxides | |

TABLE 7
Binding Agents for Inwardly Rectifying Potassium Channel (Kir)

| | | |
|---|---|---|
| Agonists | Modulators/Binders | Antagonists |

| | |
|---|---|
| Diazoxide | Acetohexamide |
| Iptakalim | Carbutamide |
| Minoxidil | Chlorpropamide |
| Nicorandil | Glibenclamide |
| Phosphatidylinositol | Glibenclamide |
| 4,5-Bisphosphate | |
| Pinacidil | Glibomuride |
| Gliclazide | |
| Glimepiride | |
| Glipizide | |
| Gliquidone | |
| Glisoxepide | |
| Glyburide | |
| Glyclopyramide | |
| Glycyclamide | |
| Metahexamide | |
| Tolazamide | |
| Tolbutamide | |
| Tolhexamide | |

TABLE 8
Binding Agents for Voltage Dependent
Potassium Channel (KCNQ/Kv7)

| | | |
|---|---|---|
| Agonists | Modulators/Binders | Antagonists |

| | |
|---|---|
| Diazoxide | Azimilide |
| Flupirtine | Amiodarone |
| Minoxidil | Bretylium |
| Nicorandil | Clofilium |
| Pinacidil | Dalfampridine |
| Retigabine | Dofetilide |
| E-4031 | |
| Ibutilide | |
| Nifekalant | |
| Sematilide | |
| Sotalol | |
| Sulfonylureas | |
| Tedisamil | |

TABLE 9
Binding Agents for Cystic Fibrosis Transmembrane
Conductance Regulator (CFTR)

| | | |
|---|---|---|
| Agonists | Modulators/Binders | Antagonists |

| | |
|---|---|
| 8-cyclopentyl-1,3-dipropylxanthine | Bumetanide |
| 8-methoxypsoralen | Crofelemer |
| Apigenin | Glyburide |
| CTP-656 | Ibuprofen |
| Genistein | |
| IBMX | |
| Ivacaftor | |
| Lumacaftor | |

### Polynucleotides

In various illustrative embodiments, the present disclosure contemplates, in part, polynucleotides, polynucleotides encoding engineered receptor polypeptides including LGICs, and subunits and muteins thereof, and fusion polypeptides, viral vector polynucleotides, and compositions comprising the same.

As used herein, the terms “polynucleotide,” “nucleotide,” “nucleotide sequence” or “nucleic acid” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Polynucleotides may be deoxyribonucleic acid (DNA), ribonucleic acid (RNA) or DNA/RNA hybrids. Polynucleotides may be single-stranded or double-stranded. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (gRNA), plus strand RNA (RNA(+)), minus strand RNA (RNA(−)), synthetic RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 (including all ranges and subranges therebetween) or more nucleotides in length, either ribonucleotides or deoxynucleotides or a modified form of either type of nucleotide, as well as all intermediate lengths. It will be readily understood that “intermediate lengths” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% (including all ranges and subranges therebetween) sequence identity to a reference sequence described herein or known in the art, typically where the variant maintains at least one biological activity of the reference sequence unless otherwise stated.

As used herein, the term “gene” may refer to a polynucleotide sequence comprising enhancers, promoters, introns, exons, and the like. In particular embodiments, the term “gene” refers to a polynucleotide sequence encoding a polypeptide, regardless of whether the polynucleotide sequence is identical to the genomic sequence encoding the polypeptide.

As used herein, a “cis-acting sequence, “cis-acting regulatory sequence”, or “cis-acting nucleotide sequence” or equivalents refers to a polynucleotide sequence that is associated with the expression, e.g. transcription and/or translation, of a gene. In one embodiment, the cis-acting sequence regulates transcription because it is a binding site for a polypeptide that represses or decreases transcription or a polynucleotide sequence associated with a transcription factor binding site that contributes to transcriptional repression. Examples of cis-acting sequences that regulate the expression of polynucleotide sequences and that may be operably linked to the polynucleotides of the present disclosure to regulate the expression of the subject engineered receptors are well known in the art and include such elements as promoter sequences (e.g CAG, CMV, SYN, CamKII, TRPV1), Kozak sequences, enhancers, posttranscriptional regulatory elements, miRNA binding elements, and polyadenylation sequences.

As one non-limiting example, a promoter sequence is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters may be used to drive the various vectors of the present invention. For example, the promoter may be a constitutively active promoter, i.e. a promoter that is active in the absence externally applied agents, e.g. the CMV IEl promoter, the SV40 promoter, GAPDH promoter, Actin promoter. The promoter may be an inducible promoter, i.e. a promoter whose activity is regulated upon the application of an agent to the cell, e.g. doxycycline, the tet-on or tet-off promoter, the estrogen receptor promoter, etc. The promoter may be a tissue-specific promoter, i.e. a promoter that is active on certain types of cells.

In some embodiments, the promoter is active in an excitable cell. By an “excitable cell”, it is meant a cell that is activated by a change in membrane potential, e.g. a neuron or myocyte, e.g. a dorsal root ganglion neuron, a motor neuron, an excitatory neuron, an inhibitory neuron, or a sensory neuron. Promoters that are active in an excitable cell that would find use in the present polynucleotide compositions would include neuronal promoters, for example, the synapsin (SYN), TRPV1, Nay1.7, Nay1.8, Nay1.9, CamKII, NSE, and Advillin promoters; myocyte promoters, e.g. the desmin (Des), alpha-myosin heavy chain (α-MHC), myosin light chain 2 (MLC-2) and cardiac troponin C (cTnC) promoters; and ubiquitous acting promoters, e.g. CAG, CBA, ElFa, Ubc, CMV, and SV40 promoters.

As used herein, a “regulatory element for inducible expression” refers to a polynucleotide sequence that is a promoter, enhancer, or functional fragment thereof that is operably linked to a polynucleotide to be expressed and that responds to the presence or absence of a molecule that binds the element to increase (turn-on) or decrease (turn-off) the expression of the polynucleotide operably linked thereto. Illustrative regulatory elements for inducible expression include, but are not limited to, a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

A “regulatory element for transient expression” refers to a polynucleotide sequence that can be used to briefly or temporarily express a polynucleotide nucleotide sequence. In particular embodiments, one or more regulatory elements for transient expression can be used to limit the duration of a polynucleotide. In certain embodiments, the preferred duration of polynucleotide expression is on the order of minutes, hours, or days. Illustrative regulatory elements for transient expression include, but are not limited to, nuclease target sites, recombinase recognition sites, and inhibitory RNA target sites. In addition, to some extent, in particular embodiments, a regulatory element for inducible expression may also contribute to controlling the duration of polynucleotide expression.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% (including all ranges and subranges therebetween) sequence identity to a reference sequence described herein or known in the art, typically where the variant maintains at least one biological activity of the reference sequence unless otherwise stated.

In one embodiment, a polynucleotide comprises a nucleotide sequence that hybridizes to a target nucleic acid sequence under stringent conditions. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant DNA, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the pre-messenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The term “flanked” refers to a polynucleotide sequence that is in between an upstream polynucleotide sequence and/or a downstream poylnucleotide sequence, i.e., 5′ and/or 3′, relative to the sequence. For example, a sequence that is “flanked” by two other elements (e.g., ITRs), indicates that one element is located 5′ to the sequence and the other is located 3′ to the sequence; however, there may be intervening sequences therebetween.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The terms “nucleic acid cassette” or “expression cassette” as used herein refers to polynucleotide sequences within a larger polynucleotide, such as a vector, which are sufficient to express one or more RNAs from a polynucleotide. The expressed RNAs may be translated into proteins, may function as guide RNAs or inhibitory RNAs to target other polynucleotide sequences for cleavage and/or degradation. In one embodiment, the nucleic acid cassette contains one or more polynucleotide(s)-of-interest. In another embodiment, the nucleic acid cassette contains one or more expression control sequences operably linked to one or more polynucleotide(s)-of-interest. Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, e.g., LGICs, and subunits and muteins thereof, as contemplated herein. In a particular embodiment, a polynucleotide-of-interest encodes a polypeptide or fusion polypeptide having one or more enzymatic activities, such as a nuclease activity and/or chromatin remodeling or epigenetic modification activities.

Vectors may comprise 1, 2, 3, 4, 5, 6, 7, 8, 9 or 10 or more nucleic acid cassettes. In a preferred embodiment of the disclosure, a nucleic acid cassette comprises one or more expression control sequences (e.g., a promoter or enhancer operable in a neuronal cell) operably linked to a polynucleotide encoding a engineered receptor, e.g., an LGIC, or subunit or muteins thereof. The cassette can be removed from or inserted into other polynucleotide sequences, e.g., a plasmid or viral vector, as a single unit.

In one embodiment, a polynucleotide contemplated herein comprises 1, 2, 3, 4, 5, 6, 7, 8, 9, or more nucleic acid cassettes any number or combination of which may be in the same or opposite orientations.

Moreover, it will be appreciated by those of ordinary skill in the art that, as a result of the degeneracy of the genetic code, there are many nucleotide sequences that may encode a polypeptide, or fragment of variant thereof, as contemplated herein. Some of these polynucleotides bear minimal homology to the nucleotide sequence of any native gene. Nonetheless, polynucleotides that vary due to differences in codon usage are specifically contemplated by the present disclosure, for example polynucleotides that are optimized for human and/or primate codon selection. In one embodiment, polynucleotides comprising particular allelic sequences are provided. Alleles are endogenous polynucleotide sequences that are altered as a result of one or more mutations, such as deletions, additions and/or substitutions of nucleotides.

### Vectors

In some aspects of the disclosure, a nucleic acid molecule, i.e., a polynucleotide encoding an engineered receptor is delivered to a subject. In some cases, the nucleic acid molecule encoding the engineered receptor is delivered to a subject by a vector. In various embodiments, a vector comprises a one or more polynucleotide sequences contemplated herein. The term “vector” is used herein to refer to a nucleic acid molecule capable of transferring or transporting another nucleic acid molecule. The transferred polynucleotide is generally linked to, e.g., inserted into, the vector nucleic acid molecule. A vector may include sequences that direct autonomous replication in a cell, or may include sequences sufficient to allow integration into host cell DNA. A vector can deliver a target polynucleotide to an organism, a cell or a cellular component. In some cases, the vector is an expression vector. An “expression vector” as used herein refers to a vector, for example, a plasmid, that is capable of promoting expression, as well as replication of a polynucleotide incorporated therein. Typically, the nucleic acid sequence to be expressed is operably linked to cis-acting regulatory sequence, e.g. a promoter and/or enhancer sequence, and is subject to transcription regulatory control by the promoter and/or enhancer. In particular cases, a vector is used to deliver a nucleic acid molecule encoding an engineered receptor of the disclosure to a subject.

In particular embodiments, any vector suitable for introducing an expression cassette or polynucleotide encoding an engineered receptor into a neuronal cell can be employed. Illustrative examples of suitable vectors include plasmids (e.g., DNA plasmids or RNA plasmids), transposons, cosmids, bacterial artificial chromosomes, and viral vectors. In some cases, the vector is a circular nucleic acid, for e.g., a plasmid, a BAC, a PAC, a YAC, a cosmid, a fosmid, and the like. In some cases, circular nucleic acid molecules can be utilized to deliver a nucleic acid molecule encoding an engineered receptor to a subject. For example, a plasmid DNA molecule encoding an engineered receptor can be introduced into a cell of a subject whereby the DNA sequence encoding the engineered receptor is transcribed into mRNA and the mRNA “message” is translated into a protein product. The circular nucleic acid vector will generally include regulatory elements that regulate the expression of the target protein. For example, the circular nucleic acid vector may include any number of promoters, enhancers, terminators, splice signals, origins of replication, initiation signals, and the like.

In some cases, the vector can include a replicon. A replicon may be any nucleic acid molecule capable of self-replication. In some cases, the replicon is an RNA replicon derived from a virus. A variety of suitable viruses (e.g. RNA viruses) are available, including, but not limited to, alphavirus, picornavirus, flavivirus, coronavirus, pestivirus, rubivirus, calcivirus, and hepacivirus.

In some embodiments, the vector is a non-viral vector. By a “non-viral vector”, it is meant any delivery vehicle that does not comprise a viral capsid or envelope, e.g. lipid nanoparticles (anionic (negatively charged), neutral, or cationic (positively charged)), heavy metal nanoparticles, polymer-based particles, plasmid DNA, minicircle DNA, minivector DNA, ccDNA, synthetic RNA, exosomes, and the like. Non-viral vectors may be delivered by any suitable method as would be well understood in the art, including, e.g., nanoparticle delivery, particle bombardment, electroporation, sonication, or microinjection. See, e.g. Chen et al. Mol. Therapy, Methods and Clinical Development. 2016 January; Vol 3, issue 1; and Hardy, C E et al. Genes (Basel). 2017 February; 8(2): 6.5

In other embodiments, the vector is a viral vector. By a “viral vector” it is meant a delivery vehicle that comprises a viral capsid or envelop surrounding a polynucleotide encoding an RNA or polypeptide of interest. In some cases, the viral vector is derived from a replication-deficient virus. Non-limiting examples of viral vectors suitable for delivering a nucleic acid molecule of the disclosure to a subject include those derived from adenovirus, retrovirus (e.g., lentivirus), adeno-associated virus (AAV), and herpes simplex-1 (HSV-1). Illustrative examples of suitable viral vectors include, but are not limited to, retroviral vectors (e.g., lentiviral vectors), herpes virus based vectors and parvovirus based vectors (e.g., adeno-associated virus (AAV) based vectors, AAV-adenoviral chimeric vectors, and adenovirus-based vectors).

The term “parvovirus” as used herein encompasses all parvoviruses, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, mouse minute virus, bovine parvovirus, canine parvovirus, chicken parvovirus, feline panleukopenia virus, feline parvovirus, goose parvovirus, and B19 virus. Other autonomous parvoviruses are known to those skilled in the art. See, e.g., Fields et al., 1996 Virology, volume 2, chapter 69 (3d ed., Lippincott-Raven Publishers).

The genus Dependovirus contains the adeno-associated viruses (AAV), including but not limited to, AAV type 1, AAV type 2, AAV type 3, AAV type 4, AAV type 5, AAV type 6, AAV type 7, AAV type 8, AAV type 9, AAV type rh10, avian AAV, bovine AAV, canine AAV, equine AAV, and ovine AAV.

In a preferred embodiment, the vector is an AAV vector. In particular cases, the viral vector is an AAV-5, AAV-6 or AAV-9 vector.

The genomic organization of all known AAV serotypes is similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VPl, -2 and -3) form the capsid and contribute to the tropism of the virus. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type (wt) AAV infection in mammalian cells the Rep genes are expressed and function in the replication of the viral genome.

In some cases, the outer protein “capsid” of the viral vector occurs in nature, e.g. AAV1, AAV2, AAV3, AAV4, AAV5, AAV6, AAV7, AAV8, AAV9, or AAV10. In particular cases, the capsid is synthetically engineered (e.g. through directed evolution or rational design) to possess certain unique characteristics not present in nature such as altered tropism, increased transduction efficiency, or immune evasion. An example of a rationally designed capsid is the mutation of one or more surface-exposed tyrosine (Y), serine (S), threonine (T), and lysine (K) residues on the VP3 viral capsid protein. Non-limiting examples of viral vectors whose VP3 capsid proteins have been synthetically engineered and are amenable for use with the compositions and methods provided herein include: AAV1(Y705+731F+T492V), AAV2(Y444+500+730F+T491V), AAV3(Y705+731F), AAV5(Y436+693+719F), AAV6(Y705+731F+T492V), AAV8(Y733F), AAV9(Y731F), and AAV10(Y733F). Non-limiting examples of viral vectors that have been engineered through directed evolution and are amenable for use with the compositions and methods provided herein include AAV-7m8 and AAV-ShH10. In some embodiments, the viral vector comprises an AAV capsid protein comprising amino acid mutation at one or more positions corresponding to T492, Y705, Y731, or any combination thereof, of AAV6 capsid protein, wherein the AAV capsid protein is of serotype AAV1, AAV2, AAV3, AAV4, AAV5, AAV6, AAV7, AAV8, AAV9, AAV10, or another AAV serotype. In some embodiments, the one or more positions are two or more positions, two positions, or three positions. In some embodiments, the viral vector comprises an AAV capsid protein comprising one or more amino acid substitutions corresponding to T492V, Y705F or Y731F, or any combination thereof, of AAV6 capsid protein, wherein the AAV capsid protein is of serotype AAV1, AAV2, AAV3, AAV4, AAV5, AAV6, AAV7, AAV8, AAV9, AAV10, or another AAV serotype. In some embodiments, the one or more substitutions are two or more substitutions, two substitutions, or three substitutions.

In some embodiments, the viral vector comprises an AAV6 capsid protein comprising a mutation at one or more amino acid positions selected from T492, Y705 and Y731. In some embodiments, the viral vector comprises an AAV6 capsid protein comprising one or more mutations selected from T492V, Y705F, and Y731F. In some embodiments, the viral vector comprises an AAV6 capsid protein comprising the amino acid mutation T492V+Y705F+Y731F. In some embodiments, the AAV6 capsid protein comprises or consists of an amino acid sequence having at least 85%, at least 90%, at least 95%, at least 96%, at least 97%, at least 98%, at least 99%, or 100% sequence identity to SEQ ID NO: 93.

In some embodiments, the viral vector comprises an AAV9 capsid protein comprising a mutation at one or more amino acid positions selected from T492, Y705 and Y731. In some embodiments, the viral vector comprises an AAV9 capsid protein comprising one or more mutations selected from T492V, Y705F, and Y731F. In some embodiments, the viral vector comprises an AAV9 capsid protein comprising the amino acid mutation T492V+Y705F+Y731F. In some embodiments, the AAV9 capsid protein comprises or consists of an amino acid sequence having at least 85%, at least 90%, at least 95%, at least 96%, at least 97%, at least 98%, at least 99%, or 100% sequence identity to SEQ ID NO: 94.

In some embodiments, the viral vector comprises an AAV5 capsid protein comprising a mutation at one or more amino acid positions selected from Y693 and Y719. In some embodiments, the viral vector comprises an AAV5 capsid protein comprising one or more mutations selected from Y693F and Y719F. In some embodiments, the viral vector comprises an AAV5 capsid protein comprising the amino acid mutation Y693F+Y719F. In some embodiments, the AAV5 capsid protein comprises or consists of an amino acid sequence having at least 85%, at least 90%, at least 95%, at least 96%, at least 97%, at least 98%, at least 99%, or 100% sequence identity to SEQ ID NO: 92.

In some embodiments, the viral vector comprising the AAV capsid protein (or a mutant of the disclosure) contributes to targeted expression of an engineered receptor to a sub-population of cells or neurons in a subject. In some embodiments, the neurons are nociceptors.

A “recombinant parvoviral or AAV vector” (or “rAAV vector”) herein refers to a vector comprising one or more polynucleotides contemplated herein that are flanked by one or more AAV ITRs. Such polynucleotides are said to be “heterologous” to the ITRs, as such combinations do not ordinarily occur in nature. Such rAAV vectors can be replicated and packaged into infectious viral particles when present in an insect host cell that is expressing AAV rep and cap gene products (i.e., AAV Rep and Cap proteins). When an rAAV vector is incorporated into a larger nucleic acid construct (e.g., in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

In particular embodiments, any AAV ITR may be used in the AAV vectors, including ITRs from AAV1, AAV2, AAV3, AAV4, AAV5, AAV6, AAV7, AAV8, AAV9, AAV10, AAV11, AAV12, AAV13, AAV14, AAV15, and AAV16. In one preferred embodiment, an AAV vector contemplated herein comprises one or more AAV2 ITRs.

rAAV vectors comprising two ITRs have a payload capacity of about 4.4 kB. Self-complementary rAAV vectors contain a third ITR and package two strands of the recombinant portion of the vector leaving only about 2.1 kB for the polynucleotides contemplated herein. In one embodiment, the AAV vector is an scAAV vector.

Extended packaging capacities that are roughly double the packaging capacity of an rAAV (about 9 kB) have been achieved using dual rAAV vector strategies. Dual vector strategies useful in producing rAAV contemplated herein include, but are not limited to splicing (trans-splicing), homologous recombination (overlapping), or a combination of the two (hybrid). In the dual AAV trans-splicing strategy, a splice donor (SD) signal is placed at the 3′ end of the 5′-half vector and a splice acceptor (SA) signal is placed at the 5′ end of the 3′-half vector. Upon co-infection of the same cell by the dual AAV vectors and inverted terminal repeat (ITR)-mediated head-to-tail concatemerization of the two halves, trans-splicing results in the production of a mature mRNA and full-size protein (Yan et al., 2000). Trans-splicing has been successfully used to express large genes in muscle and retina (Reich et al., 2003; Lai et al., 2005). Alternatively, the two halves of a large transgene expression cassette contained in dual AAV vectors may contain homologous overlapping sequences (at the 3′ end of the 5′-half vector and at the 5′ end of the 3′-half vector, dual AAV overlapping), which will mediate reconstitution of a single large genome by homologous recombination (Duan et al., 2001). This strategy depends on the recombinogenic properties of the transgene overlapping sequences (Ghosh et al., 2006). A third dual AAV strategy (hybrid) is based on adding a highly recombinogenic region from an exogenous gene (i.e., alkaline phosphatase; Ghosh et al., 2008, Ghosh et al., 2011)) to the trans-splicing vectors. The added region is placed downstream of the SD signal in the 5′-half vector and upstream of the SA signal in the 3′-half vector in order to increase recombination between the dual AAVs.

A “hybrid AAV” or “hybrid rAAV” refers to an rAAV genome packaged with a capsid of a different AAV serotype (and preferably, of a different serotype from the one or more AAV ITRs), and may otherwise be referred to as a pseudotyped rAAV. For example, an rAAV type 1, 2, 3, 4, 5, 6, 7, 8, 9, 10, 11, 12, 13, 14, 15, or 16 genome may be encapsidated within an AAV type 1, 2, 3, 4, 5, 6, 7, 8, 9, 10, 11, 12, 13, 14, 15, or 16 capsid or variants thereof, provided that the AAV capsid and genome (and preferably, the one or more AAV ITRs) are of different serotypes. In certain embodiments, a pseudotyped rAAV particle may be referred to as being of the type “x/y”, where “x” indicates the source of ITRs and “y” indicates the serotype of capsid, for example a 2/5 rAAV particle has ITRs from AAV2 and a capsid from AAV6.

A “host cell” includes cells transfected, infected, or transduced in vivo, ex vivo, or in vitro with a recombinant vector or a polynucleotide of the disclosure. Host cells may include virus producing cells and cells infected with viral vectors. In particular embodiments, host cells in vivo are infected with viral vector contemplated herein. In certain embodiments, the term “target cell” is used interchangeably with host cell and refers to infected cells of a desired cell type.

High titer AAV preparations can be produced using techniques known in the art, e.g., as described in U.S. Pat. Nos. 5,658,776; 6,566,118; 6,989,264; and 6,995,006; U.S. 2006/0188484; WO98/22607; WO2005/072364; and WO/1999/011764; and Viral Vectors for Gene Therapy: Methods and Protocols, ed. Machida, Humana Press, 2003; Samulski et al., (1989) J. Virology 63, 3822; Xiao et al., (1998) J. Virology 72, 2224; lnoue et al., (1998) J. Virol. 72, 7024. Methods of producing pseudotyped AAV vectors have also been reported (e.g., WO 00/28004), as well as various modifications or formulations of AAV vectors, to reduce their immunogenicity upon in vivo administration (see e.g., WO 01/23001; WO 00/73316; WO 04/1 12727; WO 05/005610; WO 99/06562).

### Pharmaceutical Compositions

Also provided are pharmaceutical preparations, including pharmaceutical preparations of vector and pharmaceutical preparations of binding agent. Pharmaceutical preparations include the subject polynucleotide (RNA or DNA) encoding an engineered receptor, vector carrying a polynucleotide (RNA or DNA) encoding a subject engineered receptor, or binding agent present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the disclosure is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compounds and compositions of the disclosure and pharmaceutically acceptable vehicles, excipients, or diluents may be sterile. In some instances, an aqueous medium is employed as a vehicle when the compound of the disclosure is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

The choice of excipient will be determined in part by the particular vector, as well as by the particular method used to administer the composition. Accordingly, there is a wide variety of suitable formulations of the pharmaceutical composition of the present disclosure.

For example, the vector may be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

As another example, the vector may be formulated into a preparation suitable for oral administration, including (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

As another example, the subject formulations of the present disclosure can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (i.e., sunblocking agents), etc.

A compound of the disclosure may be formulated for topical administration. The vehicle for topical application may be in one of various forms, e.g. a lotion, cream, gel, ointment, stick, spray, or paste. They may contain various types of carriers, including, but not limited to, solutions, aerosols, emulsions, gels, and liposomes. The carrier may be formulated, for example, as an emulsion, having an oil-in-water or water-in-oil base. Suitable hydrophobic (oily) components employed in emulsions include, for example, vegetable oils, animal fats and oils, synthetic hydrocarbons, and esters and alcohols thereof, including polyesters, as well as organopolysiloxane oils. Such emulsions also include an emulsifier and/or surfactant, e.g. a nonionic surfactant to disperse and suspend the discontinuous phase within the continuous phase.

Suppository formulations are also provided by mixing with a variety of bases such as emulsifying bases or water-soluble bases. Formulations suitable for vaginal administration may be presented as pessaries, tampons, creams, gels, pastes, foams.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present disclosure calculated in an amount sufficient to produce the desired effect in association with a pharmaceutically acceptable diluent, carrier or vehicle. The specifications for the novel unit dosage forms of the present disclosure depend on the particular compound employed and the effect to be achieved, and the pharmacodynamics associated with each compound in the host.

Dose levels can vary as a function of the specific compound, the nature of the delivery vehicle, and the like. Desired dosages for a given compound are readily determinable by a variety of means.

The dose administered to an animal, particularly a human, in the context of the present disclosure should be sufficient to effect a prophylactic or therapeutic response in the animal over a reasonable time frame, e.g., as described in greater detail below. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

In pharmaceutical dosage forms, the ASC inducer compounds may be administered in the form of a free base, their pharmaceutically acceptable salts, or they may also be used alone or in appropriate association, as well as in combination, with other pharmaceutically active compounds.

### Clinical Applications and Methods of Treatment

The compositions and methods disclosed herein can be utilized to treat a neurological disease or disorder. In some aspects of the disclosure, a method of treating a neurological disease or disorder in a subject is provided, the method comprising the introducing an engineered receptor into a neuronal cell and providing a ligand that activates the engineered receptor in an effective amount to control the activity of the cell, thereby relieving pain in the subject. In some aspects, vectors or compositions disclosed herein are used in the manufacture of a medicament for treating a neurological disease or disorder.

In some cases, the methods and compositions of the disclosure are utilized to treat epilepsy. Compositions described herein may be used to prevent or control epileptic seizures. Epileptic seizures may be classified as tonic-clonic, tonic, clonic, myoclonic, absence or atonic seizures. In some cases, the compositions and methods herein may prevent or reduce the number of epileptic seizures experienced by a subject by about 5%, about 10%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or 100%, including all ranges and subranges therebetween.

In some cases, the methods and compositions of the disclosure are utilized to treat an eating disorder. An eating disorder may be a mental disorder defined by abnormal eating behaviors that negatively affect a subject's physical or mental health. In some cases, the eating disorder is anorexia nervosa. In other cases, the eating disorder is bulimia nervosa. In some cases, the eating disorder is pica, rumination disorder, avoidant/restrictive food intake disorder, binge eating disorder (BED), other specified feeding and eating disorder (OSFED), compulsive overeating, diabulimia, orthorexia nervosa, selective eating disorder, drunkorexia, pregorexia, or Gourmand syndrome. In some cases, the composition includes a G-protein coupled receptor that increases or decreases the production of one or more molecules associated with an eating disorder. In other cases, the composition includes a ligand-gated ion channel that alters the production of one or more molecules associated with an eating disorder. The one or more molecules associated with an eating disorder may include, without limitation, a molecule of the hypothalamus-pituitary-adrenal (HPA) axis, including vasopressin, corticotropin-releasing hormone (CRH), adrenocorticotropic hormone (ACTH), cortisol, epinephrine, or norepinephrine; as well as serotonin, dopamine, neuropeptide Y, leptin, or ghrelin.

In some cases, the compositions and methods are utilized to treat post-traumatic stress disorder (PTSD), gastroesophageal reflex disease (GERD), addiction (e.g., alcohol, drugs), anxiety, depression, memory loss, dementia, sleep apnea, stroke, urinary incontinence, narcolepsy, essential tremor, movement disorder, atrial fibrillation, cancer (e.g., brain tumors), Parkinson's disease, or Alzheimer's disease. Other non-limiting examples of neurological diseases or disorders that can be treated by the compositions and methods herein include: Abulia, Agraphia, Alcoholism, Alexia, Aneurysm, Amaurosis fugax, Amnesia, Amyotrophic lateral sclerosis (ALS), Angelman syndrome, Aphasia, Apraxia, Arachnoiditis, Arnold-Chiari malformation, Asperger syndrome, Ataxia, Ataxia-telangiectasia, Attention deficit hyperactivity disorder, Auditory processing disorder, Autism spectrum, Bipolar disorder, Bell's palsy, Brachial plexus injury, Brain damage, Brain injury, Brain tumor, Canavan disease, Capgras delusion, Carpal tunnel syndrome, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy (CADASIL), Cerebral gigantism, Cerebral palsy, Cerebral vasculitis, Cervical spinal stenosis, Charcot-Marie-Tooth disease, Chiari malformation, Chorea, Chronic fatigue syndrome, Chronic inflammatory demyelinating polyneuropathy (CIDP), Chronic pain, Coffin-Lowry syndrome, Coma, Complex regional pain syndrome, Compression neuropathy, Congenital facial diplegia, Corticobasal degeneration, Cranial arteritis, Craniosynostosis, Creutzfeldt-Jakob disease, Cumulative trauma disorders, Cushing's syndrome, Cyclothymic disorder, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Developmental coordination disorder, Diabetic neuropathy, Diffuse sclerosis, Diplopia, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalotrigeminal angiomatosis, Encopresis, Enuresis, Epilepsy, Epilepsy-intellectual disability in females, Erb's palsy, Erythromelalgia, Exploding head syndrome, Fabry's disease, Fahr's syndrome, Fainting, Familial spastic paralysis, Febrile seizures, Fisher syndrome, Friedreich's ataxia, Fibromyalgia, Foville's syndrome, Fetal alcohol syndrome, Fragile X syndrome, Fragile X-associated tremor/ataxia syndrome (FXTAS), Gaucher's disease, Generalized epilepsy with febrile seizures plus, Gerstmann's syndrome, Giant cell arteritis, Giant cell inclusion disease, Globoid Cell Leukodystrophy, Gray matter heterotopia, Guillain-Barre syndrome, Generalized anxiety disorder, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Hirschsprung's disease, Holmes-Adie syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile Refsum disease, Infantile spasms, Inflammatory myopathy, Intracranial cyst, Intracranial hypertension, Isodicentric 15, Joubert syndrome, Karak syndrome, Kearns-Sayre syndrome, Kinsbourne syndrome, Kleine-Levin Syndrome, Klippel Feil syndrome, Krabbe disease, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Leukoencephalopathy with vanishing white matter, Lewy body dementia, Lissencephaly, Locked-In syndrome, Lumbar disc disease, Lumbar spinal stenosis, Lyme disease—Neurological Sequelae, Machado-Joseph disease (Spinocerebellar ataxia type 3), Macrencephaly, Macropsia, Mal de debarquement, Megalencephalic leukoencephalopathy with subcortical cysts, Megalencephaly, Melkersson-Rosenthal syndrome, Menieres disease, Meningitis, Menkes disease, Metachromatic leukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Motor skills disorder, Moyamoya disease, Mucopolysaccharidoses, Multi-infarct dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neuro-Behget's disease, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Neuropathy, Neurosis, Niemann-Pick disease, Non-24-hour sleep-wake disorder, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Otosclerosis, Overuse syndrome, Palinopsia, Paresthesia, Parkinson's disease, Paramyotonia Congenita, Paraneoplastic diseases, Paroxysmal attacks, Parry-Romberg syndrome, PANDAS, Pelizaeus-Merzbacher disease, Periodic Paralyses, Peripheral neuropathy, Pervasive developmental disorders, Photic sneeze reflex, Phytanic acid storage disease, Pick's disease, Pinched nerve, Pituitary tumors, PMG, Polyneuropathy, Polio, Polymicrogyria, Polymyositis, Porencephaly, Post-Polio syndrome, Postherpetic Neuralgia (PHN), Postural Hypotension, Prader-Willi syndrome, Primary Lateral Sclerosis, Prion diseases, Progressive hemifacial atrophy, Progressive multifocal leukoencephalopathy, Progressive Supranuclear Palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III, Rasmussen encephalitis, Reflex neurovascular dystrophy, Refsum disease, REM sleep behavior disorder, Repetitive stress injury, Restless legs syndrome, Retrovirus-associated myelopathy, Rett syndrome, Reye's syndrome, Rhythmic Movement Disorder, Romberg syndrome, Saint Vitus dance, Sandhoff disease, Schilder's disease, Schizencephaly, Sensory processing disorder, Septo-optic dysplasia, Shaken baby syndrome, Shingles, Shy-Drager syndrome, Sjögren's syndrome, Sleep apnea, Sleeping sickness, Snatiation, Sotos syndrome, Spasticity, Spina bifida, Spinal cord injury, Spinal cord tumors, Spinal muscular atrophy, Spinal and bulbar muscular atrophy, Spinocerebellar ataxia, Split-brain, Steele-Richardson-Olszewski syndrome, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tarsal tunnel syndrome, Tardive dyskinesia, Tardive dysphrenia, Tarlov cyst, Tay-Sachs disease, Temporal arteritis, Temporal lobe epilepsy, Tetanus, Tethered spinal cord syndrome, Thomsen disease, Thoracic outlet syndrome, Tic Douloureux, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Von Hippel-Lindau disease (VHL), Viliuisk Encephalomyelitis (VE), Wallenberg's syndrome, West syndrome, Whiplash, Williams syndrome, Wilson's disease, or Zellweger syndrome.

In some cases, the compositions and methods disclosed herein can be used to treat brain cancer or brain tumors. Non-limiting examples of brain cancers or tumors that may be amenable to treatment with vectors and compositions described herein include: gliomas including anaplastic astrocytoma (grade III glioma), astrocytoma (grade II glioma), brainstem glioma, ependymoma, ganglioglioma, ganglioneuroma, glioblastoma (grade IV glioma), glioma, juvenile pilocytic astrocytoma (JPA), low-grade astrocytoma (LGA), medullablastoma, mixed glioma, oligodendroglioma, optic nerve glioma, pilocytic astrocytoma (grade I glioma), and primitive neuroectodermal (PNET); skull base tumors including acoustic neuroma (vestibular schwannoma), acromegaly, adenoma, chondrosarcoma, chordoma, craniopharyngioma, epidermoid tumor, glomus jugulare tumor, infratentorial meningioma, meningioma, pituitary adenoma, pituitary tumor, Rathke's cleft cyst; metastatic cancer including brain metastasis, metastatic brain tumor; other brain tumors including brain cyst, choroid plexus papilloma, CNS lymphoma, colloid cyst, cystic tumor, dermoid tumor, germinoma, lymphoma, nasal carcinoma, naso-pharyngeal tumor, pineal tumor, pineoblastoma, pineocytoma, supratentorial meningioma, and vascular tumor; spinal cord tumors including astrocytoma, ependymoma, meningioma, and schwannoma.

The present disclosure contemplates, in part, compositions and methods for controlling, managing, preventing, or treating pain in a subject. “Pain” refers to an uncomfortable feeling and/or an unpleasant sensation in the body of a subject. Feelings of pain can range from mild and occasional to severe and constant. Pain can be classified as acute pain or chronic pain. Pain can be nociceptive pain (i.e., pain caused by tissue damage), neuropathic pain or psychogenic pain. In some cases, the pain is caused by or associated with a disease (e.g., cancer, arthritis, diabetes). In other cases, the pain is caused by injury (e.g., sports injury, trauma). Non-limiting examples of pain that are amenable to treatment with the compositions and methods herein include: neuropathic pain including peripheral neuropathy, diabetic neuropathy, post herpetic neuralgia, trigeminal neuralgia, back pain, neuropathy associated with cancer, neuropathy associated with HIV/AIDS, phantom limb pain, carpal tunnel syndrome, central post-stroke pain, pain associated with chronic alcoholism, hypothyroidism, uremia, pain associated with multiple sclerosis, pain associated with spinal cord injury, pain associated with Parkinson's disease, epilepsy, osteoarthritic pain, rheumatoid arthritic pain, visceral pain, and pain associated with vitamin deficiency; and nociceptive pain including pain associated with central nervous system trauma, strains/sprains, and burns; myocardial infarction, acute pancreatitis, post-operative pain, posttraumatic pain, renal colic, pain associated with cancer, pain associated with fibromyalgia, pain associated with carpal tunnel syndrome, and back pain.

The compositions and methods herein may be utilized to ameliorate a level of pain in a subject. In some cases, a level of pain in a subject is ameliorated by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least 99% or about 100%, including all ranges and subranges therebetween. A level of pain in a subject can be assessed by a variety of methods. In some cases, a level of pain is assessed by self-reporting (i.e., a human subject expresses a verbal report of the level of pain he/she is experiencing). In some cases, a level of pain is assessed by behavioral indicators of pain, for example, facial expressions, limb movements, vocalization, restlessness and guarding. These types of assessments may be useful for example when a subject is unable to self-report (e.g., an infant, an unconscious subject, a non-human subject). A level of pain may be assessed after treatment with a composition of the disclosure as compared to the level of pain the subject was experiencing prior to treatment with the composition.

In various embodiments, a method for controlling, managing, preventing, or treating pain in a subject comprises administering to the subject an effective amount of an engineered receptor contemplated herein. Without wishing to be bound by any particular theory, the present disclosure contemplates using the vectors disclosed herein to modulate neuronal activity to alleviate pain in the subject.

In various embodiments, a vector encoding an engineered receptor that activates or depolarizes neuronal cells is administered to (or introduced into) one or more neuronal cells that decrease pain sensation, e.g., inhibitory interneurons. In the presence of ligand the neuronal cell expressing the engineered receptor, is activated and decreases the sensitivity to pain potentiating the analgesic effect of stimulating these neuronal cells.

In various embodiments, a vector encoding an engineered receptor that deactivates or hyperpolarizes neuronal cells is administered to (or introduced into) one or more neuronal cells that increase pain sensation or sensitivity to pain, e.g., nociceptor, peripheral sensory neurons, C-fibers, Aδ fibers, Aβ fibers, DRG neurons, TGG neurons, and the like. In the presence of ligand the neuronal cell expressing the engineered receptor, is deactivated and decreases the sensitivity to pain and potentiating an analgesic effect.

Targeting expression of an engineered receptor to a sub-population of nociceptors can be achieved by one or more of: selection of the vector (e.g., AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10); selection of a promoter; and delivery means.

In particular embodiments, the compositions and methods contemplated herein are effective in reducing pain. Illustrative examples of pain that are amenable to treatment with the vectors, compositions, and methods contemplated herein, include but are not limited to acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritic pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, and menstrual pain.

Pain can be classified as acute or chronic. “Acute pain” refers to pain that begins suddenly and is usually sharp in quality. Acute pain might be mild and last just a moment, or it might be severe and last for weeks or months. In most cases, acute pain does not last longer than three months, and it disappears when the underlying cause of pain has been treated or has healed. Unrelieved acute pain, however, may lead to chronic pain. “Chronic pain” refers to ongoing or recurrent pain, lasting beyond the usual course of acute illness or injury or lasting for more than three to six months, and which adversely affects the individual's well-being. In particular embodiments, the term “chronic pain” refers to pain that continues when it should not. Chronic pain can be nociceptive pain or neuropathic pain.

In some embodiments, the pain is expected or anticipated to develop in association with or as a result of an injury, an infection, or a medical intervention. In some embodiments, the infection causes nerve damage. In some embodiments, the medical intervention is a surgery, such as surgery to the central core of the body. In some embodiments, the medical intervention is a surgery to remove parts or whole of one or more tissues, tumors or organs in the body. In some embodiments, the medical intervention is an amputation. In particular embodiments, the compositions and methods contemplated herein are effective in reducing acute pain. In particular embodiments, the compositions and methods contemplated herein are effective in reducing chronic pain.

Clinical pain is present when discomfort and abnormal sensitivity feature among the patient's symptoms. Individuals can present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia-Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive pain, inflammatory pain, and neuropathic pain.

In particular embodiments, the compositions and methods contemplated herein are effective in reducing nociceptive pain. In particular embodiments, the compositions and methods contemplated herein are effective in reducing inflammatory pain. In particular embodiments, the compositions and methods contemplated herein are effective in reducing neuropathic pain.

Nociceptive pain is induced by tissue injury or by intense stimuli with the potential to cause injury. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g., bone pain, headache, facial pain or visceral pain) or pain associated with cancer therapy (e.g., post chemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain can be defined as pain initiated or caused by a primary lesion or dysfunction in the nervous system. Etiologies of neuropathic pain include, e.g., peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy, and vitamin deficiency.

Neuropathic pain can be related to a pain disorder, a term referring to a disease, disorder or condition associated with or caused by pain. Illustrative examples of pain disorders include arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

The inflammatory process is a complex series of biochemical and cellular events, activated in response to tissue injury or the presence of foreign substances, which results in swelling and pain. Arthritic pain is a common inflammatory pain.

Other types of pain that are amenable to treatment with the vectors, compositions, and methods contemplated herein, include but are not limited to pain resulting from musculoskeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome, and temporomandibular myofascial pain.

The effective amount of the compositions and methods contemplated herein to reduce the amount of pain experienced by a human subject can be determined using a variety of pain scales. Patient self-reporting can be used to assess whether pain is reduced; see, e.g., Katz and Melzack (1999) Surg. Clin. North Am. 79:231. Alternatively, an observational pain scale can be used. The LANSS Pain Scale can be used to assess whether pain is reduced; see, e.g., Bennett (2001) Pain 92:147. A visual analog pain scale can be used; see, e.g., Schmader (2002) Clin. J. Pain 18:350. The Likert pain scale can be used; e.g., where 0 is no pain, 5 is moderate pain, and 10 is the worst pain possible. Self-report pain scales for children include, e.g., Faces Pain Scale; Wong-Baker FACES Pain Rating Scale; and Colored Analog Scale. Self-report pain scales for adults include, e.g., Visual Analog Scale; Verbal Numerical Rating Scale; Verbal Descriptor Scale; and Brief Pain Inventory. Pain measurement scales include, e.g., Alder Hey Triage Pain Score (Stewart et al. (2004) Arch. Dis. Child. 89:625); Behavioral Pain Scale (Payen et al. (2001) Critical Care Medicine 29:2258); Brief Pain Inventory (Cleeland and Ryan (1994) Ann. Acad. Med. Singapore 23: 129); Checklist of Nonverbal Pain Indicators (Feldt (2000) Pain Manag. Nurs. 1: 13); Critical-Care Pain Observation Tool (Gelinas et al. (2006) Am. J. Crit. Care 15:420); COMFORT scale (Ambuel et al. (1992) J. Pediatric Psychol. 17:95); Dallas Pain Questionnaire (Ozguler et al. (2002) Spine 27:1783); Dolorimeter Pain Index (Hardy et al. (1952) Pain Sensations and Reactions Baltimore: The Williams & Wilkins Co.); Faces Pain Scale—Revised (Hicks et al. (2001) Pain 93:173); Face Legs Activity Cry Consolability Scale; McGill Pain Questionnaire (Melzack (1975) Pain 1:277); Descriptor Differential Scale (Gracely and Kwilosz (1988) Pain 35:279); Numerical 1 1 point Box (Jensen et al. (1989) Clin. J. Pain 5: 153); Numeric Rating Scale (Hartrick et al. (2003) Pain Pract. 3:310); Wong-Baker FACES Pain Rating Scale; and Visual Analog Scale (Huskisson (1982) J. Rheumatol. 9:768).

In particular embodiments, a method of relieving pain in a subject is provided, the method comprising introducing an engineered receptor into a neuronal cell and controlling the activity of the cell by providing an effective amount of a ligand that activates the engineered receptor, thereby relieving pain in the subject. The method provides significant analgesia for pain without off-target effects, such as general central nervous system depression. In certain embodiments, the method provides a 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more (including all ranges and subranges therebetween) reduction in the neuropathic pain in a subject compared to an untreated subject. In some embodiments, the method comprises the step of measuring pain in the subject before and after the administration of the binding agent, wherein the pain in the subject is reduced 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more, including all ranges and subranges therebetween. In such instances, the measuring may occur 4 hours or more after administration of the binding agent, e.g. 8 hours 12 hours, 16 hours, 24 hours, 36 hours, 48 hours, 3 days, or 4 days or more after administration of the binding agent.

In particular embodiments, the vectors contemplated herein are administered or introduced into one or more neuronal cells. The neuronal cells may be the same type of neuronal cells, or a mixed population of different types of neuronal cells. In one embodiment, the neuronal cell is a nociceptor or peripheral sensory neuron. Illustrative examples of sensory neurons include, but are not limited to, dorsal root ganglion (DRG) neurons and trigeminal ganglion (TGG) neurons. In one embodiment, the neuronal cell is an inhibitory interneuron involved in the neuronal pain circuit.

In some cases, a vector encoding an engineered receptor is administered to a subject in need thereof. Non-limiting examples of methods of administration include subcutaneous administration, intravenous administration, intramuscular administration, intradermal administration, intraperitoneal administration, oral administration, infusion, intracranial administration, intrathecal administration, intranasal administration, intraganglionic administration, intraspinal administration, cisterna magna administration and intraneural administration. In some cases, administration can involve injection of a liquid formulation of the vector. In other cases, administration can involve oral delivery of a solid formulation of the vector. In some cases, the oral formulation can be administered with food. In particular embodiments, a vector is parenterally, intravenously, intramuscularly, intraperitoneally, intrathecally, intraneurally, intraganglionicly, intraspinally, or intraventricularly administered to a subject in order to introduce the vector into one or more neuronal cells. In various embodiments, the vector is rAAV.

In one embodiment, AAV is administered to sensory neuron or nociceptor, e.g., DRG neurons, TGG neurons, etc. by intrathecal (IT) or intraganglionic (IG) administration. The IT route delivers AAV to the cerebrospinal fluid (CSF). This route of administration may be suitable for the treatment of e.g., chronic pain or other peripheral nervous system (PNS) or central nervous system (CNS) indications. In animals, IT administration has been achieved by inserting an IT catheter through the cisterna magna and advancing it caudally to the lumbar level. In humans, IT delivery can be easily performed by lumbar puncture (LP), a routine bedside procedure with excellent safety profile.

In a particular case, a vector may be administered to a subject by intraganglionic administration. Intraganglionic administration may involve an injection directly into one or more ganglia. The IG route may deliver AAV directly into the DRG or TGG parenchyma. In animals, IG administration to the DRG is performed by an open neurosurgical procedure that is not desirable in humans because it would require a complicated and invasive procedure. In humans, a minimally invasive, CT imaging-guided technique to safely target the DRG can be used. A customized needle assembly for convection enhanced delivery (CED) can be used to deliver AAV into the DRG parenchyma. In a non-limiting example, a vector of the disclosure may be delivered to one or more dorsal root ganglia and/or trigeminal ganglia for the treatment of chronic pain. In another non-limiting example, a vector of the disclosure may be delivered to the nodose ganglion (vagus nerve) to treat epilepsy.

In yet another particular case, a vector may be administered to the subject by intracranial administration (i.e., directly into the brain). In non-limiting examples of intracranial administration, a vector of the disclosure may be delivered into the cortex of the brain to treat e.g., an epileptic seizure focus, into the paraventricular hypothalamus to treat e.g., a satiety disorder, or into the amygdala central nucleus to treat e.g., a satiety disorder. In another particular case, a vector may be administered to a subject by intraneural injection (i.e., directly into a nerve). The nerve may be selected based on the indication to be treated, for example, injection into the sciatic nerve to treat chronic pain or injection into the vagal nerve to treat epilepsy or a satiety disorder. In yet another particular case, a vector may be administered to a subject by subcutaneous injection, for example, into the sensory nerve terminals to treat chronic pain.

A vector dose may be expressed as the number of vector genome units delivered to a subject. A “vector genome unit” as used herein refers to the number of individual vector genomes administered in a dose. The size of an individual vector genome will generally depend on the type of viral vector used. Vector genomes of the disclosure may be from about 1.0 kilobase, 1.5 kilobases, 2.0 kilobases, 2.5 kilobases, 3.0 kilobases, 3.5 kilobases, 4.0 kilobases, 4.5 kilobases, 5.0 kilobases, 5.5 kilobases, 6.0 kilobases, 6.5 kilobases, 7.0 kilobases, 7.5 kilobases, 8.0 kilobases, 8.5 kilobases, 9.0 kilobases, 9.5 kilobases, 10.0 kilobases, to more than 10.0 kilobases. Therefore, a single vector genome may include up to or greater than 10,000 base pairs of nucleotides. In some cases, a vector dose may be about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹, 1×10¹, 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, 1×10¹⁷, 2×10¹⁷, 3×10¹⁷, 4×10¹⁷, 5×10¹⁷, 6×10¹⁷, 7×10¹⁷, 8×10¹⁷, 9×10¹⁷, 1×10¹⁸, 2×10¹⁸, 3×10¹⁸, 4×10¹⁸, 5×10¹⁸, 6×10¹⁸, 7×10¹⁸, 8×10¹⁸, 9×10¹⁸, 1×10¹⁹, 2×10¹⁹, 3×10¹⁹, 4×10¹⁹, 5×10¹⁹, 6×10¹⁹, 7×10¹⁹, 8×10¹⁹, 9×10¹⁹, 1×10²⁰, 2×10²⁰, 3×10²⁰, 4×10²⁰, 5×10²⁰, 6×10²⁰, 7×10²⁰, 8×10²⁰, 9×10²⁰ or more vector genome units.

In particular embodiments, a vector contemplated herein is administered to a subject at a titer of at least about 1×10⁹ genome particles/mL, at least about 1×10¹⁰ genome particles/mL, at least about 5×10¹⁰ genome particles/mL, at least about 1×10¹¹ genome particles/mL, at least about 5×10¹¹ genome particles/mL, at least about 1×10¹² genome particles/mL, at least about 5×10¹² genome particles/mL, at least about 6×10¹² genome particles/mL, at least about 7×10¹² genome particles/mL, at least about 8×10¹² genome particles/mL, at least about 9×10¹² genome particles/mL, at least about 10×10¹² genome particles/mL, at least about 15×10¹² genome particles/mL, at least about 20×10¹² genome particles/mL, at least about 25×10¹² genome particles/mL, at least about 50×10¹² genome particles/mL, or at least about 100×10¹² genome particles/mL. The terms “genome particles (gp)” or “genome equivalents” or “genome copies” (gc) as used in reference to a viral titer, refer to the number of virions containing the recombinant AAV DNA genome, regardless of infectivity or functionality. The number of genome particles in a particular vector preparation can be measured by well understood methods in the art, for example, quantitative PCR of genomic DNA or for example, in Clark et al. (1999) Hum. Gene Ther., 10:1031-1039; Veldwijk et al. (2002) Mol. Ther., 6:272-278.

A vector of the disclosure may be administered in a volume of fluid. In some cases, a vector may be administered in a volume of about 0.1 mL, 0.2 mL, 0.3 mL, 0.4 mL, 0.5 mL, 0.6 mL, 0.7 mL, 0.8 mL, 0.9 mL, 1.0 mL, 2.0 mL, 3.0 mL, 4.0 mL, 5.0 mL, 6.0 mL, 7.0 mL, 8.0 mL, 9.0 mL, 10.0 mL, 11.0 mL, 12.0 mL, 13.0 mL, 14.0 mL, 15.0 mL, 16.0 mL, 17.0 mL, 18.0 mL, 19.0 mL, 20.0 mL or greater than 20.0 mL. In some cases, a vector dose may be expressed as a concentration or titer of vector administered to a subject. In this case, a vector dose may be expressed as the number of vector genome units per volume (i.e., genome units/volume).

In particular embodiments, a vector contemplated herein is administered to a subject at a titer of at least about 5×10⁹ infectious units/mL, at least about 6×10⁹ infectious units/mL, at least about 7×10⁹ infectious units/mL, at least about 8×10⁹ infectious units/mL, at least about 9×10⁹ infectious units/mL, at least about 1×10¹⁰ infectious units/mL, at least about 1.5×10¹⁰ infectious units/mL, at least about 2×10¹⁰ infectious units/mL, at least about 2.5×10¹⁰ infectious units/mL, at least about 5×10¹⁰ infectious units/mL, at least about 1×10¹¹ infectious units/mL, at least about 2.5×10¹¹ infectious units/mL, at least about 5×10¹¹ infectious units/mL, at least about 1×10¹² infectious units/mL, at least about 2.5×10¹² infectious units/mL, at least about 5×10¹² infectious units/mL, at least about 1×10¹³ infectious units/mL, at least about 5×10¹³ infectious units/mL, at least about 1×10¹⁴ infectious units/mL. The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Virol., 62:1963-1973.

In particular embodiments, a vector contemplated herein is administered to a subject at a titer of at least about 5×10¹⁰ transducing units/mL, at least about 1×10¹¹ transducing units/mL, at least about 2.5×10¹¹ transducing units/mL, at least about 5×10¹¹ transducing units/mL, at least about 1×10¹² transducing units/mL, at least about 2.5×10¹² transducing units/mL, at least about 5×10¹² transducing units/mL, at least about 1×10¹³ transducing units/mL, at least about 5×10¹³ transducing units/mL, at least about 1×10¹⁴ transducing units/mL. The term “transducing unit” (tu)” as used in reference to a viral titer, refers to the number of infectious recombinant AAV vector particles that result in the production of a functional transgene product as measured in functional assays such as described in, for example, in Xiao et al. (1997) Exp. Neurobiol., 144:113-124; or in Fisher et al. (1996) J. Virol., 70:520-532 (LFU assay).

The vector dose will generally be determined by the route of administration. In a particular example, an intraganglionic injection may include from about 1×10⁹ to about 1×10¹³ vector genomes in a volume from about 0.1 mL to about 1.0 mL. In another particular case, an intrathecal injection may include from about 1×10¹⁰ to about 1×10¹⁵ vector genomes in a volume from about 1.0 mL to about 12.0 mL. In yet another particular case, an intracranial injection may include from about 1×10⁹ to about 1×10¹³ vector genomes in a volume from about 0.1 mL to about 1.0 mL. In another particular case, an intraneural injection may include from about 1×10⁹ to about 1×10¹³ vector genomes in a volume from about 0.1 mL to about 1.0 mL. In another particular example, an intraspinal injection may include from about 1×10⁹ to about 1×10¹³ vector genomes in a volume from about 0.1 mL to about 1.0 mL. In yet another particular case, a cisterna magna infusion may include from about 5×10⁹ to about 5×10¹³ vector genomes in a volume from about 0.5 mL to about 5.0 mL. In yet another particular case, a subcutaneous injection may include from about 1×10⁹ to about 1×10¹³ vector genomes in a volume from about 0.1 mL to about 1.0 mL.

In some cases, a vector is delivered to a subject by infusion. A vector dose delivered to a subject by infusion can be measured as a vector infusion rate. Non-limiting examples of vector infusion rates include: 1-10 μL/min for intraganglionic, intraspinal, intracranial or intraneural administration; and 10-1000 μL/min for intrathecal or cisterna magna administration. In some cases, the vector is delivered to a subject by MRI-guided Convection Enhanced Delivery (CED). This technique enables increased viral spread and transduction distributed throughout large volumes of the brain, as well as reduces reflux of the vector along the needle path.

In various embodiments, a method is provided comprising administering a vector encoding a engineered receptor, that deactivates or hyperpolarizes neuronal cells, to one or more neuronal cells that increase pain sensation or sensitivity to pain, and administering a ligand that specifically binds the neuronal cell expressing the engineered receptor to the subject, thereby deactivating the cell, decreasing the sensitivity to pain and potentiating an analgesic effect.

In various embodiments, a method is provided comprising administering a vector encoding a engineered receptor, that activates or polarizes neuronal cells, to one or more neuronal cells that decrease pain sensation or sensitivity to pain, and administering a ligand that specifically binds the neuronal cell expressing the engineered receptor to the subject, thereby activating the cell, decreasing the sensitivity to pain and potentiating an analgesic effect.

Formulations of ligands may be administered to a subject by various routes. Non-limiting examples of methods of administration include subcutaneous administration, intravenous administration, intramuscular administration, transdermal administration, intradermal administration, intraperitoneal administration, oral administration, infusion, intracranial administration, intrathecal administration, intranasal administration, intraganglionic administration, and intraneural administration. In some cases, administration can involve injection of a liquid formulation of the ligand. In other cases, administration can involve oral delivery of a solid formulation of the ligand. In a particular case, a ligand is administered by oral administration (e.g., a pill, tablet, capsule and the like). In some cases, the oral composition can be administered with food. In another particular case, a ligand is administered by intrathecal injection (i.e., into the subarachnoid space of the spinal cord) for delivery to the cerebrospinal fluid (CSF) of the subject. In another particular case, a ligand is administered topically (e.g., dermal patch, cream, lotion, ointment and the like).

The dosages of the ligands administered to a subject are not subject to absolute limits, but will depend on the nature of the composition and its active ingredients and its unwanted side effects (e.g., immune response against the antibody), the subject being treated and the type of condition being treated and the manner of administration. Generally, the dose will be a therapeutically effective amount, such as an amount sufficient to achieve a desired biological effect, for example an amount that is effective to decrease or attenuate the level of pain experienced by the subject. In particular embodiments, the dose can also be a prophylactic amount or an effective amount. A therapeutically effective amount of ligand may depend on the route of administration, the indication being treated, and/or the ligand selected for use.

In one embodiment, the ligand is first administered to the subject prior to administration of the vector. A therapeutically effective amount of ligand may be administered to a subject at some time after delivery of a vector. Generally, after delivery of a vector, there will be a period of time required for one or more cells of the subject to generate a protein (i.e., engineered receptor) encoded by the vector. During this period of time, administration of a ligand to the subject may not be beneficial to the subject. In this situation, it may be suitable to administer the ligand after an amount of engineered receptor has been produced by one or more cells of the subject.

In one embodiment, the ligand is first administered to the subject at about the same time that the vector is administered to the subject.

In one embodiment, the ligand is first administered 1, 2, 3, 4, 5, 6, 7, 8, 9, 11, or 12 hours, days, weeks, months, or years after administration of the vector to the subject. In some cases, a therapeutically effective amount of a ligand may be administered to a subject at least one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more than 30 days after delivery of the vector. In a particular example, a therapeutically effective amount of a ligand is administered to a subject at least one week after delivery of a vector. In a further example, the therapeutically effective amount of ligand is administered to the subject daily for at least three consecutive days.

A therapeutically effective amount or dose of a ligand of the disclosure can be expressed as mg or μg of the ligand per kg of subject body mass. In some instances, a therapeutically effective amount of a ligand may be about 0.001 μg/kg, about 0.005 μg/kg, about 0.01 μg/kg, about 0.05 μg/kg, about 0.1 μg/kg, about 0.5 μg/kg, about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 120 μg/kg, about 140 μg/kg, about 160 μg/kg, about 180 μg/kg, about 200 μg/kg, about 220 μg/kg, about 240 μg/kg, about 260 μg/kg, about 280 μg/kg, about 300 μg/kg, about 320 μg/kg, about 340 μg/kg, about 360 μg/kg, about 380 μg/kg, about 400 μg/kg, about 420 μg/kg, about 440 μg/kg, about 460 μg/kg, about 480 μg/kg, about 500 μg/kg, about 520 μg/kg, about 540 μg/kg, about 560 μg/kg, about 580 μg/kg, about 600 μg/kg, about 620 μg/kg, about 640 μg/kg, about 660 μg/kg, about 680 μg/kg, about 700 μg/kg, about 720 μg/kg, about 740 μg/kg, about 760 μg/kg, about 780 μg/kg, about 800 μg/kg, about 820 μg/kg, about 840 μg/kg, about 860 μg/kg, about 880 μg/kg, about 900 μg/kg, about 920 μg/kg, about 940 μg/kg, about 960 μg/kg, about 980 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or greater than 10 mg/kg.

In particular embodiments, the dose of ligand administered to a subject is at least about 0.001 micrograms per kilogram (μg/kg), at least about 0.005 μg/kg, at least about 0.01 μg/kg, at least about 0.05 μg/kg, at least about 0.1 μg/kg, at least about 0.5 μg/kg, 0.001 milligrams per kilogram (mg/kg), at least about 0.005 mg/kg, at least about 0.01 mg/kg, at least about 0.05 mg/kg, at least about 0.1 mg/kg, at least about 0.5 mg/kg, at least about 1 mg/kg, at least about 2 mg/kg, at least about 3 mg/kg, at least about 4 mg/kg, at least about 5 mg/kg, at least about 5 mg/kg, at least about 6 mg/kg, at least about 7 mg/kg, at least about 8 mg/kg, at least about 8 mg/kg, at least about 9 mg/kg, or at least about 10 or more mg/kg.

In particular embodiments, the dose of ligand administered to a subject is at least about 0.001 μg/kg to at least about 10 mg/kg, at least about 0.01 μg/kg to at least about 10 mg/kg, at least about 0.1 μg/kg to at least about 10 mg/kg, at least about 1 μg/kg to at least about 10 mg/kg, at least about 0.01 mg/kg to at least about 10 mg/kg, at least about 0.1 mg/kg to at least about 10 mg/kg, or at least about 1 mg/kg to at least about 10 mg/kg, or any intervening range thereof.

In some aspects, a therapeutically effective amount of a ligand can be expressed as a molar concentration (i.e., M or mol/L). In some cases, a therapeutically effective amount of a ligand can be about 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, 1000 mM or greater.

A therapeutically effective amount of a ligand can be administered once or more than once each day. In some cases, a therapeutically effective amount of a ligand is administered as needed (e.g., when pain relief is needed). The ligand may be administered serially (e.g., every day without a break for the duration of the treatment regimen). In some cases, the treatment regimen can be less than a week, a week, two weeks, three weeks, a month, or greater than a month. In some cases, a therapeutically effective amount of a ligand is administered for a day, at least two consecutive days, at least three consecutive days, at least four consecutive days, at least five consecutive days, at least six consecutive days, at least seven consecutive days, at least eight consecutive days, at least nine consecutive days, at least ten consecutive days, or at least greater than ten consecutive days. In a particular case, a therapeutically effective amount of a ligand is administered for three consecutive days. In some cases, a therapeutically effective amount of a ligand can be administered one time per week, two times per week, three times per week, four times per week, five times per week, six times per week, seven times per week, eight times per week, nine times per week, 10 times per week, 11 times per week, 12 times per week, 13 times per week, 14 times per week, 15 times per week, 16 times per week, 17 times per week, 18 times per week, 19 times per week, 20 times per week, 25 times per week, 30 times per week, 35 times per week, 40 times per week, or greater than 40 times per week. In some cases, a therapeutically effective amount of a ligand can be administered one time per day, two times per day, three times per day, four times per day, five times per day, six times per day, seven times per day, eight times per day, nine times per day, 10 times per day, or greater than 10 times per day. In some cases, a therapeutically effective amount of a ligand is administered at least every hour, at least every two hours, at least every three hours, at least every four hours, at least every five hours, at least every six hours, at least every seven hours, at least every eight hours, at least every nine hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, or at least every day. The dose of ligand may be administered to the subject continuously, or 1, 2, 3, 4, or 5 times a day; 1, 2, 3, 4, 5, 6, or 7 times a week, 1, 2, 3, or 4 times a month, once every 2, 3, 4, 5, or 6 months, or once a year, or at even longer intervals. The duration of treatment can last a day, 1, 2, or 3 weeks, 1, 2, 3, 4, 5, 7, 8, 9, 10, or 11 months, 1, 2, 3, 4, 5, or more years, or longer.

A subject treated by methods and compositions disclosed herein can be a human, or can be a non-human animal. The term “treat” and its grammatical equivalents used herein generally refer to the use of a composition or method to reduce, eliminate, or prevent symptoms of a disease and includes achieving a therapeutic benefit and/or a prophylactic benefit. By therapeutic benefit is meant slowing the progression of, halting the progression of, reversing the progression of, or eradication or amelioration of the symptoms of the disorder or condition being treated. A prophylactic benefit of treatment includes reducing the risk of a condition, retarding the progress of a condition, or decreasing the likelihood of occurrence of a condition.

Non-limiting examples of non-human animals include a non-human primate, a livestock animal, a domestic pet, and a laboratory animal. For example, a non-human animal can be an ape (e.g., a chimpanzee, a baboon, a gorilla, or an orangutan), an old world monkey (e.g., a rhesus monkey), a new world monkey, a dog, a cat, a bison, a camel, a cow, a deer, a pig, a donkey, a horse, a mule, a lama, a sheep, a goat, a buffalo, a reindeer, a yak, a mouse, a rat, a rabbit, or any other non-human animal. The compositions and methods as described herein are amenable to the treatment of a veterinary animal. A veterinary animal can include, without limitation, a dog, a cat, a horse, a cow, a sheep, a mouse, a rat, a guinea pig, a hamster, a rabbit, a snake, a turtle, and a lizard. In some aspects, contacting the tissue or cell population with a composition comprises administering the composition to a cell population or subject. In some embodiments, administration occurs in vitro, for example by adding the composition to a cell culture system. In some aspects, administration occurs in vivo, for example by administration through a particular route. Wherein more than one composition is to be administered, the compositions may be administered via the same route at the same time (e.g., on the same day), or via the same route at different times. Alternatively, the compositions may be administered via different routes at the same time (e.g., on the same day) or via different routes at different times.

The number of times a composition is administered to an subject in need thereof depends on the discretion of a medical professional, the disorder, the severity of the disorder, and the subject's response to the formulation. In some aspects, administration of a composition occurs at least once. In further aspects, administration occurs more than once, for example 2, 3, 4, 5, 6, 7, 8, 9, 10 or more times in a given period. The dosage of each administration and/or frequency of administrations may be adjusted as necessary based on the patient's condition and physiologically responses.

In some embodiments, compositions may be administered a sufficient amount of times to achieve a desired physiologic effect or improvement in a subject's condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition. In the case wherein the subject's status does improve, upon the doctor's discretion the composition may administered continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%, including all ranges and subranges therebetween.

Where compositions are administered more than once, each administration may be performed by the same actor and/or in the same geographical location. Alternatively, each administration may be performed by a different actor and/or in a different geographical location.

With regard to human and veterinary treatment, the amount of a particular agent(s) that is administered may be dependent on a variety of factors, including the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician or veterinarian; and like factors known in the medical and veterinary arts. Similarly, the effective concentration of a given composition may be dependent on a variety of factors including the age, sex, weight, genetic status, and overall health of the patient or subject.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

It is to be understood that the description above as well as the examples that follow are intended to illustrate, and not limit, the scope of the invention. Other aspects, advantages and modifications within the scope of the invention will be apparent to those skilled in the art to which the invention pertains.

### EXAMPLES

### Example 1: Construction of Engineered Receptors Comprising Ion Pore Domain Derived from the Human GlyRα1, GlyRα2, GlyRα3 or GABArho Receptor

To generate LGICs that conduct anion current following exposure to non-native small molecule agonists of the human α7-nAChR, chimeric ligand gated ion channel (LGIC) receptors comprising the ligand binding domain derived from the human α7 nicotinic acetylcholine receptor (α7-nAChR) and the ion pore domain derived from the human GlyRα1, GlyRα2, Gly or GABArho receptor were constructed according to Table 12.

### Example 2: Characterization of the Engineered Receptors Using High Throughput Fluorescence Based Plate Screening

The engineered LGIC receptors in Example 1 were analyzed for their potency to their native ligand, acetylcholine (Ach), and non-native ligands such as, AZD-0328 (adisinsight.springer.com/drugs/800018503), TC-6987 (drugbank.ca/drugs/DB14854), ABT-126 (medchemexpress.com/Nelonicline.html), APN-1125 (clinicaltrials.gov/ct2/show/NCT02724917), TC-5619 (en.wikipedia.org/wiki/Bradanicline), and Facinicline/RG3487 (researchgate.net/figure/Molecular-structure-of-RG3487_fig1_47499934).

An anion reporter assay was used to assess the the response profiles of these engineered receptors to ligands. In this assay, cells expressing a YFP reporter whose fluorescence is quenched in the presence of anion are transfected with DNA encoding the engineered receptor of interest. Upon exposure to ligand, channels that are activated will flux anion, resulting in a dose-dependent quench of the YFP that can be detected on a plate reader.

Lenti-X 293T cells (LX293T, Clontech) were maintained in DMEM containing 10% FBS and 1% penicillin/streptomycin (Invitrogen). For plate reader assays, LX293T cells were infected with a lentivirus to create cells that stably express a mutant YFP (H148Q/I152L) reporter, which displays enhanced sensitivity to anions. Two days before assay, cells were split at a density of 20,000 cells/well in a 96-well tissue culture plate coated with poly-d lysine (Thermo Scientific). The next day, the cells were transiently transfected with 0.1 μg of DNA per well using standard Fugene 6 protocol (Promega). On the day of assay, cells were washed 2 times in 1× extracellular solution (1×ECS: 140 mM NaCl, 5 mM KCl, 1 mM MgCl2, 2 mM CaCl2), 10 mM HEPES, 10 mM glucose, pH 7.2, mOsms 300). After the last wash, 100 μL of 1×ECS was added to the wells and the plate was incubated at 37° C. for 30 min. While incubating the plate, the drugs were diluted to a 2× concentration in 1×ECS-NaI (same components as 1×ECS except the 140 mM NaCl was replaced by 140 mM NaI). The plates were then read on a Flexstation3 (Molecular Devices). Each well, 8 wells at a time, of the plate is read for 2 min using a Flexstation3 (Molecular Devices) as follows: 1) a baseline YFP fluorescence is read for 17 sec, 2) 100 μL of ligand is added, and 3) the changes in YFP fluorescence are then measured every 1.3 sec for the remaining time.

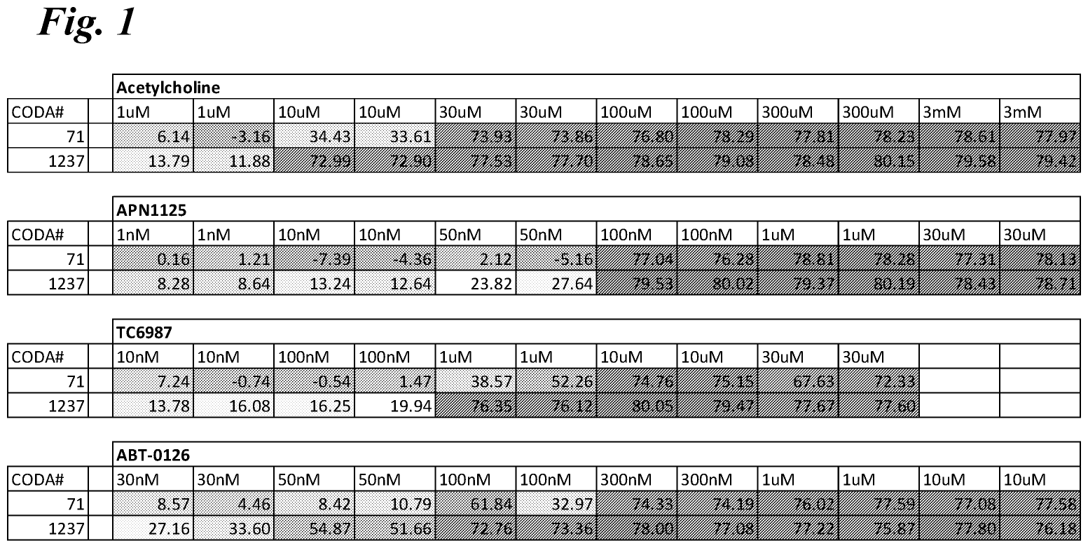

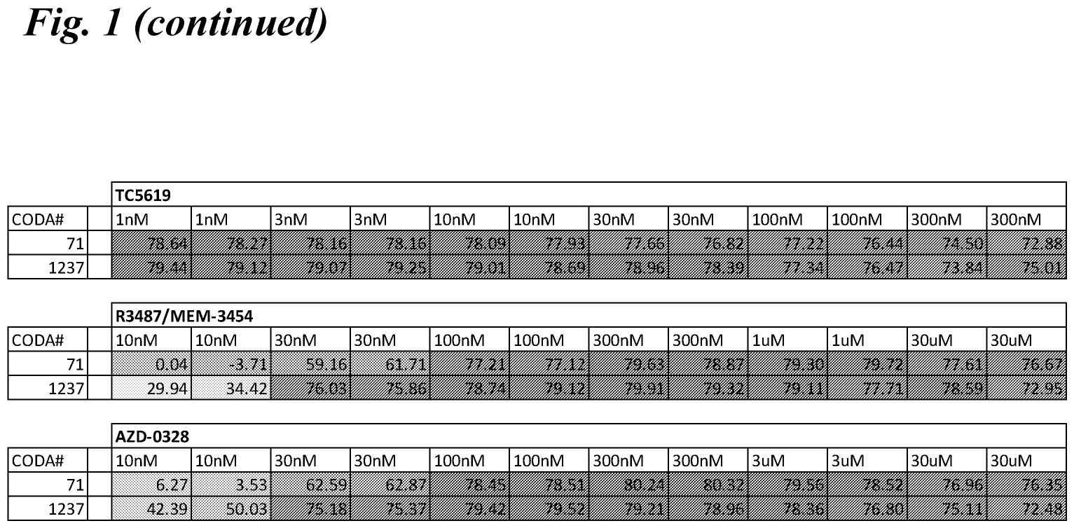

FIG. 1 provides heat maps of the percent quench of YFP fluorescence following stimulation by various doses of either acetylcholine or the non-native ligand as indicated in the figure for cells expressing CODA71 (comprising GlyRα1 ion pore domain) or CODA1237 (comprising GlyRα2 ion pore domain). Non-native ligands tested include AZD-0328, TC-6987, ABT-126, APN-1125, TC-5619, and Facinicline/RG3487. A positive fluorescence signal, as indicated by blue shaded cells, indicate that the engineered receptor is activated by the non-native ligand at that concentration. The results indicate that both groups of cells display similar responsiveness to multiple ligands. Cells expressing CODA1237 are more responsive to ABT-126, RG3487 and AZD-0328 than cells expressing CODA71.

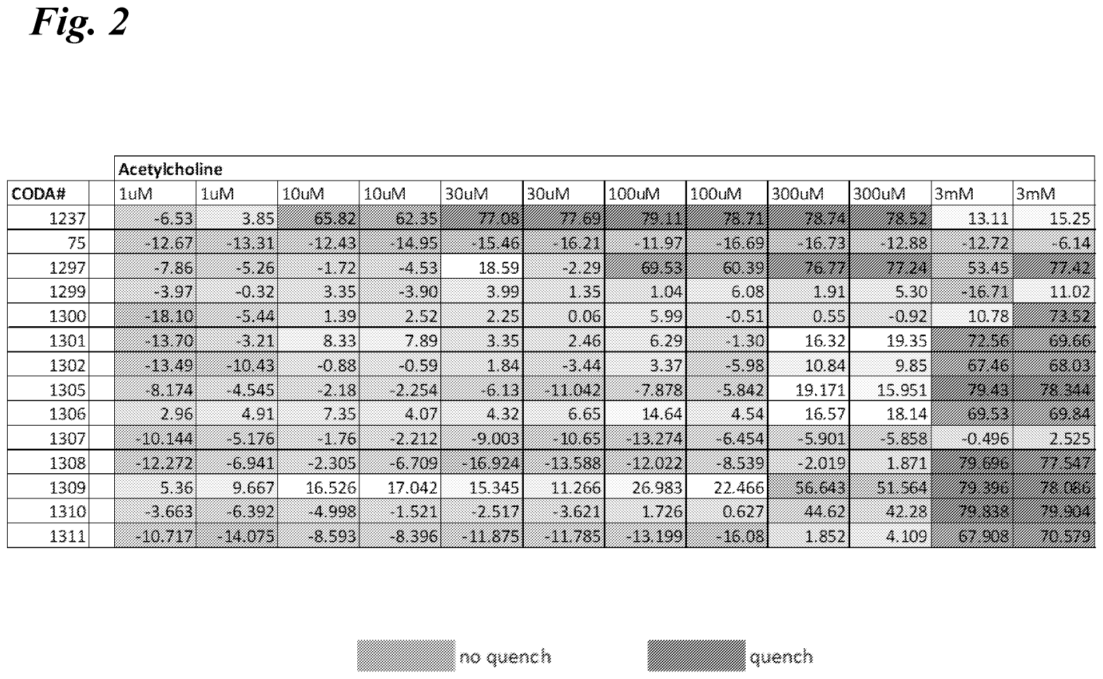

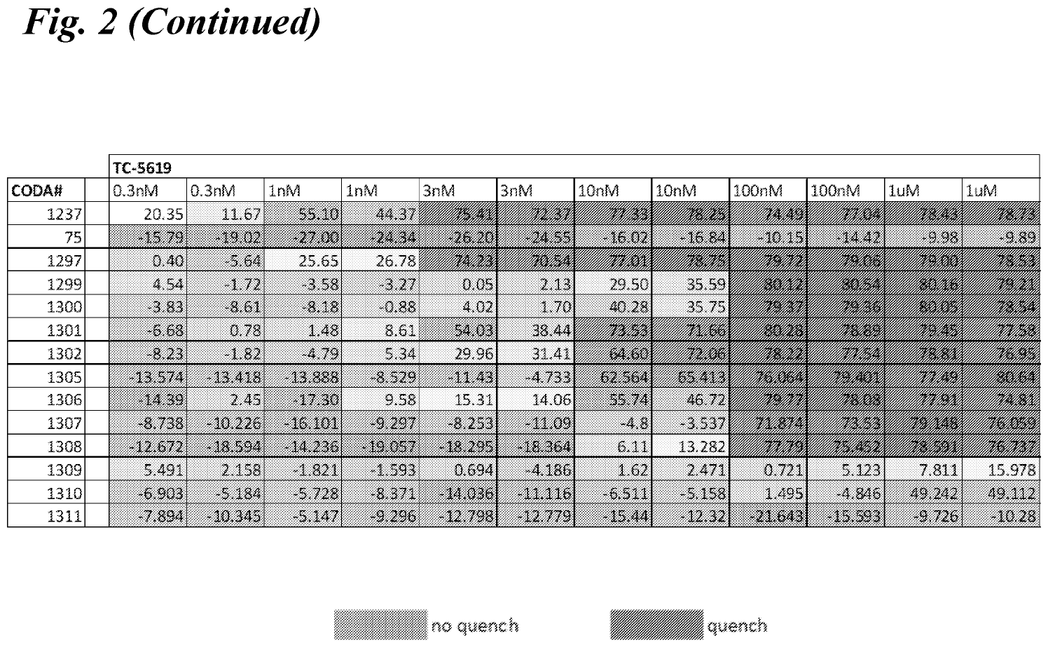

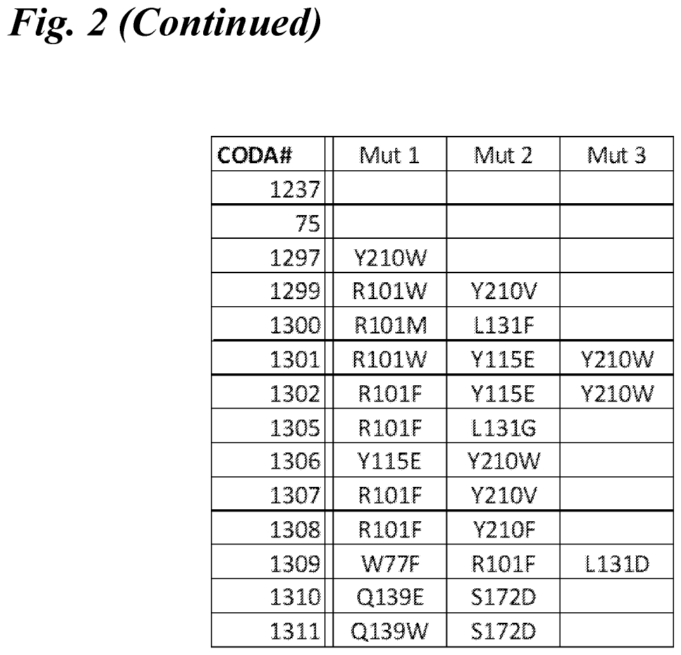

FIG. 2 provides heat maps of the percent quench of YFP fluorescence following stimulation by various doses of either acetylcholine or TC-5619 for cells expressing a CODA1237 based chimeric LGIC receptor comprising various amino acid mutations in the ligand binding domain derived from α7-nAChR, as summarized in Table 12. For most of these mutants, the corresponding cell line exhibits far right-ward shift in dose needed to elicit a response for acetylcholine (i.e., less responsive to acetylcholine), while showing minimal to no significant shift in response for TC-5619. CODA75 is a non-responding chimeric receptor used here as a negative control.

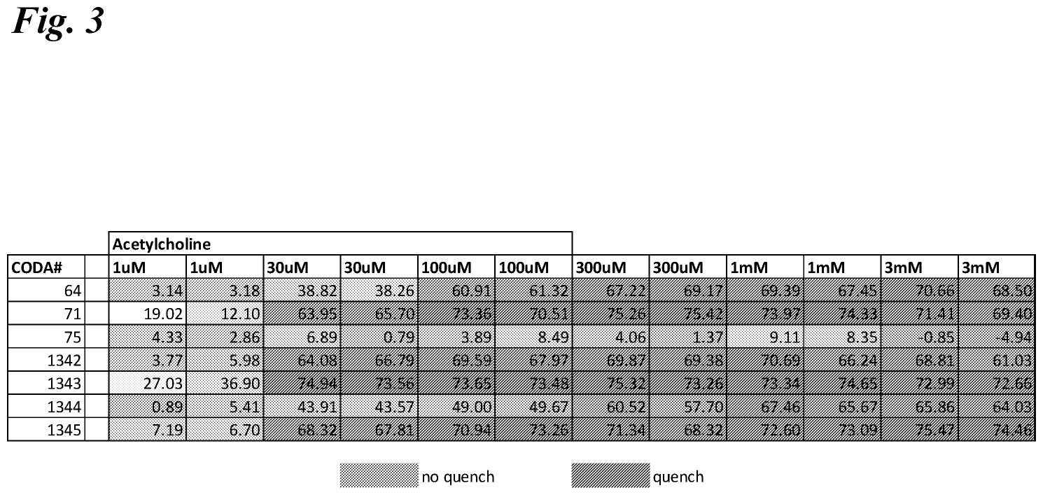

FIG. 3 provides heat maps of the percent quench of YFP fluorescence following stimulation by indicated doses of acetylcholine in cells expressing the indicated chimeric LGIC receptor. We tested four chimeric LGIC receptors comprising the ion pore domain of GlyRα3 (CODA1342, CODA1343, CODA1344 and CODA1345, as summarized in Table 12) and compared their responsiveness to acetylcholine with that of CODA71 and CODA64. CODA75 is a non-responding chimeric receptor used here as a negative control. The results indicate that, using CODA71 as the reference point, cells expressing CODA1342 and CODA1345 have similar responsiveness to acetylcholine, cells expressing CODA1344 have a lower responsiveness to acetylcholine, and cells expressing CODA1343 have a higher responsiveness to acetylcholine.

Table 13 below provides the EC50 measurements of different ligands for CODA1316 using the fluorescence plate screening method or the manual patch clamp method (described in Example 4 below). CODA1316 was capable of binding to TC-5619 with low nanomolar EC50.

TABLE 13
EC50 of Engineered Receptors

| | Description of the | Plate Reader based | | Manual Patch | |
|---|---|---|---|---|---|
| Receptor | Engineered | EC50 Measurement | | Clamp based EC50 | |
| Number | Receptor | (uM) | | Measurement (uM) | |
| Ligand: | | TC-5619 | Ach | TC-5619 | Ach |
| CODA1316 | α7-nAChR(Gly-cys) | 0.003 | 274 | 0.01 | |
| | LBD: | | | | |
| | GlyRα2 IPD (SEQ ID | | | | |
| | NO: 65) comprising | | | | |
| | R101W, Y115E, | | | | |
| | Y210W, and T225I | | | | |
| | mutations | | | | |

### Example 3: Localization of the Engineered Receptors

The efficiency with which the engineered receptors disclosed herein are localized to the surface of the cells was evaluated using fluorescently labelled α-bungarotoxin, which specifically binds to the engineered receptors, followed by flow cytometry to evaluate the surface localization of the engineered receptors.

Alpha-bungarotoxin conjugated to biotin, Alexa Fluor 647 were all purchased from Thermo/Fisher (Waltham MA). Briefly, HEK-293T or HEK293T cells transduced with Ric3, Nacho, and Gcamp6s were plated at 200,00 cells per well and transfected with Fugene 6 the next day at a 1:3 ratio of DNA to Fugene. Ric3 and Nacho are chaperones for human α7 nicotinic acetylcholine receptor and GCamp6s is a calcium reporter. Cells were analyzed the day after transfection using flow cytometry. For flow cytometry analysis, transfected HEK293T cells were lifted manually, washed and incubated in α-bungarotoxin (1:1000) for 1 hour in FACS Buffer (2% BSA, 1×PBS without Ca+ and Mg+, and 1× Penicillin Streptomycin). Cells were then washed in FACS buffer and then analyzed on a Sony SH800 FACS sorter (San Jose, CA). Subsequent analysis was done using FlowJo (San Jose, CA).

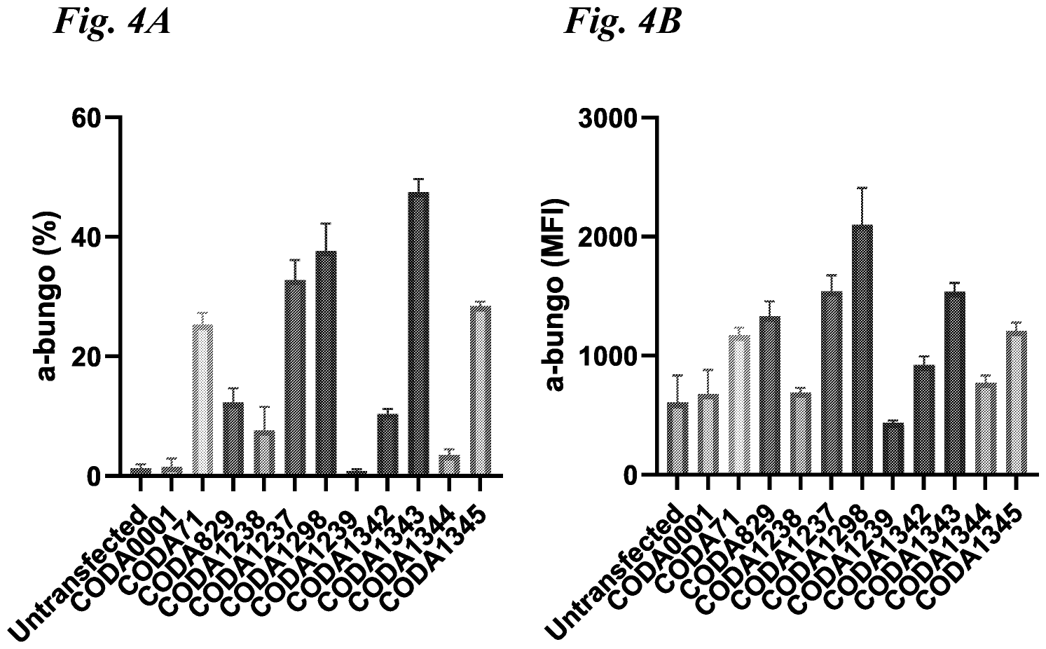

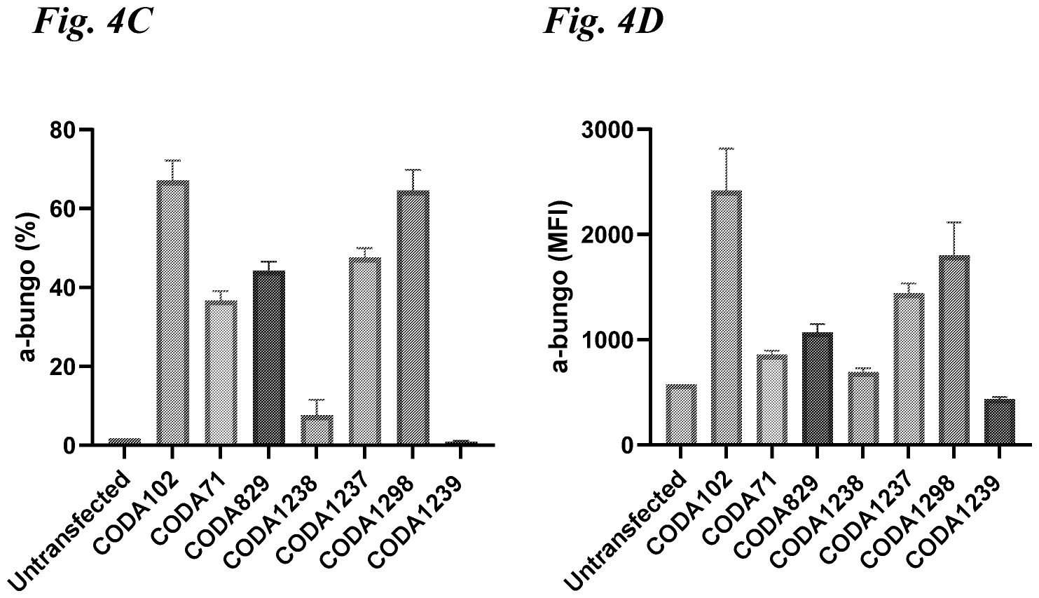

FIG. 4A shows the percentage of α-bungarotoxin staining positive HEK293T cells transiently transfected with CODA71, CODA829, CODA1237, CODA1238, CODA1298, CODA1239, CODA1342, CODA1343, CODA1344, or CODA1345. The design of the LGIC constructs are summarized in Table 12. FIG. 4B shows the mean fluorescent intensity of α-bungarotoxin staining positive cells from FIG. 4A.

FIG. 4C shows the percentage of α-bungarotoxin staining positive cells transiently transfected with CODA71, CODA102, CODA829, CODA1237, CODA1238, CODA1298, or CODA1239, using HEK293T cells transduced with Ric3, Nacho, and GCAMP6s. The design of the LGIC constructs are summarized in Table 12. FIG. 4D shows the mean fluorescent intensity of α-bungarotoxin staining positive cells from FIG. 4C.

The results in FIGS. 4A-4D show that, for cells transfected with chimeric LGIC comprising GlyRα1 IPD, T225I mutation may improve the surface expression level of LGIC in α-bungarotoxin staining positive cells, according to the comparison between CODA71 and CODA829. For cells transfected with chimeric LGIC comprising GlyRα2 IPD, T225I mutation improves the surface expression level of LGIC in α-bungarotoxin staining positive cells, according to the comparison between CODA1237 and CODA1298. In addition, the chimeric LGIC receptors comprising the ion pore domain of GlyRα3 isoform K (CODA1343 and CODA1345) have higher percentage of surface expressing cells and higher surface expression than the corresponding LGIC receptors comprising the ion pore domain of GlyRα3 isoform L (CODA1342 and CODA1344).

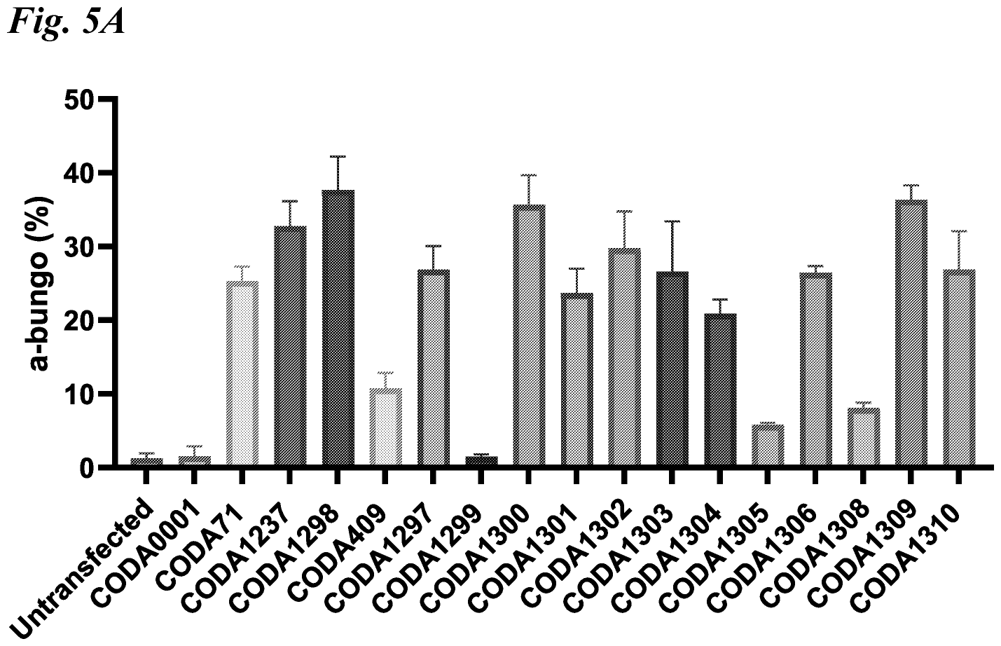

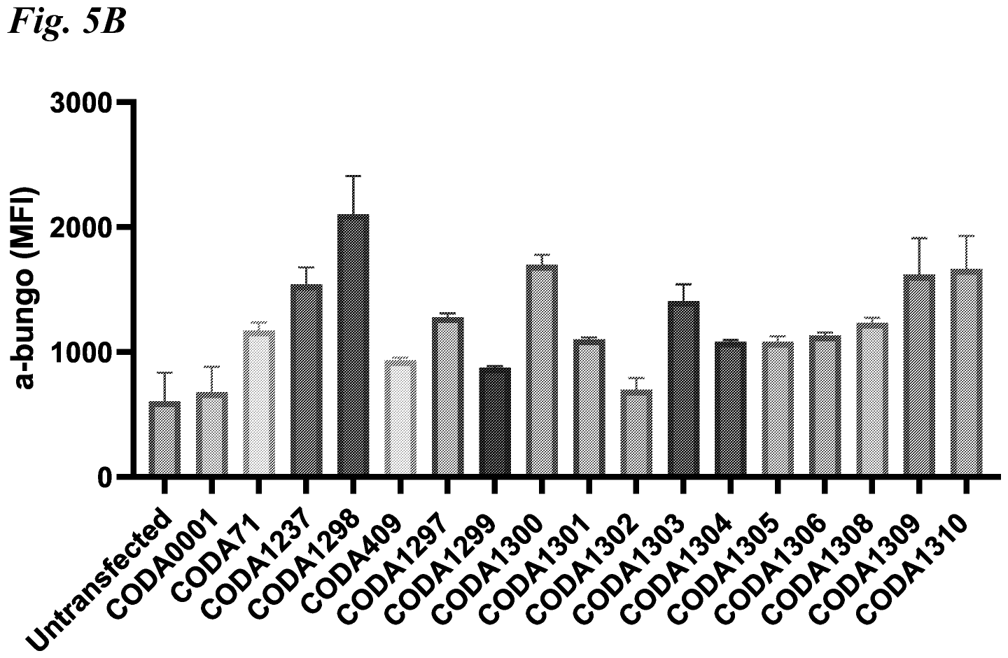

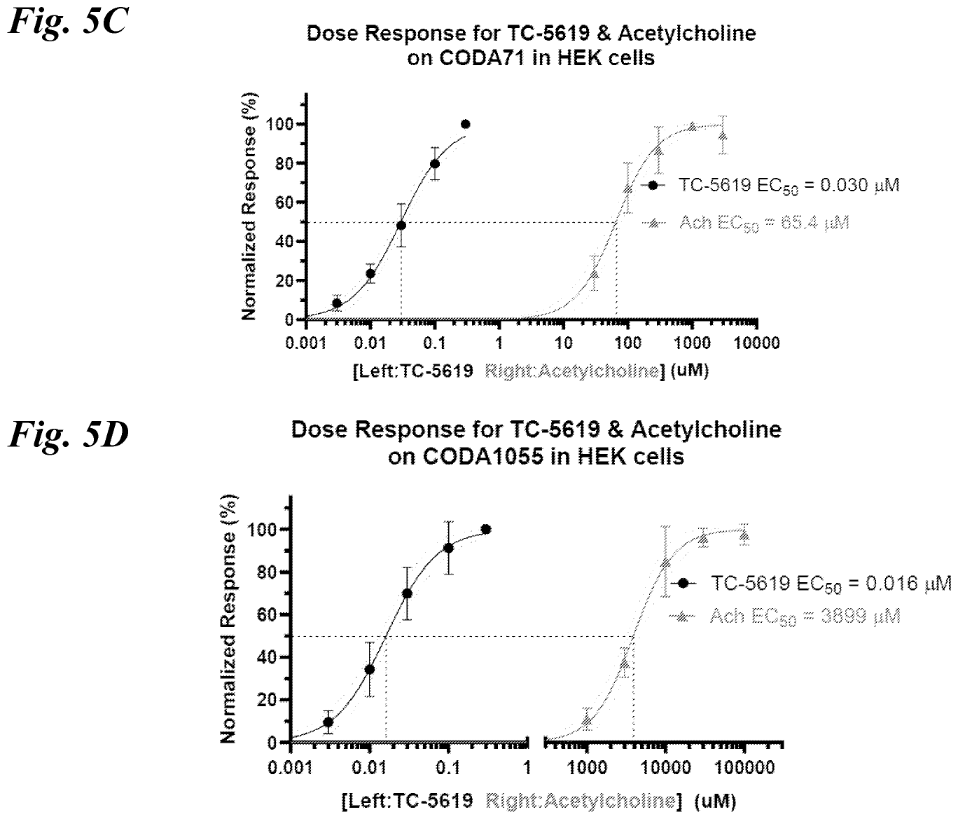

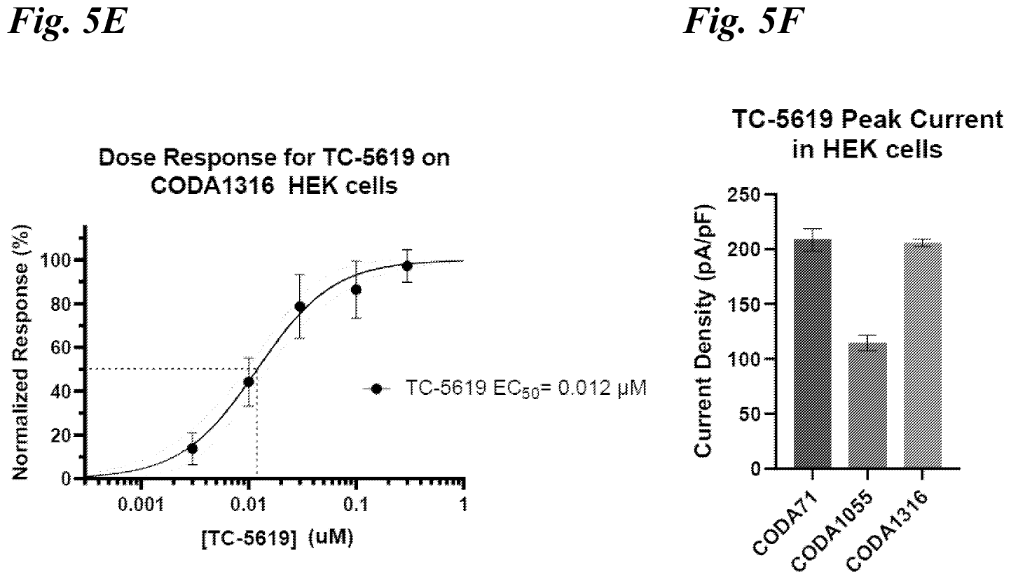

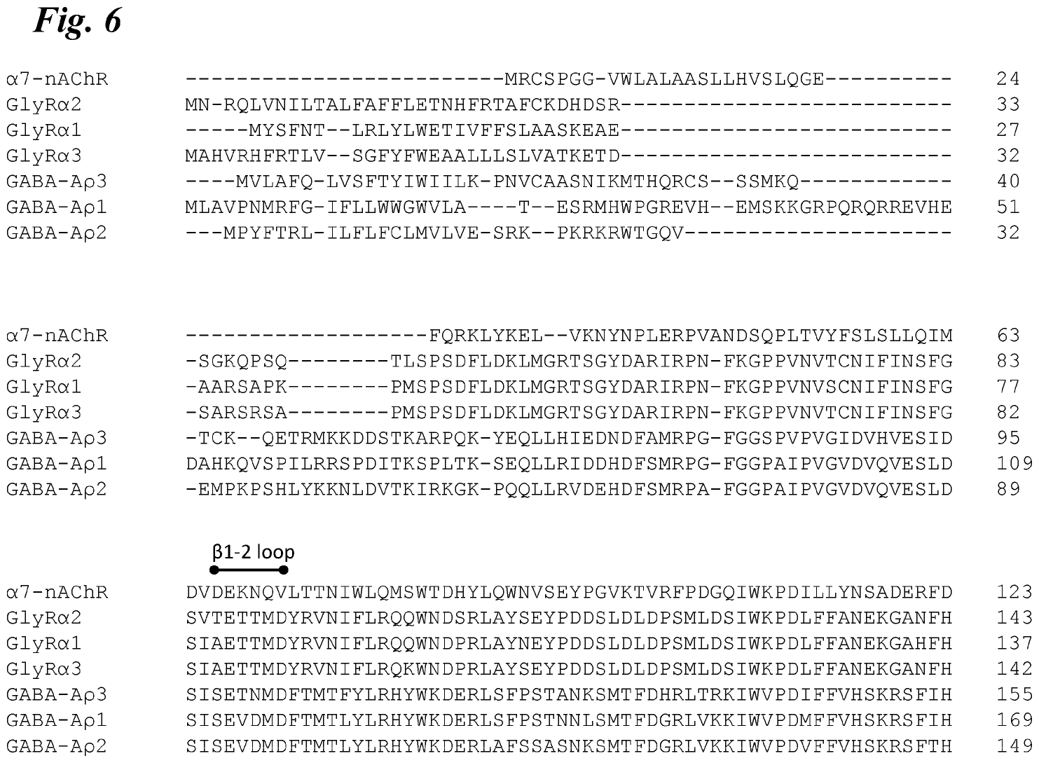

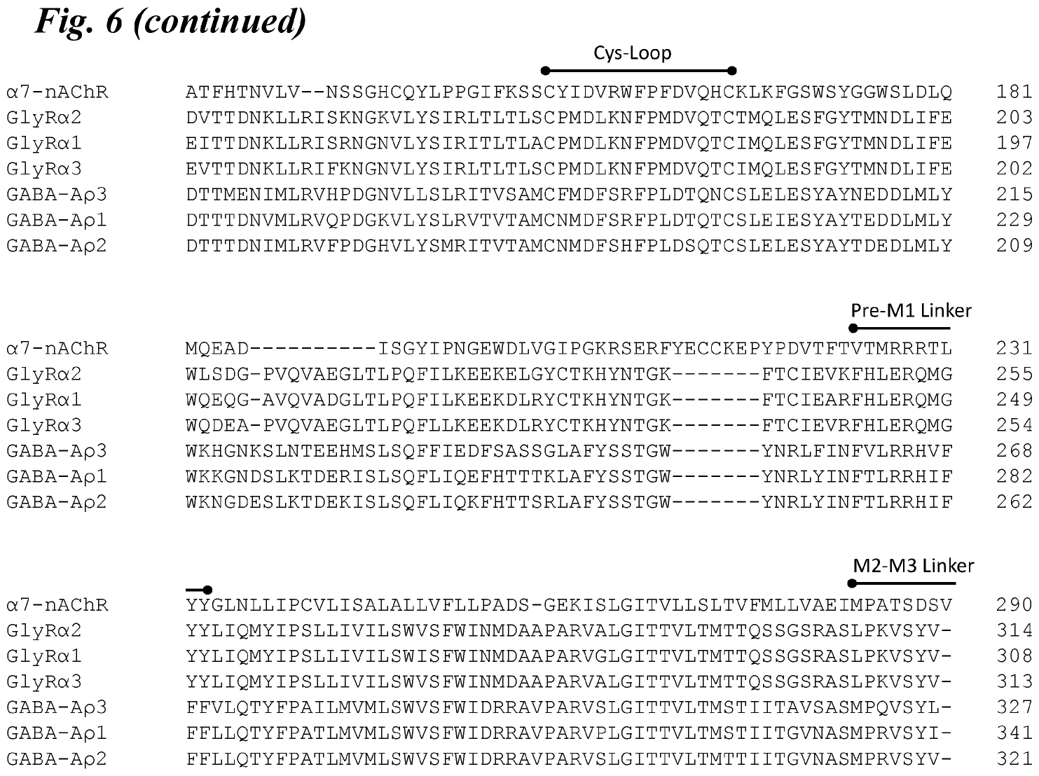

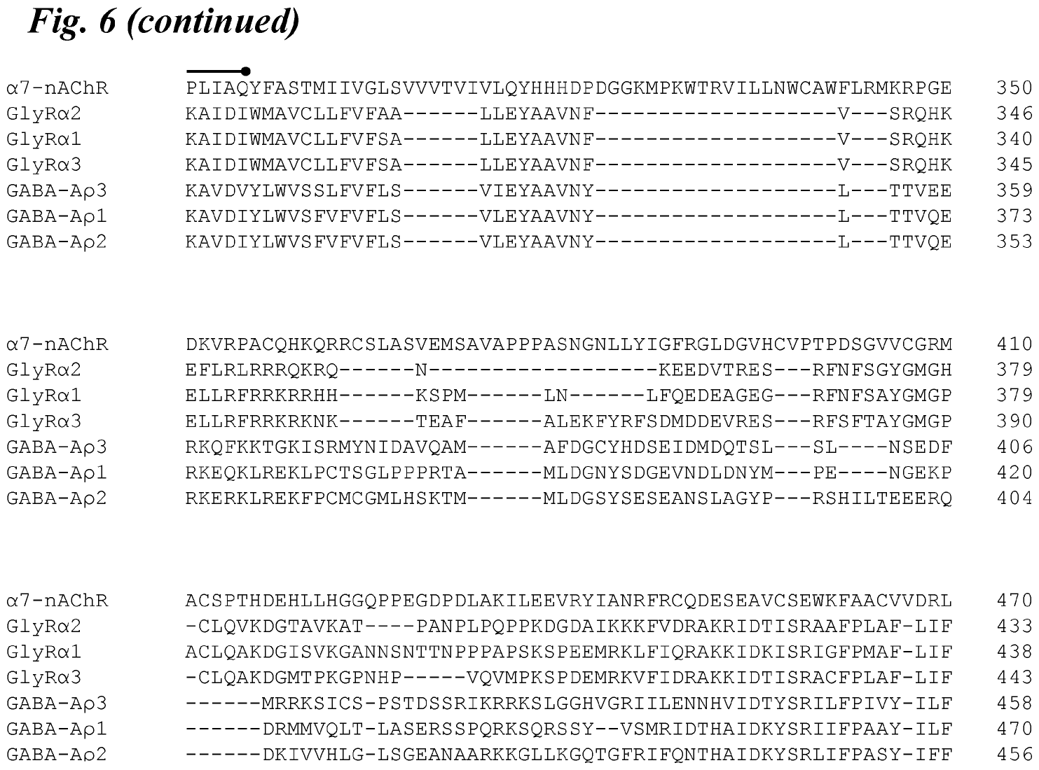

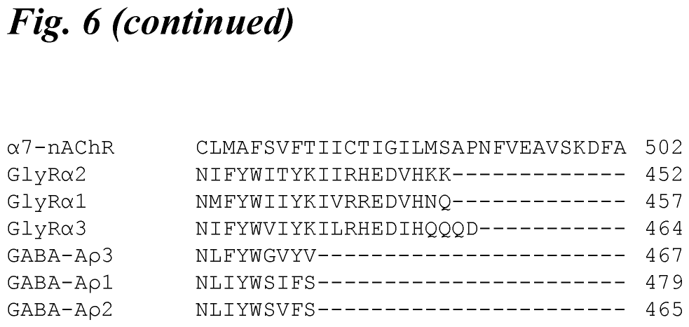

FIG. 5A shows the percentage of α-bungarotoxin staining positive HEK293T cells transiently transfected with CODA71, CODA409, CODA1237, CODA1297, CODA1298, CODA1299, CODA1300, CODA1301, CODA1302, CODA1303, CODA1304, CODA1305, CODA1306, CODA1308, CODA1309, or CODA1310. The design of the LGIC constructs are summarized in Table 12. FIG. 5B shows the mean fluorescent intensity of α-bungarotoxin staining positive cells from FIG. 5A.

The results show that CODA1237 has a higher surface expression than CODA71, and T202I mutation further increases the surface expression of the chimeric engineered receptor. Mutations in the ligand binding domain also affects the surface expression level of the chimeric engineered receptor.

### Example 4. Peak Current Measurements of Engineered Receptors Using IonFlux and Manual Patch Clamp

### Peak Current and Dose Response Measurements Using IonFlux:

HEK 293 cells were routinely subcultured in DMEM with 10% fetal bovine serum (ThermoFisher Scientific). Cells were plated in 15 cm dishes 24 hours prior to transfection with Fugene 6 (Promega, E2691) and plasmid DNA (total of 20 μg DNA). 24 hours post transfection, cells were lifted with Accutase (Sigma, A6964) then resuspended in serum free media (CHO—S-SFM II, ThermoFisher Scientific 12052 with 25 mM HEPES, ThermoFisher Scientific, 15630080) to rest for 30-90 min before being loaded onto IonFlux (Fluxion Biosciences) for experiments. Recordings were made in extracellular solution (ECS) containing (in mM): 140 sodium chloride (S7653), 4 potassium chloride (P9333), 1 magnesium chloride (M2670), 2 calcium chloride (C3881), 10 HEPES (54457), and 10 D-(+)-glucose (G7528) (pH=7.3 with sodium hydroxide, osmolarity=305-315 mOsm). Intracellular solution (ICS) contains (in mM): 140 cesium chloride (289329), 5 potassium fluoride (60238), 2 Calcium chloride (P9333), 2 magnesium chloride (M2670), 10 HEPES (54457), and 10 EGTA (3777) (pH=7.2 with cesium hydroxide, osmolarity=290-295 mOsm).

Drug-induced currents were recorded in ensemble mode (20 cells are patched per recording channel). Cells were held at −60 mV and current across the membrane was recorded at 1 kHz sampling rate. Drugs tested include acetycholine chloride (A6625) and other synthetic compounds. Drug doses were applied for 5 sec with a 2 min wash between dose applications unless otherwise stated. The holding current was subtracted from the peak to find the current amplitude. Doses were applied in half-log increments, increasing in concentration until the peak current amplitude plateaued. For each ensembled channel, amplitudes were normalized to the largest current. Normalized current amplitudes were averaged across channels and data were fit to a dose-response curve using a nonlinear regression model (Y=Bottom+(Top-Bottom)/(1+EC50/X)\hat{ }Hillslope). The maximum peak currents (absolute amplitude) from all the ensembled channels were averaged for each receptor and normalized to the mean maximum peak current from CODA71HA that was transfected and experimented in the same batch.

### Peak Current and Dose Response Measurements Using Manual Patch Clamp:

Cell culture: HEK 293 cells were routinely subcultured in DMEM with 10% fetal bovine serum (ThermoFisher Scientific). Cells were plated in six-well dishes 24 hours prior to transfection with Fugene 6 (Promega, E2691) and plasmid DNA (total of 1 μg DNA). 24 hours post transfection, cells were replated onto 10 mm PDL and collagen coated coverslips, before being used for patch clamp experiments 24-48 hours later. For dorsal root ganglion neuron experiments, adult rat lumbar ganglia were harvested and dissociated according to standard protocols, cultured on glass coverslips coated with poly-L-lysine and mouse laminin, transduced on DIV1 with ires-GFP-containing lentiviral or AAV6 vectors, and processed for immunocytochemistry on DIV7. For hippocampal neuron experiments, hippocampi were harvested from embryonic day 18 rat pups or postnatal day 0 mouse pups and dissociated according to standard protocols. Cells were cultured on glass coverslips coated with poly-L-lysine, transduced on DIV3 with ires-GFP-containing lentiviral or AAV6 vectors, and processed for immunocytochemistry on DIV14 (surface expression experiments) or stained with calcein violet and imaged on DIV16 (neuronal viability assays). 5-fluoro-deoxyuridine was added to hippocampal cultures on DIV3 (rat E18) or DIV7 (mouse PO) to inhibit glial growth. For surface expression experiments employing lentiviral expression, cultures were transduced at MOI=20 (DRG) or MOI=5 (hippocampal) with human synapsin promoter-driven lentiviral vectors. For lentiviral viability assays, cultures were transduced at MOI=2 (DRG) or MOI=1 (hippocampal) with ubiquitin promoter-driven lentiviral vectors. For all experiments employing AAV6 expression, cultures were transduced at MOI=2e5 (rat DRG and E18 hippocampal) or MOI=3e8 (mouse p0 hippocampal) with human synapsin promoter-driven AAV6 vectors.

Whole-cell patch clamp recording protocol: Cells plated on coverslips were visualized at 10× and 40× on an inverted fluorescence microscope (Olympus). Recordings were made with an Axopatch 200B amplifier and Digidata 1550B (Molecular Devices) at RT using 3-6 MOhm glass patch electrodes (Sutter, BF 150-86-10). Chemicals were acquired from Sigma except for TC-5619, which was manufactured by Attenua/Targacept. Unless otherwise stated, recordings were made in extracellular solution (ECS) containing (in mM): 140 sodium chloride (S7653), 4 potassium chloride (P9333), 1 magnesium chloride (M2670), 2 calcium chloride (C3881), 10 HEPES (54457), and 10 D-(+)-glucose (G7528) (pH=7.3 with sodium hydroxide, osmolarity=305-315 mOsm). An 8-line reservoir (AutoMate Scientific) in combination with an 8-channel zero-dead-volume perfusion pencil (AutoMate Scientific) was used for perfusion. Data was acquired using pClamp software (Molecular Devices). Traces were either analyzed in Clampfit (Molecular Devices) or using custom Python code. Plotting and statistics were performed in GraphPad Prism.

Dose-response experiments: Drug-induced currents were recorded in voltage clamp mode. For HEK cell experiments, recordings took place in the normal ECS with a cesium-based internal solution containing (in mM): 140 cesium chloride (289329), 5 potassium fluoride (60238), 2 Calcium chloride (P9333), 2 magnesium chloride (M2670), 10 HEPES (54457), and 10 EGTA (3777) (pH=7.2 with cesium hydroxide, osmolarity=290-295 mOsm). For DRG neuron experiments, cells were patched in normal ECS and then switched to an NMDG-based ECS to prevent endogenous cation currents, containing (in mM): 145 N-methyl-D-glucamine (M2004), 4 potassium chloride (P9333), 0.5 magnesium chloride (M2670), 0.5 calcium chloride (C3881), 10 HEPES (54457), and 10 D-(+)-glucose (G7528) (pH=7.4 with hydrochloric acid, osmolarity=305-315 mOsm). Internal solution for DRG neurons contained (in mM): 145 cesium chloride (289329), 1 magnesium chloride (M2670), 5 magnesium ATP (A9187), 10 HEPES (54457), and 1 EGTA (3777) (pH=7.2 with cesium hydroxide, osmolarity=290-295 mOsm). Cells were held at −60 mV and current across the membrane was recorded in gapfree mode at 3 kHz sampling rate, with Bessel filter at 1 kHz. Capacitance and series resistance were compensated. Drugs tested include acetycholine chloride (A6625) and other synthetic compounds. Drug doses were applied for 1-10 sec until the peak of the current was observed, and washes occurred for at least 2 min between dose applications. The holding current was subtracted from the peak to find the current amplitude. Doses were applied in half-log increments, increasing in concentration until the peak current amplitude plateaued. For each cell, amplitudes were normalized to the largest current. Normalized current amplitudes were averaged across cells and data were fit to a dose-response curve using a nonlinear regression model (Y=Bottom+(Top-Bottom)/(1+EC50/X)\hat{ }Hillslope). The maximum peak currents (absolute amplitude) from all the cells for each receptor were averaged and within-batch comparisons were performed to control for batch effects.

### Rheobase Experiments:

DRG neurons: DRG rheobase experiments were performed in current clamp mode using a potassium-based internal solution containing (in mM): 100 potassium D-gluconate (G4500), 28 potassium chloride (P9333), 1 magnesium chloride hexahydrate (M2670), 5 magnesium ATP (A9187), 10 HEPES (54457), and 0.5 EGTA (3777) (pH=7.2 with potassium hydroxide, osmolarity=290-295 mOsm, calculated liquid junction potential=−13.5 mV). To measure DRG input resistance and rheobase, 500 ms current steps (−200 pA to 700 pA) were applied the membrane (−400 pA to 700 pA in an early version of the protocol) in the presence and absence of a compound. No holding current was applied to the membrane for cells included in the final datasets. Data were acquired at 10 kHz, with Bessel filter at 2 kHz and series resistance compensation at 100%. Rheobase was defined as the smallest depolarizing current injection required to cause an action potential to fire in the cell. In a subset of cells, the rheobase was ambiguous, since the action potential appeared to be graded in amplitude. In these cases, a polar plot of the trace clarified the rheobase. Input resistance was measured from the negative current injections, calculated as the difference in membrane potential at steady state divided by the current injection amplitude and then averaged across the four negative current steps for each recording. Resting membrane potential was measured from the first 100 ms of each sweep, averaged for each recording, and corrected for liquid junction potential. One-way ANOVA with multiple comparisons was used to test for significance of difference in rheobase and input resistance between baseline, drug, and wash conditions.

Hippocampal neurons: Hippocampal rheobase experiments were performed in current clamp mode using a potassium-based internal solution containing (in mM): 115 potassium D-gluconate (G4500), 13 potassium chloride (P9333), 1 magnesium chloride hexahydrate (M2670), 5 magnesium ATP (A9187), 10 HEPES (54457), and 0.5 EGTA (3777) (pH=7.2 with potassium hydroxide, osmolarity=290-295 mOsm, calculated liquid junction potential=−14.9 mV. NBQX (10 μM) and AP5 (50 μM) were included in the bath to block excitatory synaptic transmission. To measure HC input resistance and rheobase, 500 ms current steps (−200 pA to 700 pA) were applied the membrane in the presence and absence of a compound. No holding current was applied to the membrane for cells included in the final datasets. Data acquisition settings and analysis methods were the same as for the DRG rheobase experiments.

Hippocampal spontaneous action potential experiments: Hippocampal spontaneous action potentials were recorded in current clamp mode using a potassium-based internal solution containing (in mM): 115 potassium D-gluconate (G4500), 13 potassium chloride (P9333), 1 magnesium chloride hexahydrate (M2670), 5 magnesium ATP (A9187), 10 HEPES (54457), and 0.5 EGTA (3777) (pH=7.2, osmolarity=290-295 mOsm, calculated liquid junction potential=−14.9 mV). Spontaneous activity was recorded in gapfree mode, in the presence and absence of a compound. Data were acquired at 10 kHz, with Bessel filter at 2 kHz and series resistance compensation at 100%. No holding current was applied to the membrane. Data were analyzed using Clampfit as well as custom Python code (within-cell comparisons of the two methods did not reveal differences in results). For Clampfit, threshold-based event detection was run separately for each epoch to detect action potentials. For the custom Python code, a peak-finding package was used to automatically detect action potentials (SciPy). For both methods, minimum height, average height, maximum height were measured and frequency was calculated as the number of events in the epoch divided by the length of time of the epoch. For Clampfit, the resting membrane potential was measured at 3 points between events and averaged for each epoch. For resting membrane potential in Python, a median filter was applied to the trace and averaged for each epoch. For both methods, membrane potential was corrected for the calculated liquid junction potential. One-way ANOVA with multiple comparisons was used to test for significance between baseline, drug, and wash conditions.

### Results:

Peak current data (both ionflux and manual patch) for CODA1055 and CODA1316 receptors in HEK cells are shown in Table 14 below.

TABLE 14
Peak Current of Engineered Receptors

| | | Peak Current on | Peak Current on |
|---|---|---|---|
| | | IonFlux in HEK | Manual Patch Clamp |
| Receptor | Description of the | Cells | in HEK cells |
| Number | Engineered Receptor | (picoamps (pA)) | (picoamps (pA)) |
| CODA1055 | α7-nAChR(Gly-cys) LBD: | −5074.3 | −2176.6 |
| | GlyRα1 IPD (SEQ ID NO: 33) | | |
| | comprising R101W, | | |
| | Y115E, Y210W mutations | | |
| CODA1316 | α7-nAChR(Gly-cys) LBD: | −5520.5 | −2779 |
| | GlyRα2 IPD (SEQ ID NO: 65) | | |
| | comprising R101W, Y115E, | | |
| | Y210W, T225I mutations | | |

In another set of experiments, the TC-5619 dose response and peak current data for CODA71, CODA1055, and CODA1316 were measured using HEK cells. According to FIGS. 5C-5E, introducing the R101W, Y115E, and Y210W (as in CODA1055 and CODA12316) resulted in increased response to TC-5619 and reduced response to acetylcholine (at lease for CODA1055). On the other hand, the peak current results of CODA1316 and CODA1055 in FIG. 5F shows that replacing the GlyRα1 ion pore domain with the GlyRα2 ion pore domain and introducing the T225I mutation in the pre-M1 linker resulted in increased peak current, which may benefit the in vivo efficacy of the engineered receptor.

### Example 5. Micro Electrode Array Measurements of the Engineered Receptors

Primary hippocampal cultures were obtained from C57/B16 mice at day E17.5. Mice were sacrificed by inhalation of CO₂ and embryos removed immediately. Embryonic brains were dissected in ice cold HBSS supplemented with 0.6% glucose and 5 mM HEPES at pH 7.4. The hippocampus of each brain was extracted and rinse with HBSS twice then treated with 0.25% trypsin for 15 minutes at 37° C. Cells were then washed three time in HBSS, resuspended in Neurobasal media supplemented with 10% FBS and 1% PenStrep and mechanically dissociated by gentle pipetting with glass Pasteur pipette. Cells we plated at a density of 60,000 cell per well in a 24 well microelectrode array plate (Axion Biosystems) for 4 hours. The plating media was then replaced with Neurobasal media, 1×B-27, 1% PenStrep, 1 mM Glutamax, 10 mM HEPES pH 7.4

Cells were maintained in a humidified 5% C02 incubator and 30% of the media was replaced every 2-3 days. On day in vitro (DIV) 1, cells were transduced with either AAV6 or AAV9 carrying the CODA receptor of interested under control of the hSYN promoter. On DIV 21 the mean firing rate (MFR) of each well was determined for 10 minutes prior to drug administration and then again 50-60 minutes post dose in a Maestro Edge microectrode array system. Axion Biosystems). Percent inhibition of the MFR in each well was calculated as 100-[(post dose MFR/pre-dose MFR)*100].

### Example 6. Characterization of Engineered Receptors in IPSC-Derived Neurons (Prophetic)

Differentiation protocols to generate IPSC-derived Aβ neurons are developed. The following markers are used to define the cells as Aβ neurons; expression of Neurofilament 200 (NF200), which delineates myelinated primary afferent neurons (Basbaum et al, 2009), Piezo 2, a marker of Low Threshold Mechanoreceptor sensory neurons (LTMRs) (Ranade et al., 2014) and TLR5, a toll-like receptor that reportedly also marks Aβ fibers (Xu et al., 2015). The characterization will also include evaluation for absence of the nociceptor specific marker TrpVl, which is expressed in many C and Aβ fibers (Caterina et al., 1997), prostatic acid phosphatase, which delineates non-peptidergic unmyelinated afferents (Zylka et al., 2009), and NaV1.1, a marker of Aβ nociceptive neurons. IPSC-derived neurons that meet the above criteria will be further characterized as either rapidly adapting LTMRs, based on expression of c-Ret and MafA/C-Maf, or slowly adapting LTMRs, based on expression of TrkB and Shox2, in the absence of c-Ret expression (Koch et al., 2018).
(1) Confirm that cells meeting the above expression marker criteria of Aβ neurons have electrophysiology properties characteristic of non-nociceptive sensory neurons. These properties include generation of current in response to ligand and that direct current injection evokes action potentials.
(2) Confirm presence of chloride currents in neurons transduced with select chemogenetic receptors (engineered receptors). Cells are transduced with a lentivirus vector encoding an HA-tagged chemogenetic receptor(s) with an IRES GFP to identify transduced cells via GFP fluorescence. Chloride currents in response to Synthetic Agonists are detected in voltage clamp mode with NMGD+as the internal/external fluid cation. As NMDG+ is impermeable to cation channels, its inclusion eliminates any endogenous nAchRa7 cation currents in response to the Synthetic Agonists studied. The EC50 for Chemogenetic Receptor-Synthetic Agonist pairs is then determined. Following recordings, expression and cell surface localization of the receptor(s) are evaluated by fluorescence microscopy in permeabilized and non-permeabilized cells using antibodies directed at the HA tag.
(3) Assess ability to inhibit current injection evoked action potentials. Cells are transduced with a lentivirus vector encoding an HA-tagged chemogenetic receptor(s) with an IRES GFP to identify transduced cells via GFP fluorescence. In current clamp mode, by escalating application of current until the cell membranes depolarizes, the rheobase in the presence and absence of the synthetic agonist is determined. Results are compared to cells that are transduced with GFP only.
(4) Assess impact on input resistance. Cells are transduced with a lentivirus vector encoding an HA-tagged chemogenetic receptor(s) with an IRES GFP to identify cells via GFP fluorescence. In current clamp mode, to calculate the input resistance, sub threshold current is injected and resulting change in membrane voltage is determined. Results are compared to cells that are transduced with GFP only.
(5) Assess impact on resting membrane potential in the presence and absence of synthetic agonist. Cells are transduced with a lentivirus vectors encoding an HA-tagged chemogenetic receptor(s) with an IRES GFP to identify transduced cells via GFP fluorescence. In voltage clamp mode the resting membrane potential is determined in the presence and absence of the synthetic agonist. Results are compared to cells that are transduced with GFP only.

The above electrophysiology properties in the IPSC-derived Aβ neurons are compared with those in IPSC-derived C-fiber neurons as well as adult rat DRG neurons. In addition, as biochemical changes in the injured Aβ fiber afferents can contribute to spontaneous pain in neuropathic states, the electrophysiological properties of the IPSC derived Aβ neurons is investigated following injury in vitro. To produce the injuries in vitro, cells are harvested and re-plated after processes have been extended in culture. The re-plating process severs the processes, mimicking an axonal injury. At various time point post injury the cells are evaluated for various electrophysiology properties including: generation of spontaneous action potentials, changes in resting membrane potentials and changes in the rheobase. The effect of the chemogenetic receptors are evaluated in the injured state as well.

### Example 7. Assessing the Efficacy of Engineered Receptors to Treat Disease in Animal Models

The engineered receptors disclosed herein are assessed for their ability to provide analgesia in a rat model of neuropathic pain following administration of a small molecule ligand. AAV expression cassettes containing a human synapsin-1 (hSYN) promoter linked to polynucleotides encoding either a wild type α7-nAChR, or any one of the engineered chimeric receptors disclosed herein are constructed using standard molecular biology techniques.

These AAV expression cassettes are subdloned into AAV bacmids, purified, transfected into Sf9 insect cells to produce recombinant baculovirus, and then amplified. Sf9 cells are double infected with the amplified recombinant baculovirus containing with either the wild type α7-nAChR, or any one of the engineered chimeric receptors cassettes described above, and another recombinant baculovirus containing the Rep and AAV6 (Y705+731F+T492V) Cap genes to produce recombinant AAV vectors. The viral vectors are purified, viral titer determined using qPCR, and SDS-PAGE used to verify the purity of AAV vectors.

Behavioral experiments and pain models: To produce mechanical hypersensitivity in a model that mimics a neuropathic pain condition, the spared nerve injury (SNI) model (a validated model of mechanical allodynia) is used (Shields et al., 2003, The Journal of Pain, 4, 465-470). This model is produced by the sectioning of the common peroneal and the sural nerves and isolating the tibial branch. Mechanical withdrawal threshold is assessed by placing rats on an elevated wire-mesh grid and stimulating the plantar surface of the hind paw with von Frey filaments.

AAV injection into the spinal cord of rats: A dorsal hemilaminectomy is made at the level of the lumbar enlargement to expose two segments (about 1.5-2 mm) of lumbar spinal cord, after which the dura mater is incised and reflected. The viral solution is loaded into a glass micropipette (prefilled with mineral oil). The micropipette is connected to a manual micro-injector mounted on a stereotactic apparatus. The viral solution is targeted to the dorsal horn (left side). Along the rostro-caudal axis within the exposed region, 6 injections of 240 nl each are performed, in an equidistant linear fashion. After each injection, 1 min of resting time is observed and then the muscle layer is sutured, the skin closed with staples, and the animals were allowed to recover with heated-pad before they were returned to their home cages. Animals are perfused for histological analysis after the last behavior test.

AAVintraganglionic injections into the dorsal root ganglion (DRG) ofrats: The injection is performed with a borosilicate glass capillary (0.78/1 mm internal/external diameters) pulled to a fine point, attached by polyethylene tubing (0.4/0.8 mm internal/external diameters) to a syringe mounted in a microinjection pump. The needle is mounted on an extended arm of a stereotaxic frame swung to the outside (used to hold and manipulate the needle only). Tubing, syringe, and needle are all filled with water. One microliter air is taken up into the needle followed by 3 μL of the viral vector solution. The needle is loaded separately with this volume for each injection. Animals are anesthetized prior to surgery. Following an incision along the dorsal midline, the L4 and L5 DRG are exposed by removal of the lateral processes of the vertebrae. The epineurium lying over the DRG is opened, and the glass needle inserted into the ganglion, to a depth of 400 m from the surface of the exposed ganglion. After a 3-minute delay to allow sealing of the tissue around the glass capillary tip, 1.1 μL virus solution was injected at a rate of 0.2 L/minute. After a further delay of 2 minutes, the needle is removed. The L4 ganglion is injected first followed by the L5 ganglion. The muscles overlying the spinal cord are loosely sutured together with a 5-0 suture and the wound closed. Animals are allowed to recover at 37° C. and received postoperative analgesia.

AAV intrathecal injections in rats: Rats are first anesthetized and then placed vertically with their head fixed in a stereotaxic frame. An incision is made in the base of the neck to expose the groove in the nuchal crest. An incision is made (1-2 mm) in the cisternal membrane to a depth such that cerebrospinal fluid leaks out. A 4 cm 32 G intrathecal catheter is then slowly inserted in the direction of the lumbar spinal cord and skin is closed by suture around the catheter. The rats are then allowed to recover. Rats are then anesthetized and the vector (6 L) is administered. The catheter is flushed with 6 μL of PBS and then removed and rats allowed to recover.

Effects of administration: This SNI model is produced by the sectioning of the common peroneal and the sural nerves and isolating the tibial branch of the rat. The up-down method of Chaplan & Yaksh is used to determine mechanical thresholds before the injection of the AAV.hSYN encoding the engineered receptor of the disclosure into the spinal cord, DRG, or intrathecal space. Three weeks after unilateral vector injection, animals are tested again to verify that their mechanical withdrawal thresholds do not change. Motor coordination is also tested before and after injection, using an accelerating rotarod (Stoelting, USA) at a maximum speed of 33 rpm. The duration that the rat spends on the rotarod is recorded, with a cut-off at 300 sec. Each rat goes through three training trials and is tested two hours later.

Subsequently, half of the rats in each chimera cohort are administered a single IP injection of AZD-0328 or Facinicline and mechanical thresholds tested using the up-down method at 1, 2, 5, 7, and 13 days post IP injection. On the third day, when the thresholds has returned to post-injury baseline, AZD-0328 is again injected IP and again a recovery to non-injury baseline thresholds is observed. These animals are followed for 48 hours. Animals are then perfused for histology.

### Example 8: Treatment of a Patient Suffering from Chronic Pain

In a non-limiting example, a patient suffering from chronic radicular pain is treated using the compositions and methods disclosed herein. The patient is treated on Day One with 10¹³ vector genomes of AAV.hSYN operably linked to a polynucleotide encoding any one of the engineered receptors disclosed herein in a volume of 1.0 mL delivered directly into one or more dorsal root ganglia (i.e., intraganglionic convection-enhanced delivery into lumbar, cervical, or thoracic DRGs). In this example, the AAV vector encodes any one of the engineered receptors disclosed herein under the control of the human Synapsin-1 (SYN1) promoter for selective neuronal expression. Two weeks post-injection, the patient returns to the clinic for a prescription for AZD-0328 or another non-native ligand. The patient self-administers 0.1 mg/kg AZD-0328 or another non-native ligand orally as needed (i.e., during a pain episode).

### Example 9. Treatment of a Patient Suffering from Chronic Pain

In a non-limiting example, a patient suffering from chronic craniofacial pain (e.g. trigeminal neuralgia or termporomandibular joint dysfunction) is treated using the compositions and methods disclosed herein. The patient is treated on Day One with 10¹³ vector genomes of AAV.hSYN operably linked to a polynucleotide encoding any one of the engineered receptors disclosed herein in a volume of 0.150 mL delivered directly into the trigeminal ganglion (i.e., intraganglionic convection enhanced delivery). In this example, the AAV vector encodes any one of the engineered receptors disclosed herein under the control of the human Synapsin-1 (SYN1) promoter for selective neuronal expression. Two weeks post-injection, the patient returns to the clinic for a prescription for AZD-0328 or another non-native ligand. The patient self-administers 0.1 mg/kg AZD-0328 or another non-native ligand orally as needed (i.e., during a pain episode).

### Example 10. Treatment of a Patient Suffering from Obesity

In a non-limiting example, a patient suffering from obesity is treated using the compositions and methods disclosed herein. The patient is treated on Day One with 10¹³ vector genomes of AAV. Ghrelin operably linked to a polynucleotide encoding any one of the engineered receptors disclosed herein in a volume of 1.0 mL delivered directly into the gastric branch of the vagus nerve (i.e., intraneural). In this example, the AAV vector encodes the engineered receptor under the control of the human Ghrelin promoter for selective neuronal expression. Two weeks post-injection, the patient returns to the clinic for a prescription for AZD-0328 or another non-native ligand. The patient self-administers 0.1 mg/kg AZD-0328 or another non-native ligand orally daily for excess weight loss (i.e. for apetitite suppression).

### Example 11. Treatment of a Patient Suffering from Obesity

In a non-limiting example, a patient suffering from obesity is treated using the compositions and methods disclosed herein. The patient is treated on Day One with 10¹³ vector genomes of AAV-TRPV1 operably linked to a polynucleotide encoding any one of the engineered receptors disclosed herein in a volume of 1.0 mL delivered directly into the dorsal root ganglia innervating the pancreas (i.e., intragangionic). In this example, the AAV vector encodes the engineered receptor under the control of the human TRPV1 promoter for selective neuronal expression in nociceptors. Two weeks post-injection, the patient returns to the clinic for a prescription for AZD-0328 or another non-native ligand. The patient self-administers 0.1 mg/kg AZD-0328 or another non-native ligand orally daily for excess weight loss.

### Example 12. Treatment of a Patient Suffering from Obesity

In a non-limiting example, a patient suffering from obesity is treated using the compositions and methods disclosed herein. The patient is treated on Day One with 10¹³ vector genomes of AAV-SIM1 operably linked to a polynucleotide encoding any one of the engineered receptors disclosed herein in a volume of 1.0 mL delivered directly into the paraventricular nucleus (PVH) in the hypothalamus (i.e., intracranial, convection enhanced delivery). In this example, the AAV vector encodes the engineered channel under the control of the human Single-Minded Family BHLH Transcription Factor 1 (SIM1) promoter for selective neuronal expression in pro-opiomelanocortin (POMC) neurons and ultimately stimulation of the anorexigenic pathway. Two weeks post-injection, the patient returns to the clinic for a prescription for AZD-0328 or another non-native ligand. The patient self-administers 0.15 mg/kg AZD-0328 or another non-native ligand orally daily for excess weight loss (i.e. for apetitite suppression).

### Example 13. Treatment of a Patient Suffering from PTSD

In a non-limiting example, a patient suffering from post-traumatic stress disorder (PTSD) is treated using the compositions and methods disclosed herein. The patient is treated on Day One with 10¹³ vector genomes of AAV-hSYN1 operably linked to a polynucleotide encoding any one of the engineered receptors disclosed herein in a volume of 1.0 mL delivered directly into the C6 stellate ganglion, (i.e., intraganglionic). In this example, the AAV vector encodes the engineered receptor under the control of the human Synapsin-1 (hSYN1) promoter for selective neuronal expression. Two weeks post-injection, the patient returns to the clinic for a prescription for AZD-0328 or another non-native ligand. The patient self-administers 0.15 mg/kg AZD-0328 or another non-native ligand orally daily for PTSD symptoms (i.e. for anxiety).

### Example 14. Treatment of a Patient Suffering from Depression

In a non-limiting example, a patient suffering from treatment-resistant depression (TRD) is treated using the compositions and methods disclosed herein. The patient is treated on Day One with 10¹³ vector genomes of AAV-hSYN1 operably linked to a polynucleotide encoding any one of the engineered receptors disclosed herein in a volume of 1.0 mL delivered directly into the vagus nerve, (i.e., intraneural). In this example, the AAV vector encodes the engineered receptor under the control of the human Synapsin-1 (hSYN1) promoter for selective neuronal expression. Two weeks post-injection, the patient returns to the clinic for a prescription for AZD-0328 or another non-native ligand. The patient self-administers 0.1 mg/kg AZD-0328 or another non-native ligand orally daily for depression symptoms.

### Example 15. Treatment of a Patient Suffering from GERD

In a non-limiting example, a patient suffering from gastroesophageal reflux disease (GERD) is treated using the compositions and methods disclosed herein. The patient is treated on Day One with 10¹³ vector genomes of AAV-hSYN1 operably linked to a polynucleotide encoding any one of the engineered receptors disclosed herein or AAV-CAG operably linked to a polynucleotide encoding any one of the engineered receptors disclosed herein in a volume of 1.0 mL delivered directly into the lower esophageal sphincter (LES) vagus nerve and myenteric plexus (i.e., intraneural) or smooth muscle (intramuscular), respectively. In this example, the AAV vector encodes the engineered receptor under the control of the human Synapsin-1 (hSYN1) promoter for selective neuronal expression or the CAG promoter for expression in LES myocytes. Two weeks post-injection, the patient returns to the clinic for a prescription for AZD-0328 or another non-native ligand. The patient self-administers 0.15 mg/kg AD-0328 or another non-native ligand orally daily for symptoms of GERD (i.e. acid reflux).

### Example 16. Treatment of a Patient Suffering from Epilepsy

In a non-limiting example, a patient suffering from seizures associated with epilepsy is treated using the compositions and methods disclosed herein. The patient is treated on Day One with 10¹³ vector genomes of AAV-CamKIIα operably linked to a polynucleotide encoding any one of the engineered receptors disclosed herein in a volume of 1.0 mL delivered directly into a pre-determined seizure focus such as the motor cortex (i.e., intracranial). In this example, the AAV vector encodes the engineered receptor under the control of the human Calcium/calmodulin-dependent protein kinase II α (CamKIIα) promoter for selective neuronal expression in excitatory neurons. Two weeks post-injection, the patient returns to the clinic for a prescription for AZD-0328. The patient self-administers 0.1 mg/kg AZD-0328 orally daily for epileptic symptoms (i.e. seizures).

### Example 17. Treatment of a Patient Suffering from a Movement Disorder

In a non-limiting example, a patient suffering from a movement disorder (e.g. Parkinsonian tremor) is treated using the compositions and methods disclosed herein. The patient is treated on Day One with 10¹³ vector genomes of AAV-CamKIIα operably linked to a polynucleotide encoding any one of the engineered receptors disclosed herein in a volume of 1.0 mL delivered directly into the subthalamic nucleus (i.e., intracranial STN). In this example, the AAV vector encodes the engineered receptor under the control of the human Calcium/calmodulin-dependent protein kinase II α (CamKIIα) promoter for selective neuronal expression in excitatory neurons. Two weeks post-injection, the patient returns to the clinic for a prescription forAZD-0328. The patient self-administers 0.1 mg/kg AZD-0328 orally daily for movement disorder symptoms (i.e. tremor).

### FURTHER NUMBERED EMBODIMENTS

Further embodiments of the instant invention are provided in the numbered embodiments below:

Embodiment 1. An engineered receptor, wherein the engineered receptor is a chimeric ligand gated ion channel (LGIC) receptor and comprises
(a) a ligand binding domain derived from a first wild type Cys-loop LGIC receptor, and
(b) an ion pore domain derived from a second wild type Cys-loop LGIC receptor.

Embodiment 2. The engineered receptor according to Embodiment 1, wherein the first wild type Cys-loop LGIC receptor comprises a nicotinic acetylcholine receptor family receptor.

Embodiment 3. The engineered receptor according to Embodiment 1 or 2, wherein the first wild type Cys-loop LGIC receptor is human α7 nicotinic acetylcholine receptor (α7-nAChR, SEQ ID NO.4).

Embodiment 4. The engineered receptor according to any one of Embodiments 1-3, wherein the second wild type Cys-loop LGIC receptor is a chloride permeable Cys-loop ligand gated ion channel receptor.

Embodiment 5. The engineered receptor according to Embodiment 4, wherein the second wild type Cys-loop LGIC receptor is a glycine receptor or a GABA-A receptor.

Embodiment 6. The engineered receptor according to Embodiment 5, wherein the second wild type Cys-loop LGIC receptor comprises a Glycine receptor α1, a Glycine receptor α2, a Glycine receptor α3, a GABA-A receptor ρ1, a GABA-A receptor ρ2, or a GABA-A receptor ρ3.

Embodiment 7. The engineered receptor according to any one of Embodiments 1-6, wherein the second wild type Cys-loop LGIC receptor is not a Glycine receptor α1.

Embodiment 8. The engineered receptor according to any one of Embodiments 1-7, wherein part or all of Cys-loop domain of the ligand binding domain is derived from the second wild type Cys-loop LGIC receptor.

Embodiment 9. The engineered receptor according to any one of Embodiments 1-8, wherein part or all of β1-2 loop domain of the ligand binding domain is derived from the second wild type Cys-loop LGIC receptor.

Embodiment 10. The engineered receptor according to any one of Embodiments 1-9, wherein the engineered receptor comprises a pre-M1 linker derived from the first wild type Cys-loop LGIC receptor or the second wild type Cys-loop LGIC receptor.

Embodiment 11. The engineered receptor according to any one of Embodiments 1-9, wherein the engineered receptor comprises a pre-M1 linker comprising a N-terminal segment derived from the first wild type Cys-loop LGIC receptor and a C-terminal segment derived from the second wild type Cys-loop LGIC receptor.

Embodiment 12. The engineered receptor according to Embodiment 10 or 11, wherein the pre-M1 linker comprises or consist of a sequence having at least 70% identity to any one of SEQ ID NOS: 72-91.

Embodiment 13. The engineered receptor according to any one of Embodiments 1-12, wherein part or all of M2-M3 linker of the ion pore domain is derived from the first wild type Cys-loop LGIC receptor.

Embodiment 14. The engineered receptor according to any one of Embodiments 1-13, wherein the M2-M3 linker of the ion pore domain comprises one or more mutations.

Embodiment 15. The engineered receptor according to any one of Embodiments 1-14, wherein the M2-M3 linker of the ion pore domain comprises an amino acid sequence having at least 70% identity according to amino acids 283-295 of SEQ ID NO:4.

Embodiment 16. The engineered receptor according to any one of Embodiments 1-15, wherein the engineered receptor forms homomeric ion channels when expressed on cell surface.

Embodiment 17. The engineered receptor according to Embodiment 16, wherein more than 50% of the engineered receptor expressed on cell surface form homomeric ion channels.

Embodiment 18. The engineered receptor according to any one of Embodiments 1-17, wherein the second wild type Cys-loop LGIC receptor is a human Glycine receptor α1 subunit (GlyRα1).

Embodiment 19. The engineered receptor according to Embodiment 18, wherein the ion pore domain comprises an amino acid sequence having at least 85% identity according to amino acids 248-457 of SEQ ID NO:2.

Embodiment 20. The engineered receptor according to Embodiment 18 or 19, wherein the Cys-loop domain of the ligand binding domain comprises an amino acid sequence having at least 80% identity according to amino acids 166-180 of SEQ ID NO:2.

Embodiment 21. The engineered receptor according to any one of Embodiments 18-20, wherein the β1-2 loop domain of the ligand binding domain comprises an amino acid sequence having at least 80% identity according to amino acids 80-85 of SEQ ID NO:2.

Embodiment 22. The engineered receptor according to any one of Embodiments 18-21, wherein the ion pore domain comprises no amino acid between the amino acid positions corresponding to K353 and E362 of SEQ ID NO:2.

Embodiment 23. The engineered receptor according to any one of Embodiments 18-21, wherein the ion pore domain comprises an amino acid sequence having less than 50% sequence identity to SEQ ID NO: 96 between the amino acid positions corresponding to K353 and E362 of SEQ ID NO:2.

Embodiment 24. The engineered receptor according to any one of Embodiments 18-23, wherein the ion pore domain comprises one or more mutations in a region corresponding to the nuclear localization signal (NLS)/ER retention signal (ERRS) sequence of the human GlyRα1.

Embodiment 25. The engineered receptor according to any one of Embodiments 18-24, wherein the engineered receptor comprises an amino acid sequence having at least 90% identity to SEQ ID NO:16 or 33.

Embodiment 26. The engineered receptor according to any one of Embodiments 1-17, wherein the second wild type Cys-loop LGIC receptor is a human Glycine receptor α2 subunit (GlyRα2).

Embodiment 27. The engineered receptor according to Embodiment 26, wherein the ion pore domain comprises an amino acid sequence having at least 85% identity according to amino acids 254-452 of SEQ ID NO:59.

Embodiment 28. The engineered receptor according to Embodiment 26 or 27, wherein the Cys-loop domain of the ligand binding domain comprises an amino acid sequence having at least 80% identity according to amino acids 172-186 of SEQ ID NO:59.

Embodiment 29. The engineered receptor according to any one of Embodiments 26-28, wherein the β1-2 loop domain of the ligand binding domain comprises an amino acid sequence having at least 80% identity according to amino acids 86-91 of SEQ ID NO:59.

Embodiment 30. The engineered receptor according to any one of Embodiments 26-29, wherein the ion pore domain comprises one or more mutations in a region corresponding to the NLS/ERRS sequence of the human GlyRα2.

Embodiment 31. The engineered receptor according to any one of Embodiments 26-30, wherein the engineered receptor comprises an amino acid sequence having at least 90% identity to SEQ ID NO:62 or 65.

Embodiment 32. The engineered receptor according to any one of Embodiments 1-17, wherein the second wild type Cys-loop LGIC receptor is the human Glycine receptor α3 subunit (GlyRα3).

Embodiment 33. The engineered receptor according to Embodiment 32, wherein the ion pore domain comprises an amino acid sequence having at least 85% identity according to amino acids 253-464 of SEQ ID NO:61 or amino acids 253-449 of SEQ ID NO: 69.

Embodiment 34. The engineered receptor according to Embodiment 32 or 33, wherein the Cys-loop domain of the ligand binding domain comprises an amino acid sequence having at least 80% identity according to amino acids 171-185 of SEQ ID NO:61 or 69.

Embodiment 35. The engineered receptor according to any one of Embodiments 32-34, wherein the β1-2 loop domain of the ligand binding domain comprises an amino acid sequence having at least 80% identity according to amino acids 85-90 of SEQ ID NO:61 or 69.

Embodiment 36. The engineered receptor according to any one of Embodiments 32-35, wherein the ion pore domain comprises no amino acid residue between the amino acid positions corresponding to K357 and D358 of SEQ ID NO:69.

Embodiment 37. The engineered receptor according to any one of Embodiments 32-35, wherein the ion pore domain comprises an amino acid sequence having less than 50% sequence identity to SEQ ID NO: 95 between the amino acid positions corresponding to K357 and D358 of SEQ ID NO:69.

Embodiment 38. The engineered receptor according to any one of Embodiments 32-37, wherein the ion pore domain comprises one or more mutations in a region corresponding to the NLS/ERRS sequence of the human GlyRα3.

Embodiment 39. The engineered receptor according to any one of Embodiments 32-38, wherein the engineered receptor comprises an amino acid sequence having at least 90% identity to any one of SEQ ID NO:63, 66, 70 and 71.

Embodiment 40. The engineered receptor according to any one of Embodiments 1-17, wherein the second wild type Cys-loop LGIC receptor is human GABA-A receptor ρ1 subunit (GABA-A ρ1).

Embodiment 41. The engineered receptor according to Embodiment 40, wherein the ion pore domain comprises an amino acid sequence having at least 85% identity according to amino acids 281-479 of SEQ ID NO:10.

Embodiment 42. The engineered receptor according to Embodiment 40 or 41, wherein the Cys-loop domain of the ligand binding domain comprises an amino acid sequence having at least 80% identity according to amino acids 198-212 of SEQ ID NO:10.

Embodiment 43. The engineered receptor according to any one of Embodiments 40-42, wherein the β1-2 loop domain of the ligand binding domain comprises an amino acid sequence having at least 80% identity according to amino acids 112-117 of SEQ ID NO:10.

Embodiment 44. The engineered receptor according to any one of Embodiments 40-43, wherein the engineered receptor comprises an amino acid sequence having at least 90% identity to SEQ ID NO:64 or 67.

Embodiment 45. The engineered receptor according to any one of Embodiments 1-44, wherein the pre-M1 linker of the ligand binding domain comprises one or more mutations.

Embodiment 46. The engineered receptor according to Embodiment 45, wherein the ligand binding domain is derived from human α7-nAChR, and the one or more mutations in the pre-M1 linker are at one or more positions corresponding to T225, M226, and/or T230 of human α7-nAChR.

Embodiment 47. The engineered receptor according to Embodiments 46, wherein the ligand binding domain is derived from human α7-nAChR, and the one or more mutations comprises a mutation corresponding to the T225I of human α7-nAChR.

Embodiment 48. The engineered receptor according to any one of Embodiments 45-47, wherein the one or more mutations increases surface expression of the engineered receptor.

Embodiment 49. The engineered receptor according to Embodiment 48, wherein the surface expression is measured by α-bungarotoxin (a-BTX) assay.

Embodiment 50. The engineered receptor according to any one of Embodiments 3-49, wherein the ligand binding domain comprises an amino acid substitution at one or more residues comprising W77, Y94, R101, W108, Y115, T128, N129, V130, L131, Q139, Y140, L141, Y151, S170, W171, S172, Y173, S188, Y190, Y210, C212, C213, E215, Y217, or any combination thereof, of human α7-nAChR.

Embodiment 51. The engineered receptor according to any one of Embodiments 3-50, wherein the ligand binding domain comprises two amino acid substitutions at a pair of residues comprising R101 and L131, Y115 and Y210, or R101 and Y210, of human α7-nAChR.

Embodiment 50. The engineered receptor according to any one of Embodiments 3-49, wherein the ligand binding domain comprises a sequence having at least 85%, at least 90%, at least 95%, at least 96%, at least 97%, at least 98%, at least 99%, at least 99.5%, or 100%, identity to amino acids 23-220 of SEQ ID NO.4.

Embodiment 51. The engineered receptor according to any one of Embodiments 3-50, wherein the ligand binding domain comprises an amino acid substitution at one or more residues comprising W77, Y94, R101, W108, Y115, T128, N129, V130, L131, Q139, Y140, L141, Y151, S170, W171, S172, Y173, S188, Y190, Y210, C212, C213, E215, Y217, or any combination thereof, of human α7-nAChR.

Embodiment 52. The engineered receptor according to any one of Embodiments 3-51, wherein the ligand binding domain comprises two amino acid substitutions at a pair of residues comprising R101 and L131, Y115 and Y210, or R101 and Y210, of human α7-nAChR.

Embodiment 53. The engineered receptor according to any one of Embodiments 3-51, wherein the ligand binding domain comprises the amino acid substitutions corresponding to L131N, W77F, and S172D of SEQ ID NO:4.

Embodiment 54. The engineered receptor according to any one of Embodiments 3-51, wherein the ligand binding domain comprises the amino acid substitutions corresponding to Q139W and S172D of SEQ ID NO:4.

Embodiment 55. The engineered receptor according to any one of Embodiments 3-51, wherein the ligand binding domain comprises one or more amino acid substitutions listed in Table 12.

Embodiment 56. The engineered receptor according to any one of Embodiments 3-55, wherein the ligand binding domain comprises the amino acid substitutions corresponding to R101W, Y115E, and Y210W of human α7-nAChR.

Embodiment 57. The engineered receptor of any one of Embodiments 1-56, wherein the engineered receptor comprises a sequence having at least 85%, at least 90%, at least 95%, at least 96%, at least 97%, at least 98%, or at least 99%, identity to SEQ ID NO:65 and comprises the amino acid substitutions corresponding to R101W, Y115E, Y210W, and T225I of SEQ ID NO:65.

Embodiment 58. The engineered receptor of any one of Embodiments 1-57, wherein the potency of the engineered receptor to a native ligand of the first wild type Cys-loop LGIC receptor is lower than the potency of the first wild type Cys-loop LGIC receptor to the native ligand.

Embodiment 59. The engineered receptor of Embodiment 58, wherein the potency of the engineered receptor to the native ligand is at least 2-fold lower than the potency of the first wild type Cys-loop LGIC receptor to the native ligand.

Embodiment 60. The engineered receptor of any one of Embodiments 1-59, wherein the potency of the engineered receptor to a non-native ligand is about the same as the potency of the first wild type Cys-loop LGIC receptor to the non-native ligand.

Embodiment 61. The engineered receptor of any one of Embodiments 1-59, wherein the potency of the engineered receptor to a non-native ligand is higher than the potency of the first wild type Cys-loop LGIC receptor to the non-native ligand.

Embodiment 62. The engineered receptor of Embodiment 61, wherein the potency of the engineered receptor to the non-native ligand is at least 2-fold higher than the potency of the first wild type Cys-loop LGIC receptor to the non-native ligand.

Embodiment 63. The engineered receptor of any one of Embodiments 58-62, wherein determining the potency comprises determining the EC50.

Embodiment 64. The engineered receptor of any one of Embodiments 1-63, wherein the efficacy of the engineered receptor in the presence of a non-native ligand is higher than the efficacy of the first wild type Cys-loop LGIC receptor in presence of the non-native ligand.

Embodiment 65. The engineered receptor of Embodiment 64, wherein the efficacy of the engineered receptor in the presence of a non-native ligand is at least 2-fold higher than the efficacy the first wild type Cys-loop LGIC receptor in presence of the non-native ligand.

Embodiment 66. The engineered receptor of Embodiment 64 or 65, wherein determining the efficacy comprises determining the amount of current passed through the engineered receptor in vitro in the presence of the non-native ligand.

Embodiment 67. The engineered receptor of any one of Embodiments 60-66, wherein the non-native ligand is selected from the group consisting of AZD-0328, TC-6987, ABT-126, APN-1125, TC-5619, and Facinicline/RG3487.

Embodiment 68. The engineered receptor of Embodiment 67, wherein the non-native ligand is selected from the group consisting of ABT-126, RG3487, and APN-1125.

Embodiment 69. The engineered receptor of Embodiment 68, wherein the non-native ligand is TC-5619.

Embodiment 70. A polynucleotide, comprising a nucleic acid encoding the engineered receptor of any one of Embodiments 1-69.

Embodiment 71. The polynucleotide of Embodiment 70, wherein the polynucleotide comprises a promoter operably linked to the nucleic acid encoding the engineered receptor.

Embodiment 72. The polynucleotide of Embodiment 71, wherein the promoter is a regulatable promoter.

Embodiment 73. The polynucleotide of Embodiment 72, wherein the regulatable promoter is active in an excitable cell.

Embodiment 74. The polynucleotide of Embodiment 73, wherein the excitable cell is a neuron or a myocyte.

Embodiment 75. The polynucleotide of Embodiment 74, wherein the excitable cell is a neuron.

Embodiment 76. A vector comprising the polynucleotide of any one of Embodiments 70-75.

Embodiment 77. The vector of Embodiment 76, wherein the vector is a plasmid, or a viral vector.

Embodiment 78. The vector of Embodiment 77, wherein the vector is a viral vector selected from the group consisting of an adenoviral vector, a retroviral vector, an adeno-associated viral (AAV) vector, and a herpes simplex-1 viral vector (HSV-1).

Embodiment 79. The vector of Embodiment 78, wherein the viral vector is an AVV vector, and wherein the AAV vector is AAV5 or a variant thereof, AAV6 or a variant thereof or AAV9 or a variant thereof.

Embodiment 80. A composition comprising the engineered receptor of any one of Embodiments 1-69, the polynucleotide of any one of Embodiments 70-75, or the vector of any one of Embodiments 76-79.

Embodiment 81. A pharmaceutical composition comprising the engineered receptor of any one of Embodiments 1-69, the polynucleotide of any one of Embodiments 70-75, or the vector of any one of Embodiments 76-79; and a pharmaceutically acceptable carrier.

Embodiment 82. A method of producing an engineered receptor in a neuron, comprising contacting the neuron with the polynucleotide of any one of Embodiments 70-75, the vector of any one of Embodiments 76-79, the composition of Embodiment 80, or the pharmaceutical composition of Embodiment 81.

Embodiment 83. The method of Embodiment 82 or the polynucleotide of Embodiment 75, wherein the neuron is a neuron of the peripheral nervous system.

Embodiment 84. The method of Embodiment 82 or 83, or the polynucleotide of Embodiment 75, wherein the neuron is a neuron of the central nervous system.

Embodiment 85. The method of any one of Embodiments 82-84 or the polynucleotide of Embodiment 75, wherein the neuron is a nociceptive neuron.

Embodiment 86. The method of any one of Embodiments 82-85 or the polynucleotide of Embodiment 75, wherein the neuron is a non-nociceptive neuron.

Embodiment 87. The method of any one of Embodiments 82-86 or the polynucleotide of Embodiment 75, wherein the neuron is a dorsal root ganglion (DRG) neuron, a trigeminal ganglion (TG) neuron, a motor neuron, an excitatory neuron, an inhibitory neuron, or a sensory neuron.

Embodiment 88. The method of any one of Embodiments 82-87 or the polynucleotide of Embodiment 75, wherein the neuron is an A6 afferent fiber, a C fiber or an Aβ afferent fiber.

Embodiment 89. The method of Embodiment 88 or the polynucleotide of Embodiment 75, wherein the neuron is Aβ afferent fiber.

Embodiment 90. The method of Embodiment 89 or the polynucleotide of Embodiment 75, wherein Aβ afferent fiber is an injured Aβ afferent fiber.

Embodiment 91. The method of Embodiment 89 or the polynucleotide of Embodiment 75, wherein Aβ afferent fiber is an uninjured Aβ afferent fiber.

Embodiment 92. The method of any one of Embodiments 82-91 or the polynucleotide of Embodiment 75, wherein the neuron expresses neurofilament 200 (NF200), piezo 2, and TLR-5.

Embodiment 93. The method of any one of Embodiments 82-92 or the polynucleotide of Embodiment 75, wherein the neuron does not express TrpV1, prostatic acid phosphatase, NaV1.1.

Embodiment 94. The method of any one of Embodiments 82-93, wherein the contacting step is performed in vitro, ex vivo, or in vivo.

Embodiment 95. The method of Embodiment 94, wherein the contacting step is performed in vivo in a subject.

Embodiment 96. The method of Embodiment 95, wherein the contacting step comprises administering the polynucleotide, the vector, the composition, or the pharmaceutical composition to the subject.

Embodiment 97. The method of Embodiment 94, wherein the contacting step is performed in vitro or ex vivo.

Embodiment 98. The method of Embodiment 97, wherein the contacting step comprises lipofection, nanoparticle delivery, particle bombardment, electroporation, sonication, or microinjection.

Embodiment 99. The method of any one of Embodiments 82-98, wherein the engineered receptor is capable of localizing to the cell surface of the neuron.

Embodiment 100. A method of inhibiting the activity of a neuron, comprising (a) contacting the neuron with the engineered receptor of any one of Embodiments 1-69, the polynucleotide of any one of Embodiments 70-75, the vector of any one of Embodiments 76-79, the composition of Embodiment 80, or the pharmaceutical composition of Embodiment 81, and (b) contacting the neuron with a non-native ligand of the engineered receptor.

Embodiment 101. The method of Embodiment 100, wherein the neuron is a neuron of the peripheral nervous system.

Embodiment 102. The method of Embodiment 100, wherein the neuron is a neuron of the central nervous system.

Embodiment 103. The method of any of the Embodiments 100-102, wherein the neuron is a nociceptive neuron.

Embodiment 104. The method of any of the Embodiments 100-102, wherein the neuron is a non-nociceptive neuron.

Embodiment 105. The method of any one of Embodiments 100-104, wherein the neuron is a dorsal root ganglion (DRG) neuron, a trigeminal ganglion (TG) neuron, a motor neuron, an excitatory neuron, an inhibitory neuron, or a sensory neuron.

Embodiment 106. The method of any one of Embodiments 100-105, wherein the neuron is an A6 afferent fiber, a C fiber or an Aβ afferent fiber.

Embodiment 107. The method of Embodiment 106, wherein the neuron is Aβ afferent fiber.

Embodiment 108. The method of Embodiment 107, wherein Aβ afferent fiber is an injured Aβ afferent fiber.

Embodiment 109. The method of Embodiment 107, wherein Aβ afferent fiber is an uninjured Aβ afferent fiber.

Embodiment 110. The method of any one of Embodiments 100-109, wherein the neuron expresses neurofilament 200 (NF200), piezo 2, and TLR-5.

Embodiment 111. The method of any one of Embodiments 100-110, wherein the neuron does not express TrpV1, prostatic acid phosphatase, NaV1.1.

Embodiment 112. The method of any one of Embodiments 100-111, wherein the contacting step (a) is performed in vitro, ex vivo, or in vivo.

Embodiment 113. The method of any one of Embodiments 100-112, wherein the contacting step (b) is performed in vitro, ex vivo, or in vivo.

Embodiment 114. The method of any one of Embodiments 100-113, wherein the contacting steps (a) and/or (b) are performed in vivo in a subject.

Embodiment 115. The method of Embodiment 114, wherein the contacting step (a) comprises administering the engineered receptor, the polynucleotide, the vector, or the pharmaceutical composition to the subject; and/or the contacting step (b) comprises administering the non-native ligand to the subject.

Embodiment 116. The method of any one of Embodiments 100-115, wherein the contacting step (a) and/or (b) comprises lipofection, nanoparticle delivery, particle bombardment, electroporation, sonication, or microinjection.

Embodiment 117. The method of any one of Embodiments 100-116, wherein the engineered receptor is capable of localizing to the cell surface of the neuron.

Embodiment 118. A method of treating and/or delaying the onset of a neurological disorder in a subject, in need thereof, comprising:
(a) administering to the subject, a therapeutically effective amount of the engineered receptor of any one of Embodiments 1-69, the polynucleotide of any one of Embodiments 70-75, the vector of any one of Embodiments 76-79, the composition of Embodiment 80, or the pharmaceutical composition of Embodiment 81, and
(b) administering to the subject a non-native ligand of the engineered receptor.

Embodiment 119. The method of Embodiment 118, wherein the subject is administered the non-native ligand after step (a).

Embodiment 120. The method of Embodiment 118, wherein the subject is administered the non-native ligand concurrently with step (a).

Embodiment 121. The method of any one of Embodiments 118-120, wherein the neurological disorder is a seizure disorder, a movement disorder, an eating disorder, a spinal cord injury, neurogenic bladder, allodynia, a spasticity disorder, pruritus, Alzheimer's disease, Parkinson's disease, post-traumatic stress disorder (PTSD), gastroesophageal reflux disease (GERD), addiction, anxiety, depression, memory loss, dementia, sleep apnea, stroke, narcolepsy, urinary incontinence, essential tremor, trigeminal neuralgia, burning mouth syndrome, or atrial fibrillation.

Embodiment 122. The method of Embodiment 121, wherein the neurological disorder is allodynia.

Embodiment 123. The method of any one of Embodiments 118-122, wherein the non-native ligand is selected from the group consisting of AZD-0328, ABT-126, TC6987, APN-1125, TC-5619, and Facinicline/RG3487.

Embodiment 124. The method of any one of Embodiments 118-123, wherein the non-native ligand is administered orally, subcutaneously, topically, or intravenously.

Embodiment 125. The method of Embodiment 124, wherein the non-native ligand is administered orally.

Embodiment 126. The method of any one of Embodiments 118-125, wherein the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna.

Embodiment 127. The method of any one of Embodiments 118-126, wherein the engineered receptor, the polynucleotide, the vector, the composition, or the pharmaceutical composition is administered by transforaminal injection or intrathecally.

Embodiment 128. The method of any one of Embodiments 118-127, wherein the subject suffers from trigeminal neuralgia, and wherein the engineered receptor, the polynucleotide, the vector, the composition, or the pharmaceutical composition is administered to the trigeminal ganglion (TG) of the subject.

Embodiment 129. The method of any one of Embodiments 118-127, wherein the subject suffers from neuropathic pain, and wherein the engineered receptor, the polynucleotide, the vector, the composition, or the pharmaceutical composition is administered to the dorsal root ganglion (DRG) of the subject.

Embodiment 130. The method of any one of Embodiments 118-129, wherein the subject is a human.

Embodiment 131. The method of any one of Embodiments 118-130, wherein the therapeutically effectively amount diminishes the severity of a sign and/or or a symptom of the neurological disorder.

Embodiment 132. The method of any one of Embodiments 118-131, wherein the therapeutically effectively amount delays the onset of a sign and/or or a symptom of the neurological disorder.

Embodiment 133. The method of any one of Embodiments 118-132, wherein the therapeutically effectively amount eliminates a sign and/or or a symptom of the neurological disorder.

Embodiment 134. The method of any one of Embodiments 131-133, wherein the sign of the neurological disorder is nerve damage, nerve atrophy, and/or seizure.

Embodiment 135. The method of Embodiment 134, wherein the nerve damage is peripheral nerve damage.

Embodiment 136. The method of any one of Embodiments 131-135, wherein the symptom of the neurological disorder is pain.

Embodiment 137. A method of treating and/or delaying the onset of pain in a subject, in need thereof, comprising:
(a) administering to the subject, a therapeutically effective amount of the engineered receptor of any one of Embodiments 1-69, the polynucleotide of any one of Embodiments 70-75, the vector of any one of Embodiments 76-79, the composition of Embodiment 80, or the pharmaceutical composition of Embodiment 81, and
(b) administering to the subject a non-native ligand of the engineered receptor.

Embodiment 138. The method of Embodiment 137, wherein the subject is administered the non-native ligand after step (a).

Embodiment 139. The method of Embodiment 137, wherein the subject is administered the non-native ligand concurrently with step (a).

Embodiment 140. The method of any one of Embodiments 137-139, wherein the non-native ligand is selected from the group consisting of AZD-0328, ABT-126, TC6987, APN-1125, TC-5619, and Facinicline/RG3487.

Embodiment 141. The method of any one of Embodiments 137-140, wherein the non-native ligand is administered orally, subcutaneously, topically, or intravenously.

Embodiment 142. The method of Embodiment 141, wherein the non-native ligand is administered orally.

Embodiment 143. The method of any one of Embodiments 137-142, wherein the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna.

Embodiment 144. The method of any one of Embodiments 137-143, wherein the engineered receptor, the polynucleotide, the vector, the composition, or the pharmaceutical composition is administered by transforaminal injection or intrathecally.

Embodiment 145. The method of any one of Embodiments 137-144, wherein the subject suffers from trigeminal neuralgia, and wherein the engineered receptor, the polynucleotide, the vector, the composition, or the pharmaceutical composition is administered to the trigeminal ganglion (TG) of the subject.

Embodiment 146. The method of any one of Embodiments 137-145, wherein the subject suffers from neuropathic pain, and wherein the engineered receptor, the polynucleotide, the vector, the composition, or the pharmaceutical composition is administered to the dorsal root ganglion (DRG) of the subject.

Embodiment 147. The method of any one of Embodiments 137-146, wherein the subject is a human.

Embodiment 148. The method of any one of Embodiments 137-147, wherein the pain is neuropathic pain.

Embodiment 149. The method of any one of Embodiments 137-148, wherein the pain is associated with, caused by, or resulting from chemotherapy.

Embodiment 150. The method of any one of Embodiments 137-149, wherein the pain is associated with, caused by, or resulting from trauma.

Embodiment 151. The method of any one of Embodiments 137-150, wherein the subject suffers from allodynia.

Embodiment 152. The method of any one of Embodiments 137-151, wherein the pain manifests after a medical procedure.

Embodiment 153. The method of any one of Embodiments 137-152, wherein the pain is associated with, is caused by, or resulting from childbirth or Caesarean section.

Embodiment 154. The method of any one of Embodiments 137-153, wherein the pain is associated with, is caused by, or resulting from migraine.

Embodiment 155. The method of any one of Embodiments 137-154, wherein the therapeutically effectively amount diminishes pain in the subject transiently, diminishes pain in the subject permanently, prevents the onset of pain in the subject, and/or eliminates pain in the subject.

Embodiment 156. The method of any one of Embodiments 137-155, wherein steps (a) and (b) are performed before the manifestation of pain in the subject.

The preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of the present disclosure is embodied by the appended claims.

What is claimed is:

1. An engineered receptor, wherein the engineered receptor is a chimeric ligand gated ion channel (LGIC) receptor and comprises
(a) a ligand binding domain derived from a first wild type Cys-loop LGIC receptor, and
(b) an ion pore domain derived from a second wild type Cys-loop LGIC receptor.

2. The engineered receptor according to claim 1, wherein the first wild type Cys-loop LGIC receptor comprises a nicotinic acetylcholine receptor family receptor.

3. The engineered receptor according to claim 1 or 2, wherein the first wild type Cys-loop LGIC receptor is human α7 nicotinic acetylcholine receptor (α7-nAChR, SEQ ID NO.4).

4. The engineered receptor according to any one of claims 1-3, wherein the second wild type Cys-loop LGIC receptor is a chloride permeable Cys-loop ligand gated ion channel receptor.

5. The engineered receptor according to claim 4, wherein the second wild type Cys-loop LGIC receptor is a glycine receptor or a GABA-A receptor.

6. The engineered receptor according to claim 5, wherein the second wild type Cys-loop LGIC receptor comprises a Glycine receptor α1, a Glycine receptor α2, a Glycine receptor α3, a GABA-A receptor ρ1, a GABA-A receptor ρ2, or a GABA-A receptor ρ³.

7. The engineered receptor according to any one of claims 1-6, wherein the second wild type Cys-loop LGIC receptor is not a Glycine receptor α1.

8. The engineered receptor according to any one of claims 1-7, wherein part or all of Cys-loop domain of the ligand binding domain is derived from the second wild type Cys-loop LGIC receptor.

9. The engineered receptor according to any one of claims 1-8, wherein part or all of β1-2 loop domain of the ligand binding domain is derived from the second wild type Cys-loop LGIC receptor.

10. The engineered receptor according to any one of claims 1-9, wherein the engineered receptor comprises a pre-M1 linker derived from the first wild type Cys-loop LGIC receptor or the second wild type Cys-loop LGIC receptor.

11. The engineered receptor according to any one of claims 1-9, wherein the engineered receptor comprises a pre-M1 linker comprising a N-terminal segment derived from the first wild type Cys-loop LGIC receptor and a C-terminal segment derived from the second wild type Cys-loop LGIC receptor.

12. The engineered receptor according to claim 10 or 11, wherein the pre-M1 linker comprises or consist of a sequence having at least 70% identity to any one of SEQ ID NOS: 72-91.

13. The engineered receptor according to any one of claims 1-12, wherein part or all of M2-M3 linker of the ion pore domain is derived from the first wild type Cys-loop LGIC receptor.

14. The engineered receptor according to any one of claims 1-13, wherein the M2-M3 linker of the ion pore domain comprises one or more mutations.

15. The engineered receptor according to any one of claims 1-14, wherein the M2-M3 linker of the ion pore domain comprises an amino acid sequence having at least 70% identity according to amino acids 283-295 of SEQ ID NO:4.

16. The engineered receptor according to any one of claims 1-15, wherein the engineered receptor forms homomeric ion channels when expressed on cell surface.

17. The engineered receptor according to claim 16, wherein more than 50% of the engineered receptor expressed on cell surface form homomeric ion channels.

18. The engineered receptor according to any one of claims 1-17, wherein the second wild type Cys-loop LGIC receptor is a human Glycine receptor α1 subunit (GlyRα1).

19. The engineered receptor according to claim 18, wherein the ion pore domain comprises an amino acid sequence having at least 85% identity according to amino acids 248-457 of SEQ ID NO:2.

20. The engineered receptor according to claim 18 or 19, wherein the Cys-loop domain of the ligand binding domain comprises an amino acid sequence having at least 80% identity according to amino acids 166-180 of SEQ ID NO:2.

21. The engineered receptor according to any one of claims 18-20, wherein the β1-2 loop domain of the ligand binding domain comprises an amino acid sequence having at least 80% identity according to amino acids 80-85 of SEQ ID NO:2.

22. The engineered receptor according to any one of claims 18-21, wherein the ion pore domain comprises no amino acid between the amino acid positions corresponding to K353 and E362 of SEQ ID NO:2.

23. The engineered receptor according to any one of claims 18-21, wherein the ion pore domain comprises an amino acid sequence having less than 50% sequence identity to SEQ ID NO: 96 between the amino acid positions corresponding to K353 and E362 of SEQ ID NO:2.

24. The engineered receptor according to any one of claims 18-23, wherein the ion pore domain comprises one or more mutations in a region corresponding to the nuclear localization signal (NLS)/ER retention signal (ERRS) sequence of the human GlyRα1.

25. The engineered receptor according to any one of claims 18-24, wherein the engineered receptor comprises an amino acid sequence having at least 90% identity to SEQ ID NO:16 or 33.

26. The engineered receptor according to any one of claims 1-17, wherein the second wild type Cys-loop LGIC receptor is a human Glycine receptor α2 subunit (GlyRα2).

27. The engineered receptor according to claim 26, wherein the ion pore domain comprises an amino acid sequence having at least 85% identity according to amino acids 254-452 of SEQ ID NO:59.

28. The engineered receptor according to claim 26 or 27, wherein the Cys-loop domain of the ligand binding domain comprises an amino acid sequence having at least 80% identity according to amino acids 172-186 of SEQ ID NO:59.

29. The engineered receptor according to any one of claims 26-28, wherein the β1-2 loop domain of the ligand binding domain comprises an amino acid sequence having at least 80% identity according to amino acids 86-91 of SEQ ID NO:59.

30. The engineered receptor according to any one of claims 26-29, wherein the ion pore domain comprises one or more mutations in a region corresponding to the NLS/ERRS sequence of the human GlyRα2.

31. The engineered receptor according to any one of claims 26-30, wherein the engineered receptor comprises an amino acid sequence having at least 90% identity to SEQ ID NO:62 or 65.

32. The engineered receptor according to any one of claims 1-17, wherein the second wild type Cys-loop LGIC receptor is the human Glycine receptor α3 subunit (GlyRα3).

33. The engineered receptor according to claim 32, wherein the ion pore domain comprises an amino acid sequence having at least 85% identity according to amino acids 253-464 of SEQ ID NO:61 or amino acids 253-449 of SEQ ID NO: 69.

34. The engineered receptor according to claim 32 or 33, wherein the Cys-loop domain of the ligand binding domain comprises an amino acid sequence having at least 80% identity according to amino acids 171-185 of SEQ ID NO:61 or 69.

35. The engineered receptor according to any one of claims 32-34, wherein the β1-2 loop domain of the ligand binding domain comprises an amino acid sequence having at least 80% identity according to amino acids 85-90 of SEQ ID NO:61 or 69.

36. The engineered receptor according to any one of claims 32-35, wherein the ion pore domain comprises no amino acid residue between the amino acid positions corresponding to K357 and D358 of SEQ ID NO:69.

37. The engineered receptor according to any one of claims 32-35, wherein the ion pore domain comprises an amino acid sequence having less than 50% sequence identity to SEQ ID NO: 95 between the amino acid positions corresponding to K357 and D358 of SEQ ID NO:69.

38. The engineered receptor according to any one of claims 32-37, wherein the ion pore domain comprises one or more mutations in a region corresponding to the NLS/ERRS sequence of the human GlyRα3.

39. The engineered receptor according to any one of claims 32-38, wherein the engineered receptor comprises an amino acid sequence having at least 90% identity to any one of SEQ ID NO:63, 66, 70 and 71.

40. The engineered receptor according to any one of claims 1-17, wherein the second wild type Cys-loop LGIC receptor is human GABA-A receptor ρ1 subunit (GABA-A ρ1).

41. The engineered receptor according to claim 40, wherein the ion pore domain comprises an amino acid sequence having at least 85% identity according to amino acids 281-479 of SEQ ID NO:10.

42. The engineered receptor according to claim 40 or 41, wherein the Cys-loop domain of the ligand binding domain comprises an amino acid sequence having at least 80% identity according to amino acids 198-212 of SEQ ID NO:10.

43. The engineered receptor according to any one of claims 40-42, wherein the R1-2 loop domain of the ligand binding domain comprises an amino acid sequence having at least 80% identity according to amino acids 112-117 of SEQ ID NO:10.

44. The engineered receptor according to any one of claims 40-43, wherein the engineered receptor comprises an amino acid sequence having at least 90% identity to SEQ ID NO:64 or 67.

45. The engineered receptor according to any one of claims 1-44, wherein the pre-M1 linker of the ligand binding domain comprises one or more mutations.

46. The engineered receptor according to claim 45, wherein the ligand binding domain is derived from human α7-nAChR, and the one or more mutations in the pre-M1 linker are at one or more positions corresponding to T225, M226, and/or T230 of human α7-nAChR.

47. The engineered receptor according to claim 46, wherein the ligand binding domain is derived from human α7-nAChR, and the one or more mutations comprises a mutation corresponding to the T225I of human α7-nAChR.

48. The engineered receptor according to any one of claims 45-47, wherein the one or more mutations increases surface expression of the engineered receptor.

49. The engineered receptor according to claim 48, wherein the surface expression is measured by α-bungarotoxin (a-BTX) assay.

50. The engineered receptor according to any one of claims 3-49, wherein the ligand binding domain comprises a sequence having at least 85%, at least 90%, at least 95%, at least 96%, at least 97%, at least 98%, at least 99%, at least 99.5%, or 100%, identity to amino acids 23-220 of SEQ ID NO:4.

51. The engineered receptor according to any one of claims 3-50, wherein the ligand binding domain comprises an amino acid substitution at one or more residues comprising W77, Y94, R101, W108, Y115, T128, N129, V130, L131, Q139, Y140, L141, Y151, S170, W171, S172, Y173, S188, Y190, Y210, C212, C213, E215, Y217, or any combination thereof, of human α7-nAChR.

52. The engineered receptor according to any one of claims 3-51, wherein the ligand binding domain comprises two amino acid substitutions at a pair of residues comprising R101 and L131, Y115 and Y210, or R101 and Y210, of human α7-nAChR.

53. The engineered receptor according to any one of claims 3-51, wherein the ligand binding domain comprises the amino acid substitutions corresponding to L131N, W77F, and S172D of SEQ ID NO:4.

54. The engineered receptor according to any one of claims 3-51, wherein the ligand binding domain comprises the amino acid substitutions corresponding to Q139W and S172D of SEQ ID NO:4.

55. The engineered receptor according to any one of claims 3-51, wherein the ligand binding domain comprises one or more amino acid substitutions listed in Table 12.

56. The engineered receptor according to any one of claims 3-55, wherein the ligand binding domain comprises the amino acid substitutions corresponding to R101W, Y115E, and Y210W of human α7-nAChR.

57. The engineered receptor of any one of claims 1-56, wherein the engineered receptor comprises a sequence having at least 85%, at least 90%, at least 95%, at least 96%, at least 97%, at least 98%, or at least 99%, identity to SEQ ID NO:65 and comprises the amino acid substitutions corresponding to R101W, Y115E, Y210W, and T225I of SEQ ID NO:65.

58. The engineered receptor of any one of claims 1-57, wherein the potency of the engineered receptor to a native ligand of the first wild type Cys-loop LGIC receptor is lower than the potency of the first wild type Cys-loop LGIC receptor to the native ligand.

59. The engineered receptor of claim 58, wherein the potency of the engineered receptor to the native ligand is at least 2-fold lower than the potency of the first wild type Cys-loop LGIC receptor to the native ligand.

60. The engineered receptor of any one of claims 1-59, wherein the potency of the engineered receptor to a non-native ligand is about the same as the potency of the first wild type Cys-loop LGIC receptor to the non-native ligand.

61. The engineered receptor of any one of claims 1-59, wherein the potency of the engineered receptor to a non-native ligand is higher than the potency of the first wild type Cys-loop LGIC receptor to the non-native ligand.

62. The engineered receptor of claim 61, wherein the potency of the engineered receptor to the non-native ligand is at least 2-fold higher than the potency of the first wild type Cys-loop LGIC receptor to the non-native ligand.

63. The engineered receptor of any one of claims 58-62, wherein determining the potency comprises determining the EC50.

64. The engineered receptor of any one of claims 1-63, wherein the efficacy of the engineered receptor in the presence of a non-native ligand is higher than the efficacy of the first wild type Cys-loop LGIC receptor in presence of the non-native ligand.

65. The engineered receptor of claim 64, wherein the efficacy of the engineered receptor in the presence of a non-native ligand is at least 2-fold higher than the efficacy the first wild type Cys-loop LGIC receptor in presence of the non-native ligand.

66. The engineered receptor of claim 64 or 65, wherein determining the efficacy comprises determining the amount of current passed through the engineered receptor in vitro in the presence of the non-native ligand.

67. The engineered receptor of any one of claims 60-66, wherein the non-native ligand is selected from the group consisting of AZD-0328, TC-6987, ABT-126, APN-1125, TC-5619, and Facinicline/RG3487.

68. The engineered receptor of claim 67, wherein the non-native ligand is selected from the group consisting of ABT-126, RG3487, and APN-1125.

69. The engineered receptor of claim 68, wherein the non-native ligand is TC-5619.

70. A polynucleotide, comprising a nucleic acid encoding the engineered receptor of any one of claims 1-69.

71. The polynucleotide of claim 70, wherein the polynucleotide comprises a promoter operably linked to the nucleic acid encoding the engineered receptor.

72. The polynucleotide of claim 71, wherein the promoter is a regulatable promoter.

73. The polynucleotide of claim 72, wherein the regulatable promoter is active in an excitable cell.

74. The polynucleotide of claim 73, wherein the excitable cell is a neuron or a myocyte.

75. The polynucleotide of claim 74, wherein the excitable cell is a neuron.

76. A vector comprising the polynucleotide of any one of claims 70-75.

77. The vector of claim 76, wherein the vector is a plasmid, or a viral vector.

78. The vector of claim 77, wherein the vector is a viral vector selected from the group consisting of an adenoviral vector, a retroviral vector, an adeno-associated viral (AAV) vector, and a herpes simplex-1 viral vector (HSV-1).

79. The vector of claim 78, wherein the viral vector is an AVV vector, and wherein the AAV vector is AAV5 or a variant thereof, AAV6 or a variant thereof or AAV9 or a variant thereof.

80. A composition comprising the engineered receptor of any one of claims 1-69, the polynucleotide of any one of claims 70-75, or the vector of any one of claims 76-79.

81. A pharmaceutical composition comprising the engineered receptor of any one of claims 1-69, the polynucleotide of any one of claims 70-75, or the vector of any one of claims 76-79; and a pharmaceutically acceptable carrier.

82. A method of producing an engineered receptor in a neuron, comprising contacting the neuron with the polynucleotide of any one of claims 70-75, the vector of any one of claims 76-79, the composition of claim 80, or the pharmaceutical composition of claim 81.

83. The method of claim 82 or the polynucleotide of claim 75, wherein the neuron is a neuron of the peripheral nervous system.

84. The method of claim 82 or 83, or the polynucleotide of claim 75, wherein the neuron is a neuron of the central nervous system.

85. The method of any one of claims 82-84 or the polynucleotide of claim 75, wherein the neuron is a nociceptive neuron.

86. The method of any one of claims 82-85 or the polynucleotide of claim 75, wherein the neuron is a non-nociceptive neuron.

87. The method of any one of claims 82-86 or the polynucleotide of claim 75, wherein the neuron is a dorsal root ganglion (DRG) neuron, a trigeminal ganglion (TG) neuron, a motor neuron, an excitatory neuron, an inhibitory neuron, or a sensory neuron.

88. The method of any one of claims 82-87 or the polynucleotide of claim 75, wherein the neuron is an A6 afferent fiber, a C fiber or an Aβ afferent fiber.

89. The method of claim 88 or the polynucleotide of claim 75, wherein the neuron is Aβ afferent fiber.

90. The method of claim 89 or the polynucleotide of claim 75, wherein Aβ afferent fiber is an injured Aβ afferent fiber.

91. The method of claim 89 or the polynucleotide of claim 75, wherein Aβ afferent fiber is an uninjured Aβ afferent fiber.

92. The method of any one of claims 82-91 or the polynucleotide of claim 75, wherein the neuron expresses neurofilament 200 (NF200), piezo 2, and TLR-5.

93. The method of any one of claims 82-92 or the polynucleotide of claim 75, wherein the neuron does not express TrpV1, prostatic acid phosphatase, NaV1.1.

94. The method of any one of claims 82-93, wherein the contacting step is performed in vitro, ex vivo, or in vivo.

95. The method of claim 94, wherein the contacting step is performed in vivo in a subject.

96. The method of claim 95, wherein the contacting step comprises administering the polynucleotide, the vector, the composition, or the pharmaceutical composition to the subject.

97. The method of claim 94, wherein the contacting step is performed in vitro or ex vivo.

98. The method of claim 97, wherein the contacting step comprises lipofection, nanoparticle delivery, particle bombardment, electroporation, sonication, or microinjection.

99. The method of any one of claims 82-98, wherein the engineered receptor is capable of localizing to the cell surface of the neuron.

100. A method of inhibiting the activity of a neuron, comprising (a) contacting the neuron with the engineered receptor of any one of claims 1-69, the polynucleotide of any one of claims 70-75, the vector of any one of claims 76-79, the composition of claim 80, or the pharmaceutical composition of claim 81, and (b) contacting the neuron with a non-native ligand of the engineered receptor.

101. The method of claim 100, wherein the neuron is a neuron of the peripheral nervous system.

102. The method of claim 100, wherein the neuron is a neuron of the central nervous system.

103. The method of any of the claims 100-102, wherein the neuron is a nociceptive neuron.

104. The method of any of the claims 100-102, wherein the neuron is a non-nociceptive neuron.

105. The method of any one of claims 100-104, wherein the neuron is a dorsal root ganglion (DRG) neuron, a trigeminal ganglion (TG) neuron, a motor neuron, an excitatory neuron, an inhibitory neuron, or a sensory neuron.

106. The method of any one of claims 100-105, wherein the neuron is an A6 afferent fiber, a C fiber or an Aβ afferent fiber.

107. The method of claim 106, wherein the neuron is Aβ afferent fiber.

108. The method of claim 107, wherein Aβ afferent fiber is an injured Aβ afferent fiber.

109. The method of claim 107, wherein Aβ afferent fiber is an uninjured Aβ afferent fiber.

110. The method of any one of claims 100-109, wherein the neuron expresses neurofilament 200 (NF200), piezo 2, and TLR-5.

111. The method of any one of claims 100-110, wherein the neuron does not express TrpV1, prostatic acid phosphatase, NaV1.1.

112. The method of any one of claims 100-111, wherein the contacting step (a) is performed in vitro, ex vivo, or in vivo.

113. The method of any one of claims 100-112, wherein the contacting step (b) is performed in vitro, ex vivo, or in vivo.

114. The method of any one of claims 100-113, wherein the contacting steps (a) and/or (b) are performed in vivo in a subject.

115. The method of claim 114, wherein the contacting step (a) comprises administering the engineered receptor, the polynucleotide, the vector, or the pharmaceutical composition to the subject; and/or the contacting step (b) comprises administering the non-native ligand to the subject.

116. The method of any one of claims 100-115, wherein the contacting step (a) and/or (b) comprises lipofection, nanoparticle delivery, particle bombardment, electroporation, sonication, or microinjection.

117. The method of any one of claims 100-116, wherein the engineered receptor is capable of localizing to the cell surface of the neuron.

118. A method of treating and/or delaying the onset of a neurological disorder in a subject, in need thereof, comprising:
(a) administering to the subject, a therapeutically effective amount of the engineered receptor of any one of claims 1-69, the polynucleotide of any one of claims 70-75, the vector of any one of claims 76-79, the composition of claim 80, or the pharmaceutical composition of claim 81, and
(b) administering to the subject a non-native ligand of the engineered receptor.

119. The method of claim 118, wherein the subject is administered the non-native ligand after step (a).

120. The method of claim 118, wherein the subject is administered the non-native ligand concurrently with step (a).

121. The method of any one of claims 118-120, wherein the neurological disorder is a seizure disorder, a movement disorder, an eating disorder, a spinal cord injury, neurogenic bladder, allodynia, a spasticity disorder, pruritus, Alzheimer's disease, Parkinson's disease, post-traumatic stress disorder (PTSD), gastroesophageal reflux disease (GERD), addiction, anxiety, depression, memory loss, dementia, sleep apnea, stroke, narcolepsy, urinary incontinence, essential tremor, trigeminal neuralgia, burning mouth syndrome, or atrial fibrillation.

122. The method of claim 121, wherein the neurological disorder is allodynia.

123. The method of any one of claims 118-122, wherein the non-native ligand is selected from the group consisting of AZD-0328, ABT-126, TC6987, APN-1125, TC-5619, and Facinicline/RG3487.

124. The method of any one of claims 118-123, wherein the non-native ligand is administered orally, subcutaneously, topically, or intravenously.

125. The method of claim 124, wherein the non-native ligand is administered orally.

126. The method of any one of claims 118-125, wherein the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna.

127. The method of any one of claims 118-126, wherein the engineered receptor, the polynucleotide, the vector, the composition, or the pharmaceutical composition is administered by transforaminal injection or intrathecally.

128. The method of any one of claims 118-127, wherein the subject suffers from trigeminal neuralgia, and wherein the engineered receptor, the polynucleotide, the vector, the composition, or the pharmaceutical composition is administered to the trigeminal ganglion (TG) of the subject.

129. The method of any one of claims 118-127, wherein the subject suffers from neuropathic pain, and wherein the engineered receptor, the polynucleotide, the vector, the composition, or the pharmaceutical composition is administered to the dorsal root ganglion (DRG) of the subject.

130. The method of any one of claims 118-129, wherein the subject is a human.

131. The method of any one of claims 118-130, wherein the therapeutically effectively amount diminishes the severity of a sign and/or or a symptom of the neurological disorder.

132. The method of any one of claims 118-131, wherein the therapeutically effectively amount delays the onset of a sign and/or or a symptom of the neurological disorder.

133. The method of any one of claims 118-132, wherein the therapeutically effectively amount eliminates a sign and/or or a symptom of the neurological disorder.

134. The method of any one of claims 131-133, wherein the sign of the neurological disorder is nerve damage, nerve atrophy, and/or seizure.

135. The method of claim 134, wherein the nerve damage is peripheral nerve damage.

136. The method of any one of claims 131-135, wherein the symptom of the neurological disorder is pain.

137. A method of treating and/or delaying the onset of pain in a subject, in need thereof, comprising:
(a) administering to the subject, a therapeutically effective amount of the engineered receptor of any one of claims 1-69, the polynucleotide of any one of claims 70-75, the vector of any one of claims 76-79, the composition of claim 80, or the pharmaceutical composition of claim 81, and
(b) administering to the subject a non-native ligand of the engineered receptor.

138. The method of claim 137, wherein the subject is administered the non-native ligand after step (a).

139. The method of claim 137, wherein the subject is administered the non-native ligand concurrently with step (a).

140. The method of any one of claims 137-139, wherein the non-native ligand is selected from the group consisting of AZD-0328, ABT-126, TC6987, APN-1125, TC-5619, and Facinicline/RG3487.

141. The method of any one of claims 137-140, wherein the non-native ligand is administered orally, subcutaneously, topically, or intravenously.

142. The method of claim 141, wherein the non-native ligand is administered orally.

143. The method of any one of claims 137-142, wherein the engineered receptor, the polynucleotide, the vector, the composition, or the pharmaceutical composition is administered subcutaneously, orally, intrathecally, topically, intravenously, intraganglioncally, intraneurally, intracranially, intraspinally, or to the cisterna magna.

144. The method of any one of claims 137-143, wherein the engineered receptor, the polynucleotide, the vector, the composition, or the pharmaceutical composition is administered by transforaminal injection or intrathecally.

145. The method of any one of claims 137-144, wherein the subject suffers from trigeminal neuralgia, and wherein the engineered receptor, the polynucleotide, the vector, the composition, or the pharmaceutical composition is administered to the trigeminal ganglion (TG) of the subject.

146. The method of any one of claims 137-145, wherein the subject suffers from neuropathic pain, and wherein the engineered receptor, the polynucleotide, the vector, the composition, or the pharmaceutical composition is administered to the dorsal root ganglion (DRG) of the subject.

147. The method of any one of claims 137-146, wherein the subject is a human.

148. The method of any one of claims 137-147, wherein the pain is neuropathic pain.

149. The method of any one of claims 137-148, wherein the pain is associated with, caused by, or resulting from chemotherapy.

150. The method of any one of claims 137-149, wherein the pain is associated with, caused by, or resulting from trauma.

151. The method of any one of claims 137-150, wherein the subject suffers from allodynia.

152. The method of any one of claims 137-151, wherein the pain manifests after a medical procedure.

153. The method of any one of claims 137-152, wherein the pain is associated with, is caused by, or resulting from childbirth or Caesarean section.

154. The method of any one of claims 137-153, wherein the pain is associated with, is caused by, or resulting from migraine.

155. The method of any one of claims 137-154, wherein the therapeutically effectively amount diminishes pain in the subject transiently, diminishes pain in the subject permanently, prevents the onset of pain in the subject, and/or eliminates pain in the subject.

156. The method of any one of claims 137-155, wherein steps (a) and (b) are performed before the manifestation of pain in the subject.


SEQUENCE LISTING (99 sequences)

SEQ ID NO: 1 - skipped

SEQ ID NO: 2 - protein, 457 aa, Homo sapiens
MYSFNTLRLYLWETIVFFSLAASKEAEAARSAPKPMSPSDFLDKLMGRTSGYDARIRPNFKGPPVNVSCNIFINSFGSIAETTMDYRVNIFLRQQWNDPRLAYNEYPDDSLDLDPSMLDSIWKPDLFFANEKGAHFHEITTDNKLLRISRNGNVLYSIRITLTLACPMDLKNFPMDVQTCIMQLESFGYTMNDLIFEWQEQGAVQVADGLTLPQFILKEEKDLRYCTKHYNTGKFTCIEARFHLERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 3 - DNA, 3356 bp, Homo sapiens
  source 1..3356: mol_type = other DNA
tccttaaaggcgcgcgagccgagcggcgaggtgcctctgtggccgcaggcgcaggcccgggcgacagccgagacgtggagcgcgccggctcgctgcagctccgggactcaacatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaattcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggacgctctactatggcctcaacctgctgatcccctgtgtgctcatctccgccctcgccctgctggtgttcctgcttcctgcagattccggggagaagatttccctggggataacagtcttactctctcttaccgtcttcatgctgctcgtggctgagatcatgcccgcaacatccgattcggtaccattgatagcccagtacttcgccagcaccatgatcatcgtgggcctctcggtggtggtgacagtgatcgtgctgcagtaccaccaccacgaccccgacgggggcaagatgcccaagtggaccagagtcatccttctgaactggtgcgcgtggttcctgcgaatgaagaggcccggggaggacaaggtgcgcccggcctgccagcacaagcagcggcgctgcagcctggccagtgtggagatgagcgccgtggcgccgccgcccgccagcaacgggaacctgctgtacatcggcttccgcggcctggacggcgtgcactgtgtcccgacccccgactctggggtagtgtgtggccgcatggcctgctcccccacgcacgatgagcacctcctgcacggcgggcaaccccccgagggggacccggacttggccaagatcctggaggaggtccgctacattgccaaccgcttccgctgccaggacgaaagcgaggcggtctgcagcgagtggaagttcgccgcctgtgtggtggaccgcctgtgcctcatggccttctcggtcttcaccatcatctgcaccatcggcatcctgatgtcggctcccaacttcgtggaggccgtgtccaaagactttgcgtaaccacgcctggttctgtacatgtggaaaactcacagatgggcaaggcctttggcttggcgagatttgggggtgctaatccaggacagcattacacgccacaactccagtgttcccttctggctgtcagtcgtgttgcttacggtttctttgttactttaggtagtagaatctcagcactttgtttcatattctcagatgggctgatagatatccttggcacatccgtaccatcggtcagcagggccactgagtagtcattttgcccattagcccactgcctggaaagcccttcggagagctccccatggctcctcaccaccgagacagttggttttgcatgtctgcatgaaggtctacctgaaaattcaacatttgctttttgcttgtgtacaaacccagattgaagctaaaataaaccagactcactaaatcctttccaataattgactggtggaaggaaaacaaaaaacaaaaactaaaaacctcttagcttttctgcaattcaactttttatttttatttttatttctatcaaagacggtagagagaaacagcttgatgctgtttctacattaaaaaaaaaaaaaaaagacagactgttggtcttactaaggatgtttttaccagcctgcctgacttctgcaaacctaccctgtcaaggagatcaaagggacgcaggtttctgtttattctgaacaagggccaggccccgcggagtgtctttggtggatcccagataactcctaggtgctgctctcagacactgaggagttgagcaaatctgttctattctgcagaacccacaggacaaataagagttctactagaattaacagcccaaaagaatagctacagctaagtgaagccacttacgtgggctttaaaaaaataatgtgttagctgattcacatgcactggagttaattagtcttagaaatgtgtgcatccatacaaatgcacaacataaagtgaacatattcctaggccctttctgcctgtgtcagggccaggaagtagaggctgggaactcttctggtccccagtatggcaggcgccagggaggggatggtgtggcccatcccttctctggatacctggccagtggcaggcagcagggaggagctggccgaccctcagtgactgacaagccagcaattctgagttctggcctttgggagtctgcctgctccaagccagtccaccccagctgcagccccaaaagctggctcaaagtccttgggtggattcactggagatgggcaacttaaaacaagagaaactttaatttttaaacctaagtgatgatacagctcttcccttagattatcgcccaggctggagtgcagtggcatgatctcagctcactgcaagctccacctcctgggttcatgccattctcctgcctcagcctccccccgagtaactgggaatacaggcgcccgccaccatgcctggctaattttttgtatttttagtagagatagggtttcatcatgttagccaggatggtctcattcttattctttaatgagatcagagggtaattcaccaagaaagacctctcctgttccattgtgtcatccaacaactgctcagagctcaaaattatagaaggcttctgagcccctagagatttttaatttgcttctaatccctgaggtgggaacatcatgagggaagatttgattttcagagttaaataaattgtatgtgcttttccagccaaaaaaaaaaa

SEQ ID NO: 4 - protein, 502 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLASVEMSAVAPPPASNGNLLYIGFRGLDGVHCVPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 5 - DNA, 2343 bp, Homo sapiens
  source 1..2343: mol_type = other DNA
ctctcagctgtcccctcccctttcctcccgcctgaaacatgatccagctgaaggactgattgcaggaaaacttggcagctccccaaccttggtggcccagggagtgtgaggctgcagcctcagaaggtgtgagcagtggccacgagaggcaggctggctgggacatgaggttggcagagggcaggcaagctggcccttggtgggcctcgtcctgagcactcggaggcactcctatgcttggaaagctcgctatgctgctgtgggtccagcaggcgctgctcgccttgctcctccccacactcctggcacagggagaagccaggaggagccgaaacaccaccaggcccgctctgctgaggctgtcggattaccttttgaccaactacaggaagggtgtgcgccccgtgagggactggaggaagccaaccaccgtatccattgacgtcattgtctatgccatcctcaacgtggatgagaagaatcaggtgctgaccacctacatctggtaccggcagtactggactgatgagtttctccagtggaaccctgaggactttgacaacatcaccaagttgtccatccccacggacagcatctgggtcccggacattctcatcaatgagttcgtggatgtggggaagtctccaaatatcccgtacgtgtatattcggcatcaaggcgaagttcagaactacaagccccttcaggtggtgactgcctgtagcctcgacatctacaacttccccttcgatgtccagaactgctcgctgaccttcaccagttggctgcacaccatccaggacatcaacatctctttgtggcgcttgccagaaaaggtgaaatccgacaggagtgtcttcatgaaccagggagagtgggagttgctgggggtgctgccctactttcgggagttcagcatggaaagcagtaactactatgcagaaatgaagttctatgtggtcatccgccggcggcccctcttctatgtggtcagcctgctactgcccagcatcttcctcatggtcatggacatcgtgggcttctacctgccccccaacagtggcgagagggtctctttcaagattacactcctcctgggctactcggtcttcctgatcatcgtttctgacacgctgccggccactgccatcggcactcctctcattggtaaggcccctcctggcagcagagctcagtctggtgagaaacccgccccctcccacctcctgcatgtgtctcttgcctctgccctgggctgcacaggtgtctactttgtggtgtgcatggctctgctggtgataagtttggccgagaccatcttcattgtgcggctggtgcacaagcaagacctgcagcagcccgtgcctgcttggctgcgtcacctggttctggagagaatcgcctggctactttgcctgagggagcagtcaacttcccagaggcccccagccacctcccaagccaccaagactgatgactgctcagccatgggaaaccactgcagccacatgggaggaccccaggacttcgagaagagcccgagggacagatgtagccctcccccaccacctcgggaggcctcgctggcggtgtgtgggctgctgcaggagctgtcctccatccggcaattcctggaaaagcgggatgagatccgagaggtggcccgagactggctgcgcgtgggctccgtgctggacaagctgctattccacatttacctgctagcggtgctggcctacagcatcaccctggttatgctctggtccatctggcagtacgcttgagtgggtacagcccagtggaggagggggtacagtcctggttaggtggggacagaggatttctgcttaggcccctcaggacccagggaatgccagggacattttcaagacacagacaaagtcccgtgccctgtttccaatgccaattcatctcagcaatcacaagccaaggtctgaacccttccaccaaaaactgggtgttcaaggcccttacacccttgtcccacccccagcagctcaccatggctttaaaacatgctgtcttagatcaggagaaactcgggcactccctaagtccactctagttgtggacttttccccattgaccctcacctgaataagggactttggaattctgcttctctttcacaactttgcttttaggttgaaggcaaaaccaactctctactacacaggcctgataactctgtacgaggcttctctaacccctagtgtcttttttttcttcacctcacttgtggcagcttccctgaacactcatcccccatcagatgatgggagtgggaagaataaaatgcagtgaaaccctaaaaaaaaaaaa

SEQ ID NO: 6 - protein, 516 aa, Homo sapiens
MLGKLAMLLWVQQALLALLLPTLLAQGEARRSRNTTRPALLRLSDYLLTNYRKGVRPVRDWRKPTTVSIDVIVYAILNVDEKNQVLTTYIWYRQYWTDEFLQWNPEDFDNITKLSIPTDSIWVPDILINEFVDVGKSPNIPYVYIRHQGEVQNYKPLQVVTACSLDIYNFPFDVQNCSLTFTSWLHTIQDINISLWRLPEKVKSDRSVFMNQGEWELLGVLPYFREFSMESSNYYAEMKFYVVIRRRPLFYVVSLLLPSIFLMVMDIVGFYLPPNSGERVSFKITLLLGYSVFLIIVSDTLPATAIGTPLIGKAPPGSRAQSGEKPAPSHLLHVSLASALGCTGVYFVVCMALLVISLAETIFIVRLVHKQDLQQPVPAWLRHLVLERIAWLLCLREQSTSQRPPATSQATKTDDCSAMGNHCSHMGGPQDFEKSPRDRCSPPPPPREASLAVCGLLQELSSIRQFLEKRDEIREVARDWLRVGSVLDKLLFHIYLLAVLAYSITLVMLWSIWQYA

SEQ ID NO: 7 - DNA, 5723 bp, Homo sapiens
  source 1..5723: mol_type = other DNA
gctccgggccagcgcggcggcggcggcggcggcggcagcagcaggagcagccccggctgcgggtcgcgacggcggcggggcgccccctcccccgtgccggggcgcggcggagggatgtggggccttgcgggaggaaggcttttcggcatcttctcggccccggtgctggtggctgtggtgtgctgcgcccagagtgtgaacgatcccgggaacatgtcctttgtgaaggagacggtggacaagctgttgaaaggctacgacattcgcctaagacccgacttcgggggtcccccggtctgcgtggggatgaacatcgacatcgccagcatcgacatggtttccgaagtcaacatggattataccttaaccatgtattttcaacaatattggagagataaaaggctcgcctattctgggatccctctcaacctcacgcttgacaatcgagtggctgaccagctatgggtgcccgacacatatttcttaaatgacaaaaagtcatttgtgcatggagtgacagtgaaaaaccgcatgatccgtcttcaccctgatgggacagtgctgtatgggctcagaatcaccacgacagcagcatgcatgatggacctcaggagataccccctggacgagcagaactgcactctggaaattgaaagctatggctacaccacggatgacattgagttttactggcgaggcggggacaaggctgttaccggagtggaaaggattgagctcccgcagttctccatcgtggagcaccgtctggtctcgaggaatgttgtcttcgccacaggtgcctatcctcgactgtcactgagctttcggttgaagaggaacattggatacttcattcttcagacttatatgccctctatactgataacgattctgtcgtgggtgtccttctggatcaattatgatgcatctgctgctagagttgccctcgggatcacaactgtgctgacaatgacaaccatcaacacccaccttcgggagaccttgcccaaaatcccctatgtcaaagccattgacatgtaccttatgggctgcttcgtctttgtgttcctggcccttctggagtatgcctttgtcaactacattttctttggaagaggccctcaaaggcagaagaagcttgcagaaaagacagccaaggcaaagaatgaccgttcaaagagcgaaagcaaccgggtggatgctcatggaaatattctgttgacatcgctggaagttcacaatgaaatgaatgaggtctcaggcggcattggcgataccaggaattcagcaatatcctttgacaactcaggaatccagtacaggaaacagagcatgcctcgagaagggcatgggcgattcctgggggacagaagcctcccgcacaagaagacccatctacggaggaggtcttcacagctcaaaattaaaatacctgatctaaccgatgtgaatgccatagacagatggtccaggatcgtgtttccattcactttttctcttttcaacttagtttactggctgtactatgttaactgagtgactgtacttgatttttcaaagacttcatttaacactgagtgaaatattactctgcctgtcaagtttttatacctgtacacacacagacacacaagcagacacacacatatatacatacgcaattgtatatatatgtgaactttctcagcatatatataaaatacacgtgtatatgaggatgtatgtgtatatgtttatacacacaggagtcagtgcccatgtgtatggaagacaaatacacatacatatatacattttgcagctatggacaatttaccacaggatgcatattaaagaaagtcatagtttttttcttttttaattgaaagggacaagtatcatctaaatattatgccttgagaatgagggcgtgaaacacaatatcatccccaaatgtgtcttgtattatcataagttagatgttttagtttaaaaatcagaaagacattcttagttaatctttgaaaactcatacagtggtattgctagtttaaaatgagtcacttacttcatatcctctcgttcagtttagtaagcaaaggcttcttggcttctctggtgatggggtttgttttcatcgggcatacgttttctgcaatggtttagtggctggggtgagccactggcagtgtgcttacctgttgtctgaaacatagatagatcccacgttgatgtctgaacgaccgtcttttgaaaactcatcgggagtgaatggcatctcgttgtaagtactctaatatacagtgtgtagtttgtttctgttgttcacttggagtggatccagcttcactgtcatgtgcgaacacagtgacacgtttggccagtgacatttcaatcactgaaaatgtgctctacatctcgtatggatttctaggcctgatatccaacagaaagcatagacgtctcaggttattcgttactctaaggtaaaaccatctaggatgattttcccccttgcagttatgttatcattcttataacattgtatgttaatagaaaatatatttgcataatatgcatatatatgtatatttaccaagattttgtttcttacgcttgctatcatggcagcatgcgatgtcatattttcctttatgtgatgtaactactttctgttatctagaaattaagattgaagctaaaacacttctactgttcaatttcagaaactaagaatcatcctcatgcctttatttctgtatctgacatatttcataagcacatccaactactcctagactgactaggattctgcaggaacatgacccgtacacaccacgcgtcacccaacgacccatgaccgttctctgaggcaaaggagggcaacctgacagcaaacacagtcactgttggttccttctgatccacagcctcatcagtatttggactttttaaagctcgtagaaacaagacaaggtgcaccggtttcatagacgcaaccttaacttactatttagatgagatctttctaaagaaaaaaaaaagagatgatatattttttgtaaacaatatttctatcacaggcatccataaactgaaatgactacagttgtgcaaacaggtgtcacagtgaagttgagcatttggagaaaaaaataaaaagcaaaatttgcaggaagaactgctaaattaatactttatcccaaaatgccacgtatgcctcaccctctctgttctatccaaaaccaaggaccagagtgctccaatggtaggccccagttgtctcgatgtaggtagaggcaccaccctccccgaggatgcgtgtggtgtggactatccccatgagctgaccactacttgatttttctttggtggccgtaacaacctttaatttgtgggcatctgcaacagttcaaaacccaccatcagataaaacataatccacaaaatctcacgctagaggcaattacctacttttagacccttttccctctttcattcctatctttttcctcctaatcgctgctctctggttttattttcatctggagactagccagggatttctttggctttggcttttctctgaccattttttccatgggtaacaatgggggatcctaaaagttaaacagattgggaaaaaccttgttaagtggccttatcattattatcatgttaaagaaaaaaaatacatttgcaaagaccttatccctttcaatacttcaggtatctttttctgtatccagtaattaaaggtgtgaggtccacatgcagaagagggaccccaaaatgtaaatggatgtggacaaaaacagtcaatggtctatttaagtgtataatttatactattcagaactgtgacattaattctttctgggagaaaagtgatttaaaacttttttgatgcatagttgaggtacccaaatatcaaaggcagagaccccatggggctccaaagagctgcagtctccttcccaaggttttctggatataaatttgcatggtatagtcataatagcttttgagctttttatatgcatttggcaccaagactgggatccacaactttgtagacactgcgatgaagttaacatattagctatactataaatagtgtttgtaactacacacacacacacacacacacacacaaatacatacatatattctgtaaaaacaaaaaaaaactttttaagatccttgggtatgtgtttgctttactccatttcagaagaaaattacttttcttctaacaaaattattttatagcttctccatttttaaaacttgtgagagcattgagaagagaactctgactgtgttaacagaagagagttgaattggagtctctgttgtgttaaaatgacctctcgttactccacagtagttatttgagccagtgactgagtcgcgttgaggaattctgaacccggacctctgacgttgttgggaggtggctgttacccacacccagacctcttgagtaaggacagaaacgttatcattggggatataatgagatttctttcttaacaattgaaagtaataaagagttaattttctcagtagtcctgtctttccaaaatgcgccacaggggctcaagatctacagaagaatcttgttatgacagtttgttattacccatattcagatatccttgagataatggaagagccctgctacataactccctacagagaaagactgatagacttgagtagtccttaaaacaagtgttattcctgttcaccaaccccgggactgtggaggggtttcactgtctataccatgcgacatccatttcccctgaatctcaaacgaactagaaagtatgtcatgataatatttccattagatttggaagctacctgtacatctgcaatattgtgtttttaacgccagcacccagaactttgacatgttcagtcactccctgaaaggcacttctctcttgtccaaacacagcgttgacatttttactgaggagatcatctcaaaggtgatgccaaacgagtctggggctggttttaaggggacaggcacattgcagttgtaggtgttcattttcagcatctagtagataatccattggtgtttgtcccaccattggtgtattctaacaagaatgtgtccaactttgaatctcctggcttgaaagtataaaccatcacttaattcttattttaactctccacctgaaaaccagttcatatttctggccattttatgtaagcaaaactgaacaattggtgaaacattttgttactcttggaattgactctggctgtcagtgtgagccattagagtaacatcgaatcttggggcaaagaactgcccaggtgaattaaatttttccaggacactagctagtgtgccttggattgattacctcttctactgcattgaaaggcgccatgttttcctgaaatactaaattcccaacacctgggtaaacaatgaccttccagagagtggctcccgtatgcctctccctaggaccaaccccatgaacatgttttgtcacgtttgtctcatgtttctacttcacaagtcagtgagtgtgtttaaggtaagtacaggattattctagtaggaataggcgattgctgtcataatcaattctcatgttgatttcattttattgtaaagataaatttaaacccagttttgcttaagcacattgatgtaattttttggtattatttgacatgaaaaaacagcaaaattgagtgatagatac

SEQ ID NO: 8 - protein, 473 aa, Homo sapiens [sequence omitted]

SEQ ID NO: 9 - DNA, 3174 bp, Homo sapiens
  source 1..3174: mol_type = other DNA
taataatggccgtaagcttaaaatagatccagggaggagctcattaacgtgaacatagaaagcagttccgcacctctggccttactcctcttggaaattgctttggtccatttttacttccttttattcgacgcaccagaaaataagacttttaccaacatttttactgcatttgacgatgaactaatttagaccggctaaaataattgttccactgggacacaggaattcaacctcagttcagaaaatccctgacatctgacgtaggaggatttataggtttagtggaaattgctttctcctgctctccagattgcatcctgtgggttgattttttttttgcatgagtaaacatccttctaataatgaacagaccaataatgtcttaagagagaaaaagaacaatcttttcctttttgctgtttctggagagagctgtttgaatttggaaacccatgttggctgtcccaaatatgagatttggcatctttcttttgtggtggggatgggttttggccactgaaagcagaatgcactggcccggaagagaagtccacgagatgtctaagaaaggcaggccccaaagacaaagacgagaagtacatgaagatgcccacaagcaagtcagcccaattctgagacgaagtcctgacatcaccaaatcgcctctgacaaagtcagaacagcttctgaggatagatgaccatgatttcagcatgaggcctggctttggaggccctgccattcctgttggtgtggatgtgcaggtggagagtttggatagcatctcagaggttgacatggactttacgatgaccctctacctgaggcactactggaaggacgagaggctgtcttttccaagcaccaacaacctcagcatgacgtttgacggccggctggtcaagaagatctgggtccctgacatgtttttcgtgcactccaaacgctccttcatccacgacaccaccacagacaacgtcatgttgcgggtccagcctgatgggaaagtgctctatagtctcagggttacagtaactgcaatgtgcaacatggacttcagccgatttcccttggacacacaaacgtgctctcttgaaattgaaagctatgcctatacagaagatgacctcatgctgtactggaaaaagggcaatgactccttaaagacagatgaacggatctcactctcccagttcctcattcaggaattccacaccaccaccaaactggctttctacagcagcacaggctggtacaaccgtctctacattaatttcacgttgcgtcgccacatcttcttcttcttgctccaaacttatttccccgctaccctgatggtcatgctgtcctgggtgtccttctggatcgaccgcagagccgtgcctgccagagtccccttaggtatcacaacggtgctgaccatgtccaccatcatcacgggcgtgaatgcctccatgccgcgcgtctcctacatcaaggccgtggacatctacctctgggtcagctttgtgttcgtgttcctctcggtgctggagtatgcggccgtcaactacctgaccactgtgcaggagaggaaggaacagaagctgcgggagaagcttccctgcaccagcggattacctccgccccgcactgcgatgctggacggcaactacagtgatggggaggtgaatgacctggacaactacatgccagagaatggagagaagcccgacaggatgatggtgcagctgaccctggcctcagagaggagctccccacagaggaaaagtcagagaagcagctatgtgagcatgagaatcgacacccacgccattgataaatactccaggatcatctttccagcagcatacattttattcaatttaatatactggtctattttctcctagatgcttgtaattctacaaatttcacatttccatggcatgcactacagaaataactgtataatgaaaaagtatttaaggatatggttaaaaaaaaatcccaggacccacccatgttttcactatcccttctgcagctttccaaagctacattgacgagacacttactggtttaatttgcacttattaaccatctattgaatacacagcattatattaggtgctgcaggaaatacgacactgtagcgactgatgttagttgttacccagatcccctggaaaagcacactaccagtgttgtgggcacatttagttccacccgttagacccttgatgctattcacatgaataatttattttcctcaagtgtcattacattgttcaggctacgtgaacttggaagcacctacaggccatttgcatgaaattcacatgcacctaaatcctcactttgacagaaactcatgcttcagtttataacctattacctattttgtatgcgactccacctccgcatgtttattttaataaaaggcaatgataacattcacattatttttctttatatgctgtggttcacaggctttaccccttcacaagaaaagctctttagattggcgcaattgcttctgattttggtgaaattttccctggtagggaaactttgaagataagagtacacacatgcattttgtctgttgtgtcatagaggtaactaggctagaaaatttgtgtttaaatgttccctattttatataatcaccacttcatgtttcttcttcttggagcatgtccttgttcaaagagaagtgctttctcagtgatgtgatatcttcactgaggaacttgggtagagaatgatttcttctgcataaacacttcaaggaaatacataatttgggactacttgtaactcattagaatgagaaatactcacatggtttcttaagagaaaaagaacatcggaaagcaaaataaatgggaagatatcactggacatctgcatttatactcgaaataccagcattttctatggaccagaaaactgccatcacctagaccacacagcccagataccaggcagacggatggcccaatggcaactgatgtcagggcatggggtaaaggagagggttctaatctggtgtatcacttaaaaacagttatttatattatatatctgctatatagatcaacctccaccaaacttacccaaacagcatttgttttatttgaaactcactttaataaagtgaattatatacacaaaaaaaaaaaaaaa

SEQ ID NO: 10 - protein, 479 aa, Homo sapiens
MLAVPNMRFGIFLLWWGWVLATESRMHWPGREVHEMSKKGRPQRQRREVHEDAHKQVSPILRRSPDITKSPLTKSEQLLRIDDHDFSMRPGFGGPAIPVGVDVQVESLDSISEVDMDFTMTLYLRHYWKDERLSFPSTNNLSMTFDGRLVKKIWVPDMFFVHSKRSFIHDTTTDNVMLRVQPDGKVLYSLRVTVTAMCNMDFSRFPLDTQTCSLEIESYAYTEDDLMLYWKKGNDSLKTDERISLSQFLIQEFHTTTKLAFYSSTGWYNRLYINFTLRRHIFFFLLQTYFPATLMVMLSWVSFWIDRRAVPARVPLGITTVLTMSTIITGVNASMPRVSYIKAVDIYLWVSFVFVFLSVLEYAAVNYLTTVQERKEQKLREKLPCTSGLPPPRTAMLDGNYSDGEVNDLDNYMPENGEKPDRMMVQLTLASERSSPQRKSQRSSYVSMRIDTHAIDKYSRIIFPAAYILFNLIYWSIFS

SEQ ID NO: 11 - DNA, 4620 bp, Homo sapiens
  source 1..4620: mol_type = other DNA
atgccttattttacaagactcattttgttcttgttttgcttgatggttctcgtggagagcagaaaacccaagaggaagcgatggacagggcaggtggaaatgcccaagccaagtcacttatataagaagaaccttgatgtgaccaagatccggaagggaaagcctcagcagcttctcagagtggacgagcacgacttcagcatgagacccgccttcggaggccctgccatcccggtgggcgtggacgtacaggtggagagcctggacagcatctccgaggtggacatggacttcactatgaccctgtacctgcggcattactggaaggatgagaggctagctttctccagcgccagcaacaagagcatgaccttcgatggccggctggtgaagaagatctgggtccctgatgtcttctttgttcactccaaaagatcgttcactcatgacaccaccactgacaacatcatgctgagggtgttcccagatggacacgtgctgtacagcatgaggattacggtcactgccatgtgcaacatggacttcagccactttcccctggactcccagacctgttctttggagctggagagctatgcctatacagatgaagatctaatgctgtactggaagaatggggatgaatccctaaaaacagatgagaagatctccttgtctcagtttctgattcagaaatttcacacaacttccaggctggccttctacagcagcactggctggtacaaccgtctgtacattaacttcacgttgcgtcgccacatcttcttcttcttgctccaaacatatttccctgccactctgatggtcatgctgtcctgggtgtccttctggatcgaccgcagagctgtgcctgccagagtttcactgggtatcacgacggtgctgaccatgaccaccatcatcacgggcgtgaatgcctccatgccgcgcgtctcctacgtcaaggccgtggacatctacctctgggtcagctttgtgttcgtgttcctctcggtgctggagtatgcggctgtcaactacctgaccaccgtgcaggagcgcaaggaacggaagctgcgggagaagttcccgtgcatgtgtggaatgcttcattcaaaaaccatgatgctggatggaagctacagtgagtctgaggccaacagcctggctgggtaccccagaagccatatcctgacagaagaagaaaggcaagacaaaatagtggtccacctgggcctgagtggtgaagccaacgctgccagaaagaaggggcttctgaagggccagacgggttttcgtatcttccagaatacccatgccattgacaaatactctaggttgatattccctgcctcctacatatttttcaacttaatttattggtcagtgttttcctaggggctccaaggctgttcctagaagagggcatagacatcgagggggcctggccagtcattgacagacggacttgttgaccacacgcccctcaccaaacaatgcagcagctactggaccaccctgagcagcactcatctctcagagaagcccaggagccttccagctgccctgaccccagaccccgtggggctgctccatgttcatgctgtctcgcgtcacacttcacatctctctgggaccttctgttcttgtgtgtgaactaattcacaagaactcccctcctataaacaaggattcaaatgcctcctagacattcttagaccctcagattgcctaggagtcagttgtggagggaaaaggaaaaactgaagtacaagcttaggggcattctggataggagataggaaattaagaacaaaaaacaccctgtaaacccattgaacttgattaagtgcctacctagaagggaaaagagctgagatcccagacaggaaatgatttggcctgtgggagtatggcaacccacaggattctgcaatattagaggagattattcatttatccatccattcaaccattcattaaatattgaggtcccactctgtgccaggtacggtagaaatgagaaaatagtctctttccttagagatcttctctaagttgtgctcattctacatatggcatgatgtacttctgctctttcccctccttcaaccccgcttaccccacagacccattctgtttgctgttgcttttactcttaggcatataaggcagggtccaggagaggccaggtccagaactttcaactatcctcctccagtgaagttacacagatagcactaattttgcccagcaatgacatgtagcaatgtgcatggagtattgccagccagagaaacttacccaggctcaccacggtgtacagtttttttattggggtcggtcatggatttatagctgattgcccacatggctgactttagtcttttgcccctccagaggttaagctggtaccttgaggtccagggcacctatcataaatcacattgttggcatagactatctggtgtggcccaaggccccagtagacaaagacccttctatctcacaggacattccaagggcatagaggttgcttcctagtaaaggttagtcctttactacacagcaactcttgtcaattccctattgttaatgttaattgcggtgttactattatatattcaggcaataaacattttaaagtatctctgcactaggtacattggggtccaaattaagaataagagtagtcgaagctctcagagaaggaactagtaaaggaaataccacacaaatatctataatataagattaagagtgtttagtgcctcagtaatagttcagtgtgagatgggaattccaagggaggaaaaacagtcccaccagggagacagacctcatggagaaagcagccctgagatgccccttgaaggtcaggattttaagaagaagaaatggagcagaatgcatcctagatctgggctgtctcatatggtagccactagccacatgtctatttaaatttaaattagtaaaaattaaaaatattagccaggtgtggtggtgtgtgcctggggttctagctactcaggaggctgaggcgggaggattgcttgagcccaggaattccaggctgccgtgagctgtgatcaagccactgcagtccagcctgagtgacagagcgagaccctatctctaaaataataattattattatttaaattagttgaagttaaataaaattgaaaactcagttccttggttacaccatccacactcaagtgggcttagtaagcacagttgctagtagctactgcactgggctgggcagcctaaagaccaggaagctctaccagccaagagaccagcttgagcggacgcatggggagaatgggaaggtttgccattgcagagaacagggtttgtgggcaagtcattcattttggcaggatcctaggctgtgataagaggaggtgagcgactagctttttccacaacactagggatcaaggcaggtccccaccatgccggtgcttatgaggacagcttccccactatggagttgctggacccaagactactagatttcccagtctcacagaccaatgaagaacaaaaatgcataaatatgggtaattgggtggtacagggcatctggacttgtttagaaacctttttttcttttctgagacagagtttcactcttgttgctcaggctggagtgcaatggcttgatcttggctcaccacaacctctgcctcccgggttcaagcaattctcctgcctcagccctggagtagctgggattacaggcatgcgccaccatgcctggctaacttttgtattttttagtagagatggggtttctccatgttagtcagcctggtcttgaactcctgacctcaggtgatccgcctgcctcagcctcccaaagtgctgtgattacaggcgtgagccaccacacccggccagaaaccttttatctatagagactctcccaggccacagtgctgctcgaagaatgataattctattgtcattaaatatatcatattgctttgcttgtaagtcttatttaattccaacccttctctgcttctgactgcattgttttcatgcattatggctcatataacatagttgcctgttcacaaattaaaacaaatatttatttttgctagcttctatgttaaactggagttaattctatagcagacatatttagaatcataaacagtgttttatcatgcagggccatgatacaatgacaccattgtatttagcattgccttattacagtatactagtctgacactacttgagaaaagtaggaaatttaaagaggtaaagtggagacaacattggtaggagtagaaaacaggaactaaggaaatagtgagaacatgggatgattcaccctggaggcaggaagacagagaagcaaacacaattgttatcaggaatgcatgaatgatactttacttcactgaatctaagacatggtgaaatgtaagatgcacccatattttatgtactgagaaaaaatactaacaatcaaattctggaatgccaatgattataatatgaatcctgatttcaccaatatgaaactgtgaaaaaatgttaatgttacaataaaagaaatgggttttatacaaaaaaaaaaaaaaaaaaaaa

SEQ ID NO: 12 - protein, 465 aa, Homo sapiens
MPYFTRLILFLFCLMVLVESRKPKRKRWTGQVEMPKPSHLYKKNLDVTKIRKGKPQQLLRVDEHDFSMRPAFGGPAIPVGVDVQVESLDSISEVDMDFTMTLYLRHYWKDERLAFSSASNKSMTFDGRLVKKIWVPDVFFVHSKRSFTHDTTTDNIMLRVFPDGHVLYSMRITVTAMCNMDFSHFPLDSQTCSLELESYAYTDEDLMLYWKNGDESLKTDEKISLSQFLIQKFHTTSRLAFYSSTGWYNRLYINFTLRRHIFFFLLQTYFPATLMVMLSWVSFWIDRRAVPARVSLGITTVLTMTTIITGVNASMPRVSYVKAVDIYLWVSFVFVFLSVLEYAAVNYLTTVQERKERKLREKFPCMCGMLHSKTMMLDGSYSESEANSLAGYPRSHILTEEERQDKIVVHLGLSGEANAARKKGLLKGQTGFRIFQNTHAIDKYSRLIFPASYIFFNLIYWSVFS

SEQ ID NO: 13 - DNA, 1404 bp, Homo sapiens
  source 1..1404: mol_type = other DNA
atggtcctggctttccagttagtctccttcacctacatctggatcatattgaaaccaaatgtttgtgctgcttctaacatcaagatgacacaccagcggtgctcctcttcaatgaaacaaacctgcaaacaagaaactagaatgaagaaagatgacagtaccaaagcgcggcctcagaaatatgagcaacttctccatatagaggacaacgatttcgcaatgagacctggatttggagggtctccagtgccagtaggtatagatgtccatgttgaaagcattgacagcatttcagagactaacatggactttacaatgactttttatctcaggcattactggaaagacgagaggctctcctttcctagcacagcaaacaaaagcatgacatttgatcatagattgaccagaaagatctgggtgcctgatatcttttttgtccactctaaaagatccttcatccatgatacaactatggagaatatcatgctgcgcgtacaccctgatggaaacgtcctcctaagtctcaggataacggtttcggccatgtgctttatggatttcagcaggtttcctcttgacactcaaaattgttctcttgaactggaaagctatgcctacaatgaggatgacctaatgctatactggaaacacggaaacaagtccttaaatactgaagaacatatgtccctttctcagttcttcattgaagacttcagtgcatctagtggattagctttctatagcagcacaggttggtacaataggcttttcatcaactttgtgctaaggaggcatgttttcttctttgtgctgcaaacctatttcccagccatattgatggtgatgctttcatgggtttcattttggattgaccgaagagctgttcctgcaagagtttccctgggaatcaccacagtgctgaccatgtccacaatcatcactgctgtgagcgcctccatgccccaggtgtcctacctcaaggctgtggatgtgtacctgtgggtcagctccctctttgtgttcctgtcagtcattgagtatgcagctgtgaactacctcaccacagtggaagagcggaaacaattcaagaagacaggaaagatttctaggatgtacaatattgatgcagttcaagctatggcctttgatggttgttaccatgacagcgagattgacatggaccagacttccctctctctaaactcagaagacttcatgagaagaaaatcgatatgcagccccagcaccgattcatctcggataaagagaagaaaatccctaggaggacatgttggtagaatcattctggaaaacaaccatgtcattgacacctattctaggattttattccccattgtgtatattttatttaatttgttttactggggtgtatatgtatga

SEQ ID NO: 14 - protein, 467 aa, Homo sapiens
MVLAFQLVSFTYIWIILKPNVCAASNIKMTHQRCSSSMKQTCKQETRMKKDDSTKARPQKYEQLLHIEDNDFAMRPGFGGSPVPVGIDVHVESIDSISETNMDFTMTFYLRHYWKDERLSFPSTANKSMTFDHRLTRKIWVPDIFFVHSKRSFIHDTTMENIMLRVHPDGNVLLSLRITVSAMCFMDFSRFPLDTQNCSLELESYAYNEDDLMLYWKHGNKSLNTEEHMSLSQFFIEDFSASSGLAFYSSTGWYNRLFINFVLRRHVFFFVLQTYFPAILMVMLSWVSFWIDRRAVPARVSLGITTVLTMSTIITAVSASMPQVSYLKAVDVYLWVSSLFVFLSVIEYAAVNYLTTVEERKQFKKTGKISRMYNIDAVQAMAFDGCYHDSEIDMDQTSLSLNSEDFMRRKSICSPSTDSSRIKRRKSLGGHVGRIILENNHVIDTYSRILFPIVYILFNLFYWGVYV

SEQ ID NO: 15 - DNA, 1320 bp, synthetic construct
  misc_feature 1..1320: note = synthetic sequence of chimeric LGIC
  source 1..1320: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 16 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 17 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 18 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVHLERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 19 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 20 - DNA, 1329 bp, synthetic construct
  misc_feature 1..1329: note = synthetic sequence of chimeric LGIC
  source 1..1329: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtatcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 21 - protein, 442 aa, synthetic construct
  REGION 1..442: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSEIMPATSDSVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 22 - DNA, 1332 bp, synthetic construct
  misc_feature 1..1332: note = synthetic sequence of chimeric LGIC
  source 1..1332: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 23 - protein, 443 aa, synthetic construct
  REGION 1..443: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 24 - DNA, 1323 bp, synthetic construct
  misc_feature 1..1323: note = synthetic sequence of chimeric LGIC
  source 1..1323: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtccgattcggtaccattgattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 25 - protein, 440 aa, synthetic construct
  REGION 1..440: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSDSVPLIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 26 - DNA, 1332 bp, synthetic construct
  misc_feature 1..1332: note = synthetic sequence of chimeric LGIC
  source 1..1332: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatggagcggcagatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 27 - protein, 443 aa, synthetic construct
  REGION 1..443: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMERQMGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 28 - DNA, 1332 bp, synthetic construct
  misc_feature 1..1332: note = synthetic sequence of chimeric LGIC
  source 1..1332: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggacgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 29 - protein, 443 aa, synthetic construct
  REGION 1..443: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 30 - DNA, 1332 bp, synthetic construct
  misc_feature 1..1332: note = synthetic sequence of chimeric LGIC
  source 1..1332: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggacgctctactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 31 - protein, 443 aa, synthetic construct
  REGION 1..443: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 32 - DNA, 1320 bp, synthetic construct
  misc_feature 1..1320: note = synthetic sequence of chimeric LGIC
  source 1..1320: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 33 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 34 - DNA, 1320 bp, synthetic construct
  misc_feature 1..1320: note = synthetic sequence of chimeric LGIC
  source 1..1320: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaattttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 35 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 36 - DNA, 1320 bp, synthetic construct
  misc_feature 1..1320: note = synthetic sequence of chimeric LGIC
  source 1..1320: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagacaaccatggttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 37 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDVDETTMVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 38 - DNA, 1320 bp, synthetic construct
  misc_feature 1..1320: note = synthetic sequence of chimeric LGIC
  source 1..1320: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacattgctgagacaaccatggacttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 39 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDIAETTMDLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 40 - DNA, 1320 bp, synthetic construct
  misc_feature 1..1320: note = synthetic sequence of chimeric LGIC
  source 1..1320: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagacaaccatggttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 41 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDVDETTMVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 42 - DNA, 1317 bp, synthetic construct
  misc_feature 1..1317: note = synthetic sequence of chimeric LGIC
  source 1..1317: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagacaaccatggttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatggagcggcagatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccag

SEQ ID NO: 43 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDVDETTMVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 44 - DNA, 1317 bp, synthetic construct
  misc_feature 1..1317: note = synthetic sequence of chimeric LGIC
  source 1..1317: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacattgctgagacaaccatggacttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatggagcggcagatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccag

SEQ ID NO: 45 - protein, 439 aa, synthetic construct
  REGION 1..439: note = synthetic sequence of chimeric LGIC
MRCSPGGVWLALAASLLHVSLQGEFQRKLYKELVKNYNPLERPVANDSQPLTVYFSLSLLQIMDIAETTMDLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 46 - DNA, 1329 bp, synthetic construct
  misc_feature 1..1329: note = synthetic sequence of chimeric LGIC
  source 1..1329: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagacaaccatggttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccag

SEQ ID NO: 47 - protein, 443 aa, synthetic construct
  REGION 1..443: note = synthetic sequence of chimeric LGIC
MRCSPGGVWLALAASLLHVSLQGEFQRKLYKELVKNYNPLERPVANDSQPLTVYFSLSLLQIMDVDETTMVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 48 - DNA, 1332 bp, synthetic construct
  misc_feature 1..1332: note = synthetic sequence of chimeric LGIC
  source 1..1332: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctgagatcatgcccgcaacatccgattcggtaccattgatagcccaggccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 49 - protein, 443 aa, synthetic construct
  REGION 1..443: note = synthetic sequence of chimeric LGIC
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSEIMPATSDSVPLIAQAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 50 - protein, 451 aa, synthetic construct
  REGION 1..451: note = synthetic sequence of chimeric LGIC
MLLWVQQALLALLLPTLLAQGEARRSRNTTRPALLRLSDYLLTNYRKGVRPVRDWRKPTTVSIDVIVYAILNVDEKNQVLTTYIWYRQYWTDEFLQWNPEDFDNITKLSIPTDSIWVPDILINEFVDVGKSPNIPYVYIRHQGEVQNYKPLQVVTACSLDIYNFPFDVQNCSLTFTSWLHTIQDINISLWRLPEKVKSDRSVFMNQGEWELLGVLPYFREFSMESSNYYAEMKFYVVIRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 51 - protein, 451 aa, synthetic construct
  REGION 1..451: note = synthetic sequence of chimeric LGIC
MLLWVQQALLALLLPTLLAQGEARRSRNTTRPALLRLSDYLLTNYRKGVRPVRDWRKPTTVSIDVIVYAILNVDEKNQVLTTYIWYRQYWTDEFLQWNPEDFDNITKLSIPTDSIWVPDILINEFVDVGKSPNIPYVYIRHQGEVQNYKPLQVVTACSLDIYNFPFDVQNCSLTFTSWLHTIQDINISLWRLPEKVKSDRSVFMNQGEWELLGVLPYFREFSMESSNYYAEMKFYVHLERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 52 - protein, 455 aa, synthetic construct
  REGION 1..455: note = synthetic sequence of chimeric LGIC
MWGLAGGRLFGIFSAPVLVAVVCCAQSVNDPGNMSFVKETVDKLLKGYDIRLRPDFGGPPVCVGMNIDIASIDMVSEVNMDYTLTMYFQQYWRDKRLAYSGIPLNLTLDNRVADQLWVPDTYFLNDKKSFVHGVTVKNRMIRLHPDGTVLYGLRITTTAACMMDLRRYPLDEQNCTLEIESYGYTTDDIEFYWRGGDKAVTGVERIELPQFSIVEHRLVSRNVVFATGAYPRLSLSFRLKRNIGYMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 53 - protein, 31 aa, synthetic construct
  REGION 1..31: note = C-terminal amino acids of GABAR1
IDRLSRIAFPLLFGIFNLVYWATYLNREPQL

SEQ ID NO: 54 - protein, 22 aa, synthetic construct
  REGION 1..22: note = Amino acid residues 260-281 of human CHRNA7
EKISLGITVLLSLTVFMLLVAE

SEQ ID NO: 55 - protein, 23 aa, synthetic construct
  REGION 1..23: note = Synthetic polypeptide sequence
PAKIGLGITVLLSLTTFMSGVAN

SEQ ID NO: 56 - DNA, 1780 bp, Homo sapiens
  source 1..1780: mol_type = other DNA
atgttgtcaagtgtaatggctcccctgtgggcctgcatcctggtggctgcaggaattctagccacagatacacatcatccccaggattctgctctgtatcatctcagcaagcagctattacagaaatatcataaagaagtgagacctgtttacaactggaccaaggccaccacagtctacctggacctgttcgtccatgctatattggatgtggatgcagagaatcaaatattaaagacaagtgtatggtaccaagaggtctggaatgatgaatttttatcctggaactccagcatgtttgatgagattagagagatctccctacctctaagtgccatctgggcccccgatatcatcatcaatgagtttgtggacattgaaagataccctgaccttccctatgtttatgtgaactcatctgggaccattgagaactataagcccatccaggtggtctctgcgtgcagtttagagacatatgcttttccatttgatgtccagaattgcagcctgaccttcaagagcattctgcatacagtggaagacgtagacctggcctttctgaggagcccagaagacattcagcatgacaaaaaggcgtttttgaatgacagtgagtgggaacttctatctgtgtcctccacatacagcatcctgcagagcagcgctggaggatttgcacagattcagtttaatgtggtgatgcgcaggcaccccctggtctatgtcgtgagtctgctgattcctagcatctttctcatgctggtggacctggggagcttctacctgccacccaactgccgagccaggattgtgttcaagaccagtgtgctggtgggctacaccgtcttcagggtcaacatgtccaaccaggtgccacggagtgtagggagcacccctctgattgggcacttcttcaccatctgcatggccttcttggttctcagcttagctaagtccatcgtgttggtcaaattcctccatgatgagcagcgtggtggacaggagcagcccttcttgtgccttcgaggggacaccgatgctgacaggcctagagtggaacccagggcccaacgtgctgtggtaacagagtcctcgctgtatggagagcacctggcccagccaggaaccctgaaggaagtctggtcgcagcttcaatctatcagcaactacctccaaactcaggaccagacagaccaacaggaggcagagtggctggtcctcctgtcccgctttgaccgactgctcttccaaagctaccttttcatgctggggatctacaccatcactctgtgctccctctgggcactgtggggcggcgtgtgaagactgaagtgttcttcagtaattgtgctggcacttaggagagagaggagggggaataatagtgggttaaaaagctttctgggtcgggtgtggtggttcttgcctatagtcccagtgctttgggaggccatagcaggaggattgcttgagcccaggagttcgagaccagccagagcaacatagtgagaccacatctctaccagtaaataaataaataaataaataaataaataaataaataaatagctgggcatagtggctcatgcctgtactctcagctacttgggaggttgaggtgggaggattgcttgagcccaggatttcaaggctgcagtgagccatgattgcaccactgcaccccagcctgggtgacagagcaagaccctgtctcaaaaaaaataaaataaaaggctttctgccttcaaaaaaaaaaaaaaaaaaaaaaaaaaaaaaa

SEQ ID NO: 57 - protein, 441 aa, Homo sapiens
MLSSVMAPLWACILVAAGILATDTHHPQDSALYHLSKQLLQKYHKEVRPVYNWTKATTVYLDLFVHAILDVDAENQILKTSVWYQEVWNDEFLSWNSSMFDEIREISLPLSAIWAPDIIINEFVDIERYPDLPYVYVNSSGTIENYKPIQVVSACSLETYAFPFDVQNCSLTFKSILHTVEDVDLAFLRSPEDIQHDKKAFLNDSEWELLSVSSTYSILQSSAGGFAQIQFNVVMRRHPLVYVVSLLIPSIFLMLVDLGSFYLPPNCRARIVFKTSVLVGYTVFRVNMSNQVPRSVGSTPLIGHFFTICMAFLVLSLAKSIVLVKFLHDEQRGGQEQPFLCLRGDTDADRPRVEPRAQRAVVTESSLYGEHLAQPGTLKEVWSQLQSISNYLQTQDQTDQQEAEWLVLLSRFDRLLFQSYLFMLGIYTITLCSLWALWGGV

SEQ ID NO: 58 - DNA, 1359 bp, Homo sapiens
  source 1..1359: mol_type = other DNA
atgaaccggcagctagtgaacattttgacagccttgtttgcatttttcttagagacaaaccacttcaggacggctttctgcaaagaccatgactccaggtctggaaaacaaccttcacagaccctatctccttcagatttcttggacaagttaatgggaaggacatcaggatatgatgcaagaatcaggccaaattttaaaggtcctccagtaaacgttacttgcaatatttttatcaacagttttggatcagtcacagaaacgaccatggactaccgagtgaatatttttctgagacaacagtggaatgattcacggctggcgtacagtgagtacccagatgactccctggacttggacccatccatgctagactccatttggaaaccagatttgttctttgccaatgagaagggtgccaacttccacgatgtcaccactgacaacaaattgctacggatttcgaaaaatggcaaagtgctctacagtatcagactcaccttgaccttatcctgtcccatggacttgaagaactttccgatggatgtccagacctgtacaatgcagctggagagttttgggtacacgatgaatgacctgatatttgagtggttaagtgatggtccagtgcaagttgctgaaggattgaccctgccccagtttattttgaaagaagagaaggaacttggctactgtacaaagcactacaacactggaaagtttacctgcattgaggtcaagtttcatctggaacgccaaatgggatattatttgatccagatgtacatcccaagcctgcttatagtaattttgtcctgggtttccttttggataaatatggatgcagcccctgccagggtcgcactgggcatcaccacagtcttaacgatgaccacccagagttcaggctccagggcatctctgccaaaggtctcctatgtaaaagcgattgacatctggatggcggtgtgccttctgtttgtgtttgctgccttactggaatacgcagcggtgaacttcgtctccaggcaacacaaggagttcctgcgcctccgaagaagacagaagaggcagaataaggaagaagacgttactcgtgaaagtcgttttaattttagcggttatgggatgggtcactgcctccaagtgaaagatggaacagctgtcaaggccacacctgccaacccactcccacaaccgccaaaagatggagatgctatcaagaagaagtttgtggaccgggcaaaaaggattgacacgatatctcgagctgccttcccattggccttcctcattttcaacatcttttactggatcacatacaagatcattcggcatgaagatgtccacaagaaatag

SEQ ID NO: 59 - protein, 452 aa, Homo sapiens [sequence omitted]

SEQ ID NO: 60 - DNA, 1395 bp, Homo sapiens
  source 1..1395: mol_type = other DNA
atggcccacgtgagacactttcggacattagtttcgggattttacttctgggaagcagcactgttactcagtttggttgccacaaaggaaacagacagtgcaagatctcgaagtgctccaatgtcaccttctgattttctggataaattaatgggcaggacatcaggatatgatgcaagaatcagacccaattttaaaggccctccagttaatgtcacatgcaacatattcatcaacagctttggctctatcgcagagacgaccatggattacagagtgaatatctttcttcgtcagaaatggaatgatccccgcctcgcgtacagtgaatatcctgacgactctttagacctcgacccctccatgttggactccatttggaaacctgatttgttctttgccaatgaaaagggtgccaactttcatgaagtcactacagacaacaaattgctaagaattttcaaaaatggaaatgttctttattcaataagattaacattaacactttcctgtccaatggatctcaagaattttcccatggatgtacaaacatgtataatgcaactggaaagctttgggtacacaatgaatgatctcatttttgaatggcaagatgaggcacccgtacaagtggcagaaggactcactttgccccagtttctgttgaaagaagaaaaagatttacgatactgcactaaacattacaatacaggaaagtttacgtgtatagaagtgcgattccatctggagcgacaaatgggatactatctgatccagatgtacattcccagtctcctgattgttattctatcctgggtttcattctggatcaacatggatgcagcaccggccagggtagctctggggataaccactgtgctaacgatgactacacagagttcaggatcacgagcttccttgccaaaagtttcatatgtcaaagctattgatatttggatggcagtatgcctcctttttgtgttttcagcacttctggagtatgcagctgtaaattttgtatcaagacaacacaaagaacttctgagatttcgacgaaagagaaagaataagacagaagcttttgcactggagaagttttaccgtttctcagatatggatgatgaggtaagggaaagccgattcagcttcacagcctatggaatgggaccatgtctacaagcaaaggatggcatgactccaaagggccccaaccaccctgtccaggtaatgccaaaaagtcctgatgaaatgaggaaggtctttatcgaccgggccaagaagattgataccatctcccgagcctgcttcccattagcttttttgatttttaatattttctactgggttatctataaaattcttaggcatgaggatattcatcagcagcaagattaa

SEQ ID NO: 61 - protein, 464 aa, Homo sapiens
MAHVRHFRTLVSGFYFWEAALLLSLVATKETDSARSRSAPMSPSDFLDKLMGRTSGYDARIRPNFKGPPVNVTCNIFINSFGSIAETTMDYRVNIFLRQKWNDPRLAYSEYPDDSLDLDPSMLDSIWKPDLFFANEKGANFHEVTTDNKLLRIFKNGNVLYSIRLTLTLSCPMDLKNFPMDVQTCIMQLESFGYTMNDLIFEWQDEAPVQVAEGLTLPQFLLKEEKDLRYCTKHYNTGKFTCIEVRFHLERQMGYYLIQMYIPSLLIVILSWVSFWINMDAAPARVALGITTVLTMTTQSSGSRASLPKVSYVKAIDIWMAVCLLFVFSALLEYAAVNFVSRQHKELLRFRRKRKNKTEAFALEKFYRFSDMDDEVRESRFSFTAYGMGPCLQAKDGMTPKGPNHPVQVMPKSPDEMRKVFIDRAKKIDTISRACFPLAFLIFNIFYWVIYKILRHEDIHQQQD

SEQ ID NO: 62 - protein, 428 aa, synthetic construct
  REGION 1..428: note = CHRNA7/GLRA2
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWVSFWINMDAAPARVALGITTVLTMTTQSSGSRASLPKVSYVKAIDIWMAVCLLFVFAALLEYAAVNFVSRQHKEFLRLRRRQKRQNKEEDVTRESRFNFSGYGMGHCLQVKDGTAVKATPANPLPQPPKDGDAIKKKFVDRAKRIDTISRAAFPLAFLIFNIFYWITYKIIRHEDVHKK

SEQ ID NO: 63 - protein, 441 aa, synthetic construct
  REGION 1..441: note = CHRNA7/GLRA3 isoform L
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWVSFWINMDAAPARVALGITTVLTMTTQSSGSRASLPKVSYVKAIDIWMAVCLLFVFSALLEYAAVNFVSRQHKELLRFRRKRKNKTEAFALEKFYRFSDMDDEVRESRFSFTAYGMGPCLQAKDGMTPKGPNHPVQVMPKSPDEMRKVFIDRAKKIDTISRACFPLAFLIFNIFYWVIYKILRHEDIHQQQD

SEQ ID NO: 64 - protein, 428 aa, synthetic construct
  REGION 1..428: note = CHRNA7/GABRR1
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRIFFFLLQTYFPATLMVMLSWVSFWIDRRAVPARVPLGITTVLTMSTIITGVNASMPRVSYIKAVDIYLWVSFVFVFLSVLEYAAVNYLTTVQERKEQKLREKLPCTSGLPPPRTAMLDGNYSDGEVNDLDNYMPENGEKPDRMMVQLTLASERSSPQRKSQRSSYVSMRIDTHAIDKYSRIIFPAAYILFNLIYWSIFS

SEQ ID NO: 65 - protein, 428 aa, synthetic construct
  REGION 1..428: note = CHRNA7 (Cys-loop)/GLRA2
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWVSFWINMDAAPARVALGITTVLTMTTQSSGSRASLPKVSYVKAIDIWMAVCLLFVFAALLEYAAVNFVSRQHKEFLRLRRRQKRQNKEEDVTRESRFNFSGYGMGHCLQVKDGTAVKATPANPLPQPPKDGDAIKKKFVDRAKRIDTISRAAFPLAFLIFNIFYWITYKIIRHEDVHKK

SEQ ID NO: 66 - protein, 441 aa, synthetic construct
  REGION 1..441: note = CHRNA7 (Cys-loop)/GLRA3 isoform L
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWVSFWINMDAAPARVALGITTVLTMTTQSSGSRASLPKVSYVKAIDIWMAVCLLFVFSALLEYAAVNFVSRQHKELLRFRRKRKNKTEAFALEKFYRFSDMDDEVRESRFSFTAYGMGPCLQAKDGMTPKGPNHPVQVMPKSPDEMRKVFIDRAKKIDTISRACFPLAFLIFNIFYWVIYKILRHEDIHQQQD

SEQ ID NO: 67 - protein, 428 aa, synthetic construct
  REGION 1..428: note = CHRNA7 (Cys-loop)/GABRR1
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCNMDFSRFPLDTQTCKLKFGSWSYGGWSLDLQMQEADISGYIPNGEWDLVGIPGKRSERFYECCKEPYPDVTFTVTMRRRIFFFLLQTYFPATLMVMLSWVSFWIDRRAVPARVPLGITTVLTMSTIITGVNASMPRVSYIKAVDIYLWVSFVFVFLSVLEYAAVNYLTTVQERKEQKLREKLPCTSGLPPPRTAMLDGNYSDGEVNDLDNYMPENGEKPDRMMVQLTLASERSSPQRKSQRSSYVSMRIDTHAIDKYSRIIFPAAYILFNLIYWSIFS

SEQ ID NO: 68 - DNA, 1350 bp, Homo sapiens
  source 1..1350: mol_type = other DNA
atggcccacgtgagacactttcggacattagtttcgggattttacttctgggaagcagcactgttactcagtttggttgccacaaaggaaacagacagtgcaagatctcgaagtgctccaatgtcaccttctgattttctggataaattaatgggcaggacatcaggatatgatgcaagaatcagacccaattttaaaggccctccagttaatgtcacatgcaacatattcatcaacagctttggctctatcgcagagacgaccatggattacagagtgaatatctttcttcgtcagaaatggaatgatccccgcctcgcgtacagtgaatatcctgacgactctttagacctcgacccctccatgttggactccatttggaaacctgatttgttctttgccaatgaaaagggtgccaactttcatgaagtcactacagacaacaaattgctaagaattttcaaaaatggaaatgttctttattcaataagattaacattaacactttcctgtccaatggatctcaagaattttcccatggatgtacaaacatgtataatgcaactggaaagctttgggtacacaatgaatgatctcatttttgaatggcaagatgaggcacccgtacaagtggcagaaggactcactttgccccagtttctgttgaaagaagaaaaagatttacgatactgcactaaacattacaatacaggaaagtttacgtgtatagaagtgcgattccatctggagcgacaaatgggatactatctgatccagatgtacattcccagtctcctgattgttattctatcctgggtttcattctggatcaacatggatgcagcaccggccagggtagctctggggataaccactgtgctaacgatgactacacagagttcaggatcacgagcttccttgccaaaagtttcatatgtcaaagctattgatatttggatggcagtatgcctcctttttgtgttttcagcacttctggagtatgcagctgtaaattttgtatcaagacaacacaaagaacttctgagatttcgacgaaagagaaagaataaggatgatgaggtaagggaaagccgattcagcttcacagcctatggaatgggaccatgtctacaagcaaaggatggcatgactccaaagggccccaaccaccctgtccaggtaatgccaaaaagtcctgatgaaatgaggaaggtctttatcgaccgggccaagaagattgataccatctcccgagcctgcttcccattagcttttttgatttttaatattttctactgggttatctataaaattcttaggcatgaggatattcatcagcagcaagattaa

SEQ ID NO: 69 - protein, 449 aa, Homo sapiens
MAHVRHFRTLVSGFYFWEAALLLSLVATKETDSARSRSAPMSPSDFLDKLMGRTSGYDARIRPNFKGPPVNVTCNIFINSFGSIAETTMDYRVNIFLRQKWNDPRLAYSEYPDDSLDLDPSMLDSIWKPDLFFANEKGANFHEVTTDNKLLRIFKNGNVLYSIRLTLTLSCPMDLKNFPMDVQTCIMQLESFGYTMNDLIFEWQDEAPVQVAEGLTLPQFLLKEEKDLRYCTKHYNTGKFTCIEVRFHLERQMGYYLIQMYIPSLLIVILSWVSFWINMDAAPARVALGITTVLTMTTQSSGSRASLPKVSYVKAIDIWMAVCLLFVFSALLEYAAVNFVSRQHKELLRFRRKRKNKDDEVRESRFSFTAYGMGPCLQAKDGMTPKGPNHPVQVMPKSPDEMRKVFIDRAKKIDTISRACFPLAFLIFNIFYWVIYKILRHEDIHQQQD

SEQ ID NO: 70 - protein, 426 aa, synthetic construct
  REGION 1..426: note = CHRNA7/GLRA3 isoform K
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWVSFWINMDAAPARVALGITTVLTMTTQSSGSRASLPKVSYVKAIDIWMAVCLLFVFSALLEYAAVNFVSRQHKELLRFRRKRKNKDDEVRESRFSFTAYGMGPCLQAKDGMTPKGPNHPVQVMPKSPDEMRKVFIDRAKKIDTISRACFPLAFLIFNIFYWVIYKILRHEDIHQQQD

SEQ ID NO: 71 - protein, 426 aa, synthetic construct
  REGION 1..426: note = CHRNA7_(Cys-loop)/GLRA3 isoform K
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWVSFWINMDAAPARVALGITTVLTMTTQSSGSRASLPKVSYVKAIDIWMAVCLLFVFSALLEYAAVNFVSRQHKELLRFRRKRKNKDDEVRESRFSFTAYGMGPCLQAKDGMTPKGPNHPVQVMPKSPDEMRKVFIDRAKKIDTISRACFPLAFLIFNIFYWVIYKILRHEDIHQQQD

SEQ ID NO: 72 - protein, 10 aa, synthetic construct
  REGION 1..10: note = alpha 7-nAchR Pre-M1 linker
VTMRRRTLYY

SEQ ID NO: 73 - protein, 10 aa, synthetic construct
  REGION 1..10: note = GlyR alpha 1, alpha 2 and alpha3 Pre-M1 linker
FHLERQMGYY

SEQ ID NO: 74 - protein, 10 aa, synthetic construct
  REGION 1..10: note = Pre-M1 linker 1
VTMRRRTGYY

SEQ ID NO: 75 - protein, 10 aa, synthetic construct
  REGION 1..10: note = Pre-M1 linker 2
VTMRRRMGYY

SEQ ID NO: 76 - protein, 10 aa, synthetic construct
  REGION 1..10: note = Pre-M1 linker 3
VTMRRQMGYY

SEQ ID NO: 77 - protein, 10 aa, synthetic construct
  REGION 1..10: note = Pre-M1 linker 4
VTMERQMGYY

SEQ ID NO: 78 - protein, 10 aa, synthetic construct
  REGION 1..10: note = Pre-M1 linker 5
VTLERQMGYY

SEQ ID NO: 79 - protein, 10 aa, synthetic construct
  REGION 1..10: note = Pre-M1 linker 6
VHLERQMGYY

SEQ ID NO: 80 - protein, 10 aa, synthetic construct
  REGION 1..10: note = GABA-A rho1, rho2 Pre-M1 linker
FTLRRHIFFF

SEQ ID NO: 81 - protein, 10 aa, synthetic construct
  REGION 1..10: note = GABA-A rho3 pre-M1 linker
FVLRRHVFFF

SEQ ID NO: 82 - protein, 10 aa, synthetic construct
  REGION 1..10: note = Pre-M1 linker 7
VTMRRRTLYF

SEQ ID NO: 83 - protein, 10 aa, synthetic construct
  REGION 1..10: note = Pre-M1 linker 8
VTMRRRTLFF

SEQ ID NO: 84 - protein, 10 aa, synthetic construct
  REGION 1..10: note = Pre-M1 linker 9
VTMRRRTFFF

SEQ ID NO: 85 - protein, 10 aa, synthetic construct
  REGION 1..10: note = Pre-M1 linker 10
VTMRRRIFFF

SEQ ID NO: 86 - protein, 10 aa, synthetic construct
  REGION 1..10: note = Pre-M1 linker 11
VTMRRHIFFF

SEQ ID NO: 87 - protein, 10 aa, synthetic construct
  REGION 1..10: note = Pre-M1 linker 12
VTLRRHIFFF

SEQ ID NO: 88 - protein, 10 aa, synthetic construct
  REGION 1..10: note = Pre-M1 linker 13
VTMRRRVFFF

SEQ ID NO: 89 - protein, 10 aa, synthetic construct
  REGION 1..10: note = Pre-M1 linker 14
VTMRRHVFFF

SEQ ID NO: 90 - protein, 10 aa, synthetic construct
  REGION 1..10: note = Pre-M1 linker 15
VTLRRHVFFF

SEQ ID NO: 91 - protein, 10 aa, synthetic construct
  REGION 1..10: note = Pre-M1 linker 16
VVLRRHVFFF

SEQ ID NO: 92 - protein, 724 aa, synthetic construct
  REGION 1..724: note = AAV5 capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 93 - protein, 736 aa, synthetic construct
  REGION 1..736: note = AAV6 capsid protein
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 94 - protein, 736 aa, synthetic construct
  REGION 1..736: note = AAV9 capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 95 - protein, 15 aa, synthetic construct
  REGION 1..15: note = GLRA3 isoform L AA 358-372
TEAFALEKFYRFSDM

SEQ ID NO: 96 - protein, 8 aa, synthetic construct
  REGION 1..8: note = GLRA1 AA 354-361
SPMLNLFQ

SEQ ID NO: 97 - protein, 6 aa, synthetic construct
  REGION 1..6: note = GLRA1 NLS/ERRS
RRKRRH

SEQ ID NO: 98 - protein, 6 aa, synthetic construct
  REGION 1..6: note = GLRA2 NLS/ERRS
RRRQKR

SEQ ID NO: 99 - protein, 6 aa, synthetic construct
  REGION 1..6: note = GLRA3 NLS/ERRS
RRKRKN